(19) United States
(12) Patent Application Publication
(10) Pub. No.: US 2023/0036370 A1
(43) Pub. Date: Feb. 2, 2023

(54) ENGINEERING CIRCULAR GUIDE RNAS

(71) Applicant: The Regents of the University of California, Oakland, CA (US)

(72) Inventors: Prashant Mali, La Jolla, CA (US); Dhruva Katrekar, La Jolla, CA (US)

(21) Appl. No.: 17/781,686

(22) PCT Filed: Dec. 1, 2020
(86) PCT No.: PCT/US2020/062748

§ 371 Date: Jun. 1, 2022

Related U.S. Application Data
(60) Provisional application No. 62/942,725, filed on Dec. 2, 2019.
(60) Provisional application No. 63/112,492, filed on Nov. 11, 2020.

(51) Int. Cl. C12N 15/113; C12N 9/78; C12N 9/22

(52) U.S. Cl. CPC C12N 15/113; C12N 9/78; C12N 9/22; C12N 2310/11; C12N 2310/532; C12N 2310/531; C12N 2320/51

(57) ABSTRACT

Disclosed herein are engineered guide RNAs, constructs for forming engineered guide RNAs, pharmaceutical compositions thereof, methods of making the engineered guide RNAs, and methods of treating or preventing a diseases and disorders of a subject by administering one or more of the engineered guide RNAs or the constructs for forming the engineered guide RNAs.

### CROSS REFERENCE TO RELATED APPLICATIONS

This application claim priority under 35 U.S.C. §119(e) to U.S. Provisional Application No. 62/942,725, filed Dec. 2, 2019, and U.S. Provisional Application No. 63/112,492, filed Nov. 11, 2020, the disclosures of which are incorporated herein by reference for all purposes.

### STATEMENT OF GOVERNMENT SUPPORT

This invention was made with Government support under Grant Nos. R01GM123313, R01CA222826, and R01HG009285, awarded by the National Institutes of Health. The Government has certain rights in the invention.

### FIELD OF THE INVENTION

The disclosure provides for engineered guide RNAs, pharmaceutical compositions thereof, methods of making the engineered guide RNAs, and methods of treating a subject by administering one or more engineered guide RNAs.

### INCORPORATION BY REFERENCE OF SEQUENCE LISTING

Accompanying this filing is a Sequence Listing entitled, “00015-380WO1_SL25” created on Dec. 1, 2020 and having 374,077 bytes of data, machine formatted on IBM-PC, MS-Windows operating system. The sequence listing is hereby incorporated by reference in its entirety for all purposes.

### BACKGROUND

A significant hurdle in the RNA editing space is guide stability. An adRNA should be present for extended periods of time in order to successfully recruit endogenous ADARs, but single stranded RNAs have a half-life of about 30 minutes or less in mammalian cells. This may be due to their susceptibility to exonucleases that degrade single stranded RNA from the 5′ or 3′ ends.

### SUMMARY

An aspect of the disclosure provides an engineered guide RNA for editing of a nucleotide. In some cases, the engineered guide RNA can comprise a targeting domain. In some cases, the engineered guide RNA can comprise an RNA editing entity recruiting domain. In some cases, the engineered guide RNA may not comprise an RNA editing entity recruiting domain. In some cases, an RNA editing entity recruiting domain may be capable of recruiting an RNA editing entity that performs a chemical transformation on a base of a nucleotide in an RNA sequence thereby generating an edited RNA sequence. In some cases, the engineered guide RNA may be circular. In some cases, the engineered guide RNA may be capable of form a secondary structure comprising a stemloop, a cruciform, a toe hold, a mismatch bulge, more than one of any of these, or any combination thereof. In some cases, the RNA editing entity recruiting domain may comprise at least about 80% sequence homology to: an Alu domain, an APOBEC recruiting domain, or a GluR2 domain. In some cases, the engineered guide RNA may comprise more than one RNA editing entity recruiting domains. In some cases, the engineered guide RNA may comprise a Cas13 recruiting domain. In some cases, the RNA editing entity may be an endogenous enzyme. In some cases, the RNA editing entity may be a recombinant enzyme. In some cases, at least one base of the engineered guide RNA may be a modified base. In some cases, the modified base may comprise a sugar modification. In some cases, the engineered guide RNA may be genetically encodable. In some cases, when the engineered guide RNA is contacted with an RNA editing entity and a target nucleic acid complementary to at least a portion of the targeting domain, the engineered guide RNA: (i) may modify at least one base pair of the target nucleic acid at an efficiency of at least about 4 times greater than a comparable nucleic acid that is not circular; (ii) may retain a half-life at least about 4 times longer than a comparable nucleic acid that is not circular; (iii) may reduce a therapeutically effective amount of the engineered guide RNA dosed to a subject by at least about 4 times as compared to a comparable nucleic acid dosed to a subject that is not circular; or (iv) any combination thereof. Another aspect of the disclosure provides for a vector. In some cases, the vector may comprise the engineered guide RNA. In some cases, the vector may comprise a liposome, a viral vector, a nanoparticle, or any combination thereof. In some cases, the vector may be an AAV vector. In some cases, the vector may comprise DNA. In some cases, the DNA may be double stranded. Another aspect of the disclosure provides for a nucleic acid that may encode for the engineered guide RNA. In some cases, the nucleic acid may be double stranded. Another aspect of the disclosure provides for an isolated cell that may comprise the engineered guide RNA.

Another aspect of the disclosure provides an engineered guide RNA. The engineered guide RNA may comprise a targeting domain. In some cases, the engineered guide RNA can comprise a recruiting domain capable of recruiting an RNA editing entity that performs an editing of a target nucleotide. In some cases, the engineered guide RNA may not comprise a recruiting domain capable of recruiting an RNA editing entity that performs an editing of a target nucleotide. In some cases, the engineered guide RNA may not comprise a 5′ reducing hydroxyl capable of being exposed to a solvent. Another aspect of the disclosure provides for a vector. In some cases, the vector may comprise the engineered guide RNA. In some cases, the vector may comprise a liposome, a viral vector, a nanoparticle, or any combination thereof. In some cases, the vector may be an AAV vector. In some cases, the vector may comprise DNA. In some cases, the DNA may be double stranded. Another aspect of the disclosure provides for a nucleic acid that may encode for the engineered guide RNA. In some cases, the nucleic acid may be double stranded. Another aspect of the disclosure provides for an isolated cell that may comprise the engineered guide RNA.

Another aspect of the disclosure provides an engineered guide RNA. The engineered guide RNA may comprise a targeting domain. In some cases, the engineered guide RNA can comprise a recruiting domain capable of recruiting an RNA editing entity that performs an editing of a target nucleotide. In some cases, the engineered guide RNA may not comprise a recruiting domain capable of recruiting an RNA editing entity that performs an editing of a target nucleotide. In some cases, the engineered guide RNA may comprise a secondary structure that is less susceptible to hydrolytic degradation than an mRNA naturally present in a human cell. Another aspect of the disclosure provides for a vector. In some cases, the vector may comprise the engineered guide RNA. In some cases, the vector may comprise a liposome, a viral vector, a nanoparticle, or any combination thereof. In some cases, the vector may be an AAV vector. In some cases, the vector may comprise DNA. In some cases, the DNA may be double stranded. Another aspect of the disclosure provides for a nucleic acid that may encode for the engineered guide RNA. In some cases, the nucleic acid may be double stranded. Another aspect of the disclosure provides for an isolated cell that may comprise the engineered guide RNA.

Another aspect of the disclosure provides for a method of forming a circular RNA. In some cases, the method may comprise directly or indirectly forming a covalent linkage between more than one end of an engineered guide RNA to form the circular RNA. The engineered guide RNA may comprise a targeting domain. In some cases, the engineered guide RNA can comprise a recruiting domain capable of recruiting an RNA editing entity that performs an editing of a target nucleotide. In some cases, the engineered guide RNA may not comprise a recruiting domain capable of recruiting an RNA editing entity that performs an editing of a target nucleotide. In some cases, the method may employ an enzyme to form the covalent linkage.

Another aspect of the disclosure provides a pharmaceutical composition in unit dose form comprising the engineered guide RNA or the vector. In some cases, the pharmaceutical composition may comprise a pharmaceutically acceptable excipient, diluent, or carrier. In some cases, the pharmaceutical composition may comprise a modified transfer RNA (tRNA). In some cases, the modified tRNA may be an orthogonal tRNA. In some cases, the pharmaceutical composition may comprise an RNA editing entity. In some cases, the RNA editing entity may be a recombinant RNA editing entity. In some cases, the RNA editing entity may be directly or indirectly linked to the engineered guide RNA. In some cases, a linkage between the RNA editing entity and the engineered guide RNA may be a covalent linkage.

Another aspect of the disclosure privides kits comprising engineered guide RNAs disclosed herein, vectors disclosed herein, or pharmaceutical compositions disclosed herein and a container.

Another aspect of the disclosure privides methods of making kits comprising engineered guide RNAs disclosed herein, vectors disclosed herein, or pharmaceutical compositions disclosed herein and packaging into a packaging.

Another aspect of the disclosure provides a method of treating a disease or condition in a subject. In some cases, the method may comprise administering to the subject the engineered guide RNA, the vector, or the pharmaceutical composition. In some cases, the method may comprise administering a modified transfer RNA, an RNA editing entity, or a combination thereof to the subject. In some cases, the modified transfer RNA, the RNA editing entity, or the combination thereof may be co-administered with the engineered guide RNA, the vector, or the pharmaceutical composition. In some cases, the modified transfer RNA, the RNA editing entity, or the combination thereof may be directly or indirectly linked to the engineered guide RNA, the vector, or the pharmaceutical composition. In some cases, a linkage to the engineered guide RNA, the vector, or the pharmaceutical composition may be a covalent linkage. In some cases, the administering may be by intravenous injection, intramuscular injection, an intrathecal injection, an intraorbital injection, a subcutaneous injection, or any combination thereof. In some cases, the method may comprise administering a second therapy to the subject. In some cases, the disease or condition may be selected from the group consisting of: a neurodegenerative disorder, a muscular disorder, a metabolic disorder, an ocular disorder, and any combination thereof. In some cases, the disease or condition may be Alzheimer's disease, muscular dystrophy, retinitis pigmentosa, Parkinson disease, pain, Stargardt macular dystrophy, Charcot-Marie-Tooth disease, or Rett syndrome. In some cases, the subject may be a mammal. In some cases, the mammal may be a human. In some cases, the subject may have been diagnosed with the disease or condition by a diagnostic.

Another aspect of the disclosure provides for a method of making the engineered guide RNA. In some cases, the method may comprise genetically encoding the engineered guide RNA or chemically synthesizing the engineered guide RNA. Another aspect of the disclosure provides for a method of making the pharmaceutical composition. In some cases, the method may comprise formulating the pharmaceutical composition in unit dose form.

Another aspect of the disclosure provides for a method of making the engineered guide RNA. In some cases, the method may comprise directly or indirectly forming a covalent linkage between more than one end of the engineered guide RNA to form a circular RNA. In some cases, the engineered guide RNA may be processed using a cleaving entity. In some cases, the cleaving entity may be a ribozyme. In some cases, the ribozyme may be a RNase P. In some cases, the cleaving entity may be a tRNA. In some cases, the method may further comprise recruiting an enzyme to form the covalent bond between the more than one end of the engineered guide RNA.

Another aspect of the disclosure provides for a method of making the engineered guide RNA. In some cases, the method may comprise ligating more than one end of the engineered guide RNA using a linkage element. In some cases, the linkage element may employ click chemistry to form a circular sequence. In some cases, the linkage element may be an azide-based linkage.

Another aspect of the disclosure provides for a construct for forming a circular guide RNA sequence. In some cases, the construct may comprise: a nucleotide sequence encoding for: (a) a guide RNA sequence for circularization; (b) a ligation sequence; and (c) a ribozyme. In some cases, a guide RNA sequence for circularization can comprise a targeting domain. In some cases, a guide RNA sequence for circularization can comprise an RNA editing entity recruiting domain. In some cases, a guide RNA sequence for circularization may not comprise an RNA editing entity recruiting domain. In some cases, the RNA editing entity recruiting domain may comprise an Alu domain, an APOBEC recruiting domain, a GluR2 domain, a Cas13 recruiting domain, or any combination thereof. In some cases, the RNA editing entity recruiting domain may comprise at least about 80% sequence homology to at least about 400 nucleotides of SEQ ID NO 1418 or SEQ ID NO 1419. In some cases, the RNA editing entity recruiting domain may comprise at least about 80% sequence homology to SEQ ID NO 1418 or SEQ ID NO 1419. In some cases, a 5′ end or a 3′ end of the guide RNA sequence may be flanked by the ligation sequence. In some cases, the 5′ end or the 3′ end of the ligation sequence may be flanked by the ribozyme. In some cases, the nucleotide sequence may encode for at least 2 ribozymes, at least 2 ligation sequences, or a combination thereof.

Another aspect of the disclosure provides for a construct for forming a circular RNA sequence. In some cases, the construct may comprise: a nucleotide sequence encoding for: (a) an RNA sequence for circularization; (b) a ligation sequence; and (c) a tRNA. In some cases, a 5′ end or a 3′ end of the guide RNA sequence may be flanked by the ligation sequence. In some cases, the 5′ end or the 3′ end of the ligation sequence may be flanked by the tRNA. In some cases, the nucleotide sequence may encode for at least 2 ribozymes, at least 2 ligation sequences, or a combination thereof. In some cases, the engineered guide RNA may be a pre-strained circular RNA sequence.

In some aspects, the present disclosure provides for an engineered guide RNA for editing a nucleotide in an RNA sequence, the engineered guide RNA comprising: an RNA editing entity recruiting domain, wherein the RNA editing entity recruiting domain recruits an RNA editing entity that, when associated with the engineered guide RNA, performs a chemical transformation on a base of a nucleotide in the RNA sequence, thereby generating an edited RNA sequence, wherein the engineered guide RNA is circular. In some embodiments, the engineered guide RNA further comprises a targeting domain. In some embodiments, the targeting domain comprises a sequence length from about 20 nucleotides to about 1,000 nucleotides in length. In some embodiments, the targeting domain comprises a sequence length of at least about 100 nucleotides in length. In some embodiments, the chemical transformation on the base results in at least a partial knockdown of the edited RNA sequence. In some embodiments, the partial knockdown comprises a reduced level of a protein or fragment thereof expressed from the edited RNA sequence. In some embodiments, the reduced level is from about 5% to 100%. In some embodiments, the reduced level is from about 60% to 100%. In some embodiments, the chemical transformation results in a sense codon read as a stop codon. In some embodiments, the chemical transformation results in a stop codon read as a sense codon. In some embodiments, the chemical transformation results in a first sense codon read as a second sense codon. In some embodiments, the chemical transformation results in a first stop codon read as a second stop codon. In some embodiments, the engineered guide RNA can be configured to form a secondary structure comprising: a stem-loop, a cruciform, a toe hold, a mismatch bulge, or any combination thereof. In some embodiments, the RNA editing entity recruiting domain comprises at least about 80% sequence homology to at least about 20 nucleic acids of: an Alu domain, an APOBEC recruiting domain, a GluR2 domain, or a Cas13 recruiting domain. In some embodiments, the RNA editing entity recruiting domain comprises at least about 80% sequence homology to at least about 20 nucleic acids of the Alu domain. In some embodiments, the RNA editing entity recruiting domain comprises at least about 80% sequence homology to the Alu domain. In some embodiments, the RNA editing entity recruiting domain comprises at least about 80% sequence homology to at least about 20 nucleic acids of the APOBEC recruiting domain. In some embodiments, the RNA editing entity recruiting domain comprises at least about 80% sequence homology to the APOBEC recruiting domain. In some embodiments, the RNA editing entity recruiting domain comprises the Cas13 recruiting domain that is a Cas13a recruiting domain, a Cas13b recruiting domain, a Cas13c recruiting domain, or a Cas13d recruiting domain. In some embodiments, the RNA editing entity recruiting domain comprises at least about 80% sequence homology to at least about 20 nucleic acids of the Cas13b recruiting domain. In some embodiments, the sequence comprises at least about 80% sequence homology to the Cas13b recruiting domain. In some embodiments, the RNA editing entity is an endogenous enzyme. In some embodiments, the RNA editing entity is a recombinant enzyme. In some embodiments, the engineered guide RNA comprises a modification. In some embodiments, the modification comprises a sugar modification. In some embodiments, a nucleotide of the engineered guide RNA comprises a methyl group, a fluoro group, a methoxyethyl group, an ethyl group, a phosphate group, an amide group, an ester group, or any combination thereof. In some embodiments, the engineered guide RNA comprises a protein coating. In some embodiments, the engineered guide RNA is genetically encodable. In some embodiments, the RNA editing entity is linked to the engineered guide RNA. In some embodiments, a linkage between the engineered guide RNA and the RNA editing entity is a direct or an indirect covalent linkage. In some embodiments, the engineered guide RNA retains a half-life, in an aqueous solution at a physiological pH, that is at least about 4 times longer than a comparable guide RNA that is not circular. In some embodiments, a therapeutically effective amount of the engineered guide RNA dosed to a subject in need thereof is at least about 4 times less than a comparable guide RNA that is not circular on a weight-to-weight basis.

In some aspects, the present disclosure provides for an engineered guide RNA for editing a nucleotide in an RNA sequence, the engineered guide RNA comprising: an RNA editing entity recruiting domain, wherein the RNA editing entity recruiting domain recruits an RNA editing entity that, when associated with the engineered guide RNA, performs a chemical transformation on a base of a nucleotide in the RNA sequence, and wherein the engineered guide RNA does not comprise a 5′ reducing hydroxyl capable of being exposed to a solvent, a 3′ reducing hydroxyl capable of being exposed to a solvent, or both. In some aspects, the present disclosure provides for an engineered guide RNA for editing a nucleotide in an RNA sequence, the engineered guide RNA comprising: an RNA editing entity recruiting domain, wherein the RNA editing entity recruiting domain recruits an RNA editing entity that, when associated with the engineered guide RNA, performs a chemical transformation on a base of a nucleotide in the RNA sequence, thereby generating an edited RNA sequence, wherein the engineered guide RNA comprises a secondary structure that is less susceptible to hydrolytic degradation than an mRNA naturally present in a human cell. In some embodiments, the engineered guide RNA further comprises a targeting domain. In some embodiments, the targeting domain comprises a sequence length from about 20 nucleotides to about 1,000 nucleotides in length. In some embodiments, the targeting domain comprises a sequence length of at least about 100 nucleotides in length. In some embodiments, the chemical transformation on the base results in at least a partial knockdown of the edited RNA sequence. In some embodiments, the at least partial knockdown comprises a reduced level of a protein or fragment thereof expressed from the edited RNA sequence. In some embodiments, the reduced level is from about 5% to 100%. In some embodiments, the reduced level is from about 60% to 100%. In some embodiments, the chemical transformation results in a sense codon read as a stop codon. In some embodiments, the chemical transformation results in a stop codon read as a sense codon. In some embodiments, the chemical transformation results in a first sense codon read as a second sense codon. In some embodiments, the engineered guide RNA is a pre-strained circular RNA sequence. In some embodiments, the engineered guide RNA comprises a reduced entropy as compared to a non-strained circular RNA sequence.

In some aspects, the present disclosure provides for a vector comprising any of the engineered guide RNAs described herein. In some embodiments, the vector comprises a liposome, a viral vector, a nanoparticle, or any combination thereof. In some embodiments, the vector is the viral vector, and wherein the viral vector is an adeno-associated virus (AAV) vector. In some embodiments, the vector comprises DNA. In some embodiments, the DNA is double stranded.

In some aspects, the present disclosure provides for a nucleic acid encoding for any of the engineered guide RNAs described herein. In some embodiments, the nucleic acid is double stranded.

In some aspects, the present disclosure provides for an isolated cell that comprises the any of the engineered guide RNAs described herein, any of the vectors described herein, or any of the nucleic acids described herein.

In some aspects, the present disclosure provides for a method of forming a circular RNA, the method comprising: directly or indirectly forming a covalent linkage between more than one end of a sequence comprising an engineered guide RNA to form the circular RNA, wherein the engineered guide RNA comprises: an RNA editing entity recruiting domain, wherein the RNA editing entity recruiting domain recruits an RNA editing entity that, when associated with the engineered guide RNA, performs a chemical transformation on a base of a nucleotide in an RNA sequence thereby generating an edited RNA sequence. In some embodiments, the method employs an enzyme to form the covalent linkage. In some embodiments, the enzyme is a ligase. In some embodiments, the engineered guide RNA further comprises a targeting domain. In some embodiments, the targeting domain comprises a sequence length from about 20 nucleotides to about 1,000 nucleotides in length. In some embodiments, the targeting domain comprises a sequence length of at least about 100 nucleotides in length. In some embodiments, the chemical transformation on the base results in at least a partial knockdown of the edited RNA sequence. In some embodiments, the at least partial knockdown comprises a reduced level of a protein or fragment thereof expressed from the edited RNA sequence. In some embodiments, the reduced level is from about 5% to 100%. In some embodiments, the reduced level is from about 60% to 100%. In some embodiments, the partial knockdown or reduced level can be determined compared to an otherwise identical unedited RNA sequence as determined in an in vitro assay. In some embodiments, the chemical transformation results in a sense codon read as a stop codon. In some embodiments, the chemical transformation results in a stop codon read as a sense codon. In some embodiments, the chemical transformation results in a first sense codon read as a second sense codon.

In some aspects, the present disclosure provides for a pharmaceutical composition comprising any of the engineered guide RNAs described herein, any of the vectors described herein, or any of the nucleic acids described herein, and a pharmaceutically acceptable: excipient, diluent, or carrier. In some embodiments, the pharmaceutical composition can be in unit dose form. In some embodiments, the composition further comprises an RNA editing entity. In some embodiments, the RNA editing entity is a recombinant RNA editing entity. In some embodiments, the RNA editing entity is directly or indirectly linked to the engineered guide RNA. In some embodiments, a linkage between the RNA editing entity and the engineered guide RNA is a covalent linkage.

In some aspects, the present disclosure provides for a method of treating a subject in need thereof comprising: administering to the subject any of the engineered guide RNAs described herein, any of the vectors described herein, or any of the pharmaceutical compositions described herein. In some embodiments, the method further comprises administering a modified transfer RNA, an RNA editing entity, or a combination thereof to the subject in need thereof. In some embodiments, the modified transfer RNA, the RNA editing entity, or the combination thereof is co-administered with the engineered guide RNA, the vector, or the pharmaceutical composition. In some embodiments, the modified transfer RNA, the RNA editing entity, or the combination thereof are directly or indirectly linked to the engineered guide RNA, the vector, or the pharmaceutical composition. In some embodiments, a linkage to the engineered guide RNA, the vector, or the pharmaceutical composition is a covalent linkage. In some embodiments, the administering is by intravenous injection, intramuscular injection, an intrathecal injection, an intraorbital injection, a subcutaneous injection, or any combination thereof. In some embodiments, the method further comprises administering a second therapy to the subject. In some embodiments, the subject has or is suspected of having a disease or condition selected from the group consisting of: a neurodegenerative disorder, a muscular disorder, a metabolic disorder, an ocular disorder, and any combination thereof. In some embodiments, the disease or condition is Alzheimer's disease, muscular dystrophy, retinitis pigmentosa, Parkinson's disease, pain, Stargardt macular dystrophy, Charcot-Marie-Tooth disease, or Rett syndrome. In some embodiments, the disease or condition is the muscular dystrophy that is Duchenne muscular dystrophy (DMD). In some embodiments, the subject is a mammal. In some embodiments, the mammal is a human. In some embodiments, the subject has been diagnosed with a disease or condition by a diagnostic.

In some aspects, the present disclosure provides for a method of making any of the pharmaceutical compositions described herein, the method comprising formulating the pharmaceutical composition in unit dose form.

In some aspects, the present disclosure provides for a method of making any of the engineered guide RNAs described herein, the method comprising genetically encoding the engineered guide RNA or chemically synthesizing the engineered guide RNA.

In some aspects, the present disclosure provides for a method of making any of the engineered guide RNAs described herein, the method comprising directly or indirectly forming a covalent linkage between more than one end of the engineered guide RNA to form a circular RNA, wherein the engineered guide RNA is processed using a self-cleaving entity. In some embodiments, the self-cleaving entity is a ribozyme. In some embodiments, the ribozyme is a RNase P. In some embodiments, the self-cleaving entity is a tRNA. In some embodiments, the self-cleaving entity is an aptamer or catalytically active fragment thereof. In some embodiments, the method further comprises recruiting an enzyme to form the covalent bond between the more than one end of the engineered guide RNA.

In some aspects, the present disclosure provides for a method of making any of the engineered guide RNAs described herein, the method comprising ligating more than one end of the engineered guide RNA using a linkage element. In some embodiments, the linkage element employs click chemistry to form a circular sequence. In some embodiments, the linkage element is an azide-based linkage.

In some aspects, the present disclosure provides for a construct for forming a circular guide RNA sequence, the construct comprising: a nucleotide sequence encoding for:(a) a guide RNA sequence for circularization; (b) a ligation sequence; and (c) a ribozyme. In some embodiments, the engineered guide RNA can further comprise an RNA editing entity recruiting domain. In some embodiments, the engineered guide RNA may not comprise an RNA editing entity recruiting domain. In some embodiments, the engineered guide RNA further comprises a targeting domain. In some embodiments, the targeting domain comprises a sequence length from about 20 nucleotides to about 1,000 nucleotides in length. In some embodiments, the targeting domain comprises a sequence length of at least about 100 nucleotides in length. In some embodiments, the RNA editing entity recruiting domain comprises an Alu domain, an APOBEC recruiting domain, a GluR2 domain, a Cas13 recruiting domain, or any combination thereof. In some embodiments, the RNA editing entity recruiting domain comprises at least about 80% sequence homology to at least about 400 nucleotides of SEQ ID NO 1418 or SEQ ID NO 1419. In some embodiments, the RNA editing entity recruiting domain comprises at least about 80% sequence homology to SEQ ID NO 1418 or SEQ ID NO 1419. In some embodiments, a 5′ end or a 3′ end of the guide RNA sequence is flanked by the ligation sequence. In some embodiments, the 5′ end or the 3′ end of the ligation sequence is flanked by the ribozyme. In some embodiments, the nucleotide sequence encodes for at least 2 ribozymes, at least 2 ligation sequences, or a combination thereof. In some embodiments, the nucleotide sequence comprises a sequence with at least about 80%, at least 85%, at least 90%, at least 92%, at least 95%, at least 97%, at least 99%, or 100% sequence homology to any one of the polynucleotides in Tables 1-12.

In some aspects, the present disclosure provides for a construct for forming a circular RNA sequence, the construct comprising: a nucleotide sequence encoding for: (a) an RNA sequence for circularization; (b) a ligation sequence; and (c) a tRNA. In some embodiments, a 5′ end or a 3′ end of the guide RNA sequence is flanked by the ligation sequence, and wherein the 5′ end or the 3′ end of the ligation sequence is flanked by the tRNA. In some embodiments, the nucleotide sequence encodes for at least 2 ribozymes, at least 2 ligation sequences, or a combination thereof.

In some aspects, the present disclosure provides for a construct for forming a circular RNA sequence, the construct comprising: a nucleotide sequence encoding for: (a) an RNA sequence for circularization; (b) a ligation sequence; and (c) an aptamer or catalytically active fragment thereof. In some embodiments, a 5′ end or a 3′ end of the guide RNA sequence is flanked by the ligation sequence, and wherein the 5′ end or the 3′ end of the ligation sequence is flanked by the aptamer or the catalytically active fragment thereof. In some embodiments, the nucleotide sequence encodes for at least 2 ribozymes, at least 2 ligation sequences, or a combination thereof.

In some aspects, the present disclosure provides for an engineered polynucleotide comprising: a targeting domain that is at least partially complementary to a target RNA, wherein the engineered polynucleotide comprises a structure of Formula (I):

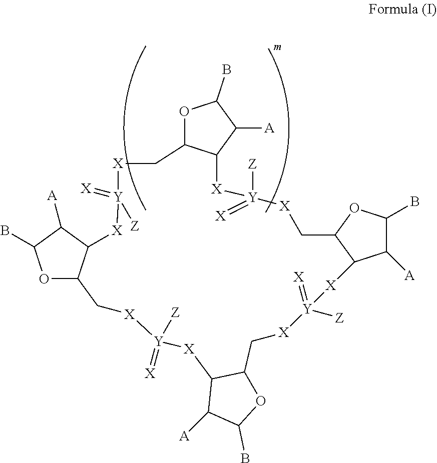

wherein: each X is O; each Y is P; each Z is O, or S; each A is independently H, D, halogen, OM, SM, NRM, or NRR′; each B is independently uracil, thymine, adenine, cytosine, guanine, a salt of any of these, or a derivative of any of these; each M is independently an inorganic or organic cation, H, or D; and each R and R′ is independently H, D, halogen, or C₁-C₆ alkyl; and m is independently an integer from 0-1,000; wherein the targeting domain is configured to at least partially associate with a coding region of the target RNA, and wherein the association of the targeting domain with the coding region of the target RNA facilitates an edit of a base of a nucleotide of the target RNA by an RNA editing entity. In some embodiments, the edit of the base of the nucleotide of the target RNA by the RNA editing entity is determined in an in vitro assay comprising: (i) directly or indirectly introducing the target RNA into a primary cell line, (ii) directly or indirectly introducing the engineered polynucleotide into the primary cell line, and (iii) sequencing the target RNA. In some embodiments, each unit m is independently in the (D)- or (L)- configuration. In some embodiments, Formula (I) is according to Formula (II):

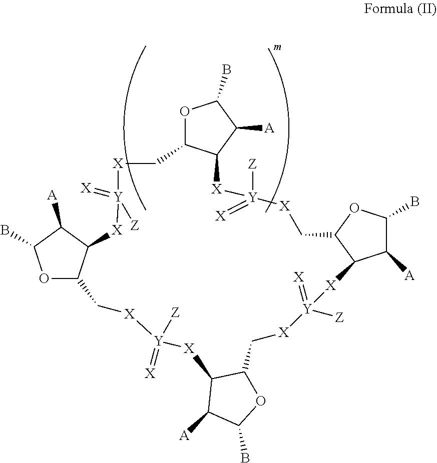

In some embodiments, each Z is O and each R is H. In some embodiments, m is an independent integer from about 30-600. In some embodiments, at least partially complementary comprises the targeting domain that comprises a polynucleotide sequence with at least about 80% sequence homology to a reverse complement to the target RNA. In some embodiments, the RNA editing entity comprises an ADAR protein, an APOBEC protein, or both. In some embodiments, the RNA editing entity comprises ADAR and wherein ADAR comprises ADAR1 or ADAR2. In some embodiments, the edit of a base converts a sense codon into a stop codon. In some embodiments, the edit of a base converts a stop codon into a sense codon. In some embodiments, the edit of a base converts a first sense codon into a second sense codon. In some embodiments, the edit of a base, coverts a sense codon specifying a first amino acid into a second sense codon specifying a second amino acid. In some embodiments, the first amino acid is a protease cleavage site.

In some aspects, the present disclosure provides for an engineered polynucleotide comprising a targeting domain that is at least partially complementary to a target RNA; wherein the engineered polynucleotide comprises a structure of Formula (III):

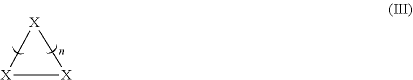

wherein in the engineered polynucleotide, each X is a nucleotide comprising a base that is independently uracil, thymine, adenine, cytosine, guanine, a salt of any of these, or a derivative of any of these; n is independently an integer from 0-1,000; and wherein each nucleotide is connected to two adjacent nucleotides by, independently for each connection, a phosphoester, phosphothioester, phosphothioate, or phosphoramidite linkage; and wherein the targeting domain is configured to at least partially associate with a coding region of the target RNA, wherein the association of the targeting domain with the coding region of the target RNA facilitates an edit of a base of a nucleotide of the target RNA by an RNA editing entity. In some embodiments, the edit of the base of the nucleotide of the target RNA by the RNA editing entity is determined in an in vitro assay comprising: (i) directly or indirectly introducing the target RNA into a primary cell line, (ii) directly or indirectly introducing the engineered polynucleotide into the primary cell line, and (iii) sequencing the target RNA. In some embodiments, the RNA editing entity comprises an ADAR protein, an APOBEC protein, or both. In some embodiments, the RNA editing entity comprises ADAR and wherein ADAR comprises ADAR1 or ADAR2. In some embodiments, the primary cell line comprises a neuron cell, a photoreceptor cell, a retinal pigment epithelium cell, a glia cell, a myoblast cell, a myotube cell, a hepatocyte, a lung epithelial cell, or a fibroblast cell. In some embodiments, the engineered polynucleotide does not comprise a 5′ reducing hydroxyl, a 3′ reducing hydroxyl, or both, capable of being exposed to a solvent. In some embodiments, each 5′ hydroxyl, and each 3′ hydroxyl is independently bonded to a phosphorous by a covalent oxygen phosphorous bond. In some embodiments, the phosphorous is contained in a phosphodiester group. In some embodiments, the engineered polynucleotide further comprises an RNA editing entity recruiting domain. In some embodiments, the targeting domain is about 20 nucleotides to about 150 nucleotides. In some embodiments, the target RNA comprises a nonsense mutation. In some embodiments, the targeting domain comprises at least a single nucleotide that is mismatched to the target RNA. In some embodiments, the mismatched nucleotide is adjacent to two nucleotides, one on each side of the mismatched nucleotide, that are complementary to the target RNA. In some embodiments, the targeting domain at least partially binds to a target RNA that is implemented in a disease or condition. In some embodiments, the disease or condition which comprises Rett syndrome, Huntington's disease, Parkinson's Disease, Alzheimer's disease, a muscular dystrophy, or Tay-Sachs Disease. In some embodiments, the edit of a base results in an increased level of a protein or fragment thereof, an increased length of a protein or fragment thereof, an increased functionality of a protein or fragment thereof, increased stability of a protein or fragment thereof, or any combination thereof after translation of the target RNA with the edit of the base, relative to a translated protein of an otherwise comparable target RNA lacking the edit. In some embodiments, the increased level is from about 5% to about 100%. In some embodiments, the increased length is from about 5% to about 100% of the protein or fragment thereof. In some embodiments, the increased stability is an increased half-life of the protein or fragment thereof. In some embodiments, the engineered polynucleotide comprises a polynucleotide sequence with at least about 80%, at least 85%, at least 90%, at least 92%, at least 95%, at least 97%, at least 99%, or 100% sequence homology to any one of the polynucleotides in Tables 1-12. In some embodiments, the engineered polynucleotide comprises a polynucleotide sequence having a length that is at least about 80%, at least 85%, at least 90%, at least 92%, at least 95%, at least 97%, at least 99%, or 100% of the sequence length of any one of the polynucleotides in Tables 1-12. In some embodiments, the engineered polynucleotide comprises a polynucleotide sequence with at least about 80%, at least 85%, at least 90%, at least 92%, at least 95%, at least 97%, at least 99%, or 100% sequence homology and at least about 80%, at least 85%, at least 90%, at least 92%, at least 95%, at least 97%, at least 99%, or 100% sequence length to any one of the polynucleotides in Tables 1-12.

In some aspects, the present disclosure provides for an engineered guide RNA comprising a targeting domain that is at least partially complementary to a target RNA, wherein the engineered guide RNA comprises a backbone comprising a plurality of sugar and phosphate moieties covalently linked together, and wherein the backbone does not comprise a 5′ reducing hydroxyl, a 3′ reducing hydroxyl, or both, capable of being exposed to a solvent, wherein the targeting domain is configured to at least partially associate with a coding region of the target RNA, wherein the association of the targeting domain with the coding region of the target RNA facilitates an edit of a base of a nucleotide of the target RNA by an RNA editing entity.

In some aspects, the present disclosure provides for an engineered guide RNA that can comprise a targeting domain that is at least partially complementary to a target RNA. In some cases, an engineered guide RNA can further comprise an RNA editing entity recruiting domain, wherein the RNA editing entity recruiting domain is configured to at least transiently associate with an RNA editing entity. In some cases, an engineered guide RNA may not comprise an RNA editing entity recruiting domain. In some cases, the engineered guide RNA can comprise a backbone comprising a plurality of sugar and phosphate moieties covalently linked together, and wherein the backbone does not comprise a 5′ reducing hydroxyl, a 3′ reducing hydroxyl, or both, capable of being exposed to a solvent, wherein the targeting domain is configured to at least partially associate with a coding region of the target RNA, wherein the association of the targeting domain with the coding region of the target RNA facilitates an edit of a base of a nucleotide of the target RNA by an RNA editing entity. In some embodiments, at least partially complementary comprises the targeting domain that comprises a polynucleotide sequence with at least about at least about 80%, at least 85%, at least 90%, at least 92%, or at least 95% sequence homology to a reverse complement to the target RNA. In some embodiments, the edit of the base of a nucleotide of the target RNA by an RNA editing entity is determined in an in vitro assay comprising: (i) transfecting the target RNA into a primary cell line, (ii) transfecting the engineered polynucleotide into a primary cell line, and (iii) sequencing the target RNA.

The disclosure provides for an engineered circular guide RNA having the general structure:

-(aptamer)-(RNA editing entity recruiting domain)-(RNA targeting domain)-(optional RNA editing entity recruiting domain)-(aptamer)-

In some cases, the engineered circular guide RNA may not (lacks) comprise the RNA editing entity recruiting domain. In one embodiment, the aptamer comprising an apatamer suitable for ligation, such that the aptamer on either end of the engineered circular guide RNA are ligated to one another thereby forming the circular construct. The apatamers can be ligated using an endogenous ligase or a recombinantly provided ligase. In another embodiment, the RNA editing entity recruiting domain can recruit endongenous RNA editing entities or can recruit recombinantly provided RNA editing entities. In one embodiment, the RNA editing entity recruiting domain can be selected from an Alu domain, an APOBEC recruiting domain, a GluR2 domain, a Cas13 domain, and functional fragments of any of the foregoing. In another embodiment, the RNA editing entity recruiting domain can be selected from an APOBEC protein (e.g., APOBEC1, APOBEC2, APOBEC3A, APOBEC3B, APOBEC3C, APOBEC3E, APOBEC3F, APOBEC3G, APOBEC3H, or APOBEC4 protein) or an ADAR protein (e.g., ADAR1, ADAR2, or ADAR3 protein). In one embodiment, there circular guide RNA comprises two RNA editing entity recruiting domains. In a further embodiment, the two RNA editing entity recruiting domains flank an RNA targeting domain (one at the 5′ and one and 3′ end of the RNA targeting domain. In another embodiment, the RNA targeting domain is complementary to a target RNA sequence to be edited except for the base to be modified. In another embodiment, the RNA targeting domain includes a plurality of mismatch bases one of which is the target base to be chemically modified while others are mismatches of bases that are susceptible to hyperediting. In one embodiment, the target mismatch basepair of the RNA targeting domain is the site of chemical modification by an RNA editing entity of the target RNA sequence. In one embodiment, the engineered circular guide RNA as described in the foregoing embodiments is encoded by a recombinant polynucleotide. In a further embodiment, a vector can comprise the recombinant polynucleotide.

### BRIEF DESCRIPTION OF THE DRAWINGS

A better understanding of certain features and advantages of the disclosure will be obtained by reference to the following detailed description that sets forth illustrative embodiments, in which the principles of the disclosure are utilized, and the accompanying drawings of which:

FIG. 1A-C shows exemplary construct designs of circular guide RNAs and methods of making circular guide RNAs.

FIG. 2 shows an agarose gel having a PCR amplification product only if forming a circular guide RNA occurred. The wells containing the Alu 100-50 guide show bands of amplification product.

FIG. 3A shows exemplary sequences (e.g. circularized Alu 60-30 R7A guide and circularized Alu 100-50 R7A guide) of Alu-v2-circular constructions.

FIG. 3B shows circular adRNA designs to improve recruitment of endogenous ADARs. Editing inducer elements (EIE) or Alu elements are added to the circular adRNA via a F30 junction.

FIG. 4 shows a schematic of the luciferase assay for RNA editing with two different scenarios of no ADAR recruitment and ADAR recruitment to permit ribosomal read-through that results in normal luciferase expression.

FIG. 5 shows examples of pre-strained and unstrained circular adRNAs targeting mRNA.

FIG. 6 shows a bar graph of relative light units indicating the ability of circular adRNAs of various lengths to restore luciferase activity in cells by RNA editing with or without the addition of exogenous ADAR enzymes.

FIG. 7 shows amplification bands in an agarose gel from various lengths of circular adRNA. The adRNA was amplified with reverse transcriptase (RT)-PCR to confirm that adRNAs were circularized in cells.

FIG. 8 shows a bar graph of the percent edited mRNA of different constructs that may be employed for circular adRNA construction with or without overexpression of ADAR enzymes. Additionally, FIG. 8 also shows a schematic of an Alu element structure with an antisense domain that includes a C mismatch for RNA editing by ADAR.

FIG. 9A-E shows various construct comparisons and resulting data obtained using constructs of the disclosure. (A) A comparison of the RNA editing efficiencies achieved in the 3′ UTR of the RAB7A transcript using various adRNA designs. Values represent mean+/−SEM (n=3). (B) Confirmation of adRNA circularization. (C) RNA editing efficiencies achieved 2 and 4 days post transfection using various adRNA designs. Values represent mean+/−SEM (n=3). (D) The ability of adRNA to effect RNA editing of the cluc transcript is assessed in the presence of siRNA targeting ADAR1. Values represent mean+/−SEM (n=3). (E) siRNA based knockdown of ADAR1. Values represent mean+/−SEM (n=3). All experiments were carried out in HEK293FT cells.

FIG. 10A-C shows results and efficiencies of circular guide RNAs. (A) RNA editing efficiencies in the CDS of various transcripts observed 2 days post plasmid transfections. Values represent mean (n=2). (B) RNA editing efficiencies observed via IVT circular adRNA and plasmid delivered (U6 transcribed) circular adRNA 1 day post transfection in the 3′ UTR of the RAB7A transcript. Values represent mean+/−SEM (n=3). (C) Confirmation of circularization of IVT adRNA and plasmid delivered (U6 transcribed) circular adRNA. Values represent mean+/−SEM (n=3).

FIG. 11A-B shows editing efficiencies and expression of constructs of the disclosure. (A) In vivo RNA editing efficiencies in mice livers via systemic delivery of U6 transcribed circular long antisense RNA packaged into AAV8. Values represent mean+/−SEM (n=3). (B) Relative expression levels of circ adRNA. Values represent mean+/−SEM (n=3).

FIG. 12A-C shows Alu and adRNA designs and their edited mRNA activities. (A) Schematic of an Alu element and 3 adRNA designs engineered from the Alu element. (B) The Alu-adRNAs are assayed for their ability to recruit endogenous and exogenous ADARs. Values represent mean+/−SEM (n=3). (C) Antisense domain evaluation with the Alu-v2 design. Values represent mean+/−SEM (n=3). All experiments were carried out in HEK293FT cells.

FIG. 13A-B shows (A) Schematic of an Alu element and 3 adRNA designs engineered from the Alu element. (B) The Alu-adRNAs are assayed for their ability to recruit endogenous and exogenous ADARs. Values represent mean+/−SEM (n=3). (c) Antisense domain evaluation with the Alu-v2 design. Values represent mean+/−SEM (n=3). All experiments were carried out in HEK293FT cells.

FIG. 14A-B shows (A) Sample trace of the RAB7A cDNA around the target site (highlighted by the large arrow). The smaller arrows point at editing of off-target adenosines. (B) Linear forms of circular adRNA are transcribed in vitro using a T7 polymerase, purified using LiCl and transfected into cells where they are circularized by ligases.

FIG. 15A-F shows (A) In vivo RNA editing efficiencies in mice livers via systemic delivery of U6 transcribed linear and circular long antisense RNA packaged into AAV8. Values represent mean+/−SEM (n=3). (B) Relative expression levels of circ adRNA, (C) GFP and (D) human ADAR2 in the various groups of AAV8 or PBS injected mice. AAV vectors delivering the adRNA alone also contain a GFP. Values represent mean+/−SEM (n=3). (E) PCSK9 expression levels in the various groups of AAV8 or PBS injected mice. Values represent mean+/−SEM (n=3). (F) Heatmap depicting hyper-editing observed with the circular adRNA corresponding to the plot in SI FIG. 4a. The arrow indicates the target adenosine.

FIG. 16 provides 2D histograms comparing the transcriptome-wide A-to-G editing yields observed with each long antisense adRNA construct (y-axis) to the yields observed with the control sample (x-axis). Each histogram represents the same set of reference sites, where read coverage was at least 10 and at least one putative editing event was detected in at least one sample. Highlighted bins contain sites with significant changes in A-to-G editing yields when comparing treatment to control sample. Crosses in each plot indicate the 100 sites with the smallest adjusted P values. Clockwise from top left, Circular 100,50; and 0,100,50.

FIG. 17A-D shows circular adRNA antisense designs for reducing hyperediting. The bottom strand represents the antisense domain while the top strand is the target mRNA. The target adenosine is indicated opposite a mismatch; (A) Loops of size 6-12 bp are created 30-40 bp apart with the target adenosine located within this region. (B) Single bp bulges are created all across the antisense following the pattern: . . . −34, −24, −14, −10, 0, 10, 14, 24, 34 . . . ; (C) Single bp bulges are created all across the antisense following the pattern: . . . −30, −20, −10, 0, 10, 20, 30 . . . ; (D) Bulges are created by positioning guanosines opposite hyperedited adenosines.

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the fragment” includes reference to one or more fragments and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of ” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The terms “adenine”, “guanine”, “cytosine”, “thymine”, “uracil” and “hypoxanthine” (the nucleobase in inosine) as used herein refer to the nucleobases as such.

The terms “adenosine”, “guanosine”, “cytidine”, “thymidine”, “uridine” and “inosine”, refer to the nucleobases linked to the (deoxy)ribosyl sugar.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11, sequentially numbered, are disclosed in the prior art. Non-limiting exemplary serotypes useful for the purposes disclosed herein include any of the 11 serotypes, e.g., AAV2 and AAV8. The term “lentivirus” as used herein refers to a member of the class of viruses associated with this name and belonging to the genus lentivirus, family Retroviridae. While some lentiviruses are known to cause diseases, other lentivirus are known to be suitable for gene delivery. See, e.g., Tomás et al. (2013) Biochemistry, Genetics and Molecular Biology: “Gene Therapy—Tools and Potential Applications,” ISBN 978-953-51-1014-9, DOI: 10.5772/52534.

The term “Adenosine Deaminase acting on RNA” or “ADAR” as used herein refers to an adenosine deaminase that can convert adenosines (A) to inosines (I) in an RNA sequence. ADAR1 and ADAR2 are two exemplary species of ADAR that are involved in mRNA editing in vivo. Non-limiting exemplary sequences for ADAR1 may be found under the following reference numbers: HGNC: 225; Entrez Gene: 103; Ensembl: ENSG 00000160710; OMIM: 146920; UniProtKB: P55265; and GeneCards: GC01M154554, as well as biological equivalents thereof. Non-limiting exemplary sequences for ADAR2 may be found under the following reference numbers: HGNC: 226; Entrez Gene: 104; Ensembl: ENSG00000197381; OMIM: 601218; UniProtKB: P78563; and GeneCards: GC21P045073, as well as biological equivalents thereof. Related orthologs and homologs will be readily identified using various sequence search tools and databases available to one of skill in the art.

The term “adRNA” stands for ADAR recruiting RNA and can refer to the forward and reverse RNA used to direct site-specific ADAR editing. In some instances, a forward RNA that recruits ADAR is referred to as “adRNA”, whereas a reverse RNA that recruits ADAR is referred to as “radRNA”.

The term “Alu domain” can refer to a sequence obtained from the Alu transposable element (“Alu element”). In some cases, the Alu element can be about 300 base pairs in length. An Alu element typically comprise a structure: cruciform-polyA5-TAC-polyA6-cruciform-polyA tail, wherein both cruciform domains are similar in nucleotide sequence. An “Alu domain” can comprise a cruciform portion of the Alu element. In some embodiments, two Alu domains comprising cruciform structures are linked by a sequence complementary to a target RNA sequence.

The term “APOBEC” as used herein can refer to any protein that falls within the family of evolutionarily conserved cytidine deaminases involved in mRNA editing—catalyzing a C to T edit, which can be interpreted as a C to U conversion—and equivalents thereof. In some aspects, the term APOBEC can refer to any one of APOBEC1, APOBEC2, APOBEC3A, APOBEC3B, APOBEC3C, APOBEC3E, APOBEC3F, APOBEC3G, APOBEC3H, APOBEC4, or equivalents each thereof. Non-limiting exemplary sequences of fusion proteins comprising one or more APOBEC domains are provided herein both fused to an ADAR domain or fused to alternative domains to render them suitable for use in an RNA editing system. To this end, APOBECs can be considered an equivalent of ADAR—catalyzing editing albeit by a different conversion. Thus, not to be bound by theory, it is believed that all embodiments contemplated herein for use with an ADAR based editing system can be adapted for use in an APOBEC based RNA editing system. In some cases, use of APOBEC can involve certain modifications, such as but not limited to the use of particular guide RNA or “gRNA” (including circularized gRNA) to recruit the enzyme.

The term “contacting” can mean direct or indirect binding or interaction between two or more entities. An example of direct interaction is binding. An example of an indirect interaction is where one entity acts upon an intermediary molecule, which in turn acts upon the second referenced entity. Contacting as used herein includes in solution, in solid phase, in vitro, ex vivo, in a cell and in vivo. In one embodiment, contacting can occur between a guide RNA and an RNA editing entity. Contacting in vivo can be referred to as administering, or administration.

The term “deficiency” as used herein can refer to lower than normal (physiologically acceptable) levels of a particular agent. In context of a protein, a deficiency can refer to lower than normal levels of the full-length protein.

As used herein the term “domain” refers to a particular region of a larger construct such that the domain is contained in or is part of the larger construct. With respect to nucleic acids a domain can refer to a coding sequence found in a larger construct containing multiple coding sequences.

The term “encode” as it is applied to polynucleotides can refer to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

An “engineered polynucleotide” or “engineered guide RNA” are used interchangeably with circular guide RNA. An engineered polynucleotide can comprise a recombinant polynucleotide of DNA or RNA or a hybrid DNA/RNA construct. The engineered polynucleotide can give rise to a guide RNA and more particularly can give rise to a circular guide RNA. Formulas I, II, and III provide exemplary structures of engineered polynucleotides.

The terms “equivalent” or “biological equivalent” are used interchangeably when referring to a particular molecule, biological or cellular material having minimal homology while still maintaining desired structure or functionality.

As used herein, “expression” can refer to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

“Homology” or “identity” or “similarity” can refer to sequence similarity between two peptides or between two nucleic acid molecules. Homology can be determined by comparing a position in each sequence which can be aligned for purposes of comparison. For example, when a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the disclosure.

Homology can refer to a percent (%) identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein, such particular peptide, polypeptide or nucleic acid sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

For example, in a specific embodiment the identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some cases, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity can be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

“Hybridization” can refer to a reaction in which one or more polynucleotides react to form a complex that is stabilized via hydrogen bonding between the bases of the nucleotide residues. The hydrogen bonding can occur by Watson-Crick base pairing, Hoogstein binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1× SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein can refer to molecules or biologicals or cellular materials being substantially free from other materials. In one aspect, the term “isolated” can refer to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also can refer to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and may not be found in the natural state. In some cases, the term “isolated” is also used herein to refer to polypeptides which are isolated from other cellular proteins and is meant to encompass both purified and recombinant polypeptides. In some cases, the term “isolated” is also used herein to refer to cells or tissues that are isolated from other cells or tissues and is meant to encompass both cultured and engineered cells or tissues.

“Messenger RNA” or “mRNA” is a nucleic acid molecule that is transcribed from DNA and then processed to remove non-coding sections known as introns. In some cases, the resulting mRNA is exported from the nucleus (or another locus where the DNA is present) and translated into a protein. The term “pre-mRNA” can refer to the strand prior to processing to remove non-coding sections.

The term “mutation” as used herein, can refer to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations are those which have no effect on the resulting protein. As used herein the term “point mutation” can refer to a mutation affecting only one nucleotide in a gene sequence. “Splice site mutations” are those mutations present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. The reference DNA sequence can be obtained from a reference database. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (i.e., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation can indicate that a tissue or sample is benign. As an alternative, absence of a mutation may not indicate that a tissue or sample is benign. Methods as described herein can comprise identifying a presence of a mutation in a sample.

The terms “polynucleotide” and “oligonucleotide” are used interchangeably and refer to a polymeric form of nucleotides of any length, either deoxyribonucleotides or ribonucleotides or analogs or combinations thereof. Polynucleotides can have any three dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also can refer to both double and single stranded molecules. Unless otherwise specified or required, any embodiment of this disclosure that is a polynucleotide can encompass both the double stranded form and each of two complementary single stranded forms known or predicted to make up the double stranded form. In some embodiments, a polynucleotide can include both RNA and DNA nucleotides.

The term “polynucleotide sequence” can be the alphabetical representation of a polynucleotide molecule. This alphabetical representation can be input into databases in a computer having a central processing unit and used for bioinformatics applications such as functional genomics and homology searching. In any alphabetic representation, the disclosure contemplates both RNA and DNA (i.e., wherein “T” is replaced with “U” or vice-a-versa).

The term “recruiting domain” refers to a polynucleotide sequence the can bind to or recruit one or more RNA editing entities. Exemplary recruiting domains can be an Alu domain, an APOBEC recruiting domain, a GluR2 domain, a Cas13 recruiting domain or any combination thereof.

The term “RNA editing entity” refers to a biological molecule that can cause a chemical modification of a nucleotide to change the nucleotide to a different nucleotide. In some embodiments, an RNA editing entity can be recruited to a particular site in a polynucleotide to cause a change in the nucleic acid sequence at a desired site. Examples of RNA editing entities include APOBEC protein (e.g., APOBEC1, APOBEC2, APOBEC3A, APOBEC3B, APOBEC3C, APOBEC3E, APOBEC3F, APOBEC3G, APOBEC3H, or APOBEC4 protein) or an ADAR protein (e.g., ADAR1, ADAR2, or ADAR3 protein).

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient.

A “targeting domain” refers to a polynucleotide sequence that can be at least partially complementary to a target RNA in a cell. The targeting domain is typically not 100% identical to the target RNA, but rather has mismatches at one or more site where a chemical reaction is desired to modify the target RNA sequence. A targeting domain includes the complementary RNA antisense sequence to the target RNA as well as DNA sequence that encode (upon transcription) the antisense RNA sequence that is complementary to the RNA target sequence. The targeting domain is typically sufficiently complementary to the target RNA sequence to hybridize under biological condition to the target RNA sequence.

“Transfer ribonucleic acid” or “tRNA” is a nucleic acid molecule that helps translate mRNA to protein. tRNA have a distinctive folded structure, comprising three hairpin loops; one of these loops comprises a “stem” portion that encodes an anticodon. The anticodon recognizes the corresponding codon on the mRNA. Each tRNA is “charged with” an amino acid corresponding to the mRNA codon; this “charging” is accomplished by the enzyme tRNA synthetase. Upon tRNA recognition of the codon corresponding to its anticodon, the tRNA transfers the amino acid with which it is charged to the growing amino acid chain to form a polypeptide or protein. Endogenous tRNA can be charged by endogenous tRNA synthetase. Accordingly, endogenous tRNA are typically charged with canonical amino acids. Orthogonal tRNA, derived from an external source, require a corresponding orthogonal tRNA synthetase. Such orthogonal tRNAs may be charged with both canonical and non-canonical amino acids. In some embodiments, the amino acid with which the tRNA is charged may be detectably labeled to enable detection in vivo. Techniques for labeling are known in the art and include, but are not limited to, click chemistry wherein an azide/alkyne containing unnatural amino acid is added by the orthogonal tRNA/synthetase pair and, thus, can be detected using alkyne/azide comprising fluorophore or other such molecule.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEI™ (Polyplus-transfection Inc., New York, N.Y.), EFFECTENE® (Qiagen, Valencia, Calif.), DREAMFECT™ (OZ Biosciences, France) and the like), or electroporation. Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals. Standard recombinant DNA and molecular cloning techniques used herein are well known in the art and are described in Sambrook, J., Fritsch, E. F. and Maniatis, T., Molecular Cloning: A Laboratory Manual, 2^{nd} ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety. Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

The terms “treat”, “treating” and “treatment”, as used herein, refers to ameliorating symptoms associated with a disease or disorder, including preventing or delaying the onset of the disease or disorder symptoms, and/or lessening the severity or frequency of symptoms of the disease or disorder.

As used herein, the term “circular” used in the context of a nucleic acid molecule (e.g. an engineered guide RNA) can generally refer to a nucleic acid molecule that can be represented as a polynucleotide sequence in a circular 2-dimensional format with one nucleotide after the other wherein the represented polynucleotide is circular. In some embodiments, a circular nucleic acid molecule does not comprise a 5′ reducing hydroxyl, a 3′ reducing hydroxyl, or both capable of being exposed to a solvent

As used herein, the term “vector” can refer to a nucleic acid construct deigned for transfer between different hosts, including but not limited to a plasmid, a virus, a cosmid, a phage, a BAC, a YAC, etc. In some embodiments, a “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct can refer to the polynucleotide comprising the retroviral genome or part thereof, and a gene of interest. Further details as to modern methods of vectors for use in gene transfer can be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.). In one aspect, the promoter is a pol III promoter.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and ‘Vector” can be used interchangeably. However, the disclosure is intended to include such other forms of expression vectors, such as viral vectors (e.g., replication defective retroviruses, adenoviruses and adeno-associated viruses), which serve equivalent functions. Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Typically, the vector or plasmid contains sequences directing transcription and translation of a gene fragment, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant coding regions in the desired host cell are numerous and familiar to those skilled in the art. Virtually any promoter capable of driving these genetic elements is suitable for use in the disclosure. Termination control regions may also be derived from various genes native to the preferred hosts.

Adenosine to inosine (A-to-I) RNA editing is a common post-transcriptional RNA modification catalyzed by Adenosine Deaminases acting on RNA (ADAR) enzymes. ADARs edit double stranded RNA (dsRNA), predominantly in non-coding regions such as Alu repetitive elements while also editing a handful of sites in coding regions, leading to major alterations in protein function. The structural similarity between inosine and guanosine accounts for the translation and splicing machinery recognizing the edited base as guanosine, thereby making ADARs attractive tools for altering protein sequences. Over the last decade, several studies have repurposed ADAR enzymes for site-specific RNA editing by recruiting them to a target RNA sequence, using engineered ADAR recruiting RNAs (adRNAs), both in vitro and in vivo. However, almost all of these studies rely on exogenously expressed ADAR enzymes and their variants. In some cases, one of the major limitations of using exogenous enzyme overexpression is its propensity to introduce large number of off-target A-to-I edits across the transcriptome. A potential solution to this problem is the engineering of adRNAs to enable recruitment of endogenous ADARs which are expressed across a variety of different cell types. It has recently been shows that using simple long antisense RNA of length 100 bp suffices to recruit endogenous ADARs and these long antisense RNA are both genetically encodable and chemically synthesizable. The use of both genetically encodable long antisense RNA as well as chemically modified antisense oligonucleotides enabled highly transcript specific RNA editing. However, the efficiency of these approaches is significantly lower than seen with enzyme overexpression. Additionally, chemically modified antisense oligonucleotides are extremely expensive to synthesize. On the contrary, genetically encodable adRNA can be delivered as DNA, and transcribed by the cell itself via an H1, U6 or similar promoter or be delivered as RNA when synthesized by in vitro transcription. The use of genetically encodable adRNA can be cheaper and more convenient than chemically modified antisense oligonucleotides.

In some cases, a hurdle in the RNA editing space can be guide stability. An adRNA may be present for extended periods of time in order to successfully recruit endogenous ADARs, but single stranded RNAs may have a half-life of about 30 minutes or less in mammalian cells. This may be due to their susceptibility to exonucleases that may degrade single stranded RNA from the 5′ or 3′ ends. Modifications may be made to a guide RNA to increase guide stability. As described herein, forming a circular guide RNA may be one type of modification to enhance guide RNA stability. Circularization may prevent exposed ends of a guide RNA from being degraded and may significantly increase the half-life of a guide RNA, such as in vivo or in vitro. In some cases, a circular guide RNA may prevent one or more exposed ends from hydrolytic degradation. In some cases, a circular guide RNA may significantly increase a half-life of the guide RNA as compared to a comparable guide RNA that is not circular. In some cases, forming a circular guide RNA may significantly increase a half-life of a guide RNA when delivered in vivo, such as to a subject, as compared to a comparable guide RNA that is not circular. In some cases, forming a circular guide RNA may significantly reduce an amount (such as a therapeutically effective amount) of the guide RNA dosed to a subject as compared to a comparable guide RNA that is not circular. In some cases, forming a circular guide RNA may significantly enhance efficiency of editing, may significantly reduce off target editing, or a combination thereof as compared to a comparable guide RNA that is not circular.

Circular guide RNAs may provide various benefits as compared to non-circular guide RNAs. Circular guides may provide greater stability, improved recruitment of RNA editing entities (such as endogenous RNA editing enzymes), longer half-lives, or any combination thereof as compared to a comparable guide RNA that is not circular. Circular guide RNA may provide one or more of these improved qualities and may retain genetic encodability as compared guide RNAs comprising other types of modifications designed to improve guide stability—such as chemical modifications or sugar additions. Circular guide RNAs may be capable of being genetically encoded, capable of being delivered by a vector, and retain improved stability. In some cases, an encoded engineered guide RNA can be codon optimized.

An engineered guide RNA may be circular. An engineered guide RNA may not comprise a 5′ reducing hydroxyl capable of being exposed to a solvent. An engineered guide RNA may not comprise a 5′ reducing hydroxyl, 3′ reducing hydroxyl, or both capable of being exposed to a solvent. An engineered guide RNA may be less susceptible to hydrolytic degradation than an mRNA naturally present in a human cell. An engineered guide RNA may be circular and may also retain a substantially similar secondary structure as a substantially similar engineered guide RNA that is not circular. The engineered guide RNA may comprise a recruiting domain, a targeting domain, or both. The engineered guide RNA may recruit an RNA editing entity, such as an enzyme, to edit a base of an RNA sequence. A circular engineered guide RNA may be pre-strained. A circular engineered guide RNA may comprise a decreased level of entropy.

A circular engineered guide RNA may comprise a recruiting domain, such as an RNA editing entity recruiting domain, that may recruit an RNA editing entity to perform a chemical transformation on a base in an RNA sequence. The recruiting domain may recruit an endogenous RNA editing entity or an exogenous RNA editing entity. In some aspects, a circular engineered guide RNA may not comprise a separate recruiting domain. The RNA editing entity may be an enzyme, such as an endogenous enzyme or a recombinant enzyme. The enzyme may perform the edit to the base. The circular engineered guide RNA may also comprise a targeting domain.

An engineered polynucleotide may comprise the structure of Formula (I):

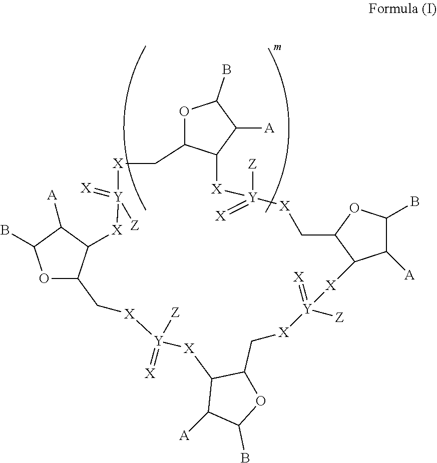

In some cases, each X may be independently O, S, or NR; each Y may be independently P or S; each Z may be independently OM, SM, or NRM; each A may be independently H, D, halogen, OM, SM, NRM, or NRR′; each B may be independently uracil, thymine, adenine, cytosine, guanine, a salt of any of these, or a derivative of any of these; each M may be independently an inorganic or organic cation, H, or D; and each R and R′ may be independently H, D, halogen, or C₁-C₆ alkyl; and m may be any integer from 1-1,000. In some cases, m may be an independent integer from about: 5 to about 250, 100 to about 500, 400 to about 800, 600 to about 1200, 800 to about 2000, 1500 to about 4000 or about 300 to about 10000. In some instances, each X can be an O. In some instances, each Z can independently be O or S. In some instances, each Y can be a P. In some cases, an engineered polynucleotide may be circular. In some cases, an engineered polynucleotide may be a guide polynucleotide, such as an engineered guide RNA. In some cases, the targeting domain may be configured to at least partially associate with a coding region of a target RNA. In some cases, a targeting domain can be at least partially complementary to a target RNA. In some cases, at least partially complementary can comprise a targeting domain that can comprise a polynucleotide sequence with at least about 80% sequence homology to a reverse complement to the target RNA. In some instances, at least partially complementary can comprise a targeting domain comprising a polynucleotide sequence with at least about 70%, at least about 80%, or at least about 90% sequence homology to the reverse complement of the target RNA. In some cases, the targeting domain can at least partially bind to a target RNA that may be implemented in a disease or condition. The association of the targeting domain and the target RNA may facilitate an edit of a base by an RNA editing entity such as ADAR1, ADAR2, APOBEC, or a combination thereof. In some cases, an engineered polynucleotide may further comprise an RNA editing entity recruiting domain. In some cases, an edit of a base may be a chemical transformation of a base. In some embodiments, the target RNA can comprise a nonsense mutation, a missense mutation, or both. In some cases, a targeting domain can comprise at least a single nucleotide that may be mismatched to the target RNA. In some instances, the mismatched nucleotide on the targeting domain can be adjacent to two nucleotides, one on each side of the mismatched nucleotide, which may be complementary to the target RNA. In some cases, RNA editing may be determined in an in vitro assay by transfecting a target RNA and an engineered polynucleotide designed to target the target RNA into the same cell. The target RNA may be sequenced to identify editing by the engineered polynucleotide. In some cases, transfecting a target RNA into a primary cell line can comprise transfecting a plasmid encoding for the target RNA into a primary cell line. In some instances, transfecting an engineered polynucleotide into a primary cell line can comprise transfecting a plasmid that encodes for an engineered polynucleotide into a primary cell line. In some cases, the percent RNA editing of a target RNA can be determined at different time points (e.g. 24 hours, 48 hours, 96 hours) after transfection with a guide RNA or engineered polynucleotide by reverse transcribing the target RNA to cDNA then using Sanger sequencing to determine the percent RNA editing of a target RNA. In some cases, the cDNA can be amplified prior to sequencing by polymerase chain reaction. Sanger traces from Sanger sequencing can be analyzed to assess the editing efficiency of guide RNAs. In some cases, a cell can be a primary cell. In some cases, a primary cell or a cell can be a neuron, a photoreceptor cell (e.g. a S cone cell, a L cone cell, a M cone cell, a rod cell), a retinal pigment epithelium cell, a glia cell (e.g. an astrocyte, an oligodendrocyte, a microglia), a muscle cell (e.g. a myoblast, a myotube), a hepatocyte, a lung epithelial cell, or a fibroblast (e.g. dermal fibroblast). In some cases, a cell can be a horizontal cell, a ganglion cell, or a bipolar cell. In some cases, a cell line can be a mammalian cell line, such as HEK293T, NCI-60, MCF-7, HL-60, RD, LHCN differentiated, LHCN undifferentiated, Saos-2, CHO, or HeLa cells. In some cases, a cell line can be an insect cell line, such as Sf9.

An engineered polynucleotide, such as an engineered guide polynucleotide may comprise the structure of Formula (III):

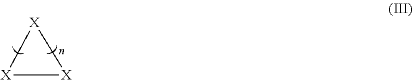

In some cases, each X may be a nucleotide comprising a base that may be independently uracil, thymine, adenine, cytosine, guanine, a salt of any of these, or a derivative of any of these; and n may be independently an integer from 0-1,000. In some cases, n may be an independently integer from about: 5 to about 250, 100 to about 500, 400 to about 800, 600 to about 1200, 800 to about 2000, 1500 to about 4000 or about 300 to about 10000. In some cases, each nucleotide may be connected to two adjacent nucleotides by, independently for each connection, a phosphoester, phosphothioester, phosphothioate, or phosphoramidite linkage. In some cases, the targeting domain can be configured to at least partially associate with a coding region of the target RNA. In some cases, the targeting domain can at least partially bind to a target RNA that may be implicated in a disease or condition. In some cases, the targeting domain may be configured to at least partially associate with a coding region of a target RNA. The association of the targeting domain and the target RNA may facilitate an edit of a base by an RNA editing entity such as ADAR1, ADAR2, APOBEC, or any combination thereof. In some cases, an edit of a base may be a chemical transformation of a base. In some embodiments, the target RNA can comprise a nonsense mutation, a missense mutation, or both. In some cases, a targeting domain can comprise at least a single nucleotide that may be mismatched to the target RNA. In some instances, the mismatched nucleotide on the targeting domain can be adjacent to two nucleotides, one on each side of the mismatched nucleotide, which may be complementary to the target RNA. In some cases, the editing may be determined by an in vitro assay as described herein. In some cases, an engineered polynucleotide can further comprise an RNA editing entity recruiting domain.

In some embodiments, a chemical transformation, such as a chemical transformation by an RNA editing entity, may comprise an edit of a base. In some embodiments, a chemical transformation, such as an edit of a base may result in an increased level of a protein or fragment thereof after translation of a target RNA with the chemical transformation, relative to an otherwise comparable target RNA lacking the chemical transformation. In some cases, an increased level can be from about: 5% to about 100%, 10% to about 50%, 25% to about 75%, or from about 40% to about 90%. In some embodiments, a chemical transformation can result in a decreased level of a protein or fragment thereof after translation of a target RNA with the chemical transformation, relative to an otherwise comparable target RNA lacking the chemical transformation. In some cases, a decreased level can be from about: 5% to about 99%, 10% to about 50%, 25% to about 75%, or from about 40% to about 90%. In some embodiments, a chemical transformation can result in an increased length of a protein or fragment thereof, an increased functionality of a protein or fragment thereof, increased stability of a protein or fragment thereof, or any combination thereof after translation of the target RNA with the edit of the base, relative to a translated protein of an otherwise comparable target RNA lacking the edit. In some cases, an increased length can be from about: 5% to about 100%, 2% to about 10%, 10% to about 25%, 25% to about 50%, 40% to about 80%, or about 75% to about 150%. In some cases, the increased length of a protein or a fragment thereof can be over 100%. In some cases, the increased stability can be an increased half-life of the protein or fragment thereof. In some cases, the increased half-life can be at least about: 2×, 3×, 4×, 5×, 6×, 7×, 8×, 9×, or 10× greater than to a translated protein of an otherwise comparable target RNA lacking the edit. In some cases, increased functionality can comprise a protein or fragment thereof, such as an enzyme, that may increase the speed of a reaction, increase the Vₘₐₓ, or both. In some cases, increased functionality may comprise a protein (e.g. an enzyme) or fragment thereof, encoded by a target RNA with the edit of the base, comprising a lower energy of activation as compared to a translated protein of an otherwise comparable target RNA lacking the edit.

In some embodiments, an engineered polynucleotide may not comprise (lacks) a 5′ reducing hydroxyl, a 3′ reducing hydroxyl, or both, capable of being exposed to a solvent. In some cases, each 5′ hydroxyl, and each 3′ hydroxyl may be independently bonded to a phosphorous by a covalent oxygen phosphorous bond. In some instances, the phosphorous may be contained in a phosphodiester group.

In some embodiments, an engineered polynucleotide may comprise chirality. In some embodiments, any center atom, which can be chiral can be independently in the R or S configuration. In some cases, chiral may comprise an atom in a molecule that may be bonded to four different types of atoms or chains of atoms. In some instances, an engineered polynucleotide, such as a guide RNA may be a single diastereomer or may be predominantly one diastereomer. In some instances, an engineered polynucleotide may have a diastereomeric excess of from about: 51% to about 100%, 51% to about 60%, 60% to about 75%, 70% to about 90% or about 80% to about 99%. Diastereomeric excess can be a measurement of purity used for chiral substances. In some cases, it may reflect the degree to which a sample contains one diastereomer in greater amounts than another diastereomer. In some cases, a single pure diastereomer may have a diastereomeric excess of 100%. A sample with 70% of one diastereomer and 30% of the other may have a diastereomeric excess of 40% (70% -30%).

An engineered polynucleotide (e.g. an engineered guide RNA) may comprise a targeting domain that may be at least partially complementary to a target RNA. In some cases, the engineered guide RNA can comprise a backbone comprising a plurality of sugar and phosphate moieties covalently linked together. In some cases, the backbone may not comprise (lacks) a 5′ reducing hydroxyl, a 3′ reducing hydroxyl, or both, capable of being exposed to a solvent. In some cases, the engineered guide RNA may have an RNA editing entity recruiting domain. The RNA editing entity recruiting domain may be configured to interact with an RNA editing entity, such as, for example, ADAR1 or ADAR2. In some cases, the engineered guide RNA may not have (lacks) an RNA editing entity recruiting domain.

The chemical transformation on the base may include editing one or more bases of the targeted RNA sequence. The chemical transformation on a base may edit a sense codon to a stop codon, a stop codon to a sense codon, a first sense codon to a second sense codon, or a first stop codon to a second stop codon. In some cases, the chemical transformation can covert a sense codon specifying a first amino acid into a second sense codon specifying a second amino acid. In some cases, the first amino acid can be a protease cleavage site.

In some instances, a polynucleotide sequence can share about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence homology to a sequence described herein. In some instances, the length of any sequence recited herein can be truncated to about: 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98%, of the original sequence.

In some instances, a targeting domain can have a sequence length of from about: 20 nucleotides to about 1,000 nucleotides, 10 nucleotides to about 100 nucleotides, 50 nucleotides to about 500 nucleotides or about 400 nucleotides to about 1000 nucleotides in length. In some instances, a targeting domain of an engineered polynucleotide, or a construct for forming an engineered polynucleotide, can comprise a polynucleotide sequence with at least about: 70%, 75%, 80%, 85%, 90%, or 95% homology to any one of the polynucleotides in SEQ ID NOs:1-1417 (See also, Tables 1-12). In some instances, the sequences in Tables 1-12 can at least in part encode for the targeting domain of an engineered polynucleotide, or a construct for forming an engineered polynucleotide. In some instances, in Tables 1-12, a T (thymine) can be substituted with a U (uracil) in a polynucleotide. In some instances, in Tables 1-12, all Ts can be substituted with Us in a polynucleotide. In some instances, a targeting domain of a construct for forming an engineered polynucleotide, or an engineered polynucleotide, can comprise a polynucleotide sequence with at least about: 70%, 75%, 80%, 85%, 90%, or 95% sequence length to any one of the polynucleotides in Tables 1-12. In some instances, a targeting domain of a construct for forming an engineered polynucleotide, or an engineered polynucleotide, can comprise a polynucleotide sequence with at least about: 70%, 75%, 80%, 85%, 90%, or 95% sequence length to any one of the polynucleotides in Tables 1-12 and a polynucleotide sequence with at least about: 70%, 75%, 80%, 85%, 90%, or 95% homology to any one of the polynucleotides in Tables 1-12. In some instances, an engineered polynucleotide can comprise a polynucleotide sequence with at least about 80% sequence homology to any one of the polynucleotides in Table 1. In some instances, an engineered polynucleotide can comprise a polynucleotide sequence with at least about 80% sequence homology to any one of the polynucleotides in Table 2. In some instances, an engineered polynucleotide can comprise a polynucleotide sequence at least about 80% sequence homology to any one of the polynucleotides in Table 3. In some instances, an engineered polynucleotide can comprise a polynucleotide sequence with at least about 80% sequence homology to any one of the polynucleotides in Table 4. In some instances, an engineered polynucleotide can comprise a polynucleotide sequence at least about 80% sequence homology to any one of the polynucleotides in Table 5. In some instances, an engineered polynucleotide can comprise a polynucleotide sequence with at least about 80% sequence homology to any one of the polynucleotides in Table 6. In some instances, an engineered polynucleotide can comprise a polynucleotide sequence at least about 80% sequence homology to any one of the polynucleotides in Table 7. In some instances, an engineered polynucleotide can comprise a polynucleotide sequence with at least about 80% sequence homology to any one of the polynucleotides in Table 8. In some instances, an engineered polynucleotide can comprise a polynucleotide sequence with at least about 80% sequence homology to any one of the polynucleotides in Table 9. In some instances, an engineered polynucleotide can comprise a polynucleotide sequence with at least about 80% sequence homology to the polynucleotide in Table 10. In some instances, an engineered polynucleotide can comprise a polynucleotide sequence with at least about 80% sequence homology to any one of the polynucleotides in Table 11. In some instances, an engineered polynucleotide can comprise a polynucleotide sequence with at least about 80% sequence homology to any one of the polynucleotides in Table 12.

TABLE 1
(it will be recognized that the sequences below can be RNA or DNA (T can be U
or vice-a-versa unless methylation clearly indicates otherwise). For example,
a DNA sequence can be expressed from a vector to produce RNA)

| SEQ | |
|---|---|
| ID | |
| NO. | Sequence 5′ -> 3 |
| 1 | G*G*G*C*A*C*A*A*G*G*G*C*A*C*A*G*A*C*T*T |
| 2 | G*G*C*A*C*A*A*G*G*G*C*A*C*A*G*A*C*T*T*C |
| 3 | G*C*A*C*A*A*G*G*G*C*A*C*A*G*A*C*T*T*C*C |
| 4 | C*A*C*A*A*G*G*G*C*A*C*A*G*A*C*T*T*C*C*A |
| 5 | A*C*A*A*G*G*G*C*A*C*A*G*A*C*T*T*C*C*A*A |
| 6 | C*A*A*G*G*G*C*A*C*A*G*A*C*T*T*C*C*A*A*A |
| 7 | mG*mG*mG*mC*mA*C*A*A*G*G*G*C*A*C*A*G*A*C*T*T |
| 8 | mG*mG*mC*mA*mC*A*A*G*G*G*C*A*C*A*G*A*C*T*T*C |
| 9 | mG*mC*mA*mU*mA*A*G*G*G*C*A*C*A*G*A*C*T*T*C*C |
| 10 | mC*mA*mC*mA*mA*G*G*G*C*A*C*A*G*A*C*T*T*C*C*A |
| 11 | mA*mC*mA*mA*mG*G*G*C*A*C*A*G*A*C*T*T*C*C*A*A |
| 12 | mC*mA*mA*mG*mG*G*C*A*C*A*G*A*C*T*T*C*C*A*A*A |
| 13 | mG*mG*mG*mC*mA*C*A*A*G*G*G*C*A*C*A*mG*mA*mC*mU*mU |
| 14 | mG*mG*mC*mA*mC*A*A*G*G*G*C*A*C*A*G*mA*mC*mU*mU*mC |
| 15 | mG*mC*mA*mC*mA*A*G*G*G*C*A*C*A*G*A*mC*mU*mU*mC*mC |
| 16 | mC*mA*mU*mA*mA*G*G*G*C*A*C*A*G*A*C*mU*mU*mC*mC*mA |
| 17 | mA*mC*mA*mA*mG*G*G*C*A*C*A*G*A*C*T*mU*mC*mC*mA*mA |
| 18 | mC*mA*mA*mG*mG*G*C*A*C*A*G*A*C*T*T*mC*mC*mA*mA*mA |
| 19 | mG*mC*mA*mC*mA*mA*mG*mG*G*C*A*C*A*G*A*mC*mU*mU*mC*mC |
| 20 | mC*mA*mC*mA*mA*mG*mG*G*C*A*C*A*G*A*mC*mU*mU*mC*mC*mA |
| 21 | mA*mC*mA*mA*mG*mG*G*C*A*C*A*G*A*mC*mU*mU*mC*mC*mA*mA |
| 22 | mC*mA*mA*mG*mG*G*C*A*C*A*G*A*mC*mU*mU*mC*mC*mA*mA*mA |
| 23 | mGmCmAmCmAmAmGmG*G*C*A*C*A*G*A*mCmUmUmCmC |
| 24 | mCmAmCmAmAmGmG*G*C*A*C*A*G*A*mCmUmUmCmCmA |
| 25 | mAmCmAmAmGmG*G*C*A*C*A*G*A*mCmUmUmCmCmAmA |
| 26 | mCmAmAmGmG*G*C*A*C*A*G*A*mCmUmUmCmCmAmAmA |
| 27 | mGmGmGmCmA*C*A*A*G*G*G*C*A*C*A*mGmAmCmUmU |
| 28 | mGmGmCmAmC*A*A*G*G*G*C*A*C*A*G*mAmCmUmUmC |
| 29 | mGmCmAmCmA*A*G*G*G*C*A*C*A*G*A*mCmUmUmCmC |
| 30 | mCmAmCmAmA*G*G*G*C*A*C*A*G*A*C*mUmUmCmCmA |
| 31 | mAmCmAmAmG*G*G*C*A*C*A*G*A*C*T*mUmCmCmAmA |
| 32 | mCmAmAmGmG*G*C*A*C*A*G*A*C*T*T*mCmCmAmAmA |
| 33 | G*SG*SG*SC*SA*SC*SA*SA*SG*SG*SG*SC*SA*SC*RA*SG*SA*SC*ST*ST |
| 34 | G*SG*SC*SA*SC*SA*SA*SG*SG*SG*SC*SA*SC*RA*SG*SA*SC*ST*ST*SC |
| 35 | G*SC*SA*SC*SA*SA*SG*SG*SG*SC*SA*SC*RA*SG*SA*SC*ST*ST*SC*SC |
| 36 | C*SA*SC*SA*SA*SG*SG*SG*SC*SA*SC*RA*SG*SA*SC*ST*ST*SC*SC*SA |
| 37 | A*SC*SA*SA*SG*SG*SG*SC*SA*SC*RA*SG*SA*SC*ST*ST*SC*SC*SA*SA |
| 38 | C*SA*SA*SG*SG*SG*SC*SA*SC*RA*SG*SA*SC*ST*ST*SC*SC*SA*SA*SA |
| 39 | mG*SmG*SmC*SmA*SmC*SA*SA*SG*SG*SG*SC*SA*SC*RA*SG*SmA*SmC*S |
| | mU*SmU*SmC |
| 40 | mG*RmG*RmC*RmA*RmC*SA*SA*SG*SG*SG*SC*SA*SC*RA*SG*SmA*RmC*R |
| | mU*RmU*RmC |
| 41 | mGmGmCmAmC*SA*SA*SG*SG*SG*SC*SA*SC*RA*SG*SmAmCmUmUmC |
| 42 | mG*SmG*SmC*SmA*SmC*SmA*SmA*SmG*SG*SG*SC*SA*SC*RA*SG*SA*SC* |
| | st*st*sc |
| 43 | mG*RmG*RmC*RmA*RmU*RmA*RmA*RmG*SG*SG*SC*SA*SC*RA*SG*SA*SC |
| | *st*st*sc |
| 44 | mGmGmCmAmCmAmAmG*SG*SG*SC*SA*SC*RA*SG*SA*SC*ST*ST*SC |
| 45 | mG*RmGmCmAmC*SA*SA*SG*SG*SG*SC*SA*SC*RA*SG*SmAmCmUmU*RmC |
| 46 | mG*SmGmCmAmC*SA*SA*SG*SG*SG*SC*SA*SC*RA*SG*SmAmCmUmU*SmC |
| 47 | G*SC*SA*SG*SG*SG*SC*SA*SC*SA*SA*SG*SG*SG*SC*SA*SC*RA*SG*SA |
| 48 | C*SA*SG*SG*SG*SC*SA*SC*SA*SA*SG*SG*SG*SC*SA*SC*RA*SG*SA*SC |
| 49 | A*SG*SG*SG*SC*SA*SC*SA*SA*SG*SG*SG*SC*SA*SC*RA*SG*SA*SC*ST |
| 50 | A*SA*SG*SG*SG*SC*SA*SC*RA*SG*SA*SC*ST*ST*SC*SC*SA*SA*SA*SG |
| 51 | A*SG*SG*SG*SC*SA*SC*RA*SG*SA*SC*ST*ST*SC*SC*SA*SA*SA*SG*SG |
| 52 | G*SG*SG*SC*SA*SC*RA*SG*SA*SC*ST*ST*SC*SC*SA*SA*SA*SG*SG*SC |
| 53 | mG*mG*mC*mA*mC*A*A*G*G*G*C*A*C*A*G*mA*mC*mU*BrdU*mC |
| 54 | mG*mG*mC*mA*mC*A*A*G*G*G*C*A*C*A*G*mA*mC*BrdU*BrdU*mC |
| 55 | G*G*G*C*A*C*A*A*G*G*G*C*d2AP*C*A*G*A*C*T*T |
| 56 | G*G*C*A*C*A*A*G*G*G*C*d2AP*C*A*G*A*C*T*T*C |
| 57 | G*C*A*C*A*A*G*G*G*C*d2AP*C*A*G*A*C*T*T*C*C |
| 58 | G*G*G*C*A*C*A*A*G*G*G*C*dDAP*C*A*G*A*C*T*T |
| 59 | G*G*C*A*C*A*A*G*G*G*C*dDAP*C*A*G*A*C*T*T*C |
| 60 | G*C*A*C*A*A*G*G*G*C*dDAP*C*A*G*A*C*T*T*C*C |
| 61 | G*SmGmCmAmC*SA*SA*SG*SG*SG*SC*SA*SC*RA*SG*SmAmCmUmU*SC |
| 62 | G*mGmCmAmC*A*A*G*G*G*C*A*C*A*G*mAmCmUmU*C |
| 63 | mG*mGmCmAmC*A*A*G*G*G*C*A*C*A*G*mAmCmUmU*mC |
| 64 | Geo*Geom5CeoAeom5Ceo*A*A*G*G*G*C*A*C*A*G*Aeom5CeoTeoTeo*m5Ceo |
| 65 | g*a*g*c*a*g*c*t*g*c*a*a*c*c*t*g*g*c*a*a |
| 66 | a*g*c*a*g*c*t*g*c*a*a*c*c*t*g*g*c*a*a*c |
| 67 | g*c*a*g*c*t*g*c*a*a*c*c*t*g*g*c*a*a*c*a |
| 68 | C*A*G*C*T*G*C*A*A*C*C*T*G*G*C*A*A*C*A*A |
| 69 | A*G*C*T*G*C*A*A*C*C*T*G*G*C*A*A*C*A*A*C |
| 70 | G*C*T*G*C*A*A*C*C*T*G*G*C*A*A*C*A*A*C*C |
| 71 | mG*mA*mG*mC*mA*G*C*T*G*C*A*A*C*C*T*G*G*C*A*A |
| 72 | mA*mG*mC*mA*mG*C*T*G*C*A*A*C*C*T*G*G*C*A*A*C |
| 73 | mG*mC*mA*mG*mC*T*G*C*A*A*C*C*T*G*G*C*A*A*C*A |
| 74 | mC*mA*mG*mC*mU*G*C*A*A*C*C*T*G*G*C*A*A*C*A*A |
| 75 | mA*mG*mC*mU*mG*C*A*A*C*C*T*G*G*C*A*A*C*A*A*C |
| 76 | mG*mC*mU*mG*mC*A*A*C*C*T*G*G*C*A*A*C*A*A*C*C |
| 77 | mG*mA*mG*mC*mA*G*C*T*G*C*A*A*C*C*T*mG*mG*mC*mA*mA |
| 78 | mA*mG*mC*mA*mG*C*T*G*C*A*A*C*C*T*G*mG*mC*mA*mA*mC |
| 79 | mG*mC*mA*mG*mC*T*G*C*A*A*C*C*T*G*G*mC*mA*mA*mC*mA |
| 80 | mC*mA*mG*mC*mU*G*C*A*A*C*C*T*G*G*C*mA*mA*mC*mA*mA |
| 81 | mA*mG*mC*mU*mG*C*A*A*C*C*T*G*G*C*A*mA*mC*mA*mA*mC |
| 82 | mG*mC*mU*mG*mG*A*A*C*C*T*G*G*C*A*A*mC*mA*mA*mC*mC |
| 83 | mG*mA*mG*mC*mA*mG*mC*T*G*C*A*A*C*C*mU*mG*mG*mC*mA*mA |
| 84 | mGmAmGmCmAmGmC*T*G*C*A*A*C*C*mUmGmGmCmAmA |
| 85 | mA*mG*mC*mA*mG*mC*T*G*C*A*A*C*C*T*G*mG*mC*mA*mA*mC |
| 86 | mAmGmCmAmGmC*T*G*C*A*A*C*C*T*G*mGmCmAmAmC |
| 87 | mG*mG*mA*mG*mC*T*G*C*A*A*C*C*mG*mG*mG*mC*mA*mA*mC*mA |
| 88 | mGmCmAmGmC*T*G*C*A*A*C*C*mUmGmGmCmAmAmCmA |
| 89 | mGmAmGmCmA*G*C*T*G*C*A*A*C*C*T*mGmGmCmAmA |
| 90 | mAmGmCmAmG*C*T*G*C*A*A*C*C*T*G*mGmCmAmAmC |
| 91 | mGmCmAmGmC*T*G*C*A*A*C*C*T*G*G*mCmAmAmCmA |
| 92 | mCmAmGmCmU*G*C*A*A*C*C*T*G*G*C*mAmAmCmAmA |
| 93 | mAmGmCmUmG*C*A*A*C*C*T*G*G*C*A*mAmCmAmAmC |
| 94 | mGmCmUmGmC*A*A*C*C*T*G*G*C*A*A*mCmAmAmCmC |
| 95 | G*SA*SG*SC*SA*SG*SC*ST*SG*SC*SA*RA*SC*SC*ST*SG*SG*SC*SA*SA |
| 96 | A*SG*SC*SA*SG*SC*Sr4SG*SC*SA*RA*SC*SC*ST*SG*SG*SC*SA*SA*SC |
| 97 | G*SC*SA*SG*SC*ST*SG*SC*SA*RA*SC*SC*ST*SG*SG*SC*SA*SA*SC*SA |
| 98 | C*SA*SG*SC*ST*SG*SC*SA*RA*SC*SC*ST*SG*SG*SC*SA*SA*SC*SA*SA |
| 99 | A*SG*SC*ST*SG*SC*SA*RA*SC*SC*ST*SG*SG*SC*SA*SA*SC*SA*SA*SC |
| 100 | G*SC*ST*SG*SC*SA*RA*SC*SC*ST*SG*SG*SC*SA*SA*SC*SA*SA*SC*SC |
| 101 | G*G*G*C*C*A*A*C*A*G*C*C*A*G*C*C*T*G*C*A |
| 102 | G*G*C*C*A*A*C*A*G*C*C*A*G*C*C*T*G*C*A*G |
| 103 | g*c*c*a*a*c*a*g*c*c*a*g*c*c*t*g*t*a*g*g |
| 104 | C*C*A*A*C*A*G*C*C*A*G*C*C*T*G*C*A*G*G*A |
| 105 | C*A*A*C*A*G*C*C*A*G*C*C*T*G*C*A*G*G*A*G |
| 106 | a*a*c*a*g*c*t*a*g*c*c*t*g*c*a*g*g*a*g*g |
| 107 | mG*mG*mG*mC*mC*A*A*C*A*G*C*C*A*G*C*C*T*G*C*A |
| 108 | mG*mG*mC*mC*mA*A*C*A*G*C*C*A*G*C*C*T*G*C*A*G |
| 109 | mG*mC*mC*mA*mA*C*A*G*C*C*A*G*C*C*T*G*C*A*G*G |
| 110 | mC*mC*mA*mA*mC*A*G*C*C*A*G*C*C*T*G*C*A*G*G*A |
| 111 | mC*mA*mA*mC*mA*G*C*C*A*G*C*C*T*G*C*A*G*G*A*G |
| 112 | mA*mA*mC*mA*mG*T*C*A*G*C*C*T*G*C*A*G*G*A*G*G |
| 113 | mG*mG*mG*mC*mC*A*A*C*A*G*C*C*A*G*C*mC*mU*mG*mC*mA |
| 114 | mG*mG*mC*mC*mA*A*C*A*G*C*C*A*G*C*C*mU*mG*mC*mA*mG |
| 115 | mG*mC*mC*mA*mA*C*A*G*C*C*A*G*C*C*T*mG*mU*mA*mG*mG |
| 116 | mC*mC*mA*mA*mC*A*G*C*C*A*G*C*C*T*G*mC*mA*mG*mG*mA |
| 117 | mC*mA*mA*mU*mA*G*C*C*A*G*C*C*T*G*C*mA*mG*mG*mA*mG |
| 118 | mA*mA*mC*mA*mG*C*C*A*G*C*C*T*G*C*A*mG*mG*mA*mG*mG |
| 119 | mG*mG*mG*mC*mC*mA*mA*C*A*G*C*C*A*G*mC*mC*mU*mG*mC*mA |
| 120 | mGmGmGmCmCmAmA*C*A*G*C*C*A*G*mCmCmUmGmCmA |
| 121 | mG*mG*mC*mC*mA*mA*C*A*G*C*C*A*G*C*C*mU*mG*mC*mA*mG |
| 122 | mGmGmCmCmAmA*C*A*G*C*C*A*G*C*C*mUmGmCmAmG |
| 123 | mG*mC*mC*mA*mA*C*A*G*C*C*A*G*mC*mC*mU*mG*mC*mA*mG*mG |
| 124 | mGmCmCmAmA*C*A*G*C*C*A*G*mCmCmUmGmCmAmGmG |
| 125 | mGmGmGmCmC*A*A*C*A*G*C*C*A*G*C*mCmUmGmCmA |
| 126 | mGmGmCmCmA*A*C*A*G*C*C*A*G*C*C*mUmGmCmAmG |
| 127 | mGmCmCmAmA*C*A*G*C*C*A*G*C*C*T*mGmCmAmGmG |
| 128 | mCmCmAmAmC*A*G*C*C*A*G*C*C*T*G*mCmAmGmGmA |
| 129 | mCmAmAmCmA*G*C*C*A*G*C*C*T*G*C*mAmGmGmAmG |
| 130 | mAmAmCmAmG*C*C*A*G*C*C*T*G*C*A*mGmGmAmGmG |
| 131 | G*SG*SG*SC*SC*SA*SA*SC*SA*SG*SC*RC*SA*SG*SC*SC*ST*SG*SC*SA |
| 132 | G*SG*SC*SC*SA*SA*SC*SA*SG*SC*RC*SA*SG*SC*SC*ST*SG*SC*SA*SG |
| 133 | G*SC*SC*SA*SA*SC*SA*SG*SC*RC*SA*SG*SC*SC*ST*SG*SC*SA*SG*SG |
| 134 | C*SC*SA*SA*SC*SA*SG*SC*RC*SA*SG*SC*SC*ST*SG*SC*SA*SG*SG*SA |
| 135 | C*SA*SA*SC*SA*SG*SC*RC*SA*SG*SC*SC*ST*SG*SC*SA*SG*SG*SA*SG |
| 136 | A*SA*SC*SA*SG*SC*RC*SA*SG*SC*SC*ST*SG*SC*SA*SG*SG*SA*SG*SG |
| 137 | A*T*T*A*A*T*A*A*A*T*T*G*T*C*A*T*C*A*C*C |
| 138 | A*ST*ST*SA*SA*ST*SA*SA*SA*ST*ST*SG*ST*SC*RA*ST*SC*SA*SC*SC |
| 139 | A*ST*ST*SA*SA*ST*SA*SA*SA*ST*ST*SG*ST*SC*SA*RT*SC*SA*SC*SC |
| 140 | mA*SmU*SmU*SmA*SmA*SmU*SA*SA*SA*ST*ST*SG*ST*SC*RA*ST*SmC*Sm |
| | A*SmC*SmC |
| 141 | mA*RmU*RmU*RmA*RmA*RmU*SA*SA*SA*ST*ST*SG*ST*SC*RA*ST*SmC*R |
| | mA*RmC*RmC |
| 142 | mA*SmU*SmU*SmA*SmA*SmU*SmA*SmA*SA*ST*ST*SG*ST*SC*RA*ST*SC*S |
| | A*SC*SC |
| 143 | mA*RmU*RmU*RmA*RmA*RmU*RmA*RmA*SA*ST*ST*SG*ST*SC*RA*ST*SC* |
| | SA*SC*SC |
| 144 | mAmUmUmAmAmUmAmA*SA*ST*ST*SG*ST*SC*RA*ST*SC*SA*SC*SC |
| 145 | mAmUmUmAmAmU*SA*SA*SA*ST*ST*SG*ST*SC*RA*ST*SmCmAmCmC |
| 146 | mA*SmUmUmAmAmU*SA*SA*SA*ST*ST*SG*ST*SC*RA*ST*SmCmAmC*SmC |
| 147 | mA*RmUmUmAmAmU*SA*SA*SA*ST*ST*SG*ST*SC*RA*ST*SmCmAmC*RmC |
| 148 | A*SmUmUmAmAmU*SA*SA*SA*ST*ST*SG*ST*SC*RA*ST*SmCmAmC*SC |
| 149 | A*mUmUmAmAmU*A*A*A*T*T*G*T*C*A*T*mCmAmC*C |
| 150 | T*G*T*C*A*T*C*A*C*C*A*G*A*A*A*mA*mA*mG*mU*mC |
| 151 | mU*T*G*T*C*A*T*C*A*C*C*A*G*A*A*mA*mA*mA*mG*mU |
| 152 | T*T*G*T*C*A*T*C*A*C*C*A*G*A*A*mA*mA*mA*mG*mU |
| 153 | mA*mU*T*G*T*C*A*T*C*A*C*C*A*G*A*mA*mA*mA*mA*mG |
| 154 | mA*T*T*G*T*C*A*T*C*A*C*C*A*G*A*mA*mA*mA*mA*mG |
| 155 | mA*mA*mU*T*G*T*C*A*T*C*A*C*C*A*G*mA*mA*mA*mA*mA |
| 156 | mA*mA*T*T*G*T*C*A*T*C*A*C*C*A*G*mA*mA*mA*mA*mA |
| 157 | mA*mA*mA*T*T*G*T*C*A*T*C*A*C*C*A*mG*mA*mA*mA*mA |
| 158 | mA*mA*mA*mU*T*G*T*C*A*T*C*A*C*C*A*mG*mA*mA*mA*mA |
| 159 | mil*mA*mA*mA*mU*T*G*T*C*A*T*C*A*C*C*A*mG*mA*mA*mA |
| 160 | mil*mA*mA*mA*mU*T*G*T*C*A*T*C*A*C*C*mA*mG*mA*mA*mA |
| 161 | mA*mU*mA*mA*mA*T*T*G*T*C*A*T*C*A*C*C*mA*mG*mA*mA |
| 162 | mA*mU*mA*mA*mA*T*T*G*T*C*A*T*C*A*C*mC*mA*mG*mA*mA |
| 163 | mA*mA*mU*mA*mA*A*T*T*G*T*C*A*T*C*A*C*C*mA*mG*mA |
| 164 | mA*mA*mU*mA*mA*A*T*T*G*T*C*A*T*C*A*C*mC*mA*mG*mA |
| 165 | mA*mA*mU*mA*mA*A*T*T*G*T*C*A*T*C*A*mC*mC*mA*mG*mA |
| 166 | mU*mA*mA*mU*mA*A*A*T*T*G*T*C*A*T*C*A*C*C*mA*mG |
| 167 | mU*mA*mA*mU*mA*A*A*T*T*G*T*C*A*T*C*A*C*mC*mA*mG |
| 168 | mU*mA*mA*mU*mA*A*A*T*T*G*T*C*A*T*C*A*mC*mC*mA*mG |
| 169 | mU*mA*mA*mU*mA*A*A*T*T*G*T*C*A*T*C*mA*mC*mC*mA*mG |
| 170 | mU*mU*mA*mA*mU*A*A*A*T*T*G*T*C*A*T*C*A*C*C*mA |
| 171 | mU*mU*mA*mA*mU*A*A*A*T*T*G*T*C*A*T*C*A*C*mC*mA |
| 172 | mU*mU*mA*mA*mU*A*A*A*T*T*G*T*C*A*T*C*A*mC*mC*mA |
| 173 | mU*mU*mA*mA*mU*A*A*A*T*T*G*T*C*A*T*C*mA*mC*mC*mA |
| 174 | mA*mU*mU*mA*mA*T*A*A*A*T*T*G*T*C*A*T*C*A*C*C |
| 175 | mA*mU*mU*mA*mA*T*A*A*A*T*T*G*T*C*A*T*C*A*C*mC |
| 176 | mA*mU*mU*mA*mA*T*A*A*A*T*T*G*T*C*A*T*C*A*mC*mC |
| 177 | mA*mU*mU*mA*mA*T*A*A*A*T*T*G*T*C*A*T*C*mA*mC*mC |
| 178 | mU*mA*mU*mU*mA*A*T*A*A*A*T*T*G*T*C*A*T*C*A*C |
| 179 | mU*mA*mU*mU*mA*A*T*A*A*A*T*T*G*T*C*A*T*C*A*mC |
| 180 | mU*mA*mU*mU*mA*A*T*A*A*A*T*T*G*T*C*A*T*C*mA*mC |
| 181 | mC*mU*mA*mU*mU*A*A*T*A*A*A*T*T*G*T*C*A*T*C*A |
| 182 | mC*mU*mA*mU*mU*A*A*T*A*A*A*T*T*G*T*C*A*T*C*mA |
| 183 | mA*mC*mU*mA*mU*T*A*A*T*A*A*A*T*T*G*T*C*A*T*C |
| 184 | T*G*T*C*A*T*C*A*C*C*A*G*A*A*A*mAmAmGmU*mC |
| 185 | mU*T*G*T*C*A*T*C*A*C*C*A*G*A*A*mAmAmAmG*mU |
| 186 | T*T*G*T*C*A*T*C*A*C*C*A*G*A*A*mAmAmAmG*mU |
| 187 | mA*mU*T*G*T*C*A*T*C*A*C*C*A*G*A*mAmAmAmA*mG |
| 188 | mA*T*T*G*T*C*A*T*C*A*C*C*A*G*A*mAmAmAmA*mG |
| 189 | mA*mAmU*T*G*T*C*A*T*C*A*C*C*A*G*mAmAmAmA*mA |
| 190 | mA*mA*T*T*G*T*C*A*T*C*A*C*C*A*G*mAmAmAmA*mA |
| 191 | mA*mAmA*T*T*G*T*C*A*T*C*A*C*C*A*mGmAmAmA*mA |
| 192 | mA*mAmAmU*T*G*T*C*A*T*C*A*C*C*A*mGmAmAmA*mA |
| 193 | mU*mAmAmAmU*T*G*T*C*A*T*C*A*C*C*A*mGmAmA*mA |
| 194 | mU*mAmAmAmU*T*G*T*C*A*T*C*A*C*C*mAmGmAmA*mA |
| 195 | mA*mUmAmAmA*T*T*G*T*C*A*T*C*A*C*C*mAmGmA*mA |
| 196 | mA*mUmAmAmA*T*T*G*T*C*A*T*C*A*C*mCmAmGmA*mA |
| 197 | mA*mAmUmAmA*A*T*T*G*T*C*A*T*C*A*C*C*mAmG*mA |
| 198 | mA*mAmUmAmA*A*T*T*G*T*C*A*T*C*A*C*mCmAmG*mA |
| 199 | mA*mAmUmAmA*A*T*T*G*T*C*A*T*C*A*mCmCmAmG*mA |
| 200 | mU*mAmAmUmA*A*A*T*T*G*T*C*A*T*C*A*C*C*mA*mG |
| 201 | mU*mAmAmUmA*A*A*T*T*G*T*C*A*T*C*A*C*mCmA*mG |
| 202 | mU*mAmAmUmA*A*A*T*T*G*T*C*A*T*C*A*mCmCmA*mG |
| 203 | mU*mAmAmUmA*A*A*T*T*G*T*C*A*T*C*mAmCmCmA*mG |
| 204 | mU*mUmAmAmU*A*A*A*T*T*G*T*C*A*T*C*A*C*C*mA |
| 205 | mU*mUmAmAmU*A*A*A*T*T*G*T*C*A*T*C*A*C*mC*mA |
| 206 | mU*mUmAmAmU*A*A*A*T*T*G*T*C*A*T*C*A*mCmC*mA |
| 207 | mU*mUmAmAmU*A*A*A*T*T*G*T*C*A*T*C*mAmCmC*mA |
| 208 | mA*mUmUmAmA*T*A*A*A*T*T*G*T*C*A*T*C*A*C*C |
| 209 | mA*mUmUmAmA*T*A*A*A*T*T*G*T*C*A*T*C*A*C*mC |
| 210 | mA*mUmUmAmA*T*A*A*A*T*T*G*T*C*A*T*C*A*mC*mC |
| 211 | mA*mUmUmAmA*T*A*A*A*T*T*G*T*C*A*T*C*mAmC*mC |
| 212 | mU*mAmUmUmA*A*T*A*A*A*T*T*G*T*C*A*T*C*A*C |
| 213 | mU*mAmUmUmA*A*T*A*A*A*T*T*G*T*C*A*T*C*A*mC |
| 214 | mU*mAmUmUmA*A*T*A*A*A*T*T*G*T*C*A*T*C*mA*mC |
| 215 | mC*mUmAmUmU*A*A*T*A*A*A*T*T*G*T*C*A*T*C*A |
| 216 | mC*mUmAmUmU*A*A*T*A*A*A*T*T*G*T*C*A*T*C*mA |
| 217 | mA*mCmUmAmU*T*A*A*T*A*A*A*T*T*G*T*C*A*T*C |
| 218 | G*SG*SG*SC*SA*SC*SA*SA*SG*SG*SG*SC*SA*SC*RA*SG*SA*SC*ST*ST |
| 219 | G*SG*SC*SA*SC*SA*SA*SG*SG*SG*SC*SA*SC*RA*SG*SA*SC*ST*ST*SC |
| 220 | G*SC*SA*SC*SA*SA*SG*SG*SG*SC*SA*SC*RA*SG*SA*SC*ST*ST*SC*SC |
| 221 | C*SA*SC*SA*SA*SG*SG*SG*SC*SA*SC*RA*SG*SA*SC*ST*ST*SC*SC*SA |
| 222 | A*SC*SA*SA*SG*SG*SG*SC*SA*SC*RA*SG*SA*SC*ST*ST*SC*SC*SA*SA |
| 223 | C*SA*SA*SG*SG*SC*SC*SA*SC*RA*SG*SA*SC*ST*ST*SC*SC*SA*SA*SA |
| 224 | mG*SmG*SmC*SmA*SmC*SA*SA*SG*SG*SG*SC*SA*SC*RA*SG*SmA*SmC |
| | *SmU*SmU*SmC |
| 225 | mG*RmG*RmC*RmA*RmC*SA*SA*SG*SG*SG*SC*SA*SC*RA*SG*SmA*RmC*R |
| | mU*RmU*RmC |
| 226 | mGmGmCmAmC*SA*SA*SG*SG*SG*SC*SA*SC*RA*SG*SmAmCmUmUmC |
| 227 | mG*SmG*SmC*SmA*SmC*SmA*SmA*SmG*SG*SG*SC*SA*SC*RA*SG*SA*SC* |
| | ST*ST*SC |
| 228 | mG*RmG*RmC*RmA*RmC*RmA*RmA*RmG*SG*SG*SC*SA*SC*RA*SG*SA*SC |
| | *st*st*sc |
| 229 | mGmGmCmAmCmAmAmG*SG*SG*SC*SA*SC*RA*SG*SA*SC*ST*ST*SC |
| 230 | mG*RmGmCmAmC*SA*SA*SG*SG*SG*SC*SA*SC*RA*SG*SmAmCmUmU*RmC |
| 231 | mG*SmGmCmAmC*SA*SA*SG*SG*SG*SC*SA*SC*RA*SG*SmAmCmUmU*SmC |
| 232 | G*SC*SA*SG*SG*SG*SC*SA*SC*SA*SA*SG*SG*SG*SC*SA*SC*RA*SG*SA |
| 233 | C*SA*SG*SG*SG*SC*SA*SC*SA*SA*SG*SG*SG*SC*SA*SC*RA*SG*SA*SC |
| 234 | A*SG*SG*SG*SC*SA*SC*SA*SA*SG*SG*SG*SC*SA*SC*RA*SG*SA*SC*ST |
| 235 | A*SA*SG*SG*SG*SC*SA*SC*RA*SG*SA*SC*ST*ST*SC*SC*SA*SA*SA*SG |
| 236 | A*SG*SG*SG*SC*SA*SC*RA*SG*SA*SC*ST*ST*SC*SC*SA*SA*SA*SG*SG |
| 237 | G*SG*SG*SC*SA*SC*RA*SG*SA*SC*ST*ST*SC*SC*SA*SA*SA*SG*SG*SC |
| 238 | G*SmGmCmAmC*SA*SA*SG*SG*SG*SC*SA*SC*RA*SG*SmAmCmUmU*SC |
| 239 | G*SG*SG*SC*SC*SA*SA*SC*SA*SG*SC*RC*SA*SG*SC*SC*ST*SG*SC*SA |
| 240 | G*SG*SC*SC*SA*SA*SC*SA*SG*SC*RC*SA*SG*SC*SC*ST*SG*SC*SA*SG |
| 241 | G*SC*SC*SA*SA*SC*SA*SG*SC*RC*SA*SG*SC*SC*ST*SG*SC*SA*SG*SG |
| 242 | C*SC*SA*SA*SC*SA*SG*SC*RC*SA*SG*SC*SC*ST*SG*SC*SA*SG*SG*SA |
| 243 | C*SA*SA*SC*SA*SG*SC*RC*SA*SG*SC*SC*ST*SG*SC*SA*SG*SG*SA*SG |
| 244 | A*SA*SC*SA*SG*SC*RC*SA*SG*SC*SC*ST*SG*SC*SA*SG*SG*SA*SG*SG |
| 245 | G*SA*SG*SC*SA*SG*SC*ST*SG*SC*SA*RA*SC*SC*ST*SG*SG*SC*SA*SA |
| 246 | A*SG*SC*SA*SG*SC*ST*SG*SC*SA*RA*SC*SC*ST*SG*SG*SC*SA*SA*SC |
| 247 | G*SC*SA*SG*SC*ST*SG*SC*SA*RS*SC*SC*ST*SG*SG*SC*SA*SA*SC*SA |
| 248 | C*SA*SG*SC*ST*SG*SC*SA*RA*SC*SC*ST*SG*SG*SC*SA*SA*SC*SA*SA |
| 249 | A*SG*SC*ST*SG*SC*SA*RA*SC*SC*ST*SG*SG*SC*SA*SA*SC*SA*SA*SC |
| 250 | G*SC*ST*SG*SC*SA*RA*SC*SC*ST*SG*SG*SC*SA*SA*SC*SA*SA*SC*SC |
| 251 | A*T*T*A*A*T*A*A*A*T*T*G*T*C*A*T*C*A*C*C |
| 252 | A*ST*ST*SA*SA*ST*SA*SA*SA*ST*ST*SG*ST*SC*RA*ST*SC*SA*SC*SC |
| 253 | A*ST*ST*SA*SA*ST*SA*SA*SA*ST*ST*SG*ST*SC*SA*RT*SC*SA*SC*SC |
| 254 | mA*SmU*SmU*SmA*SmA*SmU*SA*SA*SA*ST*ST*SG*ST*SC*RA*ST*SmC*Sm |
| | A*SmC*SmC |
| 255 | mA*RmU*RmU*RmA*RmA*RmU*SA*SA*SA*ST*ST*SG*ST*SC*RA*ST*SmC*R |
| | mA*RmC*RmC |
| 256 | mA*SmU*SmU*SmA*SmA*SmU*SmA*SmA*SA*ST*ST*SG*ST*SC*RA*ST*SC |
| | *SA*SC*SC |
| 257 | mA*RmU*RmU*RmA*RmA*RmU*RmA*RmA*SA*ST*ST*SG*ST*SC*RA*ST*SC |
| | *SA*SC*SC |
| 258 | mAmUmUmAmAmUmAmA*SA*ST*ST*SG*ST*SC*RA*ST*SC*SA*SC*SC |
| 259 | mAmUmUmAmAmU*SA*SA*SA*ST*ST*SG*ST*SC*RA*ST*SmCmAmCmC |
| 260 | mA*SmUmUmAmAmU*SA*SA*SA*ST*ST*SG*ST*SC*RA*ST*SmCmAmC*SmC |
| 261 | mA*RmUmUmAmAmU*SA*SA*SA*ST*ST*SG*ST*SC*RA*ST*SmCmAmC*RmC |
| 262 | A*SmUmUmAmAmU*SA*SA*SA*ST*ST*SG*ST*SC*RA*ST*SmCmAmC*SC |

In Table 1; “*” represents a stereorandom phosphorothioate linkage; “*S” represents an Sp phosphorothioate linkage; “*R” represents an Rp phosphorothioate linkage; all non-labeled linkage is a natural phosphate linkage; “m” preceding a base represents 2′-OMe; “d2AP” represents a 2-amino purine; “dDAP” represents a 2,6-diamino purine; “eo” following a base represents 2′-MOE; and “BrdU” represents Bromodeoxyuridine.

TABLE 2

| SEQ | |
|---|---|
| ID | |
| NO. | Sequence 5′ -> 3 |
| 263 | m5C*m5C*G*T*m5C*G*m5C*m5C*m5C*T*T*m5C*A*G*m5C*A*m5C*G*m5C*A |
| 264 | m5C*Sm5C*SG*ST*Sm5C*SG*Sm5C*Sm5C*Sm5C*ST*ST*Sm5C*RA*SG*Sm5C*S |
| | A*Sm5C*SG*Sm5C*SA |
| 265 | m5Ceo*m5Ceo*Geo*Teo*m5Ceo*G*m5C*m5C*m5C*T*T*m5C*A*G*m5C*Aeo*m5 |
| | Ceo*Geo*m5Ceo*Aeo |
| 266 | m5Ceo*Sm5Ceo*SGeo*STeo*Sm5Ceo*SG*Sm5C*Sm5C*Sm5C*ST*ST*Sm5C*SA*S |
| | G*Sm5C*SAeo*Sm5Ceo*SGeo*Sm5Ceo*SAeo |
| 267 | m5Ceo*Sm5Ceo*SGeo*STeo*Sm5Ceo*SG*Sm5C*Sm5C*Sm5C*ST*ST*Sm5C*RA*S |
| | G*Sm5C*SAeo*Sm5Ceo*SGeo*Sm5Ceo*SAeo |
| 268 | m5Ceo*Rm5Ceo*RGeo*RTeo*Rm5Ceo*RG*Sm5C*Sm5C*Sm5C*5T*ST*Sm5C*RA* |
| | SG*Sm5C*SAeo*Rm5Ceo*RGeo*Rm5Ceo*RAeo |
| 269 | Geo*Teo*m5Ceo*m5Ceo*m5Ceo*T*G*A*A*G*A*T*G*T*m5C*Aeo*Aeo*Teo*Geo* |
| | m5Ceo |
| 270 | Geo*RTeo*Rm5Ceo*Rm5Ceo*Rm5Ceo*RT*RG*RA*RA*RG*RA*RT*RG*RT*Rm5C |
| | *RAeo*RAeo*RTeo*RGeo*Rm5Ceo |
| 271 | Geo*STeo*Sm5Ceo*Sm5Ceo*Sm5Ceo*ST*SG*SA*SA*SG*SA*ST*SG*ST*Sm5C*S |
| | Aeo*SAeo*STeo*SGeo*Sm5Ceo |
| 272 | Geo*RTeo*Rm5Ceo*Rm5Ceo*Rm5Ceo*RT*SG*SA*SA*SG*SA*ST*SG*ST*Sm5C* |
| | SAeo*RAeo*RTeo*RGeo*Rm5Ceo |
| 273 | Geo*STeo*Sm5Ceo*Sm5Ceo*Sm5Ceo*ST*RG*RA*RA*RG*RA*RT*RG*RT*Rm5C* |
| | RAeo*SAeo*STeo*SGeo*Sm5Ceo |
| 274 | mG*mG*mC*mA*mC*A*A*G*G*G*C*A*C*A*G*mA*mC*mU*mU*mC |
| 275 | mG*SmG*SmC*SmA*SmC*SA*SA*SG*SG*SG*SC*SA*SC*RA*SG*SmA*SmC*Sm |
| | U*SmU*SmC |
| 276 | mG*RmG*RmC*RmA*RmC*SA*SA*SG*SG*SG*SC*SA*SC*RA*SG*SmA*RmC*R |
| | mU*RmU*RmC |
| 277 | mGmGmCmAmC*SA*SA*SG*SG*SG*SC*SA*SC*RA*SG*SmAmCmUmUmC |
| 278 | mG*RmGmCmAmC*SA*SA*SG*SG*SG*SC*SA*SC*RA*SG*SmAmCmUmU*RmC |
| 279 | mG*SmGmCmAmC*SA*SA*SG*SG*SG*SC*SA*SC*RA*SG*SmAmCmUmU*SmC |
| 280 | G*SmGmCmAmC*SA*SA*SG*SG*SG*SC*SA*SC*RA*SG*SmAmCmUmU*SC |
| 281 | G*mGmCmAmC*A*A*G*G*G*C*A*C*A*G*mAmCmUmU*C |
| 282 | mG*mGmCmAmC*A*A*G*G*G*C*A*C*A*G*mAmCmUmU*mC |
| 283 | Geo*Geom5CeoAeom5Ceo*A*A*G*G*G*C*A*C*A*G*Aeom5CeoTeoTeo*m5Ceo |
| 284 | mU*mC*mA*mA*mG*mG*mA*mA*mG*mA*mU*mG*mG*mC*mA*mU*mU*mU* |
| | mC*mU |
| 285 | mU*SmC*SmA*SmA*SmG*SmG*SmA*SmA*SmG*SmA*SmU*SmG*SmG*SmC*Sm |
| | A*SmU*SmU*SmU*SmC*SmU |
| 286 | mU*RmC*RmA*RmA*RmG*RmG*RmA*RmA*RmG*RmA*RmU*RmG*RmG*RmC* |
| | RmA*RmU*RmU*RmU*RmC*RmU |
| 287 | mU*SmC*RmA*SmA*RmG*SmG*RmA*SmA*RmG*SmA*RmU*SmG*RmG*SmC*R |
| | mA*SmU*RmU*SmU*RmC*SmU |
| 288 | mU*RmC*RmA*RmA*SmG*SmG*SmA*SmA*SmG*SmA*SmU*SmG*SmG*SmC*S |
| | mA*SmU*SmU*RmU*RmC*RmU |
| 289 | mU*SmC*SmA*SmA*RmG*RmG*RmA*RmA*RmG*RmA*RmU*RmG*mG*RmC*R |
| | mA*RmU*RmU*SmU*SmC*SmU |
| 290 | mU*RmC*RmA*RmA*RmG*RmG*SmA*SmA*RmG*SmA*SmU*RmG*SmG*SmC*R |
| | mA*RmU*RmU*RmU*RmC*RmU |
| 291 | mU*SmC*SmA*SmA*SmG*SmG*RmA*RmA*SmG*RmA*RmU*SmG*RmG*RmC*S |
| | mA*SmU*SmU*SmU*SmC*SmU |
| 292 | mU*RmC*RmA*RmA*SmG*SmG*RmA*RmA*SmG*RmA*RmU*RmG*SmG*RmC* |
| | RmA*SmU*SmU*RmU*RmC*RmU |
| 293 | mU*SmC*SmA*SmA*RmG*RmG*SmA*SmA*RmG*SmA*SmU*SmG*RmG*SmC*S |
| | mA*RmU*RmU*SmU*SmC*SmU |
| 294 | mU*SmC*RmA*RmA*RmG*RmG*RmA*RmA*RmG*RmA*RmU*RmG*RmG*RmC* |
| | RmA*RmU*RmU*RmU*RmC*SmU |
| 295 | mU*RmC*SmA*SmA*SmG*SmG*SmA*SmA*SmG*SmA*SmU*SmG*SmG*SmC*Sm |
| | A*SmU*SmU*SmU*SmC*RmU |
| 296 | mU*SmC*RmA*SmA*SmG*RmG*RmA*SmA*SmG*RmA*SmU*SmG*RmG*RmC*R |
| | mA*SmU*SmU*SmU*SmC*RmU |
| 297 | mU*RmC*SmA*RmA*RmG*SmG*SmA*RmA*RmG*SmA*RmU*RmG*SmG*SmC*S |
| | mA*RmU*RmU*RmU*RmC*SmU |
| 298 | mU*SmC*SmA*RmA*RmG*RmG*RmA*RmA*RmG*RmA*RmU*SmG*RmG*RmC* |
| | SmA*RmU*SmU*SmU*SmC*SmU |
| 299 | mU*RmC*RmA*SmA*SmG*SmG*SmA*SmA*SmG*SmA*SmU*RmG*SmG*SmC*R |
| | mA*SmU*RmU*RmU*RmC*RmU |
| 300 | T*C*A*A*G*G*A*A*G*A*T*G*G*C*A*T*T*T*C*T |
| 301 | mUmCmAmAmGmGmAmAmGmAmUmGmGmCmAmUmUmUmCmU |
| 302 | T*RC*RA*RA*RG*RG*RA*RA*RG*RA*RT*RG*RG*RC*RA*RT*RT*RT*RC*RT |
| 303 | T*SC*SA*SA*SG*SG*SA*SA*SG*SA*ST*SG*SG*SC*SA*ST*ST*ST*SC*ST |
| 304 | T*SC*SA*SA*SG*SmGmAmAmGmAmUmGmGmCA*ST*ST*ST*SC*ST |
| 305 | mUmCmAmAG*SG*SA*SmAG*SA*ST*SG*SmGC*SA*ST*SmUmUmCmU |
| 306 | T*SmCA*SmAG*SmGA*SmAG*SmAT*SmGG*SmCA*SmUT*SmUC*SmU |
| 307 | mUC*SmAA*SmGG*SmAA*SmGA*SmUG*SmGC*SmAT*SmUT*SmCmU |
| 308 | T*SC*SmAmAG*SG*SmAmAG*SA*ST*SmGmGC*SA*SmUmUT*SC*SmU |
| 309 | T*SC*SA*SmAmGmGA*SA*SmGmAmUG*SG*SmCmAmUT*ST*SC*SmU |
| 310 | T*SC*SA*SA*SmGmGmAmAG*SA*ST*SmGmGmCmAT*ST*ST*SC*SmU |
| 311 | T*SC*SA*SmAG*SG*SA*SmAG*SA*ST*SmGG*SC*SA*SmUT*ST*SC*SmU |
| 312 | mUmCmAmAG*SG*SA*SA*SG*SmAmUmGmGmCA*ST*ST*ST*SC*SmU |
| 313 | T*SC*SmAmAmGmGmAmAmGmAT*SmGmGC*SmAT*ST*ST*SC*SmU |
| 314 | T*C*A*A*G*mGmAmAmGmAmUmGmGmCA*T*T*T*C*T |
| 315 | mUmCmAmAG*G*A*mAG*A*T*G*mGC*A*T*mUmUmCmU |
| 316 | T*mCA*mAG*mGA*mAG*mAT*mGG*mCA*mUT*mUC*mU |
| 317 | mUC*mAA*mGG*mAA*mGA*mUG*mGC*mAT*mUT*mCmU |
| 318 | T*C*mAmAG*G*mAmAG*A*T*mGmGC*A*mUmUT*C*mU |
| 319 | T*C*A*mAmGmGA*A*mGmAmUG*G*mCmAmUT*T*C*mU |
| 320 | T*C*A*A*mGmGmAmAG*A*T*mGmGmCmAT*T*T*C*mU |
| 321 | T*C*A*mAG*G*A*mAG*A*T*mGG*C*A*mUT*T*C*mU |
| 322 | mUmCmAmAG*G*A*A*G*mAmUmGmGmCA*T*T*T*C*mU |
| 323 | T*C*mAmAmGmGmAmAmGmAT*mGmGC*mAT*T*T*C*mU |
| 324 | fU*fC*fA*fA*fG*fG*fA*fA*fG*fA*fU*fG*fG*fC*fA*fU*fU*fU*fC*fU |
| 325 | fU*fC*mA*mA*mG*mG*mA*mA*mG*mA*fU*mG*mG*fC*mA*fU*fU*fU*fC*fU |
| 326 | mU*mC*fA*fA*fG*fG*A*A*fG*fA*mU*fG*fG*mC*fA*mU*mU*mU*mC*mU |
| 327 | mU*fC*mA*fA*mG*fG*mA*fA*mG*fA*mU*fG*mG*fC*mA*fU*mU*fU*mC*fU |
| 328 | mU*mC*mA*mA*mG*mG*fA*fA*fG*fA*fU*fG*fG*fC*mA*mU*mU*mU*mC*mU |
| 329 | fU*fC*fA*fA*fG*fG*mA*mA*mG*mA*mU*mG*mG*mC*fA*fU*fU*fU*fC*fU |
| 330 | mU*fC*mA*mA*fG*fG*mA*mA*fG*mA*mU*fG*fG*fC*mA*mU*mU*mU*fC*mU |
| 331 | fU*mC*fA*fA*mG*mG*fA*fA*mG*fA*fU*mG*mG*mC*fA*fU*fU*fU*mC*fU |
| 332 | mUmCmAmAmGmG*SmA*SmA*RmG*SmA*SmU*RmG*SmG*SmC*RmAmUmUm |
| | UmCmU |
| 333 | mUmCmAmAmGmG*RmA*RmA*SmG*RmA*RmU*SmG*RmG*RmC*SmAmUmUm |
| | UmCmU |
| 334 | mU*SmCmAmAmGmG*SmA*SmA*RmG*SmA*SmU*RmG*SmG*SmC*RmAmUmU |
| | mUmC*SmU |
| 335 | mU*RmCmAmAmGmG*RmA*RmA*SmG*RmA*RmU*SmG*RmG*RmC*SmAmUm |
| | UmUmC*RmU |
| 336 | mU*RmCmAmAmGmG*SmA*SmA*RmG*SmA*SmU*RmG*SmG*SmC*RmAmUmU |
| | mUmC*RmU |
| 337 | mU*SmCmAmAmGmG*RmA*RmA*SmG*RmA*RmU*SmG*RmG*RmC*SmAmUmU |
| | mUmC*SmU |
| 338 | mU*SmC*SmA*SmA*SmG*SmG*SmA*SmA*RmG*SmA*SmU*RmG*SmG*SmC*R |
| | mA*SmU*SmU*SmU*SmC*SmU |
| 339 | mU*SmC*RmA*SmA*SmG*RmG*SmA*SmA*RmG*SmA*SmU*RmG*SmG*SmC*R |
| | mA*SmU*SmU*RmU*SmC*SmU |
| 340 | mU*RmC*SmA*RmA*RmG*SmG*RmA*RmA*SmG*RmA*RmU*SmG*RmG*RmC* |
| | SmA*RmU*RmU*SmU*RmC*RmU |
| 341 | mUmCmAmAmGmG*SmA*SmAmG*SmA*SmUmG*SmG*SmCmAmUmUmUmCmU |
| 342 | mU*SmCmAmAmGmG*SmA*SmAmG*SmA*SmUmG*SmG*SmCmAmUmUmUmC* |
| | SmU |
| 343 | mU*SmCmAmAmGmGmAmAmGmAmUmGmGmCmAmUmUmUmC*SmU |
| 344 | mU*SmC*SmAmAmGmGmAmAmGmAmU*SmGmGmC*SmAmU*SmU*SmU*SmC* |
| | SmU |
| 345 | mU*SmC*SmA*SmA*SmG*SmGmAmAmGmAmUmGmGmCmA*SmU*SmU*SmU*S |
| | mC*SmU |
| 346 | mU*RmC*RmA*RmA*RmG*RmGmAmAmGmAmUmGmGmCmA*RmU*RmU*RmU |
| | *RmC*RmU |
| 347 | mU*SmC*SmA*SmA*SmG*SmG*RmA*RmA*RmG*RmA*RmU*RmG*RmG*RmC* |
| | RA*SmU*SmU*SmU*SmC*SmU |
| 348 | mU*RmC*RmA*RmA*RmG*RmG*SmA*SmA*SmG*SmA*SmU*SmG*SmG*SmC*S |
| | mA*RmU*RmU*RmU*RmC*RmU |
| 349 | mU*SmC*SmA*SmA*SmG*SmG*SmA*SmA*SmG*SmA*SmU*RmG*RmG*RmC*R |
| | A*RmU*RmU*RmU*RmC*RmU |
| 350 | mU*RmC*RmA*RmA*RmG*RmG*RmA*RmA*RmG*RmA*RmU*SmG*SmG*SmC* |
| | SmA*SmU*SmU*SmU*SmC*SmU |
| 351 | mU*RmC*RmA*RmA*RmG*SmG*SmA*RmA*SmG*SmA*RmU*SmG*SmG*RmC*R |
| | mA*RmU*RmU*RmU*RmC*RmU |
| 352 | mU*RmC*RmA*RmA*RmG*RmG*RmA*SmA*SmG*RmA*SmU*SmG*RmG*SmC*S |
| | mA*RmU*RmU*RmU*RmC*RmU |
| 353 | fU*SfC*SA*SfA*SfG*SfG*SA*SfA*SfG*SfA*SfU*SfG*SfG*SfC*SfA*SfU*SfU*Sf |
| | U*SfC*SfU |
| 354 | fU*RfC*RfA*RfA*RfG*RfG*RfA*RfA*RfG*RfA*RfU*RfG*RfG*RfC*RfA*RfU*RfU |
| | *RfU*RfC*RfU |
| 355 | fU*RfC*RfA*RfA*RfG*RfG*SfA*SfA*RfG*SfA*SfU*RfG*SfG*SfC*RfA*RfU*RfU* |
| | RfU*RfC*RfU |
| 356 | fU*SfC*RfA*RfA*RfG*RfG*RfA*RfA*RfG*RfA*RfU*RfG*RfG*RfC*RfA*RfU*RfU |
| | *RfU*RfC*SfU |
| 357 | fU*SfC*SfA*RfA*RfG*RfG*RfA*RfA*RfG*RfA*RfU*SfG*RfG*RfC*SfA*RfU*SfU* |
| | SfU*SfC*SfU |
| 358 | fU*SfC*SmA*SmA*SmG*SmG*SmA*SmA*SmG*SmA*SfU*SmG*SmG*SfC*SmA*S |
| | fU*SfU*SfU*SfC*SfU |
| 359 | fU*RfC*RmA*RmA*RmG*RmG*RmA*RmA*RmG*RmA*RfU*RmG*RmG*RfC*Rm |
| | A*RfU*RfU*RfU*RfC*RfU |
| 360 | fU*RfC*RmA*RmA*RmG*RmG*SmA*SmA*RmG*SmA*SfU*RmG*SmG*SfC*RmA |
| | *RfU*RfU*RfU*RfC*RfU |
| 361 | fU*SfC*RmA*RmA*RmG*RmG*RmA*RmA*RmG*RmA*RfU*RmG*RmG*RfC*Rm |
| | A*RfU*RfU*RfU*RfC*SfU |
| 362 | fU*SfC*SmA*RmA*RmG*RmG*RmA*RmA*RmG*RmA*RfU*SmG*RmG*RfC*SmA |
| | *RfU*SfU*SfU*SfC*SfU |
| 363 | mU*SmC*SfA*SfA*SfG*SfG*SfA*SfA*SfG*SfA*SmU*SfG*SfG*SmC*SfA*SmU*S |
| | mU*SmU*SmC*SmU |
| 364 | mU*RmC*RfA*RfA*RfG*RfG*RfA*RfA*RfG*RfA*RmU*RfG*RfG*RmC*RfA*RmU |
| | *RmU*RmU*RmC*RmU |
| 365 | mU*RmC*RfA*RfA*RfG*RfG*SfA*SfA*RfG*SfA*SmU*RfG*SfG*SmC*RfA*RmU* |
| | RmU*RmU*RmC*RmU |
| 366 | mU*SmC*RfA*RfA*RfG*RfG*RfA*RfA*RfG*RfA*RmU*RfG*RfG*RmC*RfA*RmU |
| | *RmU*RmU*RmC*SmU |
| 367 | mU*SmC*SfA*RfA*RfG*RfG*RfA*RfA*RfG*RfA*RmU*SfG*RfG*RmC*SfA*RmU* |
| | SmU*SmU*SmC*SmU |
| 368 | mU*SfC*SmA*SfA*SmG*SfG*SmA*SfA*SmG*SfA*SmU*SfG*SmG*SfC*SmA*SfU |
| | *SmU*SfU*SmC*SfU |
| 369 | mU*RfC*RmA*RfA*RmG*RfG*RmA*RfA*RmG*RfA*RmU*RfG*RmG*RfC*RmA* |
| | RfU*RmU*RfU*RmC*RfU |
| 370 | mU*RfC*RmA*RfA*RmG*RfG*SmA*SfA*RmG*SfA*SmU*RfG*SmG*SfC*RmA*Rf |
| | U*RmU*RfU*RmC*RfU |
| 371 | mU*SfC*RmA*RfA*RmG*RfG*RmA*RfA*RmG*RfA*RmU*RfG*RmG*RfC*RmA* |
| | RfU*RmU*RfU*RmC*SfU |
| 372 | mU*SfC*SmA*RfA*RmG*RfG*RmA*RfA*RmG*RfA*RmU*SfG*RmG*RfC*SmA*R |
| | fU*SmU*SfU*SmC*SfU |
| 373 | mU*SmC*SmA*SmA*SmG*SmG*SfA*SfA*SfG*SfA*SfU*SfG*SfG*SfC*SmA*SmU |
| | *SmU*SmU*SmC*SmU |
| 374 | mU*RmC*RmA*RmA*RmG*RmG*RfA*RfA*RfG*RfA*RfU*RfG*RfG*RfC*RmA*R |
| | mU*RmU*RmU*RmC*RmU |
| 375 | mU*RmC*RmA*RmA*RmG*RmG*SfA*SfA*RfG*SfA*SfU*RfG*SfG*SfC*RmA*Rm |
| | U*RmU*RmU*RmC*RmU |
| 376 | mU*SmC*RmA*RmA*RmG*RmG*RfA*RfA*RfG*RfA*RfU*RfG*RfG*RfC*RmA*R |
| | mU*RmU*RmU*RmC*SmU |
| 377 | mU*SmC*SmA*RmA*RmG*RmG*RfA*RfA*RfG*RfA*RfU*SfG*RfG*RfC*SmA*R |
| | mU*SmU*SmU*SmC*SmU |
| 378 | fU*SfC*SfA*SfA*SfG*SfG*SmA*SmA*SmG*SmA*SmU*SmG*SmG*SmC*SfA*SfU |
| | *SfU*SfU*SfC*SfU |
| 379 | fU*RfC*RfA*RfA*RfG*RfG*RmA*RmA*RmG*RmA*RmU*RmG*RmG*RmC*RfA* |
| | RfU*RfU*RfU*RfC*RfU |
| 380 | fU*RfC*RfA*RfA*RfG*RfG*SmA*SmA*RmG*SmA*SmU*RmG*SmG*SmC*RfA*Rf |
| | U*RfU*RfU*RfC*RfU |
| 381 | fU*SfC*RfA*RfA*RfG*RfG*RmA*RmA*RmG*RmA*RmU*RmG*RmG*RmC*RfA* |
| | RfU*RfU*RfU*RfC*SfU |
| 382 | fU*SfC*SfA*RfA*RfG*RfG*RmA*RmA*RmG*RmA*RmU*SmG*RmG*RmC*SfA*R |
| | fU*SfU*SfU*SfC*SfU |
| 383 | mU*SfC*SmA*SmA*SfG*SfG*SmA*SmA*SfG*SmA*SmU*SfG*SfG*SfC*SmA*SmU |
| | *SmU*SmU*SfC*SmU |
| 384 | mU*RfC*RmA*RmA*RfG*RfG*RmA*RmA*RfG*RmA*RmU*RfG*RfG*RfC*RmA* |
| | RmU*RmU*RmU*RfC*RmU |
| 385 | mU*RfC*RmA*RmA*RfG*RfG*SmA*SmA*RfG*SmA*SmU*RfG*SfG*SfC*RmA*R |
| | mU*RmU*RmU*RfC*RmU |
| 386 | mU*SfC*RmA*RmA*RfG*RfG*RmA*RmA*RfG*RmA*RmU*RfG*RfG*RfC*RmA* |
| | RmU*RmU*RmU*RfC*SmU |
| 387 | mU*SfC*SmA*RmA*RfG*RfG*RmA*RmA*RfG*RmA*RmU*SfG*RfG*RfC*SmA*R |
| | mU*SmU*SmU*SfC*SmU |
| 388 | fU*SmC*SfA*SfA*SmG*SmG*SfA*SfA*SmG*SfA*SfU*SmG*SmG*SmC*SfA*SfU* |
| | SfU*SfU*SmC*SfU |
| 389 | fU*RmC*RfA*RfA*RmG*RmG*RfA*RfA*RmG*RfA*RfU*RmG*RmG*RmC*RfA*R |
| | fU*RfU*RfU*RmC*RfU |
| 390 | fU*RmC*RfA*RfA*RmG*RmG*SfA*SfA*RmG*SfA*SfU*RmG*SmG*SmC*RfA*Rf |
| | U*RfU*RfU*RmC*RfU |
| 391 | fU*SmC*RfA*RfA*RmG*RmG*RfA*RfA*RmG*RfA*RfU*RmG*RmG*RmC*RfA*Rf |
| | U*RfU*RfU*RmC*SfU |
| 392 | fU*SmC*SfA*RfA*RmG*RmG*RfA*RfA*RmG*RfA*RfU*SmG*RmG*RmC*SfA*Rf |
| | U*SfU*SfU*SmC*SfU |
| 393 | mU*SmC*SmA*SmA*SmG*SmG*SmA*SmA*SmG*SmA*SmU*SmG*SmG*SmC*Sm |
| | A*SmU*SmU*SmU*SmC*SmU |
| 394 | mU*mC*mA*mA*mG*mG*mA*mA*mG*mA*mU*mG*mG*mC*mA*mU*mU*m |
| | U*mC*mU |
| 395 | mG*mG*mC*mC*mA*mA*mA*mC*mC*mU*mC*mG*mG*mC*mU*mU*mA*m |
| | C*mC*mU |
| 396 | mC*mU*mC*mC*mA*mA*mC*mA*mU*mC*mA*mA*mG*mG*mA*mA*mG*m |
| | A*mU*mG*mG*mC*mA*mU*mU*mU*mC*mU*mA*mG |
| 397 | mA*mC*mC*mA*mG*mA*mG*mU*mA*mA*mC*mA*mG*mU*mC*mU*mG*m |
| | A*mG*mU*mA*mG*mG*mA*mG |
| 398 | mC*mA*mC*mC*mA*mG*mA*mG*mU*mA*mA*mC*mA*mG*mU*mC*mU*m |
| | G*mA*mG*mU*mA*mG*mG*mA |
| 399 | mU*mC*mA*mC*mC*mA*mG*mA*mG*mU*mA*mA*mC*mA*mG*mU*mC*m |
| | U*mG*mA*mG*mU*mA*mG*mG |
| 400 | mG*mU*mC*mA*mC*mC*mA*mG*mA*mG*mU*mA*mA*mC*mA*mG*mU*m |
| | C*mU*mG*mA*mG*mU*mA*mG |
| 401 | mG*mU*mU*mG*mU*mG*mU*mC*mA*mC*mC*mA*mG*mA*mG*mU*mA*mA*m |
| | C*mA*mG*mU*mC*mU*mG |
| 402 | mG*mG*mU*mU*mG*mU*mG*mU*mC*mA*mC*mC*mA*mG*mA*mG*mU*mA*m |
| | A*mC*mA*mG*mU*mC*mU |
| 403 | mA*mG*mG*mU*mU*mG*mU*mG*mU*mC*mA*mC*mC*mA*mG*mA*mG*mU*m |
| | A*mA*mC*mA*mG*mU*mC |
| 404 | mC*mA*mG*mG*mU*mU*mG*mU*mG*mU*mC*mA*mC*mC*mA*mG*mA*mG*m |
| | U*mA*mA*mC*mA*mG*mU |
| 405 | mA*mC*mA*mG*mG*mU*mU*mG*mU*mG*mU*mC*mA*mC*mC*mA*mG*mA*m |
| | G*mU*mA*mA*mC*mA*mG |
| 406 | mC*mC*mA*mC*mA*mG*mG*mU*mU*mG*mU*mG*mU*mC*mA*mC*mC*mA*m |
| | G*mA*mG*mU*mA*mA*mC |
| 407 | mA*mC*mC*mA*mC*mA*mG*mG*mU*mU*mG*mU*mG*mU*mC*mA*mC*mC*m |
| | A*mG*mA*mG*mU*mA*mA |
| 408 | mA*mA*mC*mC*mA*mC*mA*mG*mG*mU*mU*mG*mU*mG*mU*mC*mA*mC*m |
| | C*mA*mG*mA*mG*mU*mA |
| 409 | mU*mA*mA*mC*mC*mA*mC*mA*mG*mG*mU*mU*mG*mU*mG*mU*mC*mA*m |
| | C*mC*mA*mG*mA*mG*mU |
| 410 | mG*mU*mA*mA*mC*mC*mA*mC*mA*mG*mG*mU*mU*mG*mU*mG*mU*mC*m |
| | A*mC*mC*mA*mG*mA*mG |
| 411 | mA*mG*mU*mA*mA*mC*mC*mA*mC*mA*mG*mG*mU*mU*mG*mU*mG*mU*m |
| | C*mA*mC*mC*mA*mG*mA |
| 412 | mU*mA*mG*mU*mA*mA*mC*mC*mA*mC*mA*mG*mG*mU*mU*mG*mU*mG*m |
| | U*mC*mA*mC*mC*mA*mG |
| 413 | mU*mU*mA*mG*mU*mA*mA*mC*mC*mA*mC*mA*mG*mG*mU*mU*mG*mU*m |
| | G*mU*mC*mA*mC*mC*mA |
| 414 | mC*mU*mU*mA*mG*mU*mA*mA*mC*mC*mA*mC*mA*mG*mG*mU*mU*mG*m |
| | U*mG*mU*mC*mA*mC*mC |
| 415 | mC*mC*mU*mU*mA*mG*mU*mA*mA*mC*mC*mA*mC*mA*mG*mG*mU*mU*m |
| | G*mU*mG*mU*mC*mA*mC |
| 416 | mU*mC*mC*mU*mU*mA*mG*mU*mA*mA*mC*mC*mA*mC*mA*mG*mG*mU*m |
| | U*mG*mU*mG*mU*mC*mA |
| 417 | mG*mU*mU*mU*mC*mC*mU*mU*mA*mG*mU*mA*mA*mC*mC*mA*mC*mA*m |
| | G*mG*mU*mU*mG*mU*mG |
| 418 | mA*mG*mU*mU*mU*mC*mC*mU*mU*mA*mG*mU*mA*mA*mC*mC*mA*mC*m |
| | A*mG*mG*mU*mU*mG*mU |
| 419 | mC*mA*mG*mU*mU*mU*mC*mC*mU*mU*mA*mG*mU*mA*mA*mC*mC*mA*m |
| | C*mA*mG*mG*mU*mU*mG |
| 420 | mG*mC*mA*mG*mU*mU*mU*mC*mC*mU*mU*mA*mG*mU*mA*mA*mC*mC*m |
| | A*mC*mA*mG*mG*mU*mU |
| 421 | mG*mG*mC*mA*mG*mU*mU*mU*mC*mC*mU*mU*mA*mG*mU*mA*mA*mC*m |
| | C*mA*mC*mA*mG*mG*mU |
| 422 | mU*mG*mG*mC*mA*mG*mU*mU*mU*mC*mC*mU*mU*mA*mG*mU*mA*mA*m |
| | C*mC*mA*mC*mA*mG*mG |
| 423 | mA*mU*mG*mG*mC*mA*mG*mU*mU*mU*mC*mC*mU*mU*mA*mG*mU*mA*m |
| | A*mC*mC*mA*mC*mA*mG |
| 424 | mA*mG*mA*mU*mG*mG*mC*mA*mG*mU*mU*mU*mC*mC*mU*mU*mA*mG*m |
| | U*mA*mA*mC*mC*mA*mC |
| 425 | mG*mA*mG*mA*mU*mG*mG*mC*mA*mG*mU*mU*mU*mC*mC*mU*mU*mA*m |
| | G*mU*mA*mA*mC*mC*mA |
| 426 | mG*mG*mA*mG*mA*mU*mG*mG*mC*mA*mG*mU*mU*mU*mC*mC*mU*mU*m |
| | A*mG*mU*mA*mA*mC*mC |
| 427 | mU*mG*mG*mA*mG*mA*mU*mG*mG*mC*mA*mG*mU*mU*mU*mC*mC*mU*m |
| | U*mA*mG*mU*mA*mA*mC |
| 428 | mU*mU*mG*mG*mA*mG*mA*mU*mG*mG*mC*mA*mG*mU*mU*mU*mC*mC*m |
| | U*mU*mA*mG*mU*mA*mA |
| 429 | mU*mU*mU*mG*mG*mA*mG*mA*mU*mG*mG*mC*mA*mG*mU*mU*mU*mC* |
| | mC*mU*mU*mA*mG*mU*mA |
| 430 | mA*mG*mU*mU*mU*mG*mG*mA*mG*mA*mU*mG*mG*mC*mA*mG*mU*mU* |
| | mU*mC*mC*mU*mU*mA*mG |
| 431 | mU*mA*mG*mU*mU*mU*mG*mG*mA*mG*mA*mU*mG*mG*mC*mA*mG*mU* |
| | mU*mU*mC*mC*mU*mU*mA |
| 432 | mC*mU*mA*mG*mU*mU*mU*mG*mG*mA*mG*mA*mU*mG*mG*mC*mA*mG* |
| | mU*mU*mU*mC*mC*mU*mU |
| 433 | mU*mC*mU*mA*mG*mU*mU*mU*mG*mG*mA*mG*mA*mU*mG*mG*mC*mA* |
| | mG*mU*mU*mU*mC*mC*mU |
| 434 | mU*mU*mC*mU*mA*mG*mU*mU*mU*mG*mG*mA*mG*mA*mU*mG*mG*mC* |
| | mA*mG*mU*mU*mU*mC*mC |
| 435 | mC*mA*mU*mU*mU*mC*mU*mA*mG*mU*mU*mU*mG*mG*mA*mG*mA*mU* |
| | mG*mG*mC*mA*mG*mU*mU |
| 436 | mG*mC*mA*mU*mU*mU*mC*mU*mA*mG*mU*mU*mU*mG*mG*mA*mG*mA* |
| | mU*mG*mG*mC*mA*mG*mU |
| 437 | mA*mU*mG*mG*mC*mA*mU*mU*mU*mC*mU*mA*mG*mU*mU*mU*mG*mG* |
| | mA*mG*mA*mU*mG*mG*mC |
| 438 | mG*mA*mA*mG*mA*mU*mG*mG*mC*mA*mU*mU*mU*mC*mU*mA*mG*mU* |
| | mU*mU*mG*mG*mA*mG*mA |
| 439 | mA*mG*mG*mA*mA*mG*mA*mU*mG*mG*mC*mA*mU*mU*mU*mC*mU*mA* |
| | mG*mU*mU*mU*mG*mG*mA |
| 440 | mA*mA*mG*mG*mA*mA*mG*mA*mU*mG*mG*mC*mA*mU*mU*mU*mC*mU* |
| | mA*mG*mU*mU*mU*mG*mG |
| 441 | mC*mA*mA*mG*mG*mA*mA*mG*mA*mU*mG*mG*mC*mA*mU*mU*mU*mC*m |
| | U*mA*mG*mU*mU*mU*mG |
| 442 | mC*mA*mU*mC*mA*mA*mG*mG*mA*mA*mG*mA*mU*mG*mG*mC*mA*mU*m |
| | U*mU*mC*mU*mA*mG*mU |
| 443 | mA*mC*mA*mU*mC*mA*mA*mG*mG*mA*mA*mG*mA*mU*mG*mG*mC*mA*m |
| | U*mU*mU*mC*mU*mA*mG |
| 444 | mA*mA*mC*mA*mU*mC*mA*mA*mG*mG*mA*mA*mG*mA*mU*mG*mG*mC*m |
| | A*mU*mU*mU*mC*mU*mA |
| 445 | mC*mA*mA*mC*mA*mU*mC*mA*mA*mG*mG*mA*mA*mG*mA*mU*mG*mG*m |
| | C*mA*mU*mU*mU*mC*mU |
| 446 | mC*mU*mC*mC*mA*mA*mC*mA*mU*mC*mA*mA*mG*mG*mA*mA*mG*mA*m |
| | U*mG*mG*mC*mA*mU*mU |
| 447 | mA*mC*mC*mU*mC*mC*mA*mA*mC*mA*mU*mC*mA*mA*mG*mG*mA*mA*m |
| | G*mA*mU*mG*mG*mC*mA |
| 448 | mG*mU*mA*mC*mC*mU*mC*mC*mA*mA*mC*mA*mU*mC*mA*mA*mG*mG*m |
| | A*mA*mG*mA*mU*mG*mG |
| 449 | mA*mG*mG*mU*mA*mC*mC*mU*mC*mC*mA*mA*mC*mA*mU*mC*mA*mA*m |
| | G*mG*mA*mA*mG*mA*mU |
| 450 | mA*mG*mA*mG*mC*mA*mG*mG*mU*mA*mC*mC*mU*mC*mC*mA*mA*mC*m |
| | A*mU*mC*mA*mA*mG*mG |
| 451 | mC*mA*mG*mA*mG*mC*mA*mG*mG*mU*mA*mC*mC*mU*mC*mC*mA*mA*m |
| | C*mA*mU*mC*mA*mA*mG |
| 452 | mC*mU*mG*mC*mC*mA*mG*mA*mG*mC*mA*mG*mG*mU*mA*mC*mC*mU*m |
| | C*mC*mA*mA*mC*mA*mU |
| 453 | mU*mC*mU*mG*mC*mC*mA*mG*mA*mG*mC*mA*mG*mG*mU*mA*mC*mC*m |
| | U*mC*mC*mA*mA*mC*mA |
| 454 | mA*mU*mC*mU*mG*mC*mC*mA*mG*mA*mG*mC*mA*mG*mG*mU*mA*mC*m |
| | C*mU*mC*mC*mA*mA*mC |
| 455 | mA*mA*mU*mC*mU*mG*mC*mC*mA*mG*mA*mG*mC*mA*mG*mG*mU*mA*m |
| | C*mC*mU*mC*mC*mA*mA |
| 456 | mA*mA*mA*mU*mC*mU*mG*mC*mC*mA*mG*mA*mG*mC*mA*mG*mG*mU*m |
| | A*mC*mC*mU*mC*mC*mA |
| 457 | mG*mA*mA*mA*mU*mC*mU*mG*mC*mC*mA*mG*mA*mG*mC*mA*mG*mG*m |
| | U*mA*mC*mC*mU*mC*mC |
| 458 | mU*mG*mA*mA*mA*mU*mC*mU*mG*mC*mC*mA*mG*mA*mG*mC*mA*mG*m |
| | G*mU*mA*mC*mC*mU*mC |
| 459 | mU*mU*mG*mA*mA*mA*mU*mC*mU*mG*mC*mC*mA*mG*mA*mG*mC*mA*m |
| | G*mG*mU*mA*mC*mC*mU |
| 460 | mC*mC*mC*mG*mG*mU*mU*mG*mA*mA*mA*mU*mC*mU*mG*mC*mC*mA*m |
| | G*mA*mG*mC*mA*mG*mG |
| 461 | mC*mC*mA*mA*mG*mC*mC*mC*mG*mG*mU*mU*mG*mA*mA*mA*mU*mC*m |
| | U*mG*mC*mC*mA*mG*mA |
| 462 | mU*mC*mC*mA*mA*mG*mC*mC*mC*mG*mG*mU*mU*mG*mA*mA*mA*mU*m |
| | C*mU*mG*mC*mC*mA*mG |
| 463 | mG*mU*mC*mC*mA*mA*mG*mC*mC*mC*mG*mG*mU*mU*mG*mA*mA*mA*m |
| | U*mC*mU*mG*mC*mC*mA |
| 464 | mU*mC*mU*mG*mU*mC*mC*mA*mA*mG*mC*mC*mC*mG*mG*mU*mU*mG*m |
| | A*mA*mA*mU*mC*mU*mG |
| 465 | mU*mU*mC*mU*mG*mU*mC*mC*mA*mA*mG*mC*mC*mC*mG*mG*mU*mU*m |
| | G*mA*mA*mA*mU*mC*mU |
| 466 | mG*mU*mU*mC*mU*mG*mU*mC*mC*mA*mA*mG*mC*mC*mC*mG*mG*mU*m |
| | U*mG*mA*mA*mA*mU*mC |
| 467 | mA*mG*mU*mU*mC*mU*mG*mU*mC*mC*mA*mA*mG*mC*mC*mC*mG*mG*m |
| | U*mU*mG*mA*mA*mA*mU |
| 468 | mA*mA*mG*mU*mU*mC*mU*mG*mU*mC*mC*mA*mA*mG*mC*mC*mC*mG*m |
| | G*mU*mU*mG*mA*mA*mA |
| 469 | mU*mA*mA*mG*mU*mU*mC*mU*mG*mU*mC*mC*mA*mA*mG*mC*mC*mC*m |
| | G*mG*mU*mU*mG*mA*mA |
| 470 | mG*mU*mA*mA*mG*mU*mU*mC*mU*mG*mU*mC*mC*mA*mA*mG*mC*mC*m |
| | C*mG*mG*mU*mU*mG*mA |
| 471 | mG*mG*mU*mA*mA*mG*mU*mU*mC*mU*mG*mU*mC*mC*mA*mA*mG*mC*m |
| | C*mC*mG*mG*mU*mU*mG |
| 472 | mC*mG*mG*mU*mA*mA*mG*mU*mU*mC*mU*mG*mU*mC*mC*mA*mA*mG*m |
| | C*mC*mC*mG*mG*mU*mU |
| 473 | mU*mC*mG*mG*mU*mA*mA*mG*mU*mU*mC*mU*mG*mU*mC*mC*mA*mA |
| | *mG*mC*mC*mC*mG*mG*mU |
| 474 | mG*mU*mC*mG*mG*mU*mA*mA*mG*mU*mU*mC*mU*mG*mU*mC*mC*mA |
| | *mA*mG*mC*mC*mC*mG*mG |
| 475 | mA*mG*mU*mC*mG*mG*mU*mA*mA*mG*mU*mU*mC*mU*mG*mU*mC*mC |
| | *mA*mA*mG*mC*mC*mC*mG |
| 476 | mC*mA*mG*mU*mC*mG*mG*mU*mA*mA*mG*mU*mU*mC*mU*mG*mU*m |
| | C*mC*mA*mA*mG*mC*mC*mC |
| 477 | mA*mA*mA*mG*mC*mC*mA*mG*mU*mC*mG*mG*mU*mA*mA*mG*mU*m |
| | U*mC*mU*mG*mU*mC*mC*mA |
| 478 | mG*mA*mA*mA*mG*mC*mC*mA*mG*mU*mC*mG*mG*mU*mA*mA*mG*m |
| | U*mU*mC*mU*mG*mU*mC*mC |
| 479 | mG*mU*mC*mA*mC*mC*mC*mA*mC*mC*mA*mU*mC*mA*mC*mC*mC*m |
| | U*mC*mU*mG*mU*mG*mA*mU |
| 480 | mG*mG*mU*mC*mA*mC*mC*mC*mA*mC*mC*mA*mU*mC*mA*mC*mC*m |
| | C*mU*mC*mU*mG*mU*mG*mA |
| 481 | mA*mA*mG*mG*mU*mC*mA*mC*mC*mC*mA*mC*mC*mA*mU*mC*mA*m |
| | C*mC*mC*mU*mC*mU*mG*mU |
| 482 | mC*mA*mA*mG*mG*mU*mC*mA*mC*mC*mC*mA*mC*mC*mA*mU*mC*m |
| | A*mC*mC*mC*mU*mC*mU*mG |
| 483 | mU*mC*mA*mA*mG*mG*mU*mC*mA*mC*mC*mC*mA*mC*mC*mA*mU*m |
| | C*mA*mC*mC*mC*mU*mC*mU |
| 484 | mC*mU*mC*mA*mA*mG*mG*mU*mC*mA*mC*mC*mC*mA*mC*mC*mA*m |
| | U*mC*mA*mC*mC*mC*mU*mC |
| 485 | mC*mU*mU*mG*mA*mU*mC*mA*mA*mG*mC*mA*mG*mA*mG*mA*mA*m |
| | A*mG*mC*mC*mA*mG*mU*mC |
| 486 | mA*mU*mA*mA*mC*mU*mU*mG*mA*mU*mC*mA*mA*mG*mC*mA*mG*m |
| | A*mG*mA*mA*mA*mG*mC*mC |
| 487 | mA*mG*mU*mA*mA*mC*mA*mG*mU*mC*mU*mG*mA*mG*mU*mA*mG*m |
| | G*mA*mG |
| 488 | mG*mA*mG*mU*mA*mA*mC*mA*mG*mU*mC*mU*mG*mA*mG*mU*mA*m |
| | G*mG*mA |
| 489 | mA*mG*mA*mG*mU*mA*mA*mC*mA*mG*mU*mC*mU*mG*mA*mG*mU*m |
| | A*mG*mG |
| 490 | mC*mA*mG*mA*mG*mU*mA*mA*mC*mA*mG*mU*mC*mU*mG*mA*mG*m |
| | U*mA*mG |
| 491 | mG*mU*mC*mA*mC*mC*mA*mG*mA*mG*mU*mA*mA*mC*mA*mG*mU*m |
| | C*mU*mG |
| 492 | mU*mG*mU*mC*mA*mC*mC*mA*mG*mA*mG*mU*mA*mA*mC*mA*mG*m |
| | U*mC*mU |
| 493 | mG*mU*mG*mU*mC*mA*mC*mC*mA*mG*mA*mG*mU*mA*mA*mC*mA*m |
| | G*mU*mC |
| 494 | mU*mG*mU*mG*mU*mC*mA*mC*mC*mA*mG*mA*mG*mU*mA*mA*mC*m |
| | A*mG*mU |
| 495 | mU*mU*mG*mU*mG*mU*mC*mA*mC*mC*mA*mG*mA*mG*mU*mA*mA*m |
| | C*mA*mG |
| 496 | mG*mG*mU*mU*mG*mU*mG*mU*mC*mA*mC*mC*mA*mG*mA*mG*mU*m |
| | A*mA*mC |
| 497 | mA*mG*mG*mU*mU*mG*mU*mG*mU*mC*mA*mC*mC*mA*mG*mA*mG*m |
| | U*mA*mA |
| 498 | mC*mA*mG*mG*mU*mU*mG*mU*mG*mU*mC*mA*mC*mC*mA*mG*mA*m |
| | G*mU*mA |
| 499 | mA*mC*mA*mG*mG*mU*mU*mG*mU*mG*mU*mC*mA*mC*mC*mA*mG*m |
| | A*mG*mU |
| 500 | mC*mA*mC*mA*mG*mG*mU*mU*mG*mU*mG*mU*mC*mA*mC*mC*mA*m |
| | G*mA*mG |
| 501 | mC*mC*mA*mC*mA*mG*mG*mU*mU*mG*mU*mG*mU*mC*mA*mC*mC*m |
| | A*mG*mA |
| 502 | mA*mC*mC*mA*mC*mA*mG*mG*mU*mU*mG*mU*mG*mU*mC*mA*mC*m |
| | C*mA*mG |
| 503 | mA*mA*mC*mC*mA*mC*mA*mG*mG*mU*mU*mG*mU*mG*mU*mC*mA*m |
| | C*mC*mA |
| 504 | mU*mA*mA*mC*mC*mA*mC*mA*mG*mG*mU*mU*mG*mU*mG*mU*mC*m |
| | A*mC*mC |
| 505 | mG*mU*mA*mA*mC*mC*mA*mC*mA*mG*mG*mU*mU*mG*mU*mG*mU*m |
| | C*mA*mC |
| 506 | mA*mG*mU*mA*mA*mC*mC*mA*mC*mA*mG*mG*mU*mU*mG*mU*mG*m |
| | U*mC*mA |
| 507 | mC*mU*mU*mA*mG*mU*mA*mA*mC*mC*mA*mC*mA*mG*mG*mU*mU*m |
| | G*mU*mG |
| 508 | mC*mC*mU*mU*mA*mG*mU*mA*mA*mC*mC*mA*mC*mA*mG*mG*mU*m |
| | U*mG*mU |
| 509 | mU*mC*mC*mU*mU*mA*mG*mU*mA*mA*mC*mC*mA*mC*mA*mG*mG*m |
| | U*mU*mG |
| 510 | mU*mU*mC*mC*mU*mU*mA*mG*mU*mA*mA*mC*mC*mA*mC*mA*mG*m |
| | G*mU*mU |
| 511 | mU*mU*mU*mC*mC*mU*mU*mA*mG*mU*mA*mA*mC*mC*mA*mC*mA*m |
| | G*mG*mU |
| 512 | mG*mU*mU*mU*mC*mC*mU*mU*mA*mG*mU*mA*mA*mC*mC*mA*mC*m |
| | A*mG*mG |
| 513 | mA*mG*mU*mU*mU*mC*mC*mU*mU*mA*mG*mU*mA*mA*mC*mC*mA*m |
| | C*mA*mG |
| 514 | mG*mC*mA*mG*mU*mU*mU*mC*mC*mU*mU*mA*mG*mU*mA*mA*mC*m |
| | C*mA*mC |
| 515 | mG*mG*mC*mA*mG*mU*mU*mU*mC*mC*mU*mU*mA*mG*mU*mA*mA*m |
| | C*mC*mA |
| 516 | mU*mG*mG*mC*mA*mG*mU*mU*mU*mC*mC*mU*mU*mA*mG*mU*mA*m |
| | A*mC*mC |
| 517 | mA*mU*mG*mG*mC*mA*mG*mU*mU*mU*mC*mC*mU*mU*mA*mG*mU*m |
| | A*mA*mC |
| 518 | mG*mA*mU*mG*mG*mC*mA*mG*mU*mU*mU*mC*mC*mU*mU*mA*mG*m |
| | U*mA*mA |
| 519 | mA*mG*mA*mU*mG*mG*mC*mA*mG*mU*mU*mU*mC*mC*mU*mU*mA*m |
| | G*mU*mA |
| 520 | mG*mG*mA*mG*mA*mU*mG*mG*mC*mA*mG*mU*mU*mU*mC*mC*mU*m |
| | U*mA*mG |
| 521 | mU*mG*mG*mA*mG*mA*mU*mG*mG*mC*mA*mG*mU*mU*mU*mC*mC*m |
| | U*mU*mA |
| 522 | mU*mU*mG*mG*mA*mG*mA*mU*mG*mG*mC*mA*mG*mU*mU*mU*mC*m |
| | C*mU*mU |
| 523 | mU*mU*mU*mG*mG*mA*mG*mA*mU*mG*mG*mC*mA*mG*mU*mU*mU*m |
| | C*mC*mU |
| 524 | mG*mU*mU*mU*mG*mG*mA*mG*mA*mU*mG*mG*mC*mA*mG*mU*mU*m |
| | U*mC*mC |
| 525 | mC*mU*mA*mG*mU*mU*mU*mG*mG*mA*mG*mA*mU*mG*mG*mC*mA*m |
| | G*mU*mU |
| 526 | mU*mC*mU*mA*mG*mU*mU*mU*mG*mG*mA*mG*mA*mU*mG*mG*mC*m |
| | A*mG*mU |
| 527 | mA*mU*mU*mU*mC*mU*mA*mG*mU*mU*mU*mG*mG*mA*mG*mA*mU*m |
| | G*mG*mC |
| 528 | mU*mG*mG*mC*mA*mU*mU*mU*mC*mU*mA*mG*mU*mU*mU*mG*mG*m |
| | A*mG*mA |
| 529 | mG*mA*mU*mG*mG*mC*mA*mU*mU*mU*mC*mU*mA*mG*mU*mU*mU*m |
| | G*mG*mA |
| 530 | mA*mG*mA*mU*mG*mG*mC*mA*mU*mU*mU*mC*mU*mA*mG*mU*mU*m |
| | U*mG*mG |
| 531 | mA*mA*mG*mA*mU*mG*mG*mC*mA*mU*mU*mU*mC*mU*mA*mG*mU*m |
| | U*mU*mG |
| 532 | mA*mG*mG*mA*mA*mG*mA*mU*mG*mG*mC*mA*mU*mU*mU*mC*mU*m |
| | A*mG*mU |
| 533 | mA*mA*mG*mG*mA*mA*mG*mA*mU*mG*mG*mC*mA*mU*mU*mU*mC*m |
| | U*mA*mG |
| 534 | mC*mA*mA*mG*mG*mA*mA*mG*mA*mU*mG*mG*mC*mA*mU*mU*mU*m |
| | C*mU*mA |
| 535 | mU*mC*mA*mA*mG*mG*mA*mA*mG*mA*mU*mG*mG*mC*mA*mU*mU*m |
| | U*mC*mU |
| 536 | mA*mC*mA*mU*mC*mA*mA*mG*mG*mA*mA*mG*mA*mU*mG*mG*mC*m |
| | A*mU*mU |
| 537 | mC*mA*mA*mC*mA*mU*mC*mA*mA*mG*mG*mA*mA*mG*mA*mU*mG*m |
| | G*mC*mA |
| 538 | mU*mC*mC*mA*mA*mC*mA*mU*mC*mA*mA*mG*mG*mA*mA*mG*mA*m |
| | U*mG*mG |
| 539 | mC*mC*mU*mC*mC*mA*mA*mC*mA*mU*mC*mA*mA*mG*mG*mA*mA*m |
| | G*mA*mU |
| 540 | mA*mG*mG*mU*mA*mC*mC*mU*mC*mC*mA*mA*mC*mA*mU*mC*mA*m |
| | A*mG*mG |
| 541 | mC*mA*mG*mG*mU*mA*mC*mC*mU*mC*mC*mA*mA*mC*mA*mU*mC*m |
| | A*mA*mG |
| 542 | mA*mG*mA*mG*mC*mA*mG*mG*mU*mA*mC*mC*mU*mC*mC*mA*mA*m |
| | C*mA*mU |
| 543 | mC*mA*mG*mA*mG*mC*mA*mG*mG*mU*mA*mC*mC*mU*mC*mC*mA*m |
| | A*mC*mA |
| 544 | mC*mC*mA*mG*mA*mG*mC*mA*mG*mG*mU*mA*mC*mC*mU*mC*mC*m |
| | A*mA*mC |
| 545 | mG*mC*mC*mA*mG*mA*mG*mC*mA*mG*mG*mU*mA*mC*mC*mU*mC*m |
| | C*mA*mA |
| 546 | mU*mG*mC*mC*mA*mG*mA*mG*mC*mA*mG*mG*mU*mA*mC*mC*mU*m |
| | C*mC*mA |
| 547 | mC*mU*mG*mC*mC*mA*mG*mA*mG*mC*mA*mG*mG*mU*mA*mC*mC*m |
| | U*mC*mC |
| 548 | mU*mC*mU*mG*mC*mC*mA*mG*mA*mG*mC*mA*mG*mG*mU*mA*mC*m |
| | C*mU*mC |
| 549 | mA*mU*mC*mU*mG*mC*mC*mA*mG*mA*mG*mC*mA*mG*mG*mU*mA*m |
| | C*mC*mU |
| 550 | mU*mU*mG*mA*mA*mA*mU*mC*mU*mG*mC*mC*mA*mG*mA*mG*mC*m |
| | A*mG*mG |
| 551 | mC*mC*mC*mG*mG*mU*mU*mG*mA*mA*mA*mU*mC*mU*mG*mC*mC*m |
| | A*mG*mA |
| 552 | mG*mC*mC*mC*mG*mG*mU*mU*mG*mA*mA*mA*mU*mC*mU*mG*mC*m |
| | C*mA*mG |
| 553 | mA*mG*mC*mC*mC*mG*mG*mU*mU*mG*mA*mA*mA*mU*mC*mU*mG*m |
| | C*mC*mA |
| 554 | mC*mC*mA*mA*mG*mC*mC*mC*mG*mG*mU*mU*mG*mA*mA*mA*mU*m |
| | C*mU*mG |
| 555 | mU*mC*mC*mA*mA*mG*mC*mC*mC*mG*mG*mU*mU*mG*mA*mA*mA*m |
| | U*mC*mU |
| 556 | mG*mU*mC*mC*mA*mA*mG*mC*mC*mC*mG*mG*mU*mU*mG*mA*mA*m |
| | A*mU*mC |
| 557 | mU*mG*mU*mC*mC*mA*mA*mG*mC*mC*mC*mG*mG*mU*mU*mG*mA*m |
| | A*mA*mU |
| 558 | mC*mU*mG*mU*mC*mC*mA*mA*mG*mC*mC*mC*mG*mG*mU*mU*mG*m |
| | A*mA*mA |
| 559 | mU*mC*mU*mG*mU*mC*mC*mA*mA*mG*mC*mC*mC*mG*mG*mU*mU*m |
| | G*mA*mA |
| 560 | mU*mU*mC*mU*mG*mU*mC*mC*mA*mA*mG*mC*mC*mC*mG*mG*mU*m |
| | U*mG*mA |
| 561 | mG*mU*mU*mC*mU*mG*mU*mC*mC*mA*mA*mG*mC*mC*mC*mG*mG*m |
| | U*mU*mG |
| 562 | mA*mG*mU*mU*mC*mU*mG*mU*mC*mC*mA*mA*mG*mC*mC*mC*mG*m |
| | G*mU*mU |
| 563 | mA*mA*mG*mU*mU*mC*mU*mG*mU*mC*mC*mA*mA*mG*mC*mC*mC*m |
| | G*mG*mU |
| 564 | mU*mA*mA*mG*mU*mU*mC*mU*mG*mU*mC*mC*mA*mA*mG*mC*mC*m |
| | C*mG*mG |
| 565 | mG*mU*mA*mA*mG*mU*mU*mC*mU*mG*mU*mC*mC*mA*mA*mG*mC*m |
| | C*mC*mG |
| 566 | mG*mG*mU*mA*mA*mG*mU*mU*mC*mU*mG*mU*mC*mC*mA*mA*mG*m |
| | C*mC*mC |
| 567 | mC*mA*mG*mU*mC*mG*mG*mU*mA*mA*mG*mU*mU*mC*mU*mG*mU*m |
| | C*mC*mA |
| 568 | mC*mC*mA*mG*mU*mC*mG*mG*mU*mA*mA*mG*mU*mU*mC*mU*mG*m |
| | U*mC*mC |
| 569 | mC*mC*mA*mC*mC*mA*mU*mC*mA*mC*mC*mC*mU*mC*mU*mG*mU*m |
| | G*mA*mU |
| 570 | mC*mC*mC*mA*mC*mC*mA*mU*mC*mA*mC*mC*mC*mU*mC*mU*mG*m |
| | U*mG*mA |
| 571 | mC*mA*mC*mC*mC*mA*mC*mC*mA*mU*mC*mA*mC*mC*mC*mU*mC*m |
| | U*mG*mU |
| 572 | mU*mC*mA*mC*mC*mC*mA*mC*mC*mA*mU*mC*mA*mC*mC*mC*mU*mC* |
| | mU*mG |
| 573 | mG*mU*mC*mA*mC*mC*mC*mA*mC*mC*mA*mU*mC*mA*mC*mC*mC*m |
| | U*mC*mU |
| 574 | mG*mG*mU*mC*mA*mC*mC*mC*mA*mC*mC*mA*mU*mC*mA*mC*mC*m |
| | C*mU*mC |
| 575 | mU*mC*mA*mA*mG*mC*mA*mG*mA*mG*mA*mA*mA*mG*mC*mC*mA*m |
| | G*mU*mC |
| 576 | mU*mU*mG*mA*mU*mC*mA*mA*mG*mC*mA*mG*mA*mG*mA*mA*mA*m |
| | G*mC*mC |
| 577 | mC*mA*mA*mA*mG*mA*mA*mG*mA*mU*mG*mG*mC*mA*mU*mU*mU*m |
| | C*mU*mA*mG*mU*mU*mU*mG |
| 578 | mG*mC*mA*mA*mA*mG*mA*mA*mG*mA*mU*mG*mG*mC*mA*mU*mU*m |
| | U*mC*mU |
| 579 | fG*fC*fA*fA*fA*fG*mA*mA*mG*mA*mU*mG*mG*mC*fA*fU*fU*fU*fC*fU |
| 580 | fU*fC*fA*fA*fG*mG*mA*mA*mG*mA*mU*mG*mG*mC*mA*fU*fU*fU*fC*fU |
| 581 | fU*fC*fA*fA*mG*mG*mA*mA*mG*mA*mU*mG*mG*mC*mA*mU*fU*fU*fC*fU |
| 582 | fU*fC*fA*mA*mG*mG*mA*mA*mG*mA*mU*mG*mG*mC*mA*mU*mU*f |
| | U*fC*fU |
| 583 | fU*fC*mA*mA*mG*mG*mA*mA*mG*mA*mU*mG*mG*mC*mA*mU*mU*m |
| | U*fC*fU |
| 584 | fU*mC*mA*mA*mG*mG*mA*mA*mG*mA*mU*mG*mG*mC*mA*mU*mU*m |
| | U*mC*fU |
| 585 | fU*fC*fA*fA*fG*fGmA*mA*mG*mA*mU*mG*mG*mCfA*fU*fU*fU*fC*fU |
| 586 | fU*fC*fA*fA*fGfGmA*mA*mG*mA*mU*mG*mG*mCfAfU*fU*fU*fC*fU |
| 587 | fU*fC*fA*fAfGfGmA*mA*mG*mA*mU*mG*mG*mCfAfUfU*fU*fC*fU |
| 588 | fU*fC*fAfAfGfGmA*mA*mG*mA*mU*mG*mG*mCfAfUfUfU*fC*fU |
| 589 | fU*fCfAfAfGfGmA*mA*mG*mA*mU*mG*mG*mCfAfUfUfUfC*fU |
| 590 | fUfCfAfAfGfGmA*mA*mG*mA*mU*mG*mG*mCfAfUfUfUfCfU |
| 591 | fU*fC*fA*fA*fG*fG*fA*A*fG*fA*mU*mG*mG*mC*mA*mU*mU*mU*mC*mU |
| 592 | mU*mC*mA*mA*mG*mG*mA*mA*mG*mA*fU*fG*fG*fC*fA*fU*fU*fU*fC*fU |
| 593 | fU*fC*fA*fA*fG*fG*mA*mA*mG*mA*mU*mG*mG*mC*mA*mU*mU*m |
| | U*mC*mU |
| 594 | mU*mC*mA*mA*mG*mG*mA*mA*mG*mA*mU*mG*mG*mC*fA*fU*fU*f |
| | U*fC*fU |
| 595 | mU*RmC*RmA*RmA*RmG*RmG*RmA*RmA*RmG*RmA*RmU*RmG*RmG*R |
| | mC*RmA*RmU*RmU*RmU*RmC*RmU |
| 596 | mU*SmC*RmA*SmA*RmG*SmG*RmA*SmA*RmG*SmA*RmU*SmG*RmG*S |
| | mC*RmA*SmU*RmU*SmU*RmC*SmU |
| 597 | mU*RmC*RmA*RmA*SmG*SmG*SmA*SmA*SmG*SmA*SmU*SmG*SmG*S |
| | mC*SmA*SmU*SmU*RmU*RmC*RmU |
| 598 | mU*SmC*SmA*SmA*RmG*RmG*RmA*RmA*RmG*RmA*RmU*RmG*RmG*RmC* |
| | RmA*RmU*RmU*SmU*SmC*SmU |
| 599 | mU*RmC*RmA*RmA*RmG*RmG*SmA*SmA*RmG*SmA*SmU*RmG*SmG*SmC*R |
| | mA*RmU*RmU*RmU*RmC*RmU |
| 600 | mU*SmC*SmA*SmA*SmG*SmG*RmA*RmA*SmG*RmA*RmU*SmG*RmG*RmC*S |
| | mA*SmU*SmU*SmU*SmC*SmU |
| 601 | mU*RmC*RmA*RmA*SmG*SmG*RmA*RmA*SmG*RmA*RmU*RmG*SmG*RmC* |
| | RmA*SmU*SmU*RmU*RmC*RmU |
| 602 | mU*SmC*SmA*SmA*RmG*RmG*SmA*SmA*RmG*SmA*SmU*SmG*RmG*SmC*S |
| | mA*RmU*RmU*SmU*SmC*SmU |
| 603 | mU*SmC*RmA*RmA*RmG*RmG*RmA*RmA*RmG*RmA*RmU*RmG*RmG*RmC* |
| | RmA*RmU*RmU*RmU*RmC*SmU |
| 604 | mU*RmC*SmA*SmA*SmG*SmG*SmA*SmA*SmG*SmA*SmU*SmG*SmG*SmC*Sm |
| | A*SmU*SmU*SmU*SmC*RmU |
| 605 | mU*SmC*RmA*SmA*SmG*RmG*RmA*SmA*SmG*RmA*SmU*SmG*RmG*RmC*R |
| | mA*SmU*SmU*SmU*SmC*RmU |
| 606 | mU*RmC*SmA*RmA*RmG*SmG*SmA*RmA*RmG*SmA*RmU*RmG*SmG*SmC*S |
| | mA*RmU*RmU*RmU*RmC*SmU |
| 607 | mU*SmC*SmA*RmA*RmG*RmG*RmA*RmA*RmG*RmA*RmU*SmG*RmG*RmC* |
| | SmA*RmU*SmU*SmU*SmC*SmU |
| 608 | mU*RmC*RmA*SmA*SmG*SmG*SmA*SmA*SmG*SmA*SmU*RmG*SmG*SmC*R |
| | mA*SmU*RmU*RmU*RmC*RmU |
| 609 | fG*fG*fC*fC*fA*fA*fA*fC*fC*fU*fC*fG*fG*fC*fU*fU*fA*fC*fC*fU |
| 610 | mG*mG*fC*fC*mA*mA*mA*fC*fC*fU*fC*mG*mG*fC*fU*fU*mA*fC*fC*fU |
| 611 | fG*fG*mC*mC*fA*fA*fA*mC*mC*mU*mC*fG*fG*mC*mU*mU*fA*mC*mC*mU |
| 612 | mG*fG*mC*fC*mA*fA*mA*fC*mC*fU*mC*fG*mG*fC*mU*fU*mA*fC*mC*fU |
| 613 | mG*mG*mC*mC*mA*mA*fA*fC*fC*fU*fC*fG*fG*fC*mU*mU*mA*mC*mC*mU |
| 614 | fG*fG*fC*fC*fA*fA*mA*mC*mC*mU*mC*mG*mG*mC*fU*fU*fA*fC*fC*fU |
| 615 | fG*fG*fC*fC*mA*mA*mA*fC*fC*mU*fC*fG*fG*fC*mU*mU*mA*fC*fC*mU |
| 616 | mG*mG*mC*mC*fA*fA*fA*mC*mC*fU*mC*mG*mG*mC*fU*fU*fA*mC*mC*fU |
| 617 | fU*fC*fA*fA*fG*fG*fA*fA*fG*fA*fU*fG*fG*fC*fA*fU*fU*fU*fC*fU |
| 618 | fU*fC*mA*mA*mG*mG*mA*mA*mG*mA*fU*mG*mG*fC*mA*fU*fU*fU*fC*fU |
| 619 | mU*mC*fA*fA*fG*fG*fA*fA*fG*fA*mU*fG*fG*mC*fA*mU*mU*mU*mC*mU |
| 620 | mU*fC*mA*fA*mG*tG*mA*fA*mG*fA*mU*fG*mG*fC*mA*fU*mU*fU*mC*fU |
| 621 | mU*mC*mA*mA*mG*mG*fA*fA*fG*fA*fU*fG*fG*fC*mA*mU*mU*mU*mC*mU |
| 622 | fU*fC*fA*fA*fG*fG*mA*mA*mG*mA*mU*mG*mG*mC*fA*fU*fU*fU*fC*fU |
| 623 | mU*fC*mA*mA*fG*fG*mA*mA*fG*mA*mU*fG*fG*fC*mA*mU*mU*mU*fC*mU |
| 624 | fU*mC*fA*fA*mG*mG*fA*fA*mG*fA*fU*mG*mG*mC*fA*fU*fU*fU*mC*fU |
| 625 | G*G*C*C*A*A*A*C*C*T*C*G*G*C*T*T*A*C*C*T |
| 626 | mGmGmCmCmAmAmAmCmCmUmCmGmGmCmUmUmAmCmCmU |
| 627 | G*RG*RC*RC*RA*RA*RA*RC*RC*RT*RC*RG*RG*RC*RT*RT*RA*RC*RC*RT |
| 628 | G*SG*SC*SC*SA*SA*SA*SC*SC*ST*SC*SG*SG*SC*ST*ST*SA*SC*SC*ST |
| 629 | G*SG*SC*SC*SA*SmAmAmCmCmUmCmGmGmCT*ST*SA*SC*SC*ST |
| 630 | mGmGmCmCA*SA*SA*SmCC*ST*SC*SG*SmGC*ST*ST*SmAmCmCmU |
| 631 | G*SmGC*SmCA*SmAA*SmCC*SmUC*SmGG*SmCT*SmUA*SmCC*SmU |
| 632 | mGG*SmCC*SmAA*SmAC*SmCT*SmCG*SmGC*SmUT*SmAC*SmCmU |
| 633 | G*SG*SmCmCA*SA*SmAmCC*ST*SC*SmGmGC*ST*SmUmAC*SC*SmU |
| 634 | G*SG*SC*SmCmAmAA*SC*SmCmUmCG*SG*SmCmUmUA*SC*SC*SmU |
| 635 | G*SG*SC*SC*SmAmAmAmCC*ST*SC*SmGmGmCmUT*SA*SC*SC*SmU |
| 636 | G*SG*SC*SmCA*SA*SA*SmCC*ST*SC*SmGG*SC*ST*SmUA*SC*SC*SmU |
| 637 | mGmGmCmCA*SA*SA*SC*SC*SmUmCmGmGmCT*ST*SA*SC*SC*SmU |
| 638 | G*G*C*C*A*mAmAmCmCmUmCmGmGmCT*T*A*C*C*T |
| 639 | mGmGmCmCA*A*A*mCC*T*C*G*mGC*T*T*mAmCmCmU |
| 640 | G*mGC*mCA*mAA*mCC*mUC*mGG*mCT*mUA*mCC*mU |
| 641 | mGG*mCC*mAA*mAC*mCT*mCG*mGC*mUT*mAC*mCmU |
| 642 | G*G*mCmCA*A*mAmCmCT*C*mGmGC*T*mUmAC*C*mU |
| 643 | G*G*C*mCmAmAA*C*mCmUmCG*G*mCmUmUA*C*C*mU |
| 644 | G*G*C*C*mAmAmAmCC*T*C*mGmGmCmUT*A*C*C*mU |
| 645 | G*G*C*mCA*A*A*mCC*T*C*mGG*C*T*mUA*C*C*mU |
| 646 | mGmGmCmCA*A*A*C*C*mUmCmGmGmCT*T*A*C*C*mU |
| 647 | G*G*mCmCmAmAmAmCmCmUC*mGmGC*mUT*A*C*C*mU |
| 648 | mG*mG*mC*mC*mA*mAmAmCmCmUmCmGmGmCmU*mU*mA*mC*mC*mU |
| 649 | mGmGmCmCmA*mA*mA*mCmC*mU*mC*mG*mGmC*mU*mU*mAmCmCmU |
| 650 | mG*mGmC*mCmA*mAmA*mCmC*mUmC*mGmG*mCmU*mUmA*mCmC*mU |
| 651 | mGmG*mCmC*mAmA*mAmC*mCmU*mCmG*mGmC*mUmU*mAmC*mCmU |
| 652 | mG*mG*mCmCmA*mA*mAmCmCmU*mC*mGmGmC*mU*mUmAmC*mC*mU |
| 653 | mG*mG*mC*mCmAmAmA*mC*mCmUmCmG*mG*mCmUmUmA*mC*mC*mU |
| 654 | mG*mG*mC*mC*mAmAmAmCmC*mU*mC*mGmGmCmUmU*mA*mC*mC*mU |
| 655 | mG*mG*mC*mCmA*mA*mA*mCmC*mU*mC*mGmG*mC*mU*mUmA*mC*mC |
| | *mU |
| 656 | mGmGmCmCmA*mA*mA*mC*mC*mUmCmGmGmCmU*mU*mA*mC*mC*mU |
| 657 | mG*mG*mCmCmAmAmAmCmCmUmC*mGmGmC*mUmU*mA*mC*mC*mU |
| 658 | LOOI*mG*mG*mC*mC*mA*mA*mA*mC*mC*mU*mC*mG*mG*mC*mU*mU |
| | *mA*mC*mC*mU |
| 659 | LOOl*mG*mG*mC*mC*mA*mA*mA*mC*mC*mU*mC*mG*mG*mC*mU*mU |
| | *mA*mC*mC*mU*mG*mA*mA*mA*mG*SG*SmCmCmAmAmAmCmCmUC*Sm |
| 660 | G*SG*SmCmCmAmAmAmCmCmUC*SmGmGC*SmUT*SA*SC*SC*SmU |
| 661 | T*C*A*A*G*G*A*A*G*A*T*G*G*C*A*T*T*T*C*T |
| 662 | mUmCmAmAmGmGmAmAmGmAmUmGmGmCmAmUmUmUmCmU |
| 663 | T*RC*RA*RA*RG*RG*RA*RA*RG*RA*RT*RG*RG*RC*RA*RT*RT*RT*RC*RT |
| 664 | T*SC*SA*SA*SG*SG*SA*SA*SG*SA*ST*SG*SG*SC*SA*ST*ST*ST*SC*ST |
| 665 | T*SC*SA*SA*SG*SmGmAmAmGmAmUmGmGmCA*ST*ST*ST*SC*ST |
| 666 | mUmCmAmAG*SG*SA*SmAG*SA*ST*SG*SmGC*SA*ST*SmUmUmCmU |
| 667 | T*SmCA*SmAG*SmGA*SmAG*SmAT*SmGG*SmCA*SmUT*SmUC*SmU |
| 668 | mUC*SmAA*SmGG*SmAA*SmGA*SmUG*SmGC*SmAT*SmUT*SmCmU |
| 669 | T*SC*SmAmAG*SG*SmAmAG*SA*ST*SmGmGC*SA*SmUmUT*SC*SmU |
| 670 | T*SC*SA*SmAmGmGA*SA*SmGmAmUG*SG*SmCmAmUT*ST*SC*SmU |
| 671 | T*SC*SA*SA*SmGmGmAmAG*SA*ST*SmGmGmCmAT*ST*ST*SC*SmU |
| 672 | T*SC*SA*SmAG*SG*SA*SmAG*SA*ST*SmGG*SC*SA*SmUT*ST*SC*SmU |
| 673 | mUmCmAmAG*SG*SA*SA*SG*SmAmUmGmGmCA*ST*ST*ST*SC*SmU |
| 674 | T*SC*SmAmAmGmGmAmAmGmAT*SmGmGC*SmAT*ST*ST*SC*SmU |
| 675 | T*C*A*A*G*mGmAmAmGmAmUmGmGmCA*T*T*T*C*T |
| 676 | mUmCmAmAG*G*A*mAG*A*T*G*mGC*A*T*mUmUmCmU |
| 677 | T*mCA*mAG*mGA*mAG*mAT*mGG*mCA*mUT*mUC*mU |
| 678 | mUC*mAA*mGG*mAA*mGA*mUG*mGC*mAT*mUT*mCmU |
| 679 | T*C*mAmAG*G*mAmAG*A*T*mGmGC*A*mUmUT*C*mU |
| 680 | T*C*A*mAmGmGA*A*mGmAmUG*G*mCmAmUT*T*C*mU |
| 681 | T*C*A*A*mGmGmAmAG*A*T*mGmGmCmAT*T*T*C*mU |
| 682 | T*C*A*mAG*G*A*mAG*A*T*mGG*C*A*mUT*T*C*mU |
| 683 | mUmCmAmAG*G*A*A*G*mAmUmGmGmCA*T*T*T*C*mU |
| 684 | T*C*mAmAmGmGmAmAmGmAT*mGmGC*mAT*T*T*C*mU |
| 685 | mU*mC*mA*mA*mG*mGmAmAmGmAmUmGmGmCmA*mU*mU*mU*mC*mU |
| 686 | mUmCmAmAmG*mG*mA*mAmG*mA*mU*mG*mGmC*mA*mU*mUmUmCmU |
| 687 | mU*mCmA*mAmG*mGmA*mAmG*mAmU*mGmG*mCmA*mUmU*mUmC*mU |
| 688 | mUmC*mAmA*mGmG*mAmA*mGmA*mUmG*mGmC*mAmU*mUmU*mCmU |
| 689 | mU*mC*mAmAmG*mG*mAmAmG*mA*mU*mGmGmC*mA*mUmUmU*mC*mU |
| 690 | mU*mC*mA*mAmGmGmA*mA*mGmAmUmG*mG*mCmAmUmU*mU*mC*mU |
| 691 | mU*mC*mA*mA*mGmGmAmAmG*mA*mU*mGmGmCmAmU*mU*mU*mC*mU |
| 692 | mU*mC*mA*mAmG*mG*mA*mAmG*mA*mU*mGmG*mC*mA*mUmU*mU*mC |
| | *mU |
| 693 | mUmCmAmAmG*mG*mA*mA*mG*mAmUmGmGmCmA*mU*mU*mU*mC*mU |
| 694 | mU*mC*mAmAmGmGmAmAmGmAmU*mGmGmC*mAmU*mU*mU*mC*mU |
| 695 | rArGrArArArUrGrCrCrArUrCrUrUrCrCrUrUrGrA |
| 696 | mU*SmC*SmA*RmA*RmG*RmG*RmA*RmA*RmG*RmA*RmU*RmG*RmG*RmC* |
| | RmA*RmU*RmU*RmU*SmC*SmU |
| 697 | mU*SmC*SmA*SmA*SmG*RmG*RmA*RmA*RmG*RmA*RmU*RmG*RmG*RmC* |
| | RmA*RmU*SmU*SmU*SmC*SmU |
| 698 | mU*SmC*SmA*SmA*SmG*SmG*RmA*RmA*RmG*RmA*RmU*RmG*RmG*RmC* |
| | RmA*SmU*SmU*SmU*SmC*SmU |
| 699 | mU*SmCmAmAmGmGmAmAmGmAmUmGmGmCmAmUmUmUmC*SmU |
| 700 | mU*SmC*SmAmAmGmGmAmAmGmAmUmGmGmCmAmUmUmU*SmC*SmU |
| 701 | mU*SmC*SmA*SmAmGmGmAmAmGmAmUmGmGmCmAmUmU*SmU*SmC*SmU |
| 702 | mU*SmC*SmA*SmA*SmGmGmAmAmGmAmUmGmGmCmAmU*SmU*SmU*SmC |
| | *SmU |
| 703 | mU*SmC*SmA*SmA*SmG*SmGmAmAmGmAmUmGmGmCmA*SmU*SmU*SmU |
| | *SmC*SmU |
| 704 | mU*mCmAmAmGmGmAmAmGmAmUmGmGmCmAmUmUmUmC*mU |
| 705 | mU*mC*mAmAmGmGmAmAmGmAmUmGmGmCmAmUmUmU*mC*mU |
| 706 | mU*mC*mA*mAmGmGmAmAmGmAmUmGmGmCmAmUmU*mU*mC*mU |
| 707 | mU*mC*mA*mA*mGmGmAmAmGmAmUmGmGmCmAmU*mU*mU*mC*mU |
| 708 | mU*mC*mA*mA*mG*mGmAmAmGmAmUmGmGmCmA*mU*mU*mU*mC*mU |
| 709 | fU*fC*fA*fA*fG*fG*mAmAmGmAmUmGmGmC*fA*fU*fU*fU*fC*fU |
| 710 | fU*fC*fA*fA*fG*mGmAmAmGmAmUmGmGmCmA*fU*fU*fU*fC*fU |
| 711 | fU*fC*fA*fA*mGmGmAmAmGmAmUmGmGmCmAmU*fU*fU*fC*fU |
| 712 | fU*fC*fA*mAmGmGmAmAmGmAmUmGmGmCmAmUmU*fU*fC*fU |
| 713 | fU*fC*mAmAmGmGmAmAmGmAmUmGmGmCmAmUmUmU*fC*fU |
| 714 | fU*mCmAmAmGmGmAmAmGmAmUmGmGmCmAmUmUmUmC*fU |
| 715 | fU*SfC*SfA*SfA*SfG*SfG*SmAmAmGmAmUmGmGmC*SfA*SfU*SfU*SfU*SfC |
| | *SfU |
| 716 | fU*SfC*SfA*SfA*SfG*SmGmAmAmGmAmUmGmGmCmA*SfU*SfU*SfU*SfC*SfU |
| 717 | fU*SfC*SfA*SfA*SmGmGmAmAmGmAmUmGmGmCmAmU*SfU*SfU*SfC*SfU |
| 718 | fU*SfC*SfA*SmAmGmGmAmAmGmAmUmGmGmCmAmUmU*SfU*SfC*SfU |
| 719 | fU*SfC*SmAmAmGmGmAmAmGmAmUmGmGmCmAmUmUmU*SfC*SfU |
| 720 | fU*SmCmAmAmGmGmAmAmGmAmUmGmGmCmAmUmUmUmC*SfU |
| 721 | fU*SfC*SfA*SfA*SfG*SfG*SmA*RmA*RmG*RmA*RmU*RmG*RmG*RmC*SfA*Sf |
| | u*SfU*SfU*SfC*SfU |
| 722 | fU*SfC*SfA*SfA*SfG*SmG*RmA*RmA*RmG*RmA*RmU*RmG*RmG*RmC*RmA* |
| | SfU*SfU*SfU*SfC*SfU |
| 723 | fU*SfC*SfA*SfA*SmG*RmG*RmA*RmA*RmG*RmA*RmU*RmG*RmG*RmC*RmA |
| | *RmU*SfU*SfU*SfC*SfU |
| 724 | fU*SfC*SfA*SmA*RmG*RmG*RmA*RmA*RmG*RmA*RmU*RmG*RmG*RmC*Rm |
| | A*RmU*RmU*SfU*SfC*SfU |
| 725 | fU*SfC*SmA*RmA*RmG*RmG*RmA*RmA*RmG*RmA*RmU*RmG*RmG*RmC*R |
| | mA*RmU*RmU*RmU*SfC*SfU |
| 726 | fU*SmC*RmA*RmA*RmG*RmG*RmA*RmA*RmG*RmA*RmU*RmG*RmG*RmC* |
| | RmA*RmU*RmU*RmU*RmC*SfU |
| 727 | fU*SfC*SfA*SfA*SfG*SfG*SfA*SfA*RmG*RmA*RmU*RmG*RmG*SfC*SfA*SfU*S |
| | fU*SfU*SfC*SfU |
| 728 | fU*SfC*SfA*SfA*SfG*SfG*SfA*SfA*SmG*RmA*RmU*RmG*SfG*SfC*SfA*SfU*Sf |
| | u*SfU*SfC*SfU |
| 729 | fU*SfC*SfA*SfA*SfG*SfG*SfA*SfA*SfG*SmA*RmU*SfG*SfG*SfC*SfA*SfU*SfU* |
| | SfU*SfC*SfU |
| 730 | fU*SfC*SfA*SfA*SfG*SfG*SfA*SfAmGmAmUmGmG*SfC*SfA*SfU*SfU*SfU*SfC |
| | *SfU |
| 731 | fU*SfC*SfA*SfA*SfG*SfG*SfA*SfA*SmGmAmUmG*SfG*SfC*SfA*SfU*SfU*SfU |
| | *SfC*SfU |
| 732 | fU*SfC*SfA*SfA*SfG*SfG*SfA*SfA*SfG*SmAmU*SfG*SfG*SfC*SfA*SfU*SfU |
| | *SfU*SfC*SfU |
| 733 | fU*SfC*SfA*SfA*StG*SfG*SfA*mA*mG*mA*mU*mG*mG*fC*SfA*SfU*SfU*SfU*S |
| | fC*SfU |
| 734 | mU*SmC*SmA*SmA*SmG*SmG*SmA*RmA*RmG*RmA*RmU*RmG*RmG*RmC*S |
| | mA*SmU*SmU*SmU*SmC*SmU |
| 735 | mU*SmC*SmA*SmA*SmG*SmG*SmA*SmA*RmG*RmA*RmU*RmG*RmG*SmC*S |
| | mA*SmU*SmU*SmU*SmC*SmU |
| 736 | mU*SmC*SmA*SmA*SmG*SmG*SmA*SmA*SmG*RmA*RmU*RmG*SmG*SmC*S |
| | mA*SmU*SmU*SmU*SmC*SmU |
| 737 | mU*SmC*SmA*SmA*SmG*SmG*SmA*SmA*SmG*SmA*RmU*SmG*SmG*SmC*Sm |
| | A*SmU*SmU*SmU*SmC*SmU |
| 738 | mU*SmC*SmA*SmA*SmG*SmG*SmA*mA*mG*mA*mU*mG*mG*mC*SmA* |
| | SmU*SmU*SmU*SmC*SmU |
| 739 | LOOl*mU*mC*mA*mA*mG*mG*mA*mA*mG*mA*mU*mG*mG*mC*mA*mU |
| | *mU*mU*mC*mU |
| 740 | ModO13L001*mU*mC*mA*mA*mG*mG*mA*mA*mG*mA*mU*mG*mG*mC |
| | *mA*mU*mU*mU*mC*mU |
| 741 | Mod014L001*mU*mC*mA*mA*mG*mG*mA*mA*mG*mA*mU*mG*mG*mC |
| | *mA*mU*mU*mU*mC*mU |
| 742 | Mod005L001*mU*mC*mA*mA*mG*mG*mA*mA*mG*mA*mU*mG*mG*mC |
| | *mA*mU*mU*mU*mC*mU |
| 743 | Mod015L001*mU*mC*mA*mA*mG*mG*mA*mA*mG*mA*mU*mG*mG*mC |
| | *mA*mU*mU*mU*mC*mU |
| 744 | ModO16L001*mU*mC*mA*mA*mG*mG*mA*mA*mG*mA*mU*mG*mG*mC |
| | *mA*mU*mU*mU*mC*mU |
| 745 | ModO17L001*mU*mC*mA*mA*mG*mG*mA*mA*mG*mA*mU*mG*mG*mC |
| | *mA*mU*mU*mU*mC*mU |
| 746 | Mod018L001*mU*mC*mA*mA*mG*mG*mA*mA*mG*mA*mU*mG*mG*mC |
| | *mA*mU*mU*mU*mC*mU |
| 747 | ModO19L001*mU*mC*mA*mA*mG*mG*mA*mA*mG*mA*mU*mG*mG*mC |
| | *mA*mU*mU*mU*mC*mU |
| 748 | Mod006L001*mU*mC*mA*mA*mG*mG*mA*mA*mG*mA*mU*mG*mG*mC |
| | *mA*mU*mU*mU*mC*mU |
| 749 | Mod020L001*mU*mC*mA*mA*mG*mG*mA*mA*mG*mA*mU*mG*mG*mC |
| | *mA*mU*mU*mU*mC*mU |
| 750 | Mod021*mU*mC*mA*mA*mG*mG*mA*mA*mG*mA*mU*mG*mG*mC*mA* |
| | mU*mU*mU*mC*mU |
| 751 | mU*mC*mA*mA*mG*mG*mAmAmGmAmUmGmGmC*mA*mU*mU*mU*mC*mU |
| 752 | mU*mC*mA*mA*mG*mG*mA*mAmGmAmUmGmG*mC*mA*mU*mU*mU*mC*m |
| | u |
| 753 | mU*mC*mA*mA*mG*mG*mA*mA*mGmAmUmG*mG*mC*mA*mU*mU*mU*mC* |
| | mU |
| 754 | mU*mC*mA*mA*mG*mG*mA*mA*mG*mAmU*mG*mG*mC*mA*mU*mU*mU*m |
| | C*mU |
| 755 | mU*SmC*SmA*SmA*SmG*SmG*SmAmAmGmAmUmGmGmC*SmA*SmU*SmU*S |
| | mU*SmC*SmU |
| 756 | mU*SmC*SmA*SmA*SmG*SmG*SmA*SmAmGmAmUmGmG*SmC*SmA*SmU*Sm |
| | U*SmU*SmC*SmU |
| 757 | mU*SmC*SmA*SmA*SmG*SmG*SmA*SmA*SmGmAmUmG*SmG*SmC*SmA*SmU |
| | *SmU*SmU*SmC*SmU |
| 758 | mU*SmC*SmA*SmA*SmG*SmG*SmA*SmA*SmG*SmAmU*SmG*SmG*SmC*SmA* |
| | SmU*SmU*SmU*SmC*SmU |
| 759 | fU*fC*fA*fA*fG*fG*fA*mAmGmAmUmGmG*fC*fA*fU*fU*fU*fU*fU |
| 760 | fU*fC*fA*fA*fG*fG*fA*fA*mGmAmUmG*fG*fC*fA*fU*fU*fU*fC*fU |
| 761 | fU*fC*fA*fA*fG*fG*fA*fA*fG*mAmU*fG*fG*fC*fA*fU*fU*fU*fC*fU |
| 762 | fU*SfC*SfA*SfA*SfG*SfG*SmAmAmGmA*RmUmGmGmC*SfA*SfU*SfU*SfU*SfC |
| | *SfU |
| 763 | fU*SfC*SfA*SfA*SfG*SfG*SmAmAmG*RmA*RmU*RmGmGmC*SfA*SfU*SfU*SfU |
| | *SfC*SfU |
| 764 | fU*SfC*SfA*SfA*SfG*SfG*SmAmA*RmG*RmA*RmU*RmG*RmGmC*SfA*SfU*Sf |
| | u*SfU*SfC*SfU |
| 765 | fU*SfC*SfA*SfA*SfG*SfG*SmAmAmGmAfU*SmGmG*SfC*SfA*SfU*SfU*SfU*SfC |
| | *SfU |
| 766 | fU*SfC*SfA*SfA*SmG*SmG*SfAfAmGmAfU*SmGmG*SfC*SfA*SfU*SfU*SfU*SfC |
| | *SfU |
| 767 | fU*SfC*SfA*SfA*SfG*SfG*SmA*RmA*RmGmAmUmG*RmG*RmC*SfA*SfU*SfU*S |
| | fU*SfC*SfU |
| 768 | fU*SfC*SfA*SfA*SfG*SfG*SmA*RmAmGmAmUmGmG*RmC*SfA*SfU*SfU*SfU*S |
| | fC*SfU |
| 769 | fU*SfC*SfA*SfA*SfG*SfG*SmA*SmA*SmGmAmUmG*SmG*SmC*SfA*SfU*SfU*Sf |
| | U*SfC*SfU |
| 770 | fU*SfC*SfA*SfA*SfG*SfG*SmA*SmAmGmAmUmGmG*SmC*SfA*SfU*SfU*SfU*Sf |
| | C*SfU |
| 771 | fU*SfC*SfA*SfA*SfG*SfG*SmA*SmA*SmG*SmA*SmU*SmG*SmG*SmC*SfA*SfU |
| | *SfU*SfU*SfC*SfU |
| 772 | fU*SfC*SfA*SfA*SfG*SfG*SmA*RmA*RmG*RmA*RfU*SmG*RmG*SfC*SfA*SfU* |
| | SfU*SfU*SfC*SfU |
| 773 | fU*SfC*SfA*SfA*SmG*SmG*SfA*RfA*RmG*RmA*RfU*SmG*RmG*SfC*SfA*SfU* |
| | SfU*SfU*SfC*SfU |
| 774 | fU*SfC*SfA*SfA*SfG*SfG*SfA*SmAmGmAmUmGmG*SfC*SfA*SfU*SfU*SfU*SfC |
| | *SfU |
| 775 | fU*SfC*SfA*SfA*SfG*SfG*SfA*SmA*RmG*RmA*RmU*RmG*RmG*SfC*SfA*SfU* |
| | SfU*SfU*SfC*SfU |
| 776 | mU*SmC*SmA*SfA*SfG*SfG*SmA*RmA*RmG*RmA*RmU*RmG*RmG*RmC*SfA |
| | *SfU*SfU*SmU*SmC*SmU |
| 777 | mU*SmC*SmA*SfA*SfG*SfG*SfA*SmA*RmG*RmA*RmU*RmG*RmG*SfC*SfA*Sf |
| | U*SfU*SmU*SmC*SmU |
| 778 | mU*SmC*SmA*SfA*SfG*SfG*SfA*SfA*SmG*RmA*RmU*RmG*SfG*SfC*SfA*SfU* |
| | SfU*SmU*SmC*SmU |
| 779 | mU*SmC*SmA*SfA*SfG*SfG*SfA*SfA*SfG*SmA*RmU*StG*SfG*SfC*SfA*SfU*Sf |
| | U*SmU*SmC*SmU |
| 780 | mU*SmC*SmA*SfA*SfG*SfG*SfA*SfA*SmGmAmUmG*SfG*SfC*SfA*SfU*SfU*Sm |
| | U*SmC*SmU |
| 781 | mU*SmC*SmA*SfA*SfG*SfG*SfA*SfA*SfG*SmAmU*SfG*SfG*SfC*SfA*SfU*SfU* |
| | SmU*SmC*SmU |
| 782 | fU*fC*fA*fA*fG*fG*fA*fA*mG*mA*mU*mG*mG*fC*fA*fU*fU*fU*fC*fU |
| 783 | fU*fC*fA*fA*fG*fC*fA*fA*mG*mA*mU*mG*fG*fC*fA*fU*fU*fU*fC*fU |
| 784 | fU*fC*fA*fA*fG*fG*fA*fA*fG*mA*mU*fG*fc*fA*fU*fU*fU*fC*fU |
| 785 | fU*fC*fA*fA*fG*fG*fA*mA*mG*mA*mU*mG*mG*fC*fA*fU*fU*fU*fC*fU |
| 786 | mU*mC*mA*fA*fG*fG*mA*mA*mG*mA*mU*mG*mG*mC*fA*fU*fU*mU*mC*m |
| | u |
| 787 | mU*mC*mA*fA*fG*fG*fA*mA*mG*mA*mU*mG*mG*fC*fA*fU*fU*mU*mC*mU |
| 788 | mU*mC*mA*fA*fG*fG*fA*fA*mG*mA*mU*mG*fG*fC*A*fU*fU*mU*mC*mU |
| 789 | mU*mC*mA*fA*fG*fG*fA*fA*fC*mA*mU*fG*fG*fC*fA*fU*fU*mU*mC*mU |
| 790 | mU*mC*mA*fA*fG*fG*fA*fA*mGmAmUmG*fG*fC*fA*fU*fU*mU*mC*mU |
| 791 | mU*mC*mA*fA*fG*fG*fA*fA*fG*mAmU*fG*fG*fC*fA*fU*fU*mU*mC*mU |
| 792 | Mod024L001*mU*mC*mA*mA*mG*mG*mA*mA*mG*mA*mU*mG*mG*mC*mA* |
| | mU*mU*mU*mC*mU |
| 793 | Mod026L001*mU*mC*mA*mA*mG*mG*mA*mA*mG*mA*mU*mG*mG*mC*mA* |
| | mU*mU*mU*mC*mU |
| 794 | fU*fC*fA*fA*fG*fG*mA*mA*mG*mA*BrdU*mG*mG*mC*fA*fU*fU*fU*fC*fU |
| 795 | fU*fC*fA*fA*fG*fG*fA*fA*fG*mA*BrdU*fG*fC*fC*fA*fU*fU*fU*fC*fU |
| 796 | mU*mC*mA*mA*mG*mG*mA*mA*mG*mA*BrdU*mG*mG*mC*mA*mU*mU*mU* |
| | mC*mU |
| 797 | fU*SfC*SfA*SfA*SfG*SfG*SA*SfA*SmGmABrdUmG*SfG*SfC*SfA*SfU*SfU*SfU |
| | *SfC*SfU |
| 798 | fU*fC*fA*fA*fG*fG*fA*fA*mGmABrdUmG*fG*fC*fA*fC*fU*fU*fC*fU |
| 799 | fU*SfC*SfA*SfA*SfG*SfG*SmAmAmGmABrdUmGmGmC*SfA*SfU*SfU*SfU*SfC* |
| | SfU |
| 800 | fU*fC*fA*fA*fG*fG*mAmAmGmABrdUmGmGmC*fA*fU*fU*fU*fC*fU |
| 801 | LOOI*fU*SfC*SfA*SfA*SfG*SfG*SmAmAmGmAmUmGmGmC*SfA*SfU*SfU*SfU |
| | *SfC*SfU |
| 802 | ModO15L001*fU*SfC*SfA*SfA*SfG*SfG*SmAmAmGmAmUmGmGmC*SfA*SfU*Sf |
| | U*SfU*SfC*SfU |
| 803 | Mod006L001*fU*SfC*SfA*SfA*SfG*SfG*SmAmAmGmAmUmGmGmC*SfA*SfU*Sf |
| | U*SfU*SfC*SfU |
| 804 | LOOI*fU*SfC*SfA*SfA*SfG*SfG*SfA*SfA*SmGmAmUmG*SfG*SfC*SfA*SfU |
| | *SfU*SfU*SfC*SfU |
| 805 | ModO15L001*fU*SfC*SfA*SfA*SfG*SfG*SfA*SfA*SmGmAmUmG*SfG*SfC*SfA |
| | *SfU*SfU*SfU*SfC*SfU |
| 806 | Mod006L001*fU*SfC*SfA*SfA*SfG*SfG*SfA*SfA*SmGmAmUmG*SfG*SfC*SfA |
| | *SfU*SfU*SfU*SfC*SfU |
| 807 | fU*SfC*SfA*SfA*SfG*SfG*SfA*SfA*SmGmAmUmGmG*SfC*SfA*SfU*SfU*SfU |
| | *SfC*SfU |
| 808 | LOOl*fU*fC*fA*fA*fG*fG*mA*mA*mG*mA*mU*mG*mG*mC*fA*fU*fU*fU*fC |
| | *fU |
| 809 | Mod015L001*fU*fC*fA*fA*fG*fG*mA*mA*mG*mA*mU*mG*mG*mC*fA*fU*fU |
| | *fU*fC*fU |
| 810 | Mod006L001*fU*fC*fA*fA*fG*fG*mA*mA*mG*mA*mU*mG*mG*mC*fA*fU*fU*f |
| | U*fC*fU |
| 811 | Mod020L001*fU*fC*fA*fA*fG*fG*mA*mA*mG*mA*mU*mG*mG*mC*fA*fU*fU*f |
| | U*fC*fU |
| 812 | Mod019L001*fU*fC*fA*fA*fG*fG*mA*mA*mG*mA*mU*mG*mG*mC*fA*fU*fU*f |
| | U*fC*fU |
| 813 | LOOI*fU*fC*fA*fA*fG*fG*A*fA*mGmAmUmG*fG*fC*fA*fU*fU*fU*fC*fU |
| 814 | Mod015L001*fU*fC*fA*fA*fG*fG*fA*fA*mGmAmUmG*fG*fC*fA*fU*fU*fU*fC*f |
| | u |
| 815 | Mod006L001*fU*fC*fA*fA*fG*fG*fA*fA*mGmAmUmG*fG*fC*fA*fU*fU*fU*fC*f |
| | u |
| 816 | Mod020L001*fU*ft*fA*fA*fG*fG*fA*fA*mGmAmUmG*fG*fC*fA*fU*fU*fU*fC*fU |
| 817 | Mod019L001*fU*f*fA*fA*fG*fG*A*fA*mGmAmUmG*fG*fC*fA*fU*fU*fU*fC*fU |
| 818 | fU*fC*fA*fA*fG*fG*mAmAmGmA*mUmGmGmC*fA*fU*fU*fU*fC*fU |
| 819 | fU*fC*fA*fA*fG*fG*mAmAmG*mA*mU*mGmGmC*fA*fU*fU*fU*fC*fU |
| 820 | fU*fC*fA*fA*fG*fG*mAmA*mG*mA*mU*mG*mGmC*fA*fU*fU*fU*fC*fU |
| 821 | fU*fC*fA*fA*fG*fG*mA*mA*mGmAmUmG*mG*mC*fA*fU*fU*fU*fC*fU |
| 822 | fU*fC*fA*fA*fG*fG*mA*mAmGmAmUmGmG*mC*fA*fU*fU*fU*fC*fU |
| 823 | fU*fC*fA*fA*fG*fG*mA*mA*mGmAmUmG*mG*mC*fA*fU*fU*fU*fC*fU |
| 824 | fU*fC*fA*fA*fG*PG*mA*mAmGmAmUmGmG*mC*fA*fU*fU*fU*fC*fU |
| 825 | fU*fC*fA*fA*fG*fG*mAmAmGmAfU*mGmG*fC*fA*fU*fU*fU*fC*fU |
| 826 | fU*fC*fA*fA*fG*fG*mA*mA*mG*mA*fU*mG*mG*fC*fA*fU*fU*fU*fC*fU |
| 827 | fU*fC*fA*fA*mG*mG*fAfAmGmAfU*mGmG*fC*fA*fU*fU*fU*fC*fU |
| 828 | fU*fC*fA*fA*mG*mG*fA*fA*mG*mA*fU*mG*mG*fC*fA*fU*fU*fU*fC*fU |
| 829 | fU*fC*fA*fA*fG*fG*mA*mA*mG*mA*fU*mG*mG*fC*fA*fU*fU*fU*fC*fU |
| 830 | fU*fC*fA*fA*mG*mG*fA*fA*mG*mA*fU*mG*mG*fC*fA*fU*fU*fU*fC*fU |
| 831 | fU*fC*fA*fA*mG*mG*fA*fA*mGmAmUmG*mG*fC*fA*fU*fU*fU*fC*fU |
| 832 | fU*fC*fA*fA*mG*mG*mA*mA*mGmAfUmG*mG*fC*fA*fU*fU*fU*fC*fU |
| 833 | fU*fC*fA*fA*mG*mG*fA*fA*mG*mA*mU*mG*mG*fC*fA*fU*fU*fU*fC*fU |
| 834 | fU*fC*fA*fA*mG*mG*mA*mA*mG*mA*fU*mG*mG*fC*fA*fU*fU*fU*fC*fU |
| 835 | fU*fC*fA*fA*mG*mG*fAfAmGmA*fU*mGmG*fC*fA*fU*fU*fU*fC*fU |
| 836 | fU*fC*fA*fA*mG*mG*fA*fA*mG*mA*fU*mG*mG*fC*fA*fU*fU*fU*fC*fU |
| 837 | fU*fC*fA*fA*mG*mG*fA*fA*mG*fA*fU*mG*mG*fC*fA*fU*fU*fU*fC*fU |
| 838 | fU*fC*fA*fA*mG*mG*fA*fA*mGmAmUmGmG*fC*fA*fU*fU*fU*fC*fU |
| 839 | fU*fC*fA*fA*mG*mG*fA*fA*mGmAfU*mGmG*fC*fA*fU*fU*fU*fC*fU |
| 840 | fU*fC*fA*fA*mG*mG*mA*mA*mGmAfU*mGmG*fC*fA*fU*fU*fU*fC*fU |
| 841 | fU*SfC*SfA*SfA*SfG*SfG*SmAmAmGmAmU:mGmGmC*SfA*SfU*SfU*SfU*SfC*S |
| | fU |
| 842 | fU*SfC*SfA*SfA*SfG*SfG*SmAmA:mGmAmUmG:mGmC*SfA*SfU*SfU*SfU*SfC* |
| | SfU |
| 843 | fU*SfC*SfA*SfA*SfG*SfG*SmA:mAmG:mAmUmGmG:mC*SfA*SfU*SfU*SfU*SfC |
| | *SfU |
| 844 | fU*SfC*SfA*SfA*SfG*SfG*SmA:mAmGmAmU:mGmG:mC*SfA*SfU*SfU*SfU*SfC |
| | *SfU |
| 845 | fU*SfC*SfA*SfA*fG:fG:mAmAmGmAmU:mGmGmC*SfA*SfU*SfU*SfU*SfC*SfU |
| 846 | fU*SfC*SfA*SfA*mG:mG:mAmAmGmAmU:mGmGmC*SfA*SfU*SfU*SfU*SfC*SfU |
| 847 | fU*SfC*SfA*SfA*SfG*SfG*SfA*SmAmGmAmU:mGmG*SfC*SfA*SfU*SfU*SfU*SfC |
| | *SfU |
| 848 | fU*SfC*SfA*SfA*fG:fG:fA*SmAmGmAmU:mGmG*SfC*SfA*SfU*SfU*SfU*SfC*SfU |
| 849 | fU*SfC*SfA*SfA*mG:mG:fA*SmAmGmAmU:mGmG*SfC*SfA*SfU*SfU*SfU*SfC |
| | *SfU |
| 850 | fU*SfC*SfA*SfA*SfG*SfG*SfA*SfA*SmGmAmU:mG*SfG*SfC*SfA*SfU*SfU*SfU |
| | *SfC*SfU |
| 851 | fU*SfC*SfA*SfA*SfG*SfG*SfA*SfA*SmG:mA:mU:mG*SfG*SPC*SfA*SfU*SfU*SfU |
| | *SfC*SfU |
| 852 | fU*SfC*SfA*SfA*SfG*SfG*SfA*SfA*SmG:mAmU:mG*SfG*SfC*SfA*SfU*SfU*SfU* |
| | SfC*SfU |
| 853 | fU*SfC*SfA*SfA*fG:fG:fA*SfA*SmGmAmU:mG*SfG*SfC*SfA*SfU*SfU*SfU*SfC* |
| | SfU |
| 854 | fU*SfC*SfA*SfA*mG:mG:fA*SfA*SmGmAmU:mG*SfG*SfC*SfA*SfU*SfU*SfU*SfC |
| | *SfU |
| 855 | Mod015L001mU*mC*mA*mA*mG*mG*mA*mA*mG*mA*mU*mG*mG*mC*mA*m |
| | U*mU*mU*mC*mU |
| 856 | Mod019L001mU*mC*mA*mA*mG*mG*mA*mA*mG*mA*mU*mG*mG*mC*mA*m |
| | U*mU*mU*mC*mU |
| 857 | Mod020L001mU*mC*mA*mA*mG*mG*mA*mA*mG*mA*mU*mG*mG*mC*mA*m |
| | U*mU*mU*mC*mU |
| 858 | Mod015L001:mU*mC*mA*mA*mG*mG*mA*mA*mG*mA*mU*mG*mG*mC*mA*m |
| | U*mU*mU*mC*mU |
| 859 | Mod019L001:mU*mC*mA*mA*mG*mG*mA*mA*mG*mA*mU*mG*mG*mC*mA*m |
| | U*mU*mU*mC*mU |
| 860 | Mod020L001:mU*mC*mA*mA*mG*mG*mA*mA*mG*mA*mU*mG*mG*mC*mA*m |
| | U*mU*mU*mC*mU |
| 861 | fU*SfC*SfA*SfA*SfG:fG:mAmAmGmAmU:mGmGmC*SfA*SfU*SfU*SfU*SfC*SfU |
| 862 | fU*SfC*SfA*SfA*SmG:mG:mAmAmGmAmU:mGmGmC*SfA*SfU*SfU*SfU*SfC*Sf |
| | U |
| 863 | fU*SfC*SfA*SfA*SfG:fG:fA*SmAmGmAmU:mGmG*SfC*SfA*SfU*SfU*SfU*SfC*Sf |
| | u |
| 864 | fU*SfC*SfA*SfA*SmG:mG:fA*SmAmGmAmU:mGmG*StC*SfA*SfU*SfU*SfU*SfC |
| | *SfU |
| 865 | fU*SfC*SfA*SfA*SfG:fG:fA*SfA*SmGmAmU:mG*SPG*SfC*SfA*SfU*SfU*SfU*SfC |
| | *SfU |
| 866 | fU*SPC*SfA*SfA*SmG:mG:fA*SfA*SmGmAmU:mG*SfG*SfC*SfA*SfU*SfU*SfU*S |
| | fC*SfU |
| 867 | fU*SfC*SfA*SfA*SfG*SfG*SfA*SfA*SfG*SmAmUmG*SfG*SfC*SfA*SfU*SfU*SfU* |
| | SPC*SfU |
| 868 | fU*fC*fA*fA*fG*fG*fA*fA*fG*mAmUmG*fG*fC*fA*fU*fU*fU*fC*fU |
| 869 | fU*SfC*SfA*SfA*SfG*SfG*SmAfA*SmGfA*SmUfG*SmGfC*SfA*SfU*SfU*SfU*SfC |
| | *SfU |
| 870 | fU*SfC*SfA*SfA*SfG*SfG*SfA*SfA*SmGfA*SmUPG*SmG*SfC*SfA*SfU*SfU*SfU |
| | *SfC*SfU |
| 871 | L001mU*mC*mA*mA*mG*mG*mA*mA*mG*mA*mU*mG*mG*mC*mA*mU*mU* |
| | mU*mC*mU |
| 872 | LOO1fU*SfC*SfA*SfA*SfG*SfG*SfA*SfA*SfG*SmAmU*SfG*SfG*SfC*SfA*SfU*Sf |
| | U*SfU*SfC*SfU |
| 873 | Mod013L001mU*mC*mA*mA*mG*mG*mA*mA*mG*mA*mU*mG*mG*mC*mA*m |
| | U*mU*mU*mC*mU |
| 874 | ModO13L001fU*SfC*SfA*SfA*SfG*SfG*SfA*SfA*SfG*SmAmU*SfG*SfG*SfC*SfA* |
| | SfU*SfU*SfU*SfC*SfU |
| 875 | ModO14L001fU*SfC*SfA*SfA*SfG*SfG*SfA*SfA*SfG*SmAmU*SfG*SfG*SfC*SfA* |
| | SfU*SfU*SfU*SfC*SfU |
| 876 | Mod005L001fU*SfC*SfA*SfA*SfG*SfG*SfA*SfA*SfG*SmAmU*SfG*SfG*SfC*SfA* |
| | SfU*SfU*SfU*SfC*SfU |
| 877 | ModO15L001fU*SfC*SfA*SfA*SfG*SfG*SfA*SfA*SfG*SmAmU*SfG*SfG*SfC*SfA* |
| | SfU*SfU*SfU*SfC*SfU |
| 878 | Mod020L001fU*SfC*SfA*SfA*SfG*SfG*SfA*SfA*SfG*SmAmU*SfG*SfG*SfC*SfA* |
| | SfU*SfU*SfU*SfC*SfU |
| 879 | Mod027L001fU*SfC*SfA*SfA*SfG*SfG*SfA*SfA*SfG*SmAmU*SfG*SfG*SfC*SfA* |
| | SfU*SfU*SfU*SfC*SfU |
| 880 | Mod029L001fU*SfC*SfA*SfA*SfG*SfG*SfA*SfA*SfG*SmAmU*SfG*SfG*SfC*SfA* |
| | sfU*SfU*SfU*SfC*SfU |
| 881 | fU*SfC*SfA*SfA*SfG*SfGfA*SmAfG*SmAfU*SmGfGfC*SfA*SfU*SfU*SfU*SfC*Sf |
| | U |
| 882 | fU*SfC*SfA*SfA*SfG*SfG*SfA*SfAfG*SmAfU*SmG*SmG*SfC*SfA*SfU*SfU*SfU* |
| | SfC*SfU |
| 883 | fU*SfC*SfA*SfA*SfG*SfG*SfA*SfA*SfG*SmAfU*SmG*SfG*SfC*SfA*SfU*SfU*Sf |
| | u*SfC*SfU |
| 884 | fU*SfC*SfA*SfA*SfG*SfG*SfA*SfA*SmGmAfU*SmGmG*SfC*SfA*SfU*SfU*SfU*S |
| | fC*SfU |
| 885 | fU*SfC*SfA*SfA*SfG*SfG*SfA*SfA*SfG*SmAfU*SmGfG*SfC*SU*SfC*SfU |
| 886 | fU*SfC*SfA*SfA*SfG*mAmAmGmAfU*SmGmG*SfC*SfA*SfU*SfU*SfU*SfC*SfU |
| 887 | fU*SfC*SfA*SfA*SfG*SfG*SmA*SmA*SmG*SmA*SfU*SmG*SmG*SfC*SfA*SfU*S |
| | fU*SfU*SfC*SfU |
| 888 | fU*SfC*SfA*SfA*SfG*SfG*SmAfA*SmGfA*SfUfG*SmGfC*SfA*SfU*SfU*SfU*SfC* |
| | SfU |
| 889 | fU*SfC*SfA*SfA*SfG*SfG*SmAfA*SmGfA*SfUmGmGfC*SfA*SfU*SfU*SfU*SfC*S |
| | fU |
| 890 | fU*SfC*SfA*SfA*SfG*SfG*SmAfA*SmGfA*SfU*SmGmGfC*SfA*SfU*SfU*SfU*SfC |
| | *SfU |
| 891 | fU*SfC*SfA*SfA*SfG*SfG*SmAfA*SmGmA*SfU*SmGmGfC*SfA*SfU*SfU*SfU*Sf |
| | C*SfU |
| 892 | fU*SfC*SfA*SfA*SfG*SfG*SmAfA*SmGfAfU*SmGmGfC*SfA*SfU*SfU*SfU*SfC*S |
| | fU |
| 893 | fU*SfC*SfA*SfA*SfG*SfG*SmAfA*SmGmAfU*SmGmGfC*SfA*SfU*SfU*SfU*SfC* |
| | SfU |
| 894 | fU*SfC*SfA*SfA*SfG*SfG*SmAfA*SmGfA*SfU*SmGmGfC*SfAfU*SfU*SfU*SfC*S |
| | fU |
| 895 | fU*SfC*SfA*SfA*SfG*SfG*SmAfA*SmGmA*SfU*SmGmGfC*SfAfU*SfU*SfU*SfC* |
| | SfU |
| 896 | fU*SfC*SfA*SfA*SfG*SfG*SmAfA*SmGfAfU*SmGmGfC*SmA*SfU*SfU*SfU*SfC* |
| | SfU |
| 897 | fU*SfC*SfA*SfA*SfG*SfG*SmAfA*SmGmAfU*SmGmGfC*SmA*SfU*SfU*SfU*SfC |
| | *SfU |
| 898 | fU*SfC*SfA*SfA*SfG*SfG*SmAfA*SmGfAfU*SmGmGfC*SmAfU*SfU*SfU*SfC*Sf |
| | u |
| 899 | fU*SfC*SfA*SfA*SfG*SfG*SmAfA*SmGmAfU*SmGmGfC*SmAfU*SfU*SfU*SfC*Sf |
| | u |
| 900 | fU*SfC*SfA*SfA*SfG*SfG*SmAfA*SmGfAfU*SmGmGfC*SfAfU*SfU*SfU*SfC*SfU |
| 901 | fU*SfC*SfA*SfA*SfG*SfG*SmAfA*SmGmAfU*SmGmGfC*SfAfU*SfU*SfU*SfC*Sf |
| | U |
| 902 | fU*fC*fA*fA*fG*fG*mAfA*mGfA*mUfG*mGfC*fA*fU*fU*fU*fC*fU |
| 903 | Mod030fU*fC*fA*fA*fG*fG*mAfA*mGfA*mUfG*mGfC*fA*fU*fU*fU*fC*fU |
| 904 | Mod031fU*fC*fA*fA*fG*fG*mAfA*mGfA*mUfG*mGfC*fA*fU*fU*fU*fC*fU |
| 905 | Mod032fU*fC*fA*fA*fG*fG*mAfA*mGfA*mUfG*mGfC*fA*fU*fU*fU*fC*fU |
| 906 | Mod033fU*fC*fA*fA*fG*fG*mAfA*mGfA*mUfG*mGfC*fA*fU*fU*fU*fC*fU |
| 907 | ModO13L001fU*SfC*SfA*SfA*SfG*SfG*SmAfA*SmGmA*SfU*SmGmGfC*SfA*SfU* |
| | SfU*SfU*SfC*SfU |
| 908 | Mod005L001fU*SfC*SfA*SfA*SfG*SfG*SmAfA*SmGmA*SfU*SmGmGfC*SfA*SfU* |
| | SfU*SfU*SfC*SfU |
| 909 | ModO15L001fU*SfC*SfA*SfA*SfG*SfG*SmAfA*SmGmA*SfU*SmGmGfC*SfA*SfU* |
| | SfU*SfU*SfC*SfU |
| 910 | Mod020L001fU*SfC*SfA*SfA*SfG*SfG*SmAfA*SmGmA*SfU*SmGmGfC*SfA*SfU* |
| | SfU*SfU*SfC*SfU |
| 911 | Mod027L001fU*SfC*SfA*SfA*SfG*SfG*SmAfA*SmGmA*SfU*SmGmGfC*SfA*SfU* |
| | SfU*SfU*SfC*SfU |
| 912 | Mod029L001fU*SfC*SfA*SfA*SfG*SfG*SmAfA*SmGmA*SfU*SmGmGfC*SfA*SfU* |
| | SfU*SfU*SfC*SfU |
| 913 | Mod03OfU*SfC*SfA*SfA*SfG*SfG*SmAfA*SmGmA*SfU*SmGmGfC*SfA*SfU*SfU* |
| | SfU*SfC*SfU |
| 914 | Mod032fU*SfC*SfA*SfA*SfG*SfG*SmAfA*SmGmA*SfU*SmGmGfC*SfA*SfU*SfU* |
| | SfU*SfC*SfU |
| 915 | Mod033fU*SfC*SfA*SfA*SfG*SfG*SmAfA*SmGmA*SfU*SmGmGfC*SfA*SfU*SfU* |
| | SfU*SfC*SfU |
| 916 | Mod020L001*fU*SfC*SfA*SfA*SfG*SfG*SmAfA*SmGmA*SfU*SmGmGfC*SfA*SfU |
| | *SfU*SfU*SfC* |
| 917 | Mod005L001*fU*SfC*SfA*SfA*SfG*SfG*SmAfA*SmGmA*SfU*SmGmGfC*SfA*SfU |
| | *SfU*SfU*SfC*SfU |
| 918 | ModO14L001fU*SfC*SfA*SfA*SfG*SfG*SmAfA*SmGmA*SfU*SmGmGfC*SfA*SfU* |
| | SfU*SfU*SfC*SfU |
| 919 | Mod03O*fU*SfC*SfA*SfA*SfG*SfG*SmAfA*SmGmA*SfU*SmGmGfC*SfA*SfU*SfU |
| | *SfU*SfC*SfU |
| 920 | Mod032*fU*SfC*SfA*SfA*SfG*SfG*SmAfA*SmGmA*SfU*SmGmGfC*SfA*SfU*SfU |
| | *SfU*SfC*SfU |
| 921 | Mod033*fU*SfC*SfA*SfA*SfG*SfG*SmAfA*SmGmA*SfU*SmGmGfC*SfA*SfU*SfU |
| | *SfU*SfC*SfU |
| 922 | Mod033*fU*fC*fA*fA*fG*fG*mAfA*mGfA*mUfG*mGfC*fA*fU*fU*fU*fC*fU |
| 923 | Mod020L001fU*fC*fA*fA*fG*fG*mAfA*mGfA*mUfG*mGfC*fA*fU*fU*fU*fC*fU |
| 924 | Mod020L001*fU*fC*fA*fA*fG*fG*mAfA*mGfA*mUfG*mGfC*fA*fU*fU*fU*fC*fU |
| 925 | LOOI*fU*SfC*SfA*SfA*SfG*SfG*SmAfA*SmGmA*SfU*SmGmGfC*SfA*SfU*SfU*S |
| | fU*SfC*SfU |
| 926 | LOOIfU*SfC*SfA*SfA*SfG*SfG*SmAfA*SmGmA*SfU*SmGmGfC*SfA*SfU*SfU*Sf |
| | U*SfC*SfU |
| 927 | TGrCrCrArArCrUrGrGrGrArGrCrUrGrGrArGrCrGrCrArCrCrArArCrCrArG |
| 928 | LOOl*fU*fC*fA*fA*fG*fG*mAfA*mGfA*mUfG*mGfC*fA*fU*fU*fU*fC*fU |
| 929 | LOOlfU*fC*fA*fA*fG*fG*mAfA*mGfA*mUfG*mGfC*fA*fU*fU*fU*fC*fU |
| 930 | Mod015L001*fU*fC*fA*fA*fG*fG*mAfA*mGfA*mUfG*mGfC*fA*fU*fU*fU*fC*fU |
| 931 | Mod015L001fU*fC*fA*fA*fG*fG*mAfA*mGfA*mUfG*mGfC*fA*fU*fU*fU*fC*fU |
| 932 | fC*fC*fU*fU*fC*fC*mCfU*GmAfA*mGmGfU*fU*fC*fC*fU*fC*fC |
| 933 | LOOlfC*fC*fU*fU*fC*fC*mCfU*GmAfA*mGmGfU*fU*fC*fC*fU*fC*fC |
| 934 | Mod020L001fC*fC*fU*fU*fC*fC*mCfU*GmAfA*mGmGfU*fU*fC*fC*fU*fC*fC |
| 935 | fC*fC*fU*fU*fC*fC*mCfU*mGmAfA*mGmGfU*fU*fC*fC*fU*fC*fC |
| 936 | LOOlfC*fC*fU*fU*fC*fC*mCfU*mGmAfA*mGmGfU*fU*fC*fC*fU*fC*fC |
| 937 | Mod020L001fC*fC*fU*fU*fC*fC*mCfU*mGmAfA*mGmGfU*fU*fC*fC*fU*fC*fC |
| 938 | ModO15L001*fU*SPC*SfA*SfA*SfG*SfG*SmAfA*SmGmA*SfU*SmGmGfC*SfA*Sf |
| | U*SfU*SfU*SfC*SfU |
| 939 | ModO15L001*SfU*SfC*SfA*SfA*SfG*SfG*SmAfA*SmGmA*SfU*SmGmGfC*SfA*Sf |
| | U*SfU*SfU*SfC*SfU |
| 940 | LOO1*SfU*SfC*SfA*SfA*SfG*SfG*SmAfA*SmGmA*SfU*SmGmGfC*SfA*SfU*SfU* |
| | SfU*SfC* |
| 941 | fU*SfC*SfA*SfA*SfG*SfG*SmAfA*SmGmA*SfU*SmGmGfC*SfA*SfU*SfU*SfU*Sf |
| | c |
| 942 | fU*SfC*SfA*SfA*SfG*SfG*SmAfA*SmGmA*SfU*SmGmGfC*SfA*SfU*SfU*SfU |
| 943 | fC*SfA*SfA*SfG*SfG*SmAfA*SmGmA*SfU*SmGmGfC*SfA*SfU*SfU*SfU*SfC*Sf |
| | u |
| 944 | Mod020L001mG*mG*mC*mC*mA*mASmA*mC*mC*mU*mC*mG*mG*mC* |
| | mU*mU*mA*mC*mC*mU |
| 945 | Mod015L001mG*mG*mC*mC*mA*mA*mA*mC*mC*mU*mC*mG*mG*mC* |
| | mU*mU*mA*mC*mC*mU |
| 946 | fU*fU*fC*fU*fG*fU*mA*mA*mG*mG*mU*mU*mU*mU*fU*fA*fU*fG*fU*fG |
| 947 | fA*fU*fU*fU*rC*fU*mG*mU*mA*mA*mG*mG*mU*mU*fU*fU*fU*fA*fU*fG |
| 948 | fC*fC*fA*fU*fU*fU*mC*mU*mG*mU*mA*mA*mG*mG*fU*fU*fU*fU*fU*fA |
| 949 | fA*fU*fC*fC*fA*fU*mU*mU*mC*mU*mG*mU*mA*mA*fG*fG*fU*fU*fU*fU |
| 950 | fC*fA*fU*fC*fC*fA*mU*mU*mU*mC*mU*mG*mU*mA*fA*fG*fG*fU*fU*fU |
| 951 | fC*fC*fA*fU*fC*fC*mA*mU*mU*mU*mC*mU*mG*mU*fA*fA*fG*fG*fU*fU |
| 952 | fG*fC*fC*fA*fU*fC*mC*mA*mU*mU*mU*mC*mU*mG*fU*fA*fA*fG*fG*fU |
| 953 | fA*fG*fC*fC*fA*fU*mC*mC*mA*mU*mU*mU*mC*mU*fG*fU*fA*fA*fG*fG |
| 954 | fC*fA*fG*fC*fC*fA*mU*mC*mC*mA*mU*mU*mU*mC*fU*fG*fU*fA*fA*fG |
| 955 | fU*fC*fA*fG*fC*fC*mA*mU*mC*mC*mA*mU*mU*mU*fC*fU*fG*fU*fA*fA |
| 956 | fU*fU*fC*fA*fG*fC*mC*mA*mU*mC*mC*mA*mU*mU*fU*fC*fU*fG*fU*fA |
| 957 | fC*fU*fU*fC*fA*fC*mC*mC*mA*mU*mC*mC*mA*mU*fU*fU*fC*fU*fG*fU |
| 958 | fA*fC*fU*fU*fC*fA*mG*mC*mC*mA*mU*mC*mC*mA*fU*fU*fU*fC*fU*fG |
| 959 | fA*fA*fC*fU*fU*fC*mA*mG*mC*mC*mA*mU*mC*mC*fA*fU*fU*fU*fC*fU |
| 960 | fC*fA*fA*fC*fU*fU*mC*mA*mG*mC*mC*mA*mU*mC*fC*fA*fU*fU*fU*fC |
| 961 | fU*fC*fA*fA*fC*fU*mU*mC*mA*mG*mC*mC*mA*mU*fC*fC*fA*fU*fU*fU |
| 962 | fC*fC*fA*fG*fG*fG*mC*mA*mG*mG*mC*mC*mA*mU*fU*fC*fC*fU*fC*fU |
| 963 | fC*fC*fC*fA*fG*fG*mG*mC*mA*mG*mG*mC*mC*mA*fU*fU*fC*fC*fU*fC |
| 964 | fC*fC*fC*fC*fA*fG*mG*mG*mC*mA*mG*mG*mC*mC*fA*fU*fU*fC*fC*fU |
| 965 | fC*fC*fC*fC*fC*fA*mG*mG*mG*mC*mA*mG*mG*mC*fC*fA*fU*fU*fC*fC |
| 966 | fU*fC*fc*fC*fC*fC*mA*mG*mG*mG*mC*mA*mG*mG*fC*fC*fA*fU*fU*fC |
| 967 | fA*fU*fC*fC*fC*fC*mC*mA*mG*mG*mG*mC*mA*mG*fG*fC*fC*fA*fU*fU |
| 968 | fC*fA*fU*fC*fC*fC*mC*mC*mA*mG*mG*mG*mC*mA*fG*fG*fC*fC*fA*fU |
| 969 | fG*fC*fA*fU*fC*fC*mC*mC*mC*mA*mG*mG*mG*mC*fA*fG*fG*fC*fC*fA |
| 970 | fA*fG*fC*fA*fU*fC*mC*mC*mC*mC*mA*mG*mG*mG*fC*fA*fG*fG*fC*fC |
| 971 | fC*fA*fG*fC*fA*fU*mC*mC*mC*mC*mC*mA*mG*mG*fG*fC*fA*fG*fG*fC |
| 972 | fU*fC*fA*fG*fC*fA*mU*mC*mC*mC*mC*mC*mA*mG*fG*fG*fC*fA*fG*fG |
| 973 | fU*fU*fC*fA*fG*fC*mA*mU*mC*mC*mC*mC*mC*mA*fG*fG*fG*fC*fA*fG |
| 974 | fU*fU*fU*fC*fA*fG*mC*mA*mU*mC*mC*mC*mC*mC*fA*fG*fG*fG*fC*fA |
| 975 | fA*fU*fU*fU*fC*fA*mG*mC*mA*mU*mC*mC*mC*mC*fC*fA*fG*fG*fG*fC |
| 976 | fG*fA*fU*fU*fU*fC*mA*mG*mC*mA*mU*mC*mC*mC*fC*fC*fA*fG*fG*fG |
| 977 | fG*fG*fA*fU*fU*fU*mC*mA*mG*mC*mA*mU*mC*mC*ft*fC*fC*fA*fG*fG |
| 978 | fA*fG*fG*fA*fU*fU*mU*mC*mA*mG*mC*mA*mU*mC*fC*fC*fC*fC*fA*fG |
| 979 | fC*fA*fG*fG*fA*fU*mU*mU*mC*mA*mG*mC*mA*mU*fC*fC*fC*fC*fC*fA |
| 980 | fU*fC*fA*fG*fG*fA*mU*mU*mU*mC*mA*mG*mC*mA*fU*fC*fC*fC*fC*fC |
| 981 | fU*fU*fC*fA*fG*fG*mA*mU*mU*mU*mC*mA*mG*mC*fA*fU*fC*fC*fC*fC |
| 982 | fU*fU*fU*fC*fA*fG*mG*mA*mU*mU*mU*mC*mA*mG*fC*fA*fU*fC*fC*fC |
| 983 | fU*fU*fU*fU*fC*fA*mG*mG*mA*mU*mU*mU*mC*mA*fG*fC*fA*fU*fC*fC |
| 984 | fU*fU*fU*fU*fU*fC*mA*mG*mG*mA*mU*mU*mU*mC*fA*fG*fC*fA*fU*fC |
| 985 | fU*fU*fU*fU*fU*fU*mC*mA*mG*mG*mA*mU*mU*mU*fC*fA*fG*fC*fA*fU |
| 986 | fG*fU*fU*fU*fU*fU*mU*mC*mA*mG*mG*mA*mU*mU*fU*fC*fA*fG*fC*fA |
| 987 | fU*fG*fU*fU*fU*fU*mU*mU*mC*mA*mG*mG*mA*mU*fU*fU*fC*fA*fG*fC |
| 988 | fC*fU*fG*fU*fU*fU*mU*mU*mU*mC*mA*mG*mG*mA*fU*fU*fU*fC*fA*fG |
| 989 | fG*fC*fU*fG*fU*fU*mU*mU*mU*mU*mC*mA*mG*mG*fA*fU*fU*fU*fC*fA |
| 990 | fA*fG*fC*fU*fG*fU*mU*mU*mU*mU*mU*mC*mA*mG*fG*fA*fU*fU*fU*fC |
| 991 | fG*fA*fG*fC*fU*fG*mU*mU*mU*mU*mU*mU*mC*mA*fG*fG*fA*fU*fU*fU |
| 992 | fU*fG*fA*fG*fC*fU*mG*mU*mU*mU*mU*mU*mU*mC*fA*fG*fG*fA*fU*fU |
| 993 | fU*fU*fG*fA*fG*fC*mU*mG*mU*mU*mU*mU*mU*mU*fC*fA*fG*fG*fA*fU |
| 994 | fU*fU*fU*fG*fA*fG*mC*mU*mG*mU*mU*mU*mU*mU*fU*fC*fA*fG*fG*fA |
| 995 | fG*fU*fU*fU*fG*fA*mG*mC*mU*mG*mU*mU*mU*mU*fU*fU*fC*fA*fG*fG |
| 996 | fU*fU*fG*fU*fU*fU*mG*mA*mG*mC*mU*mG*mU*mU*fU*fU*fU*fU*fC*fA |
| 997 | fC*fA*fU*fU*fG*fU*mU*mU*mG*mA*mG*mC*mU*mG*fU*fU*fU*fU*fU*fU |
| 998 | fG*fC*fA*fU*fU*fG*mU*mU*mU*mG*mA*mG*mC*mU*fG*fU*fU*fU*fU*fU |
| 999 | fU*fG*fC*fA*fU*fU*mG*mU*mU*mU*mG*mA*mG*mC*fU*fG*fU*fU*fU*fU |
| 1000 | fC*fU*fG*fC*fA*fU*mU*mG*mU*mU*mU*mG*mA*mG*fC*fU*fG*fU*fU*fU |
| 1001 | fU*fC*fU*fG*fC*fA*mU*mU*mG*mU*mU*mU*mG*mA*fG*fC*fU*fG*fU*fU |
| 1002 | fC*fU*fC*fU*fG*fC*mA*mU*mU*mG*mU*mU*mU*mG*fA*fG*fC*fU*fG*fU |
| 1003 | fA*fC*fU*fC*fU*fG*mC*mA*mU*mU*mG*mU*mU*mU*fG*fA*fG*fC*fU*fG |
| 1004 | fU*fA*fC*fU*fC*fU*mG*mC*mA*mU*mU*mG*mU*mU*fU*fG*fA*fG*fC*fU |
| 1005 | fU*fU*fA*fC*fU*fC*mU*mG*mC*mA*mU*mU*mG*mU*fU*fU*fG*fA*fG*fC |
| 1006 | fC*fU*fU*fA*fC*fU*mC*mU*mG*mC*mA*mU*mU*mG*fU*fU*fU*fG*fA*fG |
| 1007 | fU*fC*fU*fU*fA*fC*mU*mC*mU*mG*mC*mA*mU*mU*fG*fU*fU*fU*fG*fA |
| 1008 | fA*fU*fC*fU*fU*fA*mC*mU*mC*mU*mG*mC*mA*mU*fU*fG*fU*fU*fU*fG |
| 1009 | fA*fA*fU*fC*fU*fU*mA*mC*mU*mC*mU*mG*mC*mA*fU*fU*fG*fU*fU*fU |
| 1010 | fC*fA*fA*fA*fU*fC*mU*mU*mA*mC*mU*mC*mU*mG*fC*fA*fU*fU*fG*fU |
| 1011 | fG*fA*fU*fA*fC*fA*mA*mA*mU*mC*mU*mU*mA*mC*fU*fC*fU*fG*fC*fA |
| 1012 | Geo*Geo*Geo*Teo*m5Ceo*A*G*C*T*G*C*C*A*A*T*Geo*m5Ceo*Teo*Aeo*Geo |
| 1013 | Mod030Geo*Geo*Geo*Teo*m5Ceo*A*G*C*T*G*C*C*A*A*T*Geo*m5Ceo*Teo*Aeo |
| | *Geo |
| 1014 | Mod031Geo*Geo*Geo*Teo*m5Ceo*A*G*C*T*G*C*C*A*A*T*Geo*m5Ceo*Teo*Aeo |
| | *Geo |
| 1015 | Mod032Geo*Geo*Geo*Teo*m5Ceo*A*G*C*T*G*C*C*A*A*T*Geo*m5Ceo*Teo*Aeo |
| | *Geo |
| 1016 | Mod033Geo*Geo*Geo*Teo*m5Ceo*A*G*C*T*G*C*C*A*A*T*Geo*m5Ceo*Teo*Aeo |
| | *Geo |

In Table 2; “*” represents a stereorandom phosphorothioate linkage; “*S” represents an Sp phosphorothioate linkage; “*R” represents an Rp phosphorothioate linkage; all non-labeled linkage is a natural phosphate linkage; “m” preceding a base represents 2′-OMe; and “eo” following a base represents 2′-MOE. F represents a fluorinated nucleoside. LOO1 represents a C6 PO(phosphodiester) or PS(phosphorothioate) linker. Mod represents a modification attached to the nucleic acid: Lauric (in Mod013), Myristic (in Mod014), Palmitic (in Mod005), Stearic (in Mod015), Oleic (in Mod016), Linoleic (in Mod017), alpha-Linoleinc (in Mod018), gamma-Linolenic (in Mod019), DHA (in Mod006), Turbinaric (in Mod020), Dilinoleic (in Mod021), TriGlcNAc (in Mod024), TrialphaMannose (in Mod026), MonoSulfonamide (in Mod 027), TriSulfonamide (in Mod029), Lauric (in Mod030), Myristic (in Mod031), Palmitic (in Mod032), and Stearic (in Mod033): Lauric acid (for Mod013), Myristic acid (for Mod014), Palmitic acid (for Mod005), Stearic acid (for Mod015), Oleic acid (for Mod016), Linoleic acid (for Mod017), alpha-Linolenic acid (for Mod018), gamma-Linolenic acid (for Mod019), docosahexaenoic acid (for Mod006), Turbinaric acid (for Mod020), alcohol for Dilinoleyl (for Mod021), acid for TriGlcNAc (for Mod024), acid for TrialphaMannose (for Mod026), acid for MonoSulfonamide (for Mod 027), acid for Tri Sulfonamide (for Mod029), Lauryl alcohol (for Mod030), Myristyl alcohol (for Mod031), Palmityl alcohol (for Mod032), and Stearyl alcohol (for Mod033), respectively, conjugated to oligonucleotide chains through amide groups, C6 amino linker, phosphodiester linkage (PO), and/or phosphorothioate linkage (PS).

TABLE 3

| SEQ | |
|---|---|
| ID | |
| NO. | Sequence 5′ -> 3 |
| 1017 | GUGGAAUAGUAUAACAAUAUgcuaaAUGUUGUUAUAGUAUCCCACACUGAGC |
| | AAUGCcGUAGUCAG*C*A*A*U |
| 1018 | GUGGAAUAGUAUAACAAUAUgcuaaAUGUUGUUAUAGUAUCCCACGUACUGA |
| | GCAAUGCcGUAGUCAGCAA*U*C*U*U |
| 1019 | GGAAUAGUAUAACAAUAUgcuaaAUGUUGUUAUAGUAUCCCACUGAGCAAUG |
| | CcGUAGUCAG*C*A*A*U |
| 1020 | GGAAUAGUAUAACAAUAUgcuaaAUGUUGUUAUAGUAUCCCGUACUGAGCAA |
| | UGCcGUAGUCAGCAA*U*C*U*U |
| 1021 | cgcgcgttttcgcgcgGCUGAACCACUGCAC |
| 1022 | cgcgcgttttcgcgcgGAGAUACUCACAAUU |
| 1023 | cgcgcgttttcgcgcgCGUUGACCUCCACUC |
| 1024 | rGrGrArArUrArGrUrArUrArArCrArArUrArUrgrcrurararArU |
| | rGrUrUrGrUrUrArUrArGr UrArUrCrCrCmC*mA*mG*mU*mCmC |
| | mCmUmUmUmCrUrCrGmUmCmGmAmUmGmG*mU*mC*mA*mG |
| 1025 | mG*mG*mA*mA*mU*mA*mG*mU*mA*mU*mA*mA*mC*mA*mA*mU*mA*mU* |
| | mG*mC*mU*mA*mA*mA*mU*mG*mU*mU*mG*mU*mU*mA*mU*mA*mG*mU* |
| | mA*mU*mC*mC*mCmC*mA*mG*mU*mCmCmCmUmUmUmCrUrCrGmUmCmGm |
| | AmUmGmG*mU*mC*mA*mG |
| 1026 | unmodified GUGUUGGCCAUGGAACAUAUAACAAUAUgcuaaAUGUUGUUAUA |
| 1027 | 2′OMe-PS UAUAACAAUAUgcuaaAUGUUGUUAUAGUGUUGGCCAUGGAACA |
| 1028 | 2′Ome-PS GUGUUGGCCAUGGAACAUAUAACAAUAUgcuaaAUGUUGUUAUA |
| 1029 | unmodified GUGUUGGCCAUGGAACAAUAGUAUAACAAUAUgcuaaAUGUUGUU |
| | AUAGUAU |
| 1030 | unmodified GGAAUAGUAUAACAAUAUgcuaaAUGUUGUUAUAGUAUCCCGUGU |
| | UGGCCAUGGAACA |
| 1031 | unmodified GUGUUGGCCAUGGAACAGGAAUAGUAUAACAAUAUgcuaaAUGUU |
| | GUUAUAGUAUCCC |
| 1032 | 2'OMe-PSGGAAUAGUAUAACAAUAUgcuaaAUGUUGUUAUAGUAUCCCGUGUU |
| | GGCCAUGGAACA |

In Table 3; “R” represents no modification; “m” represents 2′O-Me, “PS represents phosphorothioate linkages, and “*” represents phosphorothioate linkage.

TABLE 4

| SEQ | | |
|---|---|---|
| ID | | |
| NO. | Sequence 5′ -> 3 | Base modifications |
| 1033 | u*c*a*g*ucccuuucUCGucgauggucagc*a*c*a*g | |
| 1034 | u*c*a*g*ucccuuucuCGucgauggucagc*a*c*a*g | |
| 1035 | u*c*a*g*ucccuuucuCGUcgauggucagc*a*c*a*g | |
| 1036 | u*c*a*g*ucccuuucucgucgauggucagc*a*c*a*g | |
| 1037 | u*c*a*g*ucccuuucU*CGucgauggucagc*a*C*a*g | |
| 1038 | u*c*a*g*ucccuuuc[TCG]ucgauggucagc*a*C*a*g | |
| 1039 | uu*c*a*g*ucccuuuc(ucg)ucgauggucagc*a*C*a*g | |
| 1040 | u*a*g*ucccuuuc{U}C{G}ucgauggucagc*a*C*a*g | |
| 1041 | u*c*a*g*ucccuuuc{U}X{G}ucgauggucagc*a*c*a*g | X: 5-Methylcytidine |
| 1042 | u*c*a*g*ucccuuucUCZucgauggucagc*a*c*a*g | Z: Inosine |
| 1043 | u*c*a*g*ucccuuucUXZucgauggucagc*a*c*a*g | X: 5-Methylcytidine, |
| | | Z: Inosine |
| 1044 | u*c*a*g*ucccuuucYXZucgauggucagc*a*c*a*g | Y: Pseudouridine, |
| | | X: 5-Methylcytidine, |
| | | Z: Inosine |
| 1045 | u*c*a*g*ucccuuucYXZucgauggucagc*a*c*a*g | Y: Pseudouridine, |
| | | X: 5-Methylcytidine, |
| | | Z: Inosine |
| 1046 | u*c*a*g*ucccuuucYXZucgauggucagc*a*c*a*g | Y: 5-Methyluridine, X: |
| | | 5-Methylcytidine, |
| | | Z: Inosine |
| 1047 | u*c*a*g*ucccuuucYXZucgauggucagc*a*c*a*g | Y: Thienouridine, |
| | | X: 5-Methylcytidine, |
| | | Z: Inosine |
| 1048 | u*c*a*g*ucccuuucYXZucgauggucagc*a*c*a*g | Y: 2,6-diaminopurine, |
| | | X: 5-Methylcytidine, |
| | | Z: Inosine |
| 1049 | u*c*a*g*ucccuuucUXZucgauggucagc*a*c*a*g | X: Pyrrolocytidine, Z: |
| | | Inosine |
| 1050 | u*c*a*g*ucccuuucYXZucgauggucagc*a*c*a*g | Y: Pseudouridine, |
| | | X: Pyrrolocytidine, Z: |
| | | Inosine |
| 1051 | u*c*a*g*ucccuuucYXZucgauggucagc*a*c*a*g | Y: Thienouridine, |
| | | X: Pyrrolocytidine, Z: |
| | | Inosine |
| 1052 | u*c*a*g*ucccuuucYXZucgauggucagc*a*c*a*g | Y: Pseudouridine, X: 5- |
| | | Methylcytidine, |
| | | Z: Thienoguanosine |
| 1053 | u*c*a*g*ucccuuucYXZucgauggucagc*a*c*a*g | Y: Pseudouridine, |
| | | X: Pyrrolocytidine, Z: |
| | | Thienoguanosine |
| 1054 | u*c*a*g*ucccuuucU′C′Gvucgauggucagc*a*c*a*g | |
| 1055 | u*c*a*g*ucccuuucU″C″G″ucgauggucagc*a*c*a*g | |
| 1056 | u*c*a*g*ucccuuucUACAGAucgauggucagc*a*c*a*g | |
| 1057 | u*c*a*g*ucccuuucU#C#G#ucgauggucagc*a*c*a*g | |
| 1058 | u*c*a*g*ucccuuucYXZucgauggucagc*a*c*a*g | Y: 5-Methoxyuridine, |
| | | X: 5-Methylcytidine, |
| | | Z: I nosine |
| 1059 | u*c*a*g*ucccuuucYXZucgauggucagc*a*c*a*g | Y: dihydrouridine, X: 5- |
| | | Methylcytidine, |
| | | Z: Inosine |
| 1060 | u*c*a*g*ucccuuucYXZucgauggucagc*a*c*a*g | Y: Pseudouridine, |
| | | X: 5- |
| | | Hydroxymethylcytidine, |
| | | Z: Inosine |
| 1061 | u*c*a*g*ucccuuucYXZucgauggucagc*a*c*a*g | Y: Pseudouridine, |
| | | X: 5-Methylcytidine, |
| | | Z: 7-Methylguanosine |
| 1062 | u*c*a*g*ucccuuucYXZucgauggucagc*a*c*a*g | Y: Pseudouridine, |
| | | X: 5-Methylcytidine, |
| | | Z: 7-deazaguanosine |
| 1063 | u*c*a*g*ucccuuucYXZucgauggucagc*a*c*a*g | Y: Pseudouridine, |
| | | X: 5-Methylcytidine, |
| | | Z: 8-aza-7- |
| | | deazagunaosine |
| 1064 | u*c*a*g*ucccuuucYXZucgauggucagc*a*c*a*g | Y: Pseudouridine, |
| | | X: 5-Methylcytidine, |
| | | Z: 7-aminomethyl-7- |
| | | deazaguanosine |
| 1065 | c*c*u*g*cgacacuucggcCCAgagcugcucc*u*c*a*u | |
| 1066 | c*c*u*g*cgacacuucggcXXYgagcugcucc*u*c*a*u | X: 5-Methylcytidine, |
| | | Y: 7-Methyladenosine |
| 1067 | c*c*u*g*cgacacuucggcXXYgagcugcucc*u*c*a*u | X: 5-Methylcytidine, |
| | | Y: 8-Methyladenosine |
| 1068 | c*c*u*g*cgacacuucggcXXYgagcugcucc*u*c*a*u | X: 5-Methylcytidine, |
| | | Y: 3-deazaadenosine |
| 1069 | c*c*u*g*cgacacuucggcXXYgagcugcucc*u*c*a*u | X: 5-Methylcytidine, |
| | | Y: 7-deazaadenosine |
| 1070 | c*c*u*g*cgacacuucggcXXYgagcugcucc*u*c*a*u | X: 5-Methylcytidine, |
| | | Y: 8-azidoadenosine |
| 1071 | c*c*u*g*cgacacuucggcXXYgagcugcucc*u*c*a*u | X: 5-Methylcytidine, |
| | | Y: Inosine |
| 1072 | c*a*u*u*gaagaagauaagagaaaguacugagaaguguuggCCAuggaacag*g*u*a*g | |
| 1073 | c*a*u*u*gaagaagauaagagaaaguacugagaaguguuggCCZuggaacag*g*u*a*g | Z: 2-aminopurine |
| 1074 | c*a*u*u*gaagaagauaagagaaaguacugagaaguguugg[C]C[A]uggaacag*g*u*a*g | |
| 1075 | g*a*c*u*gagguacuccuuagagaaaggug[CCA]cuucuuggcaa*a*g*g*a | |
| 1076 | g*u*a*g*gcaugggaggaaaaggugCCAcuucuuggcaa*a*g*g*a | |
| 1077 | C*u*g*u*ccaacacagccccagccuuugagaccucugcCCAgaguuguu*c*u*c*c | |
| 1078 | c*u*g*u*ccaacacagccccagccuuugagaccucuguCC[A]gaguuguu*c*u*c*c idT | |
| 1079 | C*u*g*u*ccaacacagccccagccuuugagaccucuguCC[A]gaguuguu*c*u*c*C idT | |
| 1080 | C*u*g*u*ccaacacagccccagccuuugagaccucuguXC[A]gaguuguu*c*u*c*c | X: 5-Methylcytidine |
| | idT | |
| 1081 | c*u*g*u*ccaacacagccccagccuuugagaccucugucC[X]gaguuguu*c*u*c*c | X: deoxy 2- |
| | idT | aminopurine |

| | |
|---|---|
| 1082 | c*u*g*u*ccaacacagccccagccuuugagaccucugucc[a]gaauuguu*c*u*c*c idt |
| 1083 | c*u*g*u*ccaacacagccccagccuuugagaccucugucc[aga]guuguu*c*u*c*c idt |
| 1084 | c*u*g*u*ccaacacagccccagccuuugagaccucuguc*c*[a]*g*a*guuguu*c*u*c*c idt |
| 1085 | c*u*g*u*ccaacacagccccagccuuugagaccucuguc*c*[a]*gaguuguu*c*u*c*c idt |
| 1086 | c*u*g*u*ccaacacagccccagccuuugagaccucug*u*c*c*a*g*aguuguu*c*u*c*c idt |
| 1087 | c*u*g*u*ccaacacagccccagccuuugagaccucuguc(caga)guuguu*c*u*c*c |
| 1088 | c*u*g*u*ccaacacagccccagccuuugagaccucugu[CCA]gaguuguu*c*u*c*c |
| 1089 | c*u*g*u*c*c*a*a*c*a*c*a*gccccagccuuugagaccucugu[CCA]gaguuguu*c*u*c*c |
| 1090 | c*u*g*u*ccaacacagccc*c*a*g*c*c*u*u*u*g*a*gaccucugu[CCA]gaguuguu*c*u*c*c |
| 1091 | c*u*g*u*c*c*a*a*c*a*c*a*gccc*c*a*g*c*c*u*u*u*g*a*gaccucugu[CCA]gaguuguu*c*u*c*c |
| 1092 | c*u*g*u*ccaacacagccccagccuuugagaccucuguc[CA]gaguuguu*c*u*c*c |
| 1093 | c*u*g*u*ccaacacagccccagccuuugagaccucugu[C)(C)[A]gaguuguu*c*u*c*c |
| 1094 | c*u*g*u*ccaacacagccccagccuuugagaccucugu[C*C*A*]gaguuguu*c*u*c*c |
| 1095 | c*u*g*u*ccaacacagccccagccuuugagaccucugu[CCA]gaguuguu*c*u*c*c idt |
| 1096 | a*c*a*c*a*G*cuc*c*a*g*c*c*u*u*u*G*A*gaccu*c*u*g*cCCAGaguu*g*u*u*c*u*c*c |
| 1097 | c*a*c*a*gccccagccuuugagaccucugu[CCA]gaguuguu*c*u*c*c |
| 1098 | c*a*c*a*g*c*c*c*c*a*g*c*c*u*u*u*g*agaccucugu[CCA]gaguuguu*c*u*c*c |
| 1099 | c*a*c*a*gccc*c*a*g*c*c*u*u*u*g*a*gaccucugcc*[C*A]*gaauuguu*c*u*c*c |
| 1100 | G*a*c*u*gagguacuccauagggaaaggcacc[A]cuucuuggcaa*a*g*g*a |
| 1101 | G*a*c*u*gagguacuccauagggaaaggcacc[ACU]ucuuggcaa*a*g*g*a |
| 1102 | G*a*c*u*gagguacuccauagggaaaggcacc[A]<cu>ucuuggcaa*a*g*g*a |

In Table 4, specific YXZ base modifications are mentioned in the third column. Lower case nucleotides are RNA and 2′-O-methyl modified. Upper case nucleotides are RNA, except for bracketed [NNN] nucleotides, which is DNA. Lower case nucleotides depicted as (nnn) are 2′-fluoro RNA modified nucleotides. Lower case nucleotides depicted as <nnn> are 2′-NH2 RNA modified nucleotides. Nucleotides depicted as {N} are Unlocked Nucleic Acid (UNA). “idT” indicates a 3′ inverted T modification which enhances the resistance to degradation and also blocks the 3′-terminus of AON from extension during PCR amplification. “*” represents phosphorothioate linkages; “′” =3′-methylenephosphonate linkages; “″” represents 5′-methylenephosphonate linkages; “Λ” represents 3′-phosphoroamidate linkages; and “#” represents 2′-5′ phosphodiester linkages.

TABLE 5

| SEQ | |
|---|---|
| ID | |
| NO. | Sequence 5′ -> 3 |
| 1103 | g*a*c*u*gagguacuccuuagagaaaggugCCAcuucuuggca |
| | a*a*g*g*a- |
| 1104 | mC*mA*mU*mU*mGmAmAmGmAmAmGmAmUmAmAmGmAmGmAm |
| | AmAmGmUmAmCmUmGmAmGmAmAmGmUmGmUmUmGmGCCAmUm |
| | GmGmAmAmCmAmG*mG*mU*mA*mG |
| 1105 | cauugaagaa gauaagagaa aguacugaga aguguuggcc |
| | auggaacagg uag |

In Table 5; RNA is depicted by A, C, G, or U; DNA is depicted by dA, dC, dG, or dT; 2′-Ome is depicted by mA, mC, mG, or mU; PMO (Phosphorodiamidate morpholino oligomers) are depicted by pA, pC, pG, or pT; and Phosphorothioate is depicted by “*”.

TABLE 6

| SEQ | |
|---|---|
| ID | |
| NO. | Sequence 5′ -> 3 |
| 1106 | GUGGAUAGUAUAACAAUAUGCUMAAUGUUGUUAUAGUAUCCCAC |
| 1107 | GUGGASSSGSASASCAAUAUGCUMAAUGUUGSUSUSGSSSCCCAC |
| 1108 | GUGGAASAGSASAACAAUAUGCUMAAUGUUGUUSUSGUSUCCCAC |
| 1109 | GUGGASASUAUAACAAUAUGCUMAAUGUUGUUAUAGYAYCCCAC |
| 1110 | GUGGASSSGSSSSSSSSUAUGCUMAAUGSSSSSSSSGSSSCCCAC |
| 1111 | GUGGAASSGSASASCAAUAUGCUMAAUGUUGSUSUSGSSU |
| | CCCAC |
| 1112 | GUGGSAUAGUAUAACAAUAUGCUMAAUGUUGUUAUAGUAUCCCAC |
| 1113 | ACGCAACCAAGUCAUA |
| 1114 | GCAAUGCCAUCACCUC |
| 1115 | AGGGGUCCACAUGGCA |
| 1116 | GGCUCCCCAGGCCCCU |
| 1117 | UGCCGUCCACCAGGAU |
| 1118 | CAGAUUCCAGGUGGGA |
| 1119 | UCCCUGCCAGAAUAGA |
| 1120 | CUCCGCCCACCAAAUG |
| 1121 | CCCAAACCACAACAGA |
| 1122 | ACCCACCCACCCAGGU |
| 1123 | CUGCCGCCAGCUGGAU |
| 1124 | AGGGAACCAGACAGUU |

In Table 6; “S” can be G or C, “Y” can be C or T; and “M” can be A or C.

TABLE 7

| SEQ ID | |
|---|---|
| NO. | Sequence 5′ -> 3 |
| 1125 | U*U*C*A*C*U*UcAG*U*G*U*As*Us*Gs*Cs*C* |
| 1126 | U*U*C*A*C*U*UcAG*U*G*U*As*Us*Gs*Cs*C* |
| 1127 | A*C*C*U*C*C*AcUC*A*G*U*Gs*Us*Gs*As*U* |
| 1128 | U*U*U*C*C*U*CcAC*U*G*U*Us*Gs*Cs*As*A* |
| 1129 | U*G*U*G*U*A*UcUU*G*C*U*Gs*Us*Gs*As*G* |
| 1130 | G*A*G*G*U*C*CcUG*G*G*G*Gs*Cs*Gs*Cs*U* |
| 1131 | G*A*U*C*U*U*CcUG*A*U*G*Gs*Cs*Cs*As*C* |
| 1132 | A*G*C*C*A*C*AcAC*U*C*C*Gs*Us*Cs*As*G* |
| 1133 | G*A*U*U*U*U*CcUG*A*U*A*Gs*Cs*Us*As*C* |
| 1134 | G*G*C*C*A*C*AcAU*U*C*U*Gs*Us*Cs*As*G* |
| 1135 | G*A*U*C*U*U*CcUG*A*U*G*Gs*Cs*Cs*As*C* |
| 1136 | G*G*C*C*A*C*AcAC*U*C*C*Gs*Us*Cs*As*G* |
| 1137 | G*A*U*U*U*U*CcUG*A*U*A*Gs*Cs*As*As*C* |
| 1138 | G*G*C*U*A*C*GcAC*U*C*U*Gs*Us*Cs*As*A* |
| 1139 | A*G*G*C*C*G*CcGU*C*G*U*Gs*Gs*Cs*Gs*G* |
| 1140 | C*C*G*C*U*C*CcUCcUC*A*G*C*Cs*Cs*Gs*Us*C* |
| 1141 | A*C*G*C*C*A*CcAG*C*U*C*Cs*As*As*Cs*U* |
| 1142 | G*U*C*U*C*A*CcAA*U*U*G*Cs*Us*Cs*Us*C* |
| 1143 | G*A*A*A*U*A*CcAU*C*A*G*As*Us*Us*Us*G* |
| 1144 | A*A*U*U*A*G*CcUU*C*U*G*Gs*Cs*Cs*As*U* |
| 1145 | G*A*U*C*A*G*CcUC*C*U*G*Gs*Cs*Cs*As*U* |
| 1146 | G*A*U*C*A*G*CcUU*C*U*G*Gs*Cs*Cs*As*U* |
| 1147 | G*A*U*C*A*G*CcUU*C*U*G*Gs*Cs*Cs*As*U* |
| 1148 | *A*C*U*G*C*CcAG*G*C*A*Us*Cs*As*Gs*C* |
| 1149 | C*A*C*U*G*C*CcGG*G*C*A*Us*Cs*As*Gs*C* |
| 1150 | U*C*C*G*C*C*CcGA*U*C*C*As*Cs*Gs*As*U* |
| 1151 | C*C*U*U*U*C*UcGU*C*G*A*Us*Gs*Gs*Us*C* |
| 1152 | C*C*U*U*U*C*U*cGU*C*G*A*Us*Gs*Gs*Us*C* |
| 1153 | C*U*U*G*A*U*AcAU*C*C*A*Gs*Us*Us*Cs*C* |
| 1154 | U*U*U*C*A*G*GcAU*U*U*C*Cs*Us*Cs*Cs*G* |
| 1155 | C*u*U*C*A*G*GcAU*G*G*G*Gs*Cs*As*Gs*C* |
| 1156 | A*G*G*A*A*C*AcAA*C*C*U*Us*Us*Gs*Us*C* |
| 1157 | U*U*U*C*A*C*AcAU*C*C*A*Us*Cs*As*As*C* |
| 1158 | C*u*U*C*A*C*GcAU*C*C*A*Us*Cs*As*As*C* |
| 1159 | U*G*G*G*A*C*AcAA*C*C*C*Cs*Us*Gs*Cs*C* |
| 1160 | C*G*A*C*U*C*CcUC*U*G*G*As*Us*Gs*Us*U* |
| 1161 | C*G*A*C*U*C*UcUC*U*G*G*As*Us*Gs*Us*U* |

In Table 7; Nₐ and N_{b} can form a mismatch, in some cases where Nₐ is adenosine and N_{b} is cytidine; N_{c} and N_{d} form a mismatch, in some cases wherein N_{c} and N_{d} are guanosine; “Gs” is a guanosine comprising a phosphorothioate group; “Gsl” is an LNA guanosine comprising a phosphorothioate group; and wherein an asterisk (*) indicates a modification of the nucleotide at the 2 carbon atom, in some cases with 2′-hydrogen (2′-cleoxy), 2′-0-methyl, 2′-0-methoxyethyl or 2′-fluoro; “A” is an adenosine nucleotide or a variant thereof, in some cases an adenosine ribonucleotide, an adenosine deoxynucleotide, a modified adenosine ribonucleotide or a modified adenosine deoxynucleotide; “C” is a cytidine nucleotide or a variant thereof, for example a cytidine ribonucleotide, a cytidine deoxynucleotide, a modified cytidine ribonucleotide or a modified cytidine deoxynucleotide; “G” is a guanosine nucleotide or a variant thereof, for example a guanosine ribonucleotide, a guanosine deoxynucleotide, a modified guanosine ribonucleotide or a modified guanosine deoxynucleotide; “U” is an uridine nucleotide or a variant thereof, for example a uridine ribonucleotide, a uridine deoxynucleotide, a modified uridine ribonucleotide, or a modified uridine deoxynucleotide; “A”, “C”, “G” or “U” is a nucleotide, in some cases a ribonucleotide or a deoxynucleotide as defined above, further comprising a phosphorothioate group; wherein an asterisk (*) indicates a chemical modification of the preceding nucleotide at the 2′ carbon atom, for example with 2′-hydrogen (2′-deoxy), 2′-0-methyl, 2′-0-methoxyethyl or 2′-fluoro; and wherein a lower case letter c indicates the position corresponding to a nucleotide, for example an adenosine or a cytidine, for example an adenosine, to be edited in the target sequence and wherein c represents a cytidine nucleotide or a variant thereof, a deoxycytidine nucleotide or a variant thereof, or an abasic site.

TABLE 8

| SEQ | |
|---|---|
| ID | |
| NO. | Sequence 5′ -> 3 |
| 1162 | (GUGGAAUAGUAUAACAAUAUGCUAAAUGUUGUUAUAGUAUCCCACGUGCA |
| | GCCAGCCGUCCUCUAGAGGGCCCUGAAGAGGGCCC) |
| 1163 | (GUGGAAGAGGAGAACAAUAUGCUAAAUGUUGUUCUCGUCUCCCACGUGCA |
| | GCCAGCCGUCCUCUAGAGGGCCCUGAAGAGGGCCC) |
| 1164 | (GUGGUCGAGAAGAGGAGAACAAUAUGCUAAAUGUUGUUCUCGUCUCCUCG |
| | ACCACGUGCAGCCAGCCGUCCUCUAGAGGGCCCUGAAGAGGGCCC) |
| 1165 | [GCAAUG](CCA)[UC AC][C*][U][C][C*][C] |
| 1166 | (GGUGAAUAGUAUAACAAUAUGCUAAAUGUUGUUAUAGUAUCCACC)[GCAA |
| | UG](CCA)[UCAC][C][U*][C*][C*][C] |
| 1167 | (GGUGAAGAGGAGAACAAUAUGCUAAAUGUUGUUCUCGUCUCCACC)[GCAA |
| | UG](CCA)[UCAC][C][U*][C*][C*][C] |
| 1168 | (GGUGUCGAGAAGAGGAGAACAAUAUGCUAAAUGUUGUUCUCGUCUCCUCG |
| | ACACC)[GCAAUG](CCA)[UCAC][C*][U*][C][C][C] |
| 1169 | [AGGGGU](CCA)[CAUG][G][C*][A][A][C] |
| 1170 | (GGUGAAUAGUAUAACAAUAUGCUAAAUGUUGUUAUAGUAUCCACC)[AGGG |
| | GU](CCA)[CAUG][G*][C*][A][A][C] |
| 1171 | (GGUGAAGAGGAGAACAAUAUGCUAAAUGUUGUUCUCGUCUCCACC)[AGGG |
| | GU](CCA)[CAUG][G*][C*][A*][A*][C] |
| 1172 | (GGUGUCGAGAAGAGGAGAACAAUAUGCUAAAUGUUGUUCUCGUCUCCUCG |
| | ACACC)[AGGGGU](CCA)[CAUG][G*][C*][A*][A*][C] |
| 1173 | [G*][G*][U](G)[U][C](GAGAAGAGGAGAA)[C](AA)[U] |
| 1174 | [GGGGUG](CCA)[AGCA][G*][U*][U*][G*][G] |
| 1175 | (GGUGUCGAGAAGAGGAGAACAAUAUGCUAAAUGUUGUUCUCGUCUCCUCG |
| | ACACC)[GGGGUG](CCA)[AGCA][G*][U*][U*][G][G] |
| 1176 | [GGGGUG](CCA)[AGCA][G][U*][U*][G*][G] |
| 1177 | (GGUGUCGAGAAGAGGAGAACAAUAUGCUAAAUGUUGUUCUCGUCUCCUCG |
| | ACACC)[GUUUUU](CCA)[GACG][G*][C*][A*][G*][G] |
| 1178 | [G]*[G]*[U](G)[U][C](GAGAAGAGGAGAA)[C](AA)[U] |
| 1179 | [G]*[G]*[U](GUCGAGAAGAGGAGAAC AAUAUGCUA |
| 1180 | (GGUGUCGAGAAGAGGAGAACAAUAUGCUAAAUGUUGUUCUCGUCUCCUCG |
| | ACACC)[CCUUUC](UCG)[UCGA][U*][G*][G*][U*][C] |
| 1181 | (CAUGGCCCCAGCAGCUUCAGUC)[C]{C}[UUUC](UCG) |
| 1182 | [G*][G*][U](G)[U][C](GAGAAGAGGAGAA)[C](AA)[U](A)[U](G)[C][U](AAA)[U] |
| | (G)[U][U](G)[U][U][C][U][U][C][C][U][C}[GACACCCAUGGCCCCAGCAGCUUCAG |
| | UC)[C]{C}[UUUC](UCG)[UCGA]{T*}[G*][G*]{T*}[C] |
| 1183 | [G*][G*][U](G)[U][C](GAGAAGAGGAGAA)[C](AA)[U](A)[U](G)[C][U](AAA)[U] |
| | (G)[U][U](G)[U][U][C][U][C](G)[U][C][U][C][C][U][C](GACACCCAUGGCCCCA |
| | GCAGCUUCAGUC)[C]{C}[UUUCU](CG)[UCGA]{T*}[G*][G*]{T*}[C] |
| 1184 | [G*][G*][U](G)[U][C](GAGAAGAGGAGAA)[C](AA)[U] |
| 1185 | (GGUGAAUAGUAUAACAAUAUGCUAAAUGUUGUUAUAGUAUCCACCAGGGG |
| | UCCACAUGGCAAC) |
| 1186 | (GGUGAAGAGGAGAACAAUAUGCUAAAUGUUGUUCUCGUCUCCACCAGGGG |
| | UCCACAUGGCAAC) |
| 1187 | (GGUGUCGAGAAGAGGAGAACAAUAUGCUAAAUGUUGUUCUCGUCUCCUCG |
| | ACACCAGGGGUCCACAUGGCAAC) |
| 1188 | (GGACCAACUGCUUGGCACCCCUGGCCAAGGUCAUCCAUGACAACUUUGGU |
| | AUCGUGGAAGGACC) |
| 1189 | (GGGAACUGGAUCUAUCAAGACUGAGUUGAUUUCUGUGUCUGAAGUGUAAG |
| | UGAACACAGAA) |
| 1190 | (GGACCATCGACGAGAAAGGGACTGAAGCTGCTGGGGCCATGTTTTTAGAGG |
| | CCATACCCAT) |

In Table 8; a base in parentheses e.g. “(N)” depicts an RNA base; a letter in square brackets e.g. “[N]” depicts a 2′-OMe RNA base; “*” depicts a Phosphorothioate linkage; a base in curly brackets e.g. “{N}” depicts an LNA base; “c” is a cytidine nucleotide or a variant thereof, a deoxycytidine nucleotide or a variant thereof, or an abasic site, at the position corresponding to a nucleotide, in some cases for example an adenosine or a cytidine, in some cases for example an adenosine, to be edited in the target sequence.

TABLE 9

| SEQ ID NO. | Sequence 5′ -> 3 |
|---|---|
| 1191 | UsCsAUUAAACGCCAGAGUCsCsGsGsA |
| 1192 | UsCsUGAAUAAUCCAGGAAAsAsGsCsA |
| 1193 | UsAsUAGGGGUGCCAAGCAGsUsUsGsG |
| 1194 | UsAsUGGUUUUUCCAGACGGsCsAsGsG |
| 1195 | GsGsUGCAGAUUCCAGGUGGsGsAsCsG |
| 1196 | AsCsAGACUUGGCCACUGAGsUsGsGsG |
| 1197 | UsAsUGUGUCGGCCACGGAAsCsAsGsG |
| 1198 | AsAsUAAGGGGUCCACAUGGsCsAsAsC |
| 1199 | UsCsGAGCAAUGCCAUCACCsUsCsCsC |
| 1200 | UsAsUUUCCCUGCCAGAAUAsGsAsUsG |
| 1201 | GsAsUGCUCCAACCACCACAsAsGsUsU |
| 1202 | CsGsUCUCUUGCCCACGCCAsCsCsAsG |
| 1203 | GsUsCUCUUGAUACAUCCAGsUsUsCsC |
| 1204 | CsAsCAUGGGAUUCCCAUUGsAsUsGsA |
| 1205 | UsAsUCGACCAAACCCGUUGsAsCsUsC |
| 1206 | CsAsCGUCAUGAGCCCUUCCsAsCsGsA |
| 1207 | AsAsCGAGGGAUCCCGCUCCsUsGsGsA |
| 1208 | GsAsAGAGGCUGUCGUCAUAsCsUsUsC |
| 1209 | CsAsAGAGGUCAACGAAGGGsGsUsCsA |
| 1210 | AsAsCGCCAGGGGCGCUAAGsCsAsGsU |
| 1211 | UsAsCGCAUGGACCGUGGUCsAsUsGsA |
| 1212 | UsAsCAUGACCCUCUUGGCUsCsCsCsC |
| 1213 | GsAsCUAGCCAAACUCGUUGsUsCsAsU |
| 1214 | AsGsUCGCCACAGCUUCCCGsGsAsGsG |
| 1215 | UsGsUAUAUCCACCUUACCAsGsAsGsU |
| 1216 | AsGsGAGGGGUCUCACUCCUsUsGsGsA |
| 1217 | CsUsAGGCAACAACAUCCACsUsUsUsA |
| 1218 | CsCsGAGCGCCAGCAGAGGCsAsGsGsG |
| 1219 | UsAsUGGUUUUUCCAGACGGsCsAsGsG |
| 1220 | GsAsAGAGGCUGUCGUCAUAsCsUsUsC |
| 1221 | 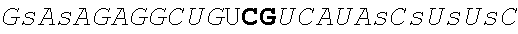 |
| 1222 | UsAsCAUGACCCUCU UGGCUsCsCsCsC |
| 1223 | 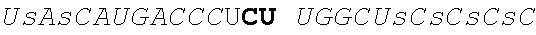 |
| 1224 | AsGsUCGCCACAGCUUCCCGsGsAsGsG |
| 1225 | 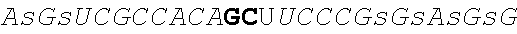 |
| 1226 | AsGsGAGGGGUCUCACUCCUsUsGsGsA |
| 1227 | 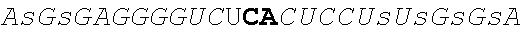 |

In Table 9; nucleotides highlighted in bold are unmodified and are placed opposite the triplet with the target adenosine in the middle. Nucleotides highlighted in italic are modified with 2′-O-methylation, 2′-fluorinated nucleotides are grayed out. The backbone contains terminal phosphorothioate linkages as indicated by “s”. The first three nucleotides at the 5′-end are not complementary to the mRNA substrate, but serve as linker sequence between gRNA and SNAP-tag.

TABLE 10

| SEQ ID NO. | Sequence 5′->3 |
|---|---|
| 1228 | TAATCTAGGAAAACTGAGAACAGAGGCCCTGAAAAAGGG |
| | CCAAATTCTTCCACCC |

TABLE 11

| SEQ | |
|---|---|
| ID | |
| NO. | Sequence 5′ -> 3 |
| 1229 | AAACCGAGGGAUCAUAGGGGACUGAAUCCACCAUUCUUCUCCCAAUCCCUG |
| | CAACUCCUUCUUCCCCUGC |
| 1230 | UGAACAGCUCCUCGCCCUUGCUCACUGGCAGAGCCCUCCAGCAUCGCGAGCA |
| | GGCGCUGCCUCCUCCGCC |
| 1231 | UCUCAGUCCAAUGUAUGGUCCGAGCACAAGCUCUAAUCAAAGUCCGCGGGU |
| | GUAGACCGGUUGCCAUAGGA |
| 1232 | ACAGCUCCUCGCCCUUGCUCACUGGCAGAGCCCUCCAGCAUCGCGAGCAGGC |
| | GCUGCCUCCUCCGCCGCUG |
| 1233 | ACAGCUCCUCGCCCUUGCUCACUGGCAGAGCCCUCAAGCAUCGCGAGCAGGC |
| | GCUGCCUCCUCCGCCGCUG |
| 1234 | ACAGCUCCUCGCCCUUGCUCACUGGCAGAGCCCUCUAGCAUCGCGAGCAGGC |
| | GCUGCCUCCUCCGCCGCUG |
| 1235 | ACAGCUCCUCGCCCUUGCUCACUGGCAGAGCCCUCGAGCAUCGCGAGCAGGC |
| | GCUGCCUCCUCCGCCGCUG |
| 1236 | ACAGCUCCUCGCCCUUGCUCACUGGCAGAGCCCUGCAGCAUCGCGAGCAGGC |
| | GCUGCCUCCUCCGCCGCUG |
| 1237 | ACAGCUCCUCGCCCUUGCUCACUGGCAGAGCCCUUCAGCAUCGCGAGCAGGC |
| | GCUGCCUCCUCCGCCGCUG |
| 1238 | ACAGCUCCUCGCCCUUGCUCACUGGCAGAGCCCUACAGCAUCGCGAGCAGGC |
| | GCUGCCUCCUCCGCCGCUG |
| 1239 | ACAGCUCCUCGCCCUUGCUCACUGGCAGAGCCCUCCUGCAUCGCGAGCAGGC |
| | GCUGCCUCCUCCGCCGCUG |
| 1240 | ACAGCUCCUCGCCCUUGCUCACUGGCAGAGCCCUGCUGCAUCGCGAGCAGGC |
| | GCUGCCUCCUCCGCCGCUG |
| 1241 | ACAGCUCCUCGCCCUUGCUCACUGGCAGAGCCCUUCUGCAUCGCGAGCAGGC |
| | GCUGCCUCCUCCGCCGCUG |
| 1242 | ACAGCUCCUCGCCCUUGCUCACUGGCAGAGCCCUACUGCAUCGCGAGCAGGC |
| | GCUGCCUCCUCCGCCGCUG |
| 1243 | ACAGCUCCUCGCCCUUGCUCACUGGCAGAGCCCUCCCGCAUCGCGAGCAGGC |
| | GCUGCCUCCUCCGCCGCUG |
| 1244 | ACAGCUCCUCGCCCUUGCUCACUGGCAGAGCCCUGCCGCAUCGCGAGCAGGC |
| | GCUGCCUCCUCCGCCGCUG |
| 1245 | ACAGCUCCUCGCCCUUGCUCACUGGCAGAGCCCUUCCGCAUCGCGAGCAGGC |
| | GCUGCCUCCUCCGCCGCUG |
| 1246 | ACAGCUCCUCGCCCUUGCUCACUGGCAGAGCCCUACCGCAUCGCGAGCAGGC |
| | GCUGCCUCCUCCGCCGCUG |
| 1247 | ACAGCUCCUCGCCCUUGCUCACUGGCAGAGCCCUCCGGCAUCGCGAGCAGGC |
| | GCUGCCUCCUCCGCCGCUG |
| 1248 | ACAGCUCCUCGCCCUUGCUCACUGGCAGAGCCCUGCUGCAUCGCGAGCAGGC |
| | GCUGCCUCCUCCGCCGCUG |
| 1249 | ACAGCUCCUCGCCCUUGCUCACUGGCAGAGCCCUUCGGCAUCGCGAGCAGGC |
| | GCUGCCUCCUCCGCCGCUG |
| 1250 | ACAGCUCCUCGCCCUUGCUCACUGGCAGAGCCCUACGGCAUCGCGAGCAGGC |
| | GCUGCCUCCUCCGCCGCUG |
| 1251 | ACUGGCAGAGCCCUCCAGCAUCGCGAGCAGG |
| 1252 | GCCCUUGCUCACUGGCAGAGCCCUCCAGCAUCGCGAGCAGGCGCUGCCUCC |
| 1253 | ACAGCUCCUCGCCCUUGCUCACUGGCAGAGCCCUCCAGCAUCGCGAGCAGGC |
| | GCUGCCUCCUCCGCCGCUG |
| 1254 | ACCCCGGUGAACAGCUCCUCGCCCUUGCUCACUGGCAGAGCCCUCCAGCAUC |
| | GCGAGCAGGCGCUGCCUCCUCCGCCGCUGCCUCCUCCGC |
| 1255 | GCUCGACCAGGAUGGGCACCACCCCGGUGAACAGCUCCUCGCCCUUGCUCAC |
| | UGGCAGAGCCCUCCAGCAUCGCGAGCAGGCGCUGCCUCCUCCGCCGCUGCCU |
| | CCUCCGCCGCUGCCUCCUCCGCCCUGCUCGCCGUCCAGCUCGACCAGGAUGG |
| | GCACCACCCCGGUGAACAGCUCCUCGCCCUUGCUCACUGGCAGAGCCCUCCA |
| | GCAUCGCGAGCAGGCGCUGCCUCCUCCGCCGCUGCCUCCUCCGCCGCUGCCU |
| | CCUCCGCCCUGCAGCUUGUACA |
| 1256 | UCGCCGUCCAGCUCGACCAGGAUGGGCACCACCCCGGUGAACAGCUCCUCGC |
| | CCUUGCUCACUGGCAGAGCCCUCCAGCAUCGCGAGCAGGCGCUGCCUCCUCC |
| | GCCGCUGCCUCCUCCGCCGCUGCCUCCUCCGCCCUGCAGCUUGUACA |
| 1257 | GCCGUUUACGUCGCCGUCCAGCUCGACCAGGAUGGGCACCACCCCGGUGAAC |
| | AGCUCCUCGCCCUUGCUCACUGGCAGAGCCCUCCAGCAUCGCGAGCAGGCGC |
| | UGCCUCCUCCGCCGCUGCCUCCUCCGCCGCUGCCUCCUCCGCCCUGCAGCUU |
| | GUACAGCUCGUCCAU |
| 1258 | UGAACUUGUGGCCGUUUACGUCGCCGUCCAGCUCGACCAGGAUGGGCACCA |
| | CCCCGGUGAACAGCUCCUCGCCCUUGCUCACUGGCAGAGCCCUCCAGCAUCG |
| | CGAGCAGGCGCUGCCUCCUCCGCCGCUGCCUCCUCCGCCGCUGCCUCCUCCG |
| | CCCUGCAGCUUGUACAGCUCGUCCAUGCCGCCGGUG |
| 1259 | CCGGACACGCUGAACUUGUGGCCGUUUACGUCGCCGUCCAGCUCGACCAGG |
| | AUGGGCACCACCCCGGUGAACAGCUCCUCGCCCUUGCUCACUGGCAGAGCCC |
| | UCCAGCAUCGCGAGCAGGCGCUGCCUCCUCCGCCGCUGCCUCCUCCGCCGCU |
| | GCCUCCUCCGCCCUGCAGCUUGUACAGCUCGUCCAUGCCGCCGGUGGAGUGG |
| | CGGC |
| 1260 | GCGACCGGGGAUCUCCACAGAUUCUUCCGGC |
| 1261 | GCUCACGGUGGCGACCGGGGAUCUCCACAGAUUCUUCCGGCGUGUAUACCU |
| 1262 | CCUCGCCCUUGCUCACGGUGGCGACCGGGGAUCUCCACAGAUUCUUCCGGCG |
| | UGUAUACCUUCUGCUGCCU |
| 1263 | GUGAACAGCUCCUCGCCCUUGCUCACGGUGGCGACCGGGGAUCUCCACAGA |
| | UUCUUCCGGCGUGUAUACCUUCUGCUGCCUCCUCCGCCGC |
| 1264 | CACCACCCCGGUGAACAGCUCCUCGCCCUUGCUCACGGUGGCGACCGGGGAU |
| | CUCCACAGAUUCUUCCGGCGUGUAUACCUUCUGCUGCCUCCUCCGCCGCUGC |
| | CUCCUCC |
| 1265 | CCAGGAUGGGCACCACCCCGGUGAACAGCUCCUCGCCCUUGCUCACGGUGGC |
| | GACCGGGGAUCUCCACAGAUUCUUCCGGCGUGUAUACCUUCUGCUGCCUCC |
| | UCCGCCGCUGCCUCCUCCGCCGCUGCCU |
| 1266 | UCCAGCUCGACCAGGAUGGGCACCACCCCGGUGAACAGCUCCUCGCCCUUGC |
| | UCACGGUGGCGACCGGGGAUCUCCACAGAUUCUUCCGGCGUGUAUACCUUC |
| | UGCUGCCUCCUCCGCCGCUGCCUCCUCCGCCGCUGCCUCCUCCGCCCU |
| 1267 | CGGCGACGUAUCCAGCUCGACCAGGAUGGGCACCACCCCGGUGAACAGCUCC |
| | UCGCCCUUGCUCACGGUGGCGACCGGGGAUCUCCACAGAUUCUUCCGGCGU |
| | GUAUACCUUCUGCUGCCUCCUCCGCCGCUGCCUCCUCCGCCGCUGCCUCCUC |
| | CGCCCUGCAGCUUGUA |
| 1268 | UGUGGCCGUUUACGUCGCCGUCCAGCUCGACCAGGAUGGGCACCACCCCGGU |
| | GAACAGCUCCUCGCCCUUGCUCACGGUGGCGACCGGGGAUCUCCACAGAUUC |
| | UUCCGGCGUGUAUACCUUCUGCUGCCUCCUCCGCCGCUGCCUCCUCCGCCGC |
| | UGCCUCCUCCGCCCUGCAGCUUGUACAGCUCGUCC |
| 1269 | ACGCUGAACUUGUGGCCGUUUACGUCGCCGUCCAGCUCGACCAGGAUGGGC |
| | ACCACCCCGGUGAACAGCUCCUCGCCCUUGCUCACGGUGGCGACCGGGGAUC |
| | UCCACAGAUUCUUCCGGCGUGUAUACCUUCUGCUGCCUCCUCCGCCGCUGCC |
| | UCCUCCGCCGCUGCCUCCUCCGCCCUGCAGCUUGUACAGCUCGUCCAUGCCG |
| | CCGG |
| 1270 | CAGCAUCGCGAGCAGGCGCUGCCUCCUCCGCCGCUGCCUCCUCCGCCGCUGC |
| | CUCCUCCGCCCUGCAGCUU |
| 1271 | CCCUCCAGCAUCGCGAGCAGGCGCUGCCUCCUCCGCCGCUGCCUCCUCCGCC |
| | GCUGCCUCCUCCGCCCUGC |
| 1272 | CAGAGCCCUCCAGCAUCGCGAGCAGGCGCUGCCUCCUCCGCCGCUGCCUCCU |
| | CCGCCGCUGCCUCCUCCGC |
| 1273 | ACUGGCAGAGCCCUCCCAGCAUCGCGAGCAGGCGCUGCCUCCUCCGCCGCUG |
| | CCUCCUCCGCCGCUGCCUCC |
| 1274 | UGCUCACUGGCAGAGCCCUCCAGCAUCGCGAGCAGGCGCUGCCUCCUCCGCC |
| | GCUGCCUCCUCCGCCGCUG |
| 1275 | GCCCUUGCUCACUGGCAGAGCCCUCCAGCAUCGCGAGCAGGCGCUGCCUCCU |
| | CCGCCGCUGCCUCCUCCGC |
| 1276 | UCCUCGCCCUUGCUCACUGGCAGAGCCCUCCAGCAUCGCGAGCAGGCGCUGC |
| | CUCCUCCGCCGCUGCCUCC |
| 1277 | GGUGAACAGCUCCUCGCCCUUGCUCACUGGCAGAGCCCUCCAGCAUCGCGAG |
| | CAGGCGCUGCCUCCUCCGC |
| 1278 | ACCCCGGUGAACAGCUCCUCGCCCUUGCUCACUGGCAGAGCCCUCCAGCAUC |
| | GCGAGCAGGCGCUGCCUCC |
| 1279 | GCACCACCCCGGUGAACAGCUCCUCGCCCUUGCUCACUGGCAGAGCCCUCCA |
| | GCAUCGCGAGCAGGCGCUG |
| 1280 | GAUGGGCACCACCCCGGUGAACAGCUCCUCGCCCUUGCUCACUGGCAGAGCC |
| | CUCCAGCAUCGCGAGCAGG |
| 1281 | ACCAGGAUGGGCACCACCCCGGUGAACAGCUCCUCGCCCUUGCUCACUGGCA |
| | GAGCCCUCCAGCAUCGCGA |
| 1282 | GCUCGACCAGGAUGGGCACCACCCCGGUGAACAGCUCCUCGCCCUUGCUCAC |
| | UGGCAGAGCCCUCCAGCAU |
| 1283 | GUCCAGCUCGACCAGGAUGGGCACCACCCCGGUGAACAGCUCCUCGCCCUUG |
| | CUCACUGGCAGAGCCCUCC |
| 1284 | CACAGAUUCUUCCGGCGUGUAUACCUUCUGCUGCCUCCUCCGCCGCUGCCUC |
| | CUCCGCCGCUGCCUCCUCC |
| 1285 | AUCUCCACAGAUUCUUCCGGCGUGUAUACCUUCUGCUGCCUCCUCCGCCGCU |
| | GCCUCCUCCGCCGCUGCCU |
| 1286 | CGGGGAUCUCCACAGAUUCUUCCGGCGUGUAUACCUUCUGCUGCCUCCUCCG |
| | CCGCUGCCUCCUCCGCCGC |
| 1287 | GCGACCGGGGAUCUCCACAGAUUCUUCCGGCGUGUAUACCUUCUGCUGCCU |
| | CCUCCGCCGCUGCCUCCUCC |
| 1288 | CGGUGGCGACCGGGGAUCUCCACAGAUUCUUCCGGCGUGUAUACCUUCUGC |
| | UGCCUCCUCCGCCGCUGCCU |
| 1289 | GCUCACGGUGGCGACCGGGGAUCUCCACAGAUUCUUCCGGCGUGUAUACCU |
| | UCUGCUGCCUCCUCCGCCGC |
| 1290 | CCCUUGCUCACGGUGGCGACCGGGGAUCUCCACAGAUUCUUCCGGCGUGUA |
| | UACCUUCUGCUGCCUCCUCC |
| 1291 | CAGCUCCUCGCCCUUGCUCACGGUGGCGACCGGGGAUCUCCACAGAUUCUUC |
| | CGGCGUGUAUACCUUCUGC |
| 1292 | GUGAACAGCUCCUCGCCCUUGCUCACGGUGGCGACCGGGGAUCUCCACAGA |
| | UUCUUCCGGCGUGUAUACCU |
| 1293 | CCCCGGUGAACAGCUCCUCGCCCUUGCUCACGGUGGCGACCGGGGAUCUCCA |
| | CAGAUUCUUCCGGCGUGUA |
| 1294 | CACCACCCCGGUGAACAGCUCCUCGCCCUUGCUCACGGUGGCGACCGGGGAU |
| | CUCCACAGAUUCUUCCGGC |
| 1295 | AUGGGCACCACCCCGGUGAACAGCUCCUCGCCCUUGCUCACGGUGGCGACCG |
| | GGGAUCUCCACAGAUUCUU |
| 1296 | CCAGGAUGGGCACCACCCCGGUGAACAGCUCCUCGCCCUUGCUCACGGUGGC |
| | GACCGGGGAUCUCCACAGA |
| 1297 | CUCGACCAGGAUGGGCACCACCCCGGUGAACAGCUCCUCGCCCUUGCUCACG |
| | GUGGCGACCGGGGAUCUCC |
| 1298 | GAUGGGCACCACCCCGGUGAACAGCUCCUCGCCCUUGCUCACUGGCAGAGCC |
| | CUCCAGCAUCGCGAGCAGGCGCUGCCUCCUCCGCCCUGCAGCUUGUACAGCU |
| | CGUCCAU |
| 1299 | GAUGGGCACCACCCCGGUGAACAGCUCCUCGCCCUUGCUCACUGGCAGAGCC |
| | CUGCAGCAUCGCGAGCAGGCGCUGCCUCCUCCGCCCUGCAGCUUGUACAGCU |
| | CGUCCAU |
| 1300 | GAUGGGCACCACCCCGGUGAACAGCUCCUCGCCCUUGCUCACUGGCAGAGCC |
| | CUUCAGCAUCGCGAGCAGGCGCUGCCUCCUCCGCCCUGCAGCUUGUACAGCU |
| | CGUCCAU |
| 1301 | GAUGGGCACCACCCCGGUGAACAGCUCCUCGCCCUUGCUCACUGGCAGAGCC |
| | CUACAGCAUCGCGAGCAGGCGCUGCCUCCUCCGCCCUGCAGCUUGUACAGCU |
| | CGUCCAU |
| 1302 | GAUGGGCACCACCCCGGUGAACAGCUCCUCGCCCUUGCUCACUGGCAGAGCC |
| | CUCCGGCAUCGCGAGCAGGCGCUGCCUCCUCCGCCCUGCAGCUUGUACAGCU |
| | CGUCCAU |
| 1303 | GAUGGGCACCACCCCGGUGAACAGCUCCUCGCCCUUGCUCACUGGCAGAGCC |
| | CUGCGGCAUCGCGAGCAGGCGCUGCCUCCUCCGCCCUGCAGCUUGUACAGCU |
| | CGUCCAU |
| 1304 | GAUGGGCACCACCCCGGUGAACAGCUCCUCGCCCUUGCUCACUGGCAGAGCC |
| | CUUCGGCAUCGCGAGCAGGCGCUGCCUCCUCCGCCCUGCAGCUUGUACAGCU |
| | CGUCCAU |
| 1305 | GAUGGGCACCACCCCGGUGAACAGCUCCUCGCCCUUGCUCACUGGCAGAGCC |
| | CUACGGCAUCGCGAGCAGGCGCUGCCUCCUCCGCCCUGCAGCUUGUACAGCU |
| | CGUCCAU |
| 1306 | GAUGGGCACCACCCCGGUGAACAGCUCCUCGCCCUUGCUCACUGGCAGAGCC |
| | CUCCUGCAUCGCGAGCAGGCGCUGCCUCCUCCGCCCUGCAGCUUGUACAGCU |
| | CGUCCAU |
| 1307 | GAUGGGCACCACCCCGGUGAACAGCUCCUCGCCCUUGCUCACUGGCAGAGCC |
| | CUGCUGCAUCGCGAGCAGGCGCUGCCUCCUCCGCCCUGCAGCUUGUACAGCU |
| | CGUCCAU |
| 1308 | GAUGGGCACCACCCCGGUGAACAGCUCCUCGCCCUUGCUCACUGGCAGAGCC |
| | CUACUGCAUCGCGAGCAGGCGCUGCCUCCUCCGCCCUGCAGCUUGUACAGCU |
| | CGUCCAU |
| 1309 | GAUGGGCACCACCCCGGUGAACAGCUCCUCGCCCUUGCUCACUGGCAGAGCC |
| | CUUCUGCAUCGCGAGCAGGCGCUGCCUCCUCCGCCCUGCAGCUUGUACAGCU |
| | CGUCCAU |
| 1310 | GAUGGGCACCACCCCGGUGAACAGCUCCUCGCCCUUGCUCACUGGCAGAGCC |
| | CUCCCGCAUCGCGAGCAGGCGCUGCCUCCUCCGCCCUGCAGCUUGUACAGCU |
| | CGUCCAU |
| 1311 | GAUGGGCACCACCCCGGUGAACAGCUCCUCGCCCUUGCUCACUGGCAGAGCC |
| | CUGCCGCAUCGCGAGCAGGCGCUGCCUCCUCCGCCCUGCAGCUUGUACAGCU |
| | CGUCCAU |
| 1312 | GAUGGGCACCACCCCGGUGAACAGCUCCUCGCCCUUGCUCACUGGCAGAGCC |
| | CUUCCGCAUCGCGAGCAGGCGCUGCCUCCUCCGCCCUGCAGCUUGUACAGCU |
| | CGUCCAU |
| 1313 | GAUGGGCACCACCCCGGUGAACAGCUCCUCGCCCUUGCUCACUGGCAGAGCC |
| | CUACCGCAUCGCGAGCAGGCGCUGCCUCCUCCGCCCUGCAGCUUGUACAGCU |
| | CGUCCAU |
| 1314 | UACCGCUACAGCCACGCUGAUUUCAGCUAUACCUGCCCGGUAUAAAGGGAC |
| | GUUCACACCGCGAUGUUCUCUGCUGGGGAAUUGCGCGAUAUUCAGGAUUAA |
| | AAGAAGUGC |
| 1315 | ACUACAGUUGCUCCGAUAUUUAGGCUACGUCAAUAGGCACUAACUUAUUGG |
| | CGCUGGUGAACGGACUUCCUCUCGAGUACCAGAAGAUGACUACAAAACUCC |
| | UUUCCAUUGCGAGUAUCGGAGUCUGGCUCAGUUUGGCCAGGGAGGCACU |
| 1316 | CGGAAGAGGGUGGGGCCGCGGUGGCCAGGGAGCCGGCGCCGCCACGCGCGG |
| 1317 | CAGCUGAGGCCGGAAGAGGGUGGGGCCGCGGUGGCCAGGGAGCCGGCGCCG |
| | CCACGCGCGGGUGGGGGGGA |
| 1318 | GGAGGCGAAAGCAGCCCGGACAGCUGAGGCCGGAAGAGGGUGGGGCCGCGG |
| | UGGCCAGGGAGCCGGCGCCGCCACGCGCGGGUGGGGGGGACUGGGGUUGCU |
| | CGCGGGCUC |
| 1319 | GAGGCGCAGCAUCCACAGGCGGAGGCGAAAGCAGCCCGGACAGCUGAGGCC |
| | GGAAGAGGGUGGGGCCGCGGUGGCCAGGGAGCCGGCGCCGCCACGCGCGGG |
| | UGGGGGGGACUGGGGUUGCUCGCGGGCUCCGGGCGGGCGGCGGGCGCCG |
| 1320 | UCUUGCCUACGCCACCAGCUCCAACCACCACAAGUUUAUAUUCAGUCAUUU |
| 1321 | GAUUCUGAAUUAGCUGUAUCGUCAAGGCACUCUUGCCUACGCCACCAGCUC |
| | CAACCACCACAAGUUUAUAUUCAGUCAUUUUCAGCAGGCCUCUCUCCCGCAC |
| | CUGGGAGC |
| 1322 | AUCAUAUUCGUCCACAAAAUGAUUCUGAAUUAGCUGUAUCGUCAAGGCACU |
| | CUUGCCUACGCCACCAGCUCCAACCACCACAAGUUUAUAUUCAGUCAUUUUC |
| | AGCAGGCCUCUCUCCCGCACCUGGGAGCCGCUGAGCCUCUGGCCCCGC |
| 1323 | UCGGCAUGGUAUGAAGUACUUCGUCCAGGAGCUGGAGGGCCCGGUGUAAGU |
| 1324 | GGGUCUGCAAUCGGCAUGGUAUGAAGUACUUCGUCCAGGAGCUGGAGGGCC |
| | CGGUGUAAGUGAAUUUCAAU |
| 1325 | GACCUCAGUCUAAAGGUUGUGGGUCUGCAAUCGGCAUGGUAUGAAGUACUU |
| | CGUCCAGGAGCUGGAGGGCCCGGUGUAAGUGAAUUUCAAUCCAGCAAGGUG |
| | UUUCUUUGA |
| 1326 | UAAGGGCCCCAACGGUAAAAGACCUCAGUCUAAAGGUUGUGGGUCUGCAAU |
| | CGGCAUGGUAUGAAGUACUUCGUCCAGGAGCUGGAGGGCCCGGUGUAAGUG |
| | AAUUUCAAUCCAGCAAGGUGUUUCUUUGAUGCUCUGUCUUGGGUAAUCC |
| 1327 | UGGGGGGUUCGGCUGCCGACAUCAGCAAUUGCUCUGCCACCAUCUCAGCCC |
| 1328 | AGCAGGGCCGUGGGGGGUUCGGCUGCCGACAUCAGCAAUUGCUCUGCCACC |
| | AUCUCAGCCCAUCCUCCGAA |
| 1329 | AGUAGAAGGCCAAGAGCCACAGCAGGGCCGUGGGGGGUUCGGCUGCCGACA |
| | UCAGCAAUUGCUCUGCCACCAUCUCAGCCCAUCCUCCGAAGUGAAUGAACA |
| | GGAACCAGC |
| 1330 | CCUCCCAUCACGGGGGCCGUAGUAGAAGGCCAAGAGCCACAGCAGGGCCGU |
| | GGGGGGUUCGGCUGCCGACAUCAGCAAUUGCUCUGCCACCAUCUCAGCCCA |
| | UCCUCCGAAGUGAAUGAACAGGAACCAGCUCUCAAAGGGACCUCCGCAG |
| 1331 | GCCAAACACCACATGCTTGCCATCTAGCCAGGCTGTCTTGACTGTCGTGATGA |
| | AGAACTGGGAGCCGTTGGTGTCCTTGCCTGCGTTGGCCATGCTCACCCAGCCA |
| | GGCCCGTAGTGCTTCAGTTTGAAGTTCTCATCGGGGAAGCGCTCA |
| 1332 | GGGAGTGGGTCCGCTCCACCAGATGCCAGCACCGGGGCCAGTGCAGCTCAGA |
| | GCCCTGTGGCGGACTACAGGGCCCGCACAGACGGTCACTCAAAGAAAGATGT |
| | CCCTGTGCCCTACTCCTTGGCGATGGCAAAGGGCTTCTCCACCTCGA |
| 1333 | TGCATTTTGTAAAATAGATACTAGCAGATTGTCCCAAGATGTGTACAGCTCATT |
| | CTCACAGCCCAGCGAGGGCACCTACTCCACAAATGCGTGGCCACAGGTCATCA |
| | CCTGTCCTGTGGCCCTGGCGAGCCTGATCCCTCACGCCGGGCAC |
| 1334 | GCTCATTCTCACAGCCCAGCGAGGGCACTTACTCCACAAATGCGTGGCCACAG |
| | GTCATCACCTGTCCTGTGGCCCCGGCGAGCCTGATCCCTCACGCCGGGCACCC |
| | ACACGGCCTGCGTGCCTTCTAGACTTGAGTTCGCAGCTCTTTAAG |
| 1335 | TCGGCCGGGCCCTGGGGGCGGTGGGCGCTGGCCAGGACGCCCACCGTGTGGTT |
| | GCTGTCCAGGACGGTCCCGGCCCGCGACACTTCGGCCCAGAGCTGCTCCTCAT |
| | CCAGCAGCGCCAGCAGCCCCATGGCCGTGAGCACCGGCTTGCGCA |
| 1336 | UGACCAGUCUUAAGAUCUUUCUUGACCUGCACCAUAAGAACUUCUCCAAAG |
| | GUACCAAAAUACUCUUUCAGGUCCUGUUCGGUUGUUUUCCAUGGGAGACCC |
| | AACACUAUU |
| 1337 | GAUGGGCACCACCCCGGUGAACAGCUCCUCGCCCUUGCUCACUGGCAGAGCC |
| | CUCGAGCAUCGCGAGCAGGCGCUGCCUCCUCCGCCCUGCAGCUUGUACAGCU |
| | CGUCCAU |
| 1338 | GAUGGGCACCACCCCGGUGAACAGCUCCUCGCCCUUGCUCACUGGCAGAGCC |
| | CUGGAGCAUCGCGAGCAGGCGCUGCCUCCUCCGCCCUGCAGCUUGUACAGCU |
| | CGUCCAU |
| 1339 | GAUGGGCACCACCCCGGUGAACAGCUCCUCGCCCUUGCUCACUGGCAGAGCC |
| | CUUGAGCAUCGCGAGCAGGCGCUGCCUCCUCCGCCCUGCAGCUUGUACAGCU |
| | CGUCCAU |
| 1340 | GAUGGGCACCACCCCGGUGAACAGCUCCUCGCCCUUGCUCACUGGCAGAGCC |
| | CUAGAGCAUCGCGAGCAGGCGCUGCCUCCUCCGCCCUGCAGCUUGUACAGCU |
| | CGUCCAU |
| 1341 | GAUGGGCACCACCCCGGUGAACAGCUCCUCGCCCUUGCUCACUGGCAGAGCC |
| | CUCGGGCAUCGCGAGCAGGCGCUGCCUCCUCCGCCCUGCAGCUUGUACAGCU |
| | CGUCCAU |
| 1342 | GAUGGGCACCACCCCGGUGAACAGCUCCUCGCCCUUGCUCACUGGCAGAGCC |
| | CUGGGGCAUCGCGAGCAGGCGCUGCCUCCUCCGCCCUGCAGCUUGUACAGCU |
| | CGUCCAU |
| 1343 | GAUGGGCACCACCCCGGUGAACAGCUCCUCGCCCUUGCUCACUGGCAGAGCC |
| | CUUGGGCAUCGCGAGCAGGCGCUGCCUCCUCCGCCCUGCAGCUUGUACAGCU |
| | CGUCCAU |
| 1344 | GAUGGGCACCACCCCGGUGAACAGCUCCUCGCCCUUGCUCACUGGCAGAGCC |
| | CUAGGGCAUCGCGAGCAGGCGCUGCCUCCUCCGCCCUGCAGCUUGUACAGCU |
| | CGUCCAU |
| 1345 | GAUGGGCACCACCCCGGUGAACAGCUCCUCGCCCUUGCUCACUGGCAGAGCC |
| | CUCGUGCAUCGCGAGCAGGCGCUGCCUCCUCCGCCCUGCAGCUUGUACAGCU |
| | CGUCCAU |
| 1346 | GAUGGGCACCACCCCGGUGAACAGCUCCUCGCCCUUGCUCACUGGCAGAGCC |
| | CUGGUGCAUCGCGAGCAGGCGCUGCCUCCUCCGCCCUGCAGCUUGUACAGCU |
| | CGUCCAU |
| 1347 | GAUGGGCACCACCCCGGUGAACAGCUCCUCGCCCUUGCUCACUGGCAGAGCC |
| | CUAGUGCAUCGCGAGCAGGCGCUGCCUCCUCCGCCCUGCAGCUUGUACAGCU |
| | CGUCCAU |
| 1348 | GAUGGGCACCACCCCGGUGAACAGCUCCUCGCCCUUGCUCACUGGCAGAGCC |
| | CUUGUGCAUCGCGAGCAGGCGCUGCCUCCUCCGCCCUGCAGCUUGUACAGCU |
| | CGUCCAU |
| 1349 | GAUGGGCACCACCCCGGUGAACAGCUCCUCGCCCUUGCUCACUGGCAGAGCC |
| | CUCGCGCAUCGCGAGCAGGCGCUGCCUCCUCCGCCCUGCAGCUUGUACAGCU |
| | CGUCCAU |
| 1350 | GAUGGGCACCACCCCGGUGAACAGCUCCUCGCCCUUGCUCACUGGCAGAGCC |
| | CUGGCGCAUCGCGAGCAGGCGCUGCCUCCUCCGCCCUGCAGCUUGUACAGCU |
| | CGUCCAU |
| 1351 | GAUGGGCACCACCCCGGUGAACAGCUCCUCGCCCUUGCUCACUGGCAGAGCC |
| | CUUGCGCAUCGCGAGCAGGCGCUGCCUCCUCCGCCCUGCAGCUUGUACAGCU |
| | CGUCCAU |
| 1352 | GAUGGGCACCACCCCGGUGAACAGCUCCUCGCCCUUGCUCACUGGCAGAGCC |
| | CUAGCGCAUCGCGAGCAGGCGCUGCCUCCUCCGCCCUGCAGCUUGUACAGCU |
| | CGUCCAU |
| 1353 | GAUGGGCACCACCCCGGUGAACAGCUCCUCGCCCUUGCUCACUGGCAGAGCC |
| | CUCGAGCAUCGCGAGCAGGCGCUGCCUCCUCCGCCCUGCAGCUUGUACAGCU |
| | CGUCCAU |
| 1354 | GAUGGGCACCACCCCGGUGAACAGCUCCUCGCCCUUGCUCACUGGCAGAGCC |
| | CUGGAGCAUCGCGAGCAGGCGCUGCCUCCUCCGCCCUGCAGCUUGUACAGCU |
| | CGUCCAU |
| 1355 | GAUGGGCACCACCCCGGUGAACAGCUCCUCGCCCUUGCUCACUGGCAGAGCC |
| | CUUGAGCAUCGCGAGCAGGCGCUGCCUCCUCCGCCCUGCAGCUUGUACAGCU |
| | CGUCCAU |
| 1356 | GAUGGGCACCACCCCGGUGAACAGCUCCUCGCCCUUGCUCACUGGCAGAGCC |
| | CUAGAGCAUCGCGAGCAGGCGCUGCCUCCUCCGCCCUGCAGCUUGUACAGCU |
| | CGUCCAU |
| 1357 | GAUGGGCACCACCCCGGUGAACAGCUCCUCGCCCUUGCUCACUGGCAGAGCC |
| | CUCGUGCAUCGCGAGCAGGCGCUGCCUCCUCCGCCCUGCAGCUUGUACAGCU |
| | CGUCCAU |
| 1358 | GAUGGGCACCACCCCGGUGAACAGCUCCUCGCCCUUGCUCACUGGCAGAGCC |
| | CUCGGGCAUCGCGAGCAGGCGCUGCCUCCUCCGCCCUGCAGCUUGUACAGCU |
| | CGUCCAU |
| 1359 | GAUGGGCACCACCCCGGUGAACAGCUCCUCGCCCUUGCUCACUGGCAGAGCC |
| | CUCGCGCAUCGCGAGCAGGCGCUGCCUCCUCCGCCCUGCAGCUUGUACAGCU |
| | CGUCCAU |
| 1360 | GAUGGGCACCACCCCGGUGAACAGCUCCUCGCCCUUGCUCACUGGCAGAGCC |
| | CUCGUGCAUCGCGAGCAGGCGCUGCCUCCUCCGCCCUGCAGCUUGUACAGCU |
| | CGUCCAU |
| 1361 | GAUGGGCACCACCCCGGUGAACAGCUCCUCGCCCUUGCUCACUGGCAGAGCC |
| | CUGGUGCAUCGCGAGCAGGCGCUGCCUCCUCCGCCCUGCAGCUUGUACAGCU |
| | CGUCCAU |
| 1362 | GAUGGGCACCACCCCGGUGAACAGCUCCUCGCCCUUGCUCACUGGCAGAGCC |
| | CUUGUGCAUCGCGAGCAGGCGCUGCCUCCUCCGCCCUGCAGCUUGUACAGCU |
| | CGUCCAU |
| 1363 | GAUGGGCACCACCCCGGUGAACAGCUCCUCGCCCUUGCUCACUGGCAGAGCC |
| | CUAGUGCAUCGCGAGCAGGCGCUGCCUCCUCCGCCCUGCAGCUUGUACAGCU |
| | CGUCCAU |
| 1364 | GAUGGGCACCACCCCGGUGAACAGCUCCUCGCCCUUGCUCACUGGCAGAGCC |
| | CUCGAGCAUCGCGAGCAGGCGCUGCCUCCUCCGCCCUGCAGCUUGUACAGCU |
| | CGUCCAU |
| 1365 | GAUGGGCACCACCCCGGUGAACAGCUCCUCGCCCUUGCUCACUGGCAGAGCC |
| | CUCGCGCAUCGCGAGCAGGCGCUGCCUCCUCCGCCCUGCAGCUUGUACAGCU |
| | CGUCCAU |
| 1366 | GAUGGGCACCACCCCGGUGAACAGCUCCUCGCCCUUGCUCACUGGCAGAGCC |
| | CUCGGGCAUCGCGAGCAGGCGCUGCCUCCUCCGCCCUGCAGCUUGUACAGCU |
| | CGUCCAU |
| 1367 | GAUUCUGAAUUAGCUGUAUCGUCAAGGCACUCGUGCCGACGCCACCAGCUC |
| | CAACCACCACAAGUGGAGAGUCAGUCAUUUUCAGCAGGCCUCUCUCCCGCAC |
| | CUGGGAGC |
| 1368 | GAUUCUGAAUUAGCUGGAUCGUCAAGGCACUCGGGCCGACGCCACCAGCUC |
| | CAACCACCACAAGUGGAGAGUCAGUCAUUUUCAGCAGGCCUCUCUCCCGCAC |
| | CGGGGAGC |
| 1369 | GGGAGCAGCCUCUGGCAUUCUGGGAGCUUCAUCUGGACCUGGGUCUUCAGU |
| | GAACCAUUGUUCAAUAUCGUCCGGGGACAGCAUCAAAUCAUCCAUUGCUUG |
| | GGACGGCAA |
| 1370 | GGGAGCAGCCUCUGGCAUUCUGGGAGCUUCAUCUGGACCUGGGUCUUCAGU |
| | GAACCAUUGUUCAAGAUCGUCCGGGGACAGCAUCAAAUCAUCCAUUGCUUG |
| | GGACGGCAA |
| 1371 | GGGAGCAGCCUCUGGCAGUCGGGGAGCUUCAUCUGGACCUGGGUCUUCAGU |
| | GAACCAUUGUUCAAGAUCGUCCGGGGACAGCAUCAAAUCAUCCAGUGCUUG |
| | GGACGGCAA |
| 1372 | CAUAUUACAGAAUACCUUGAUAGCAUCCAAUUUGCAUCCUUGGUUAGGGUC |
| | AACCCAGUAUUCUCCACUCUUGAGUUCAGGAUGGCAGAAUUUCAGGUCUCU |
| | GCAGUUUCU |
| 1373 | GUGAAGAUAAGCCAGUCCUCUAGUAACAGAAUGAGCAAGACGGCAAGAGCU |
| | UACCCAGUCACUUGUGUGGAGACUUAAAUACUUGCAUAAAGAUCCAUUGGG |
| | AUAGUACUC |
| 1374 | GUGAACGUCAAACUGUCGGACCAAUAUGGCAGAAUCUUCUCUCAUCUCAAC |
| | UUUCCAUAUCCGUAUCAUGGAAUCAUAGCAUCCUGUAACUACUAGCUCUCU |
| | UACAGCUGG |
| 1375 | GCCAAUGAUCUCGUGAGUUAUCUCAGCAGUGUGAGCCAUCAGGGUGAUGAC |
| | AUCCCAGGCGAUCGUGUGGCCUCCAGGAGCCCAGAGCAGGAAGUUGAGGAG |
| | AAGGUGCCU |
| 1376 | CAAGACGGUGAACCACUCCAUGGUCUUCUUGUCGGCUUUCUGCACUGUGUA |
| | CCCCCAGAGCUCCGUGUUGCCGACAUCCUGGGGUGGCUUCCACUCCAGAGCC |
| | ACAUUAAG |
| 1377 | AGGAUUCUCUUUUGAAGUAUUGCUCCCCCAGUGGAUUGGGUGGCUCCAUUC |
| | ACUCCAAUGCUGAGCACUUCCACAGAGUGGGUUAAAGCGGCUCCGAACACG |
| | AAACGUGUA |
| 1378 | GACGCCCACCGUGUGGUUGCUGUCCAGGACGGUCCCGGCCUGCGACACUUCG |
| | GCCCAGAGCUGCUCCUCAUCCAGCAGCGCCAGCAGCCCCAUGGCCGUGAGCA |
| | CCGGCUU |
| 1379 | GACGCCCACCGUGUGGUUGCUGUCCAGGACGGUCCCGGCCUGCGACACUUCG |
| | GCCCAGAGCUGCUCCUCAUCUGCGGGGCGGGGGGGGGCCGUCGCCGCGUGG |
| | GGUCGUUG |
| 1380 | GGGTGATGGGTGCTGGCCAGGACACCCACTGTATGATTGCTGTCCAACACAGC |
| | CCCAGCCTTTGAGACCTCTGCCCAGAGTTGTTCTCCATCCAACAGGGCCATGA |
| | GCCCCATGACTGTGAGTACTGGCTTTCGCAGCAACTGCACATGGG |
| 1381 | ACTACAGTTGCTCCGATATTTAGGCTACGTCAATAGGCACTAACTTATTGGCGC |
| | TGGTGAACGGACTTCCTCTCGAGTACCAGAAGATGACTACAAAACTCCTTTCC |
| | ATTGCGAGTATCGGAGTCTGGCTCAGTTTGGCCAGGGAGGCACT |
| 1382 | CTGCAGGGCGGAGGAGGCAGCGGCGGAGGAGGCAGCGGCGGAGGAGGCAGC |
| | AGAAGGTATACACGCCGGAAGAATCTGTAGAGATCCCCGGTCGCCACC |
| 1383 | CTGCAGGGCGGAGGAGGCAGCGGCGGAGGAGGCAGCGGCGGAGGAGGCAGC |
| | GCCTGCTCGCGATGCTAGAGGGCTCTGCCA |
| 1384 | CTGCAGGGCGGAGGAGGCAGCGCCTGCTCGCGATGCTAGAGGGCTCTGCCA |
| 1385 | AAACCGAGGGAUCAUAGGGGACUGAAUCCACCAUUCUUCUCCCAAUCCCUG |
| | CAACUCCUUCUUCCCCUGC |
| 1386 | ATGGACGAGCTGTACAAGCTGCAGGGCGGAGGAGGCAGCGCCTGCTCGCGAT |
| | GCTATAGGGCTCTGCCAGTGAGCAAGGGCGAGGAGCTGTTCACCGGGGTGGT |
| | GCCCATC |
| 1387 | UAGCUGUAUCGUCAAGGCACUCUUGCCUACGCCACCAGCUCCAACCACCACA |
| | AGUUUAUAUUCAGUCAUUUUCAGCAGGCCUCUCUCCCGC |
| 1388 | GAUUCUGAAUUAGCUGUAUCGUCAAGGCACUCUUGCCUACGCCACCAGCUC |
| | CAACUACCACAAGUUUAUAUUCAGUCAUUUUCAGCAGGCCUCUCUCCCGCA |
| | CCUGGGAGC |
| 1389 | UCCACAAAAUGAUUCUGAAUUAGCUGUAUCGUCAAGGCACUCUUGCCUACG |
| | CCACCAGCUCCAACUACCACAAGUUUAUAUUCAGUCAUUUUCAGCAGGCCU |
| | CUCUCCCGCACCUGGGAGCCGCUGAGCCU |
| 1390 | AUCAUAUUCGUCCACAAAAUGAUUCUGAAUUAGCUGUAUCGUCAAGGCACU |
| | CUUGCCUACGCCACCAGCUCCAACCACCACAAGUUUAUAUUCAGUCAUUUUC |
| | AGCAGGCCUCUCUCCCGCACCUGGGAGCCGCUGAGCCUCUGGCCCCGC |
| 1391 | CUAUUGUUGGAUCAUAUUCGUCCACAAAAUGAUUCUGAAUUAGCUGUAUCG |
| | UCAAGGCACUCUUGCCUACGCCACCAGCUCCAACCACCACAAGUUUAUAUUC |
| | AGUCAUUUUCAGCAGGCCUCUCUCCCGCACCUGGGAGCCGCUGAGCCUCUGG |
| | CCCCGCCGCCGCCUUC |
| 1392 | UAGGAAUCCUCUAUUGUUGGAUCAUAUUCGUCCACAAAAUGAUUCUGAAUU |
| | AGCUGUAUCGUCAAGGCACUCUUGCCUACGCCACCAGCUCCAACCACCACAA |
| | GUUUAUAUUCAGUCAUUUUCAGCAGGCCUCUCUCCCGCACCUGGGAGCCGC |
| | UGAGCCUCUGGCCCCGCCGCCGCCUUCAGUGCCUGCG |
| 1393 | GAGGCGCAGCAUCCACAGGCGGAGGCGAAAGCAGCCCGGACAGCUGAGGCC |
| | GGAAGAGGGUGGGGCCGCGGUGGCCAGGGAGCCGGCGCCGCCACGCGCGGG |
| | UGGGGGGGACUGGGGUUGCUCGCGGGCUCCGGGCGGGCGGCGGGCGCCG |
| 1394 | UCCUGUAGCUAAGGCCACAAAAUUAUCCACUGUUUUUGGAACAGUCUUUCC |
| | GAAGAGACCAAAGAUCACCCGGCCCACAUCUUCAUCUCCAAUUCGUAGGUC |
| | AAAAUACACCUUGACGGUGACUUUGGGCCCCUUCUUCUUCUCAUCGGCC |
| 1395 | GCCCUGGAUCAUGAAGUCCUUGAUUACACGAUGGAAUUUGCUGUUUUUGUA |
| | GCCAAAUCCUUUCUCUCCUGUAGCCAAGGCCACAAAAUUAUCCACUGUUUU |
| | UGGAACAGUCUUUCCGAAGAGACCAAAGAUCACCCGGCCUACAUCUUCA |
| 1396 | GCGCAAGUUAGGUUUUGUCAAGAAAGGGUGUAACGCAACCAAGUCAUAGUC |
| | CGCCUAGAAGCAUUUGCGGUG |
| 1397 | GCCAUGCCAAUCUCAUCUUGUUUUCUGCGCAAGUUAGGUUUUGUCAAGAAA |
| | GGGUGUAACGCAACCAAGUCAUAGUCCGCCUAGAAGCAUUUGCGGUGGACG |
| | AUGGAGGGGCCGGACUCGUCAUACUCCUG |
| 1398 | GGACUUCCUGUAACAACGCAUCUCAUAUUUGGAAUGACCAUUAAAAAAACA |
| | ACAAUGUGCAAUCAAAGUC |
| 1399 | CAAGGUGCGGCUCCGGCCCCUCCCCUCUUCAAGGGGUCCACAUGGCAACUGU |
| | GAGGAGGGGAGAUUCAGUG |
| 1400 | UAGCUGUAUCGUCAAGGCACUCGUGCCGACGCCACCAGCUCCAACCACCACA |
| | AGGGGAGAGUCAGUCAGGGUCAGCAGGCCUCUCUCCCGC |
| 1401 | UAGCUGUAUCGUCAAGGCACUCUUGCCGACGCCACCAGCUCCAACCACCACA |
| | AGUGUAUAGUCAGUCAUUUUCAGCAGGCCUCUCUCCCGC |
| 1402 | UAGCUGGAUCGUCAAGGCACUCGUGCCGACGCCACCAGCUCCAACCACCACA |
| | AGGGGAGAGGCAGUCAGGGUCAGCAGGCCUCUCUCCCGC |
| 1403 | GAUUCUGAAUUAGCUGUAUCGUCAAGGCACUCUUGCCGACGCCACCAGCUC |
| | CAACCACCACAAGUGUAUAGUCAGUCAUUUUCAGCAGGCCUCUCUCCCGCAC |
| | CUGGGAGC |
| 1404 | GAUUCUGAAUUAGCUGUAUCGUCAAGGCACUCGUGCCGACGCCACCAGCUC |
| | CAACCACCACAAGUGGAGAGUCAGUCAUUUUCAGCAGGCCUCUCUCCCGCAC |
| | CUGGGAGC |
| 1405 | GAUUCUGAAUUAGCUGUAUCGUCAAGGCACUCGUGCCGACGCCACCAGCUC |
| | CAACCACCACAAGGGGAGAGUCAGUCAGGGUCAGCAGGCCUCUCUCCCGCAC |
| | CUGGGAGC |
| 1406 | GCUCCCCGGUGCGGGAGAGAGGCCUGCUGACCCUGACUGCCUCUCCCCUUGU |
| | GGUGGUUGGAGCUGGUGGCGUCGGCACGAGUGCCUUGACGAUCCAGCUAAU |
| | UCAGAAUC |
| 1407 | GCAGAGCCUCCAGC |
| 1408 | CUCACUGGCAGAGCCUCCAGC |
| 1409 | CCCUUGCUCACUGGCAGAGCCUCCAGC |
| 1410 | CUCUCGCCCUUGCUCACUGGCAGAGCCUCCAGC |
| 1411 | CUCUCGCCCUUGCUCACUGGCAGAGCCUCCAGCAUCGC |
| 1412 | UGAACAGCUCUCGCCCUUGCUCACUGGCAGAGCCUCCAGCAUCGC |
| 1413 | UGAACAGCUCCUCGCCCUUGCUCACUGGCAGAGCCCUCCAGCAUCGCGAGCA |
| | GGCGCUGCCUCCUCCGCC |

TABLE 12

| SEQ | |
|---|---|
| ID NO. | Sequence 5' -> 3 |
| 1414 | ACAAAUGGGGACGAGGGGGGCGGGGCGGCC |
| 1415 | CGGAGAGCAGAGAGGGAGCG |
| 1416 | AAAAAAAAAAGATCTTGAAACTGTTTTAAGGTTGGCCGATCT |
| | TAAAAAA |
| 1417 | UCAUAAUCAAUUUAUUAUUUUCUUUUAUUUUAUUCACAUAAU |
| | UUUGUUUUU |

The chemical transformation on a base may result in at least a partial knockdown of the edited RNA sequence. The chemical transformation may result in a substantially complete knockdown of the edited RNA sequence. The chemical transformation may result in a partial knockdown of the edited RNA sequence that is sufficient to impart a therapeutic effect to a subject receiving an engineered polynucleotide (e.g., a circular engineered guide RNA). An at least partial knockdown of an edited RNA sequence may result in a reduced level of an expressed protein or protein fragment thereof. A reduced level may be from about 5% to 100%. A reduced level may be from about 10% to 100%. A reduced level may be from about 15% to 100%. A reduced level may be from about 20% to 100%. A reduced level may be from about 25% to 100%. A reduced level may be from about 30% to 100%. A reduced level may be from about 40% to 100%. A reduced level may be from about 50% to 100%. A reduced level may be from about 60% to 100%. A reduced level may be from about 70% to 100%. A reduced level may be from about 80% to 100%.

An engineered polynucleotide (e.g., a circular engineered guide RNA) may comprise a targeting domain for targeting a specific sequence region or base in a nucleic acid sequence for an RNA editing entity to perform a chemical transformation. The engineered polynucleotide may also comprise a recruiting domain. A targeting domain may comprise a sequence length that may be longer than an antisense RNA, a short hairpin RNA, an siRNA, miRNA, or snoRNA. A targeting domain may comprise a sequence length sufficient for the engineered guide RNA to form a secondary structure. In some cases, a base can refer to a nucleotide. In some cases, a nucleotide can refer to a base. A targeting domain may comprise a sequence length from about 20 nucleotides to about 1,000 nucleotides in length. A targeting domain may comprise a sequence length from about 50 nucleotides to about 1,000 nucleotides in length. A targeting domain may comprise a sequence length from about 100 nucleotides to about 1,000 nucleotides in length. A targeting domain may comprise a sequence length from about 200 nucleotides to about 1,000 nucleotides in length. A targeting domain may comprise a sequence length from about 500 nucleotides to about 1,000 nucleotides in length. A targeting domain may comprise a sequence length of at least about: 50, 60, 70, 80, 90, 100, 110, 120, 130, 140, 150, 160, 170, 180, 190, 200 nucleotides in length.

At least a portion of an engineered guide RNA (such as a targeting domain, a recruiting domain, or both) may comprise a secondary structure. A secondary structure may comprise a stem-loop, a cruciform, a toe hold, a mismatch bulge, more than one of any of these, or any combination thereof. A circular engineered guide RNA, although circularized, may retain a substantially similar secondary structure as compared to a substantially similar engineered guide RNA that is not circularized.

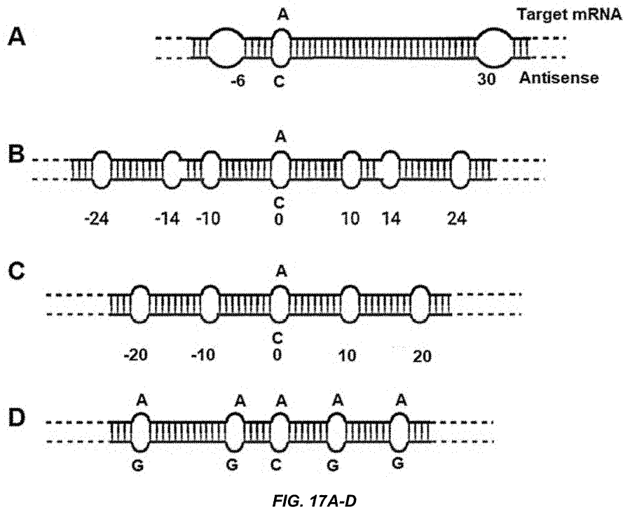

In some embodiments, the engineered polynucleotide can comprise or produce an antisense RNA sequence complementary to a target mRNA sequence to be modified except for a mismatch at the site of desired chemical modification of the target sequence. In some embodiments, the antisense RNA sequence can be circularized. In another embodiment, the antisense RNA sequence can optionally comprise additional mismatches with respect to the target RNA sequence at position with hyper-editable adenosine nucleotides. In still another embodiment, the optional mismatches can comprise a “G” instead opposite an “A” in the target RNA sequence, while the targeted “A” in the target RNA is opposed by a mismatch “C”. In still another embodiment, a circularized antisense guide RNA can comprise a mismatch at an adenosine to be chemically modified and a plurality of loops of 6-12 base pairs created 30-40 base pairs apart with the targeted adenosine located within the 30-40 base pair region. In still another embodiment, the circularized antisense guide RNA comprises a plurality of bulges that are created by positioning guanosine mismatches opposite hyperedited adenosines in the target RNA strand. In some embodiment, the bulges are created following a pattern of −34, −24, −14, −10, 0, 10, 14, 24, 34 etc., wherein 0 is the site of desired chemical modification. In some embodiment, the bulges are created following a pattern of −30, −20, −10, 0, 10, 20, 30 etc., wherein 0 is the site of desired chemical modification. Schematic representations of the foregoing are provided in FIG. 17.

An engineered guide RNA may comprise one or more modifications. A modification may include a modified base. A modification may include a sugar modification, such as adding a glucose or other sugar-based moiety to one or more bases of the engineered guide RNA. A modification may include a protein coating over at least a portion of the engineered guide RNA. One or more nucleotides of an engineered guide RNA may comprise a methyl group, a fluoro group, a methoxyethyl group, an ethyl group, a phosphate group, an amide group, an ester group, or any combination thereof. A modification may increase stability or half-life of the engineered guide RNA as compared to a substantially similar engineered guide RNA without the modification.

In some cases, an engineered guide RNA delivered to a cell or to a subject may recruit an RNA editing entity, such as an endogenous RNA editing entity. In some cases, an engineered guide RNA may be co-delivered with an RNA editing entity. In some cases, circular guide RNAs may recruit a greater amount of an RNA editing entity as compared to a guide RNA that is not circular. In some cases, an engineered guide RNA may be configured to recruit a sufficient amount of an endogenous RNA editing entity to perform the editing, such as delivery of the engineered guide RNA to a tissue location that may be comprise a low amount of endogenous RNA editing enzymes. In some cases, an engineered guide RNA may be co-delivered with an RNA editing entity. In some cases, an RNA editing entity may be separately delivered to a cell or to a subject. In some cases, an engineered guide RNA may be associated with or directly linked to an RNA editing entity and the associated or directly linked composition may be delivered to a cell or to a subject.

A guide RNA of the disclosure may not comprise (lacks) an end susceptible to hydrolytic degradation. In some cases, a guide RNA of the disclosure may comprise a secondary structure that is less susceptible to hydrolytic degradation than a mRNA naturally present in a cell. A guide RNA of the disclosure may not comprise (lacks) a reducing hydroxyl capable of being exposed to a solvent, such as a 5′ reducing hydroxyl or a 3′ hydroxyl. In some cases, a 5′ hydroxyl, a 3′ hydroxyl, or both, can be joined through a phosphorus-oxygen bond. In some embodiments, a 5′ hydroxyl, a 3′ hydroxyl, or both, can be modified into a phosphoester with a phosphorus-containing moiety. A guide RNA of the disclosure may not comprise (lacks) an exposed end. A guide RNA of the disclosure may not comprise (lacks) a 5′ end and a 3′ end. A guide RNA of the disclosure may retain a secondary structure—irrespective of whether the guide may be circular or not. For example, a circular guide RNA may comprise a secondary structure that is a stem loop, a cruciform, a toe hold, a mismatch bulge, more than one of any of these, or any combination thereof. A circular guide RNA may comprise a secondary structure that is substantially linear. A circular guide RNA may comprise a secondary structure that is modified to improve recruitment of an RNA editing entity or a secondary structure that partially mimics a native structure capable of recruiting an RNA editing entity.

In some cases, a circular guide RNA may comprise a half-life at least about: 2×, 3×, 4×, 5×, 6×, 7×, 8×, 9×, or 10× greater than a comparable guide RNA that is not circular. A half-life of a circular guide RNA may be from about 2× to about 5× greater than a comparable guide RNA that is not circular. A half-life of a circular guide RNA delivered to a cell or to a subject may be from about 3× to about 6× greater than a comparable guide RNA that is not circular.

A circular guide RNA delivered to a cell or to a subject may comprise a half-life in the cell or the subject of at least about: 40 minutes, 50 minutes, 60 minutes, 1.5 hr, 2 hr, 4 hr, 6 hr, 8 hr, 10 hr, 12 hr, 16 hr, 18 hr, 20 hr, 24 hr, 1.25 days, 1.5 days, 2 days, 3 days, 4 days, 5 days, 6 days, 7 days, 10 days, 14 days, or more. A half-life of a circular guide RNA delivered to a cell or to a subject may be from about 1 hr to about 6 hrs. A half-life of a circular guide RNA delivered to a cell or to a subject may be from about 1 hr to about 24 hrs. A half-life of a circular guide RNA delivered to a cell or to a subject may be from about 1 hr to about 2 days. A half-life of a circular guide RNA delivered to a cell or to a subject may be from about 6 hr to about 24 hrs. A half-life of a circular guide RNA delivered to a cell or to a subject may be from about 6 hr to about 5 days.

In some embodiments, an engineered guide RNA can be configured to undergo circularization in a cell. A construct for forming a circular RNA sequence may comprise a nucleotide sequence encoding for: (a) a guide RNA sequence for circularization comprising (i) an RNA editing entity recruiting domain, (ii) a ligation sequence, and (b) a ribozyme or catalytically active fragment thereof. In some cases, the nucleotide sequence may encode for two or more ligation sequences. In some cases, the nucleotide sequence may encode for two or more ribozymes. The two of more ligation sequences may be different. The two or more ribozymes may be different. A 5′ end, a 3′ end, or both of a guide RNA sequence may be flanked by a ligation sequence. A 5′ end or a 3′ end of a ligation sequence may be flanked by a ribozyme or catalytically active fragment thereof.

A construct for forming a circular RNA sequence may comprise a nucleotide sequence encoding for (a) an RNA sequence for circularization, (b) a ligation sequence, and (c) a tRNA, aptamer, or catalytically active fragment thereof. In some cases, the nucleotide sequence may encode for two ligation sequences. In some cases, the nucleotide sequence may encode for two self-cleaving entities (such as two tRNAs, two aptamers, or a combination). The nucleotide sequence may encode for two different ligation sequences. The nucleotide sequence may encode for two different self-cleaving entities, such as two different tRNAs, two different aptamers, or a combination. A 5′ end, a 3′ end, or both of a guide RNA sequence may be flanked by a ligation sequence. A 5′ end or a 3′ end of a ligation sequence may be flanked by a tRNA, aptamer, or other self-cleaving entity.

A circular RNA may be formed directly or indirectly by forming a linkage (such as a covalent linkage) between more than one end of the RNA sequence, such as a 5′ end and a 3′ end. The RNA sequence may comprise an engineered guide RNA (such as a recruiting domain, targeting domain, or both). The linkage may be formed by employing an enzyme, such as a ligase. In some cases, an enzyme can be a biologically active fragment of an enzyme. The enzyme may be recruited to the RNA sequence to form the linkage. A circular RNA may be formed by ligating more than one end of an RNA sequence using a linkage element. A linkage element may employ click chemistry to form the circular RNA. The linkage element may be an azide-based linkage. A circular RNA may be formed by genetically encoding or chemically synthesizing the circular RNA. A circular RNA may be formed by employing a self-cleaving entity, such as a ribozyme, tRNA, aptamer, catalytically active fragment of any of these, or any combination thereof. A self-cleaving ribozyme may comprise a RNase P.

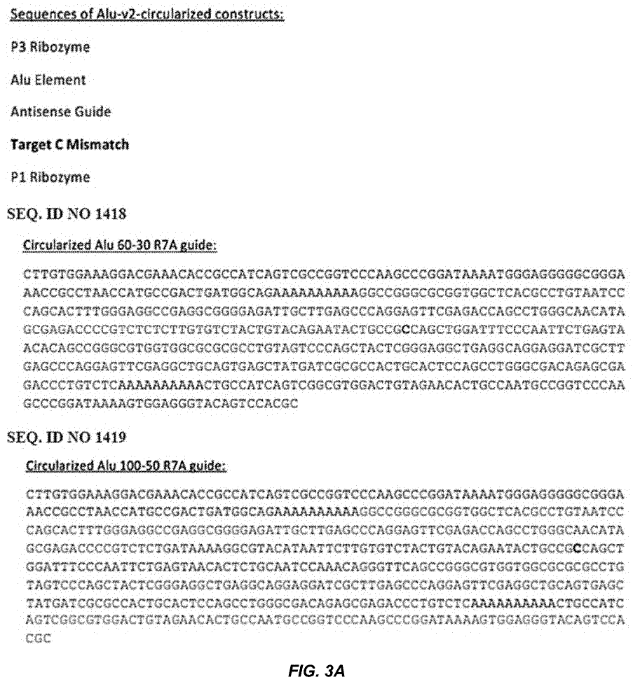

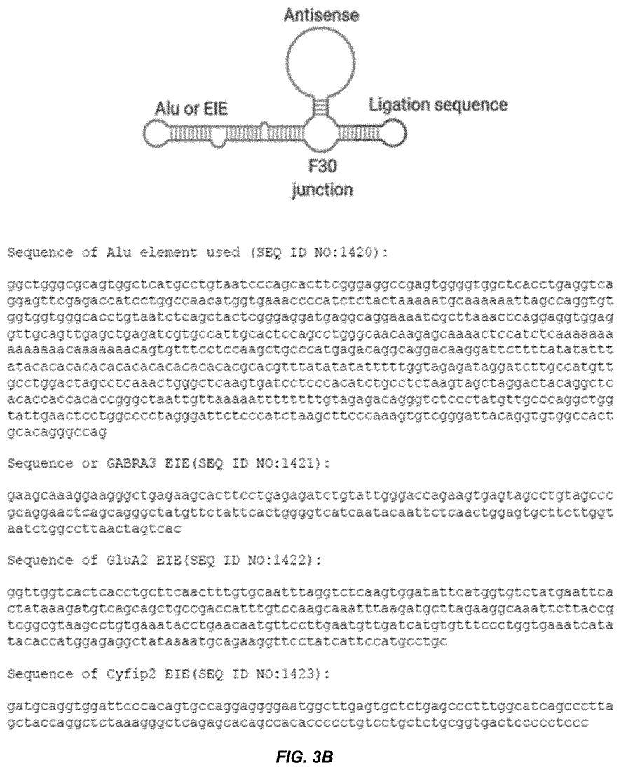

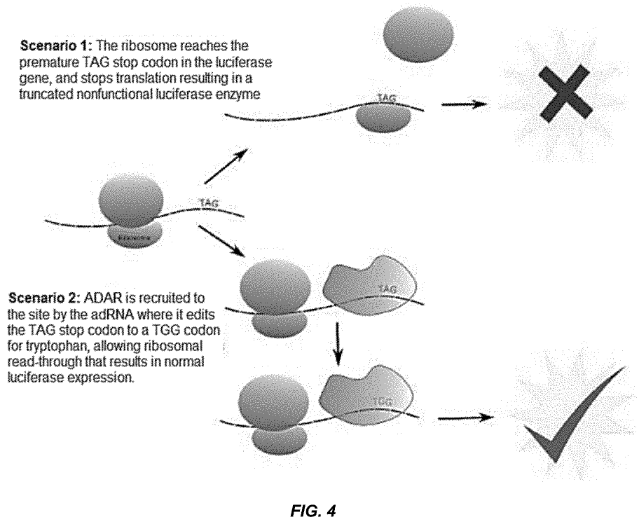

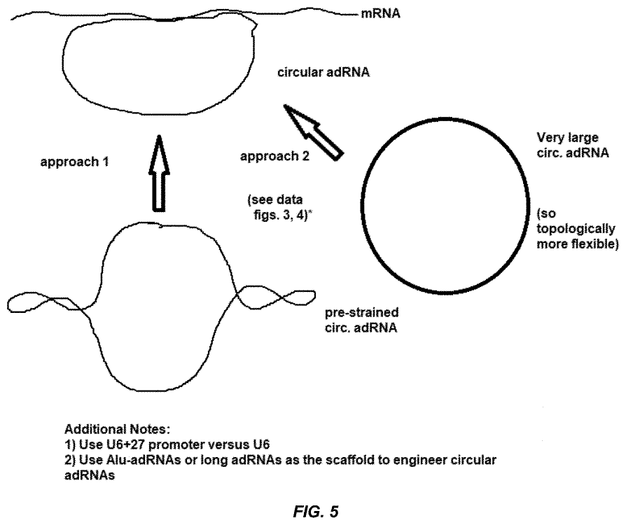

Guides may be circular guides. Referring to FIG. 3A-B, several examples of sequences having circular constructs are shown. Elements of a P3 ribozyme, Alu element, antisense guide, target C mismatch, P1 ribozyme are shown for each exemplary sequence. For example, a circular guide may comprise an Alu domain, such as an Alu 60-30 R7A guide shown in FIG. 3. A circular guide may comprise an Alu domain, such as an Alu 100-50 R7A guide shown in FIG. 3. In some cases, a circular guide or a guide utilized in any method as described herein may comprise a sequence having at least about: 70%, 75%, 80%, 85%, 90%, 95%, 98%, 99% sequence homology or more to at least a portion of a sequence of FIG. 3.

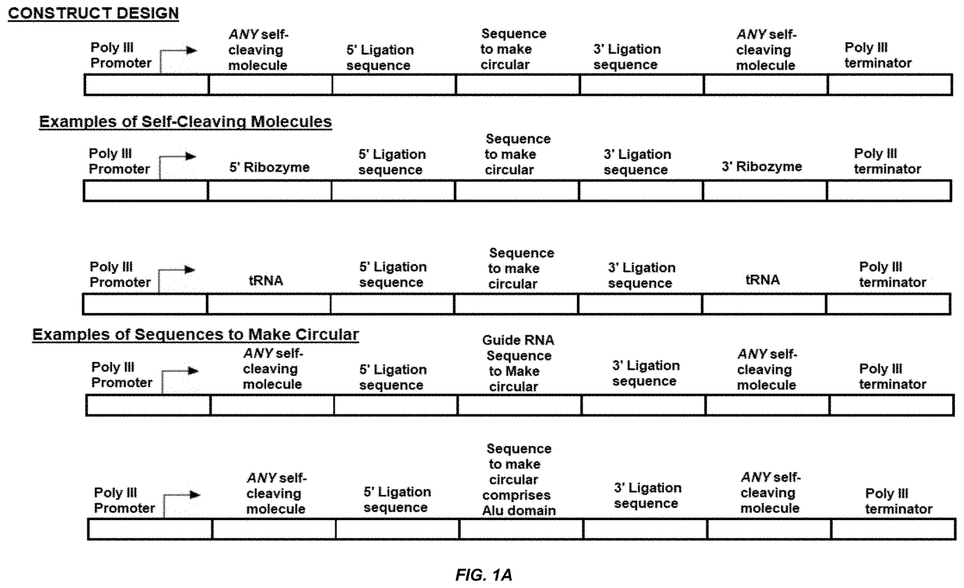

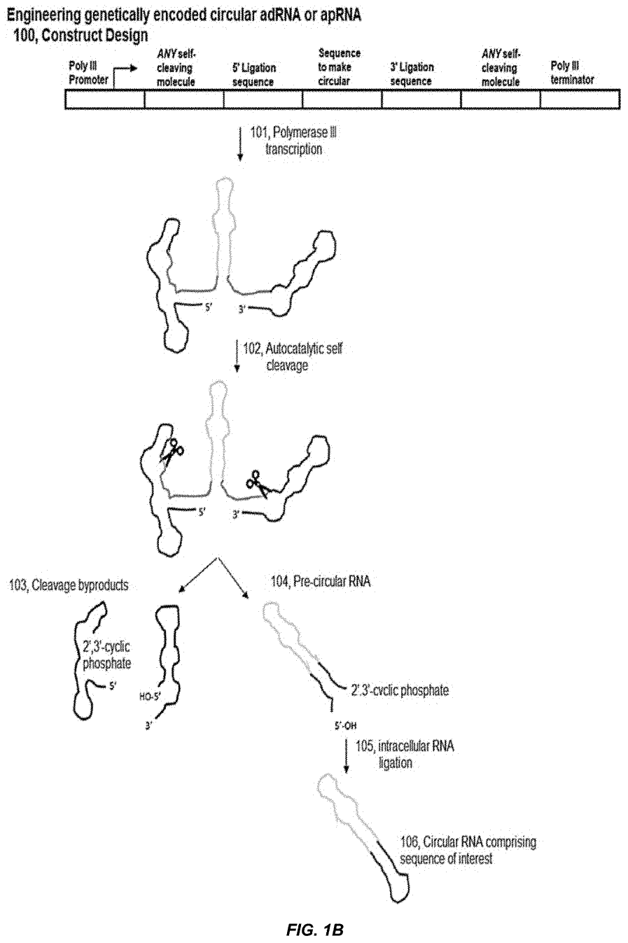

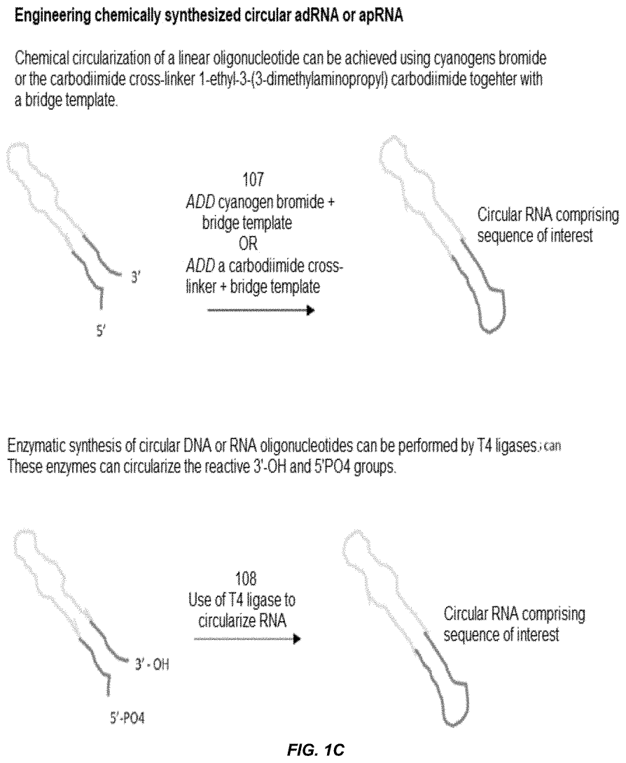

One or more methods may be employed to achieve forming a circular sequence (such as a circular RNA or circular DNA). Several methods are shown in FIG. 1A-C. A first method may include engineering genetically encoded circular adRNA or apRNA, such as shown in FIG. 1A-B. A construct may be designed to autocatalytically process the circular RNA. A construct may encode for a sequence to make circular, such as a guide RNA sequence. The guide RNA may include a targeting domain and an RNA editing entity recruiting domain. The RNA editing entity recruiting domain may include an Alu domain, an APOBEC recruiting domain, a GluR2 domain, a Cas13 recruiting domain, or any combination thereof. The construct may encode for at least one ligation sequence, in some cases two ligation sequences. The construct may encode for at least one self-cleaving molecule, in some cases two self-cleaving molecules. The self-cleaving molecule may include a ribozyme, a tRNA, or any other self-cleaving molecule. In some cases, the self-cleaving molecule may be the tRNA. In some cases, at least one of: a 5′ end or a 3′ end of the sequence to make circular may be flanked by a ligation sequence, such as a sequence recognized by a ligase, such as an endogenous ligase. In some cases, at least one of: a 5′ end or a 3′ end of the ligation sequence may be flanked by the sequence encoding the self-cleaving molecule. An example construct is shown as element 100 of FIG. 1B. Polymerase III transcription 101 results in the RNA sequence transcribed from the encoded construct, typically using an H1, U6 or 7SK promoter. This RNA sequence may then be autocatalytically self-cleaved 102 to separate the self-cleaving portions 103 (by products of the process) from the sequence to make circular 104 (the pre-circular RNA). Following self-cleavage, the sequence to make circular may be flanked by the ligation sequence with an OH group on the 5′ end and a 2′, 3′-cyclic phosphate group on the 3′ end. A ligase (such as an endogenous ligase) may recognize the ligation sequence and ligate the 5′ and 3′ ends 105 which may result in the formation of the circular RNA 106. This construct design may encode any self-cleaving molecule, such as a ribozyme, a tRNA, or other. This construct design may encode any sequence to make circular, such as an RNA for therapeutic delivery, such as an miR, a RNAi, a RNaseH, siRNA, a guide RNA (including CRISPR guides), or others. This construct design may encode any ligation sequence recognized by any ligase to ligate the ends of a linear construct to form the circular sequence. A ligase may be an endogenous ligase. A ligase may be co-delivered with the construct. The construct design may be designed for ligation by a specific ligase or multiple ligases. The method may include forming a pre-strained circular adRNA, such as shown in FIG. 5.

Another method to form a circular RNA may include engineering chemically synthesized circular adRNA or apRNA, such as shown in FIG. 1C. In this case, a chemical circularization of a linear oligonucleotide may be achieved using cyanogen bromide or the carbodiimide cross-linker 1-ethyl-3-(3-dimethylaminopropyl) carbodiimide together with a bridge template, as shown in 107. In other cases, an enzymatic synthesis of circular DNA or RNA oligonucleotides may be performed by T4 ligases. These enzymes may circularize the reactive 3′-hydroxyl (OH) and 5′-phosphate (PO4) groups on the ends of a linear sequence, as shown in 108. A construct design may include forming a pre-strained circular adRNA, such as shown in FIG. 5. The method may include forming a pre- strained circular adRNA, such as shown in FIG. 5.

A suitable self-cleaving molecule may include a ribozyme. A ribozyme may include a RNase P, a rRNA (such as a Peptidyl transferase 23S rRNA), Leadzyme, Group I intron ribozyme, Group II intron ribozyme, a GIR1 branching ribozyme, a glmS ribozyme, a hairpin ribozyme, a Hammerhead ribozyme, a HDV ribozyme, a Twister ribozyme, a Twister sister ribozyme, a VS ribozyme, a Pistol ribozyme, a Hatchet ribozyme, a viroid, or any combination thereof.

A suitable ligase (or synthetase) may include a ligase that forms a covalent bond. A covalent bond may include a carbon-oxygen bond, a carbon-sulfur bond, a carbon-nitrogen bond, a carbon-carbon bond, a phosphoric ester bond, or any combination thereof.

A pathway to construct a circular RNA sequence (such as a guide) may start with a tRNA splicing endonuclease binding to a specific recognition sequence and creating a 5′ hydroxyl group and 2′-3′ cyclic phosphate on cleaved ends. These cleaved ends may be ligated together by a ligase, such as an endogenous ligase (for example, a ubiquitously expressed RNA ligase RtcB). The advantage of employing this strategy may be the lack of additional enzymes required. The RNA transcripts may be expressed containing an RNA of interest flanked by a self-cleaving molecule, such as ribozymes. Addition of a sequence encoding a ribozyme may create an autocatalytic RNA. In some cases, the ribozymes may be P3 Twister and P1 Twister that may undergo spontaneous autocatalytic cleavage. The resulting RNA may contain the 5′ and 3′ ends that may then be ligated a ligase (such as ubiquitously expressed endogenous RNA ligase RtcB). Increasing the stability of the adRNA may have a significant impact on in vivo studies since editing depends on long term expression. The method may include forming a pre-strained circular adRNA, such as shown in FIG. 5.

An aspect of the disclosure provides for engineered guide RNAs, vectors comprising engineered guide RNAs, compositions, and pharmaceutical compositions for RNA editing. Any of the above or as described herein can be configured for an A (adenosine) to I (inosine) edit, a C (cytosine) to T (thymine) edit, or a combination thereof. In some cases, an A to I edit can be interpreted or read as a C to U mutation. In some cases, an A to I edit can be interpreted or read as an A to G mutation. Engineered guide RNAs, vectors comprising engineered guide RNAs, compositions, and pharmaceutical compositions as described herein can provide enhanced editing efficiencies as compared to native systems, reduced off-target editing, enhanced stability or in vivo half-lives, or any combination thereof.

An aspect of the disclosure provides for a vector. The vector can comprise a nucleic acid with a polynucleotide sequence encoding (i) an RNA editing entity recruiting domain, (ii) a targeting domain complementary to at least a portion of a target RNA, (iii) optionally more than one of either domain (i) and/or (ii), or (iv) any combination thereof. In some cases, the vector can be administered to a subject, such as a subject in need thereof. In some cases, the vector can be administered as part of a pharmaceutical composition to a subject, such as a subject in need thereof. In some cases, the polynucleotide sequence encodes for a circular guide RNA.

An aspect of the disclosure provides for a non-naturally occurring RNA. The non-naturally occurring RNA can comprise (i) an RNA editing entity recruiting domain, (ii) a targeting domain complementary to at least a portion of a target RNA, (iii) optionally more than one of either domain (i) and/or (ii), or (iv) any combination thereof. In some cases, the non-naturally occurring RNA is circular. In some cases, the non-naturally occurring RNA does not comprise (lacks) an exposed end or a single stranded end. In some cases, the non-naturally occurring RNA can be administered to a subject, such as a subject in need thereof. In some cases, the non-naturally occurring RNA can be administered as part of a pharmaceutical composition to a subject, such as a subject in need thereof. In some cases, the non-naturally occurring RNA can be formulated in a vector for administration. The vector can comprise a viral vector, a liposome, a nanoparticle, or any combination thereof. In some cases, the non-naturally occurring RNA can comprise at least one base, at least one sugar, more than one of either, or a combination thereof having a modification, such as a chemical modification.

An engineered guide RNA can comprise one or more domains, such as 1, 2, 3, 4, 5 or more domains. In some cases, an engineered guide RNA can comprise a recruiting domain, a targeting domain, more than one of either, or a combination thereof. In some cases, an engineered guide RNA can comprise a targeting domain and a recruiting domain. In some cases, an engineered guide RNA can comprise a targeting domain and two recruiting domains.

A domain can form a two dimensional shape or secondary structure. For example, a targeting domain, a recruiting domain or a combination thereof can form a secondary structure that can comprise a linear region, a cruciform or portion thereof, a toe hold, a stem loop, or any combination thereof. The domain itself can form a substantially linear two dimensional structure. The domain can form a secondary structure that can comprise a cruciform. The domain can form a secondary structure that can comprise a stem loop. The domain can form a secondary structure that can comprise a toehold.

In some cases, a targeting domain can be positioned adjacent to a recruiting domain, including immediately adjacent or adjacent to but separated by a number of nucleotides (e.g., 1, 2, 3, 4, 5, 6, 7, 8, 9, 10, 15, 20, 25, 30 or more nucleotides). In some cases, a targeting domain can be flanked by two recruiting domains. In some cases, two or more recruiting domains can be adjacent to one another.

Two dimensional shape or secondary structure of a domain can influence efficiency of editing, off target effects, or a combination thereof as compared to a nucleic acid that can form a different two dimension shape or secondary structure. Therefore, an aspect of the disclosure includes modifying nucleic acids such that two dimensional shapes can be advantageously designed to enhance efficiency of editing and reduce off target effects. Modifications to a sequence comprising a naturally occurring recruiting domains can also enhance editing efficiency and reduce off target effects. Therefore, an aspect of the disclosure includes modifying nucleic acids such that a sequence (such as a synthetic sequence) can be advantageously designed to enhance efficiency of editing and reduce off target effects. Modifications can include altering a length of a domain (such as extending a length), altering a native sequence that results in a change in secondary structure, adding a chemical modification, or any combination thereof. Nucleic acids as described herein can provide these advantages. Modifications can include providing the guide RNA in circular form. Modifications can include forming a circular guide RNA to remove one or more exposed ends or one or more single stranded ends. Circularization of a guide RNA may permit the guide RNA to retain a secondary structure, such as a stem loop or cruciform.

In some cases, a recruiting domain can comprise at least about: 80%, 85%, 90%, or 95% sequence homology to at least about: 15, 20, 25, 30, or 35 nucleic acids of an Alu domain. In some cases, at least a portion of a recruiting domain can comprise at least about 80% sequence homology to an Alu domain encoding sequence. In some cases, at least a portion of a recruiting domain can comprise at least about 85% sequence homology to an Alu domain encoding sequence. In some cases, at least a portion of a recruiting domain can comprise at least about 90% sequence homology to an Alu domain encoding sequence. In some cases, at least a portion of a recruiting domain can comprise at least about 95% sequence homology to an Alu domain encoding sequence. In some cases, the Alu domain encoding sequence can be a non-naturally occurring sequence. In some cases, the Alu domain encoding sequence can comprise a modified portion. In some cases, the Alu domain encoding sequence can comprise a portion of a naturally occurring Alu domain sequence.

In some cases, a recruiting domain can comprise at least about: 80%, 85%, 90%, or 95% sequence homology to at least about: 15, 20, 25, 30, or 35 nucleic acids of an APOBEC recruiting domain. In some cases, at least a portion of a recruiting domain can comprise at least about 80% sequence homology to an APOBEC recruiting domain encoding sequence. In some cases, at least a portion of a recruiting domain can comprise at least about 85% sequence homology to an APOBEC recruiting domain encoding sequence. In some cases, at least a portion of a recruiting domain can comprise at least about 90% sequence homology to an APOBEC recruiting domain encoding sequence. In some cases, at least a portion of a recruiting domain can comprise at least about 95% sequence homology to an APOBEC recruiting domain encoding sequence. In some cases, the APOBEC recruiting domain encoding sequence can be a non-naturally occurring sequence. In some cases, the APOBEC recruiting domain encoding sequence can comprise a modified portion. In some cases, the APOBEC recruiting domain encoding sequence can comprise a portion of a naturally occurring APOBEC recruiting domain sequence.

In some cases, a recruiting domain can comprise at least about: 80%, 85%, 90%, or 95% sequence homology to at least about: 15, 20, 25, 30, or 35 nucleic acids of an GluR2 domain. In some cases, at least a portion of a recruiting domain can comprise at least about 80% sequence homology to a GluR2 domain encoding sequence. In some cases, at least a portion of a recruiting domain can comprise at least about 85% sequence homology to a GluR2 domain encoding sequence. In some cases, at least a portion of a recruiting domain can comprise at least about 90% sequence homology to a GluR2 domain encoding sequence. In some cases, at least a portion of a recruiting domain can comprise at least about 95% sequence homology to a GluR2 domain encoding sequence. In some cases, the GluR2 domain encoding sequence can be a non-naturally occurring sequence. In some cases, the GluR2 domain encoding sequence can comprise a modified portion. In some cases, the GluR2 domain encoding sequence can comprise a portion of a naturally occurring GluR2 domain sequence.

In some cases, a recruiting domain can comprise at least about: 80%, 85%, 90%, or 95% sequence homology to at least about: 15, 20, 25, 30, or 35 nucleic acids of a Cas13 recruiting domain. The Cas13 recruiting domain may be a Cas13a recruiting domain, a Cas13b recruiting domain, a Cas13c recruiting domain, or a Cas 13d recruiting domain. In some cases, at least a portion of a recruiting domain can comprise at least about 80% sequence homology to a Cas13 recruiting domain encoding sequence. In some cases, at least a portion of a recruiting domain can comprise at least about 85% sequence homology to a Cas13 recruiting domain encoding sequence. In some cases, at least a portion of a recruiting domain can comprise at least about 90% sequence homology to a Cas13 recruiting domain encoding sequence. In some cases, at least a portion of a recruiting domain can comprise at least about 95% sequence homology to a Cas13 recruiting domain encoding sequence. In some cases, the Cas13 recruiting domain encoding sequence can be a non-naturally occurring sequence. In some cases, the Cas13 recruiting domain encoding sequence can comprise a modified portion. In some cases, the Cas13 recruiting domain encoding sequence can comprise a portion of a naturally occurring Cas13 recruiting domain sequence.

In some cases, at least a portion of a recruiting domain can comprise at least about 80% sequence identify to an encoding sequence that recruits an ADAR, that recruits an APOBEC, or a combination thereof.

In some cases, an engineered polynucleotide can encode for at least one RNA editing entity recruiting domain. In some cases, the engineered polynucleotide can encode for an RNA that is complementary to at least a portion of a target RNA. In some cases, the engineered polynucleotide can encode for a recruiting domain and a targeting domain. In some cases, the engineered polynucleotide can encode for a circular guide RNA. In some cases, the engineered polynucleotide can encode for a recruiting domain and a nucleic acid can encode for a targeting domain. The portion of the target RNA can comprise a single nucleotide base. The portion of the target RNA can comprise a plurality of bases. The portion of the target RNA can comprise about: 2, 3, 4, 5, 6, 7, 8, 9, 10, 20, 30, 40, 50, 60, 70, 100, 200, 300, 400, 500, 600, 700, 800, 900, 1000 base pairs or more. In some cases, the target RNA can comprise from about 1 bps to about 10 bps. In some cases, the target RNA can comprise from about 10 bps to about 100 bps. In some cases, the target RNA can comprise from about 10 bps to about 500 bps. In some cases, the target RNA can comprise from about 10 bps to about 1000 bps. An engineered polynucleotide comprising a targeting domain and a recruiting domain can comprise a contiguous sequence of at least about 200 bp in length. An engineered polynucleotide comprising a targeting domain and a recruiting domain can comprise a contiguous sequence of at least about 150 bp in length. An engineered polynucleotide comprising a targeting domain and a recruiting domain can comprise a contiguous sequence of at least about 250 bp in length. An engineered polynucleotide comprising a targeting domain and a recruiting domain can comprise a contiguous sequence of at least about 275 bp in length. An engineered polynucleotide comprising a targeting domain and a recruiting domain can comprise a contiguous sequence of at least about 300 bp in length. An engineered polynucleotide comprising a targeting domain and a recruiting domain can comprise a contiguous sequence of at least about 400 bp in length. An engineered polynucleotide comprising a targeting domain and a recruiting domain can comprise a contiguous sequence of at least about 500 bp in length.

A vector can be employed to deliver an engineered polynucleotide. A vector can comprise DNA, such as double stranded DNA or single stranded DNA. A vector can comprise RNA. In some cases, the RNA can comprise one or more base modifications. The vector can comprise a recombinant vector. In some cases, the vector can be a vector that is modified from a naturally occurring vector. The vector can comprise at least a portion of a non-naturally occurring vector. Any vector can be utilized. In some cases, the vector can comprise a viral vector, a liposome, a nanoparticle, an exosome, an extracellular vesicle, or any combination thereof. In some cases, a viral vector can comprise an adenoviral vector, an adeno-associated viral vector (AAV), a lentiviral vector, a retroviral vector, a portion of any of these, or any combination thereof. In some cases, a nanoparticle vector can comprise a polymeric-based nanoparticle, an aminolipid based nanoparticle, a metallic nanoparticle (such as gold-based nanoparticle), a portion of any of these, or any combination thereof. In some cases, a vector can comprise an AAV vector. A vector can be modified to include a modified VP1 protein (such as an AAV vector modified to include a VP1 protein). An AAV can comprise a serotype—such as an AAV1 serotype, an AAV2 serotype, AAV3 serotype, an AAV4 serotype, AAV5 serotype, an AAV6 serotype, AAV7 serotype, an AAV8 serotype, an AAV9 serotype, a derivative of any of these, or any combination thereof.

Administration of an engineered polynucleotide comprising a guide RNA can be effected in one dose, continuously or intermittently throughout the course of treatment. Methods of determining the most effective means and dosage of administration are known to those of skill in the art and can vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and can vary with the composition used for treatment, the purpose of the treatment, the health condition or disease stage of the subject being treated, and target cell or tissue. Non-limiting examples of routes of administration include oral administration, nasal administration, injection, and topical application.

Administration can refer to methods that can be used to enable delivery of compounds or compositions to the desired site of biological action (such as DNA constructs, viral vectors, or others). These methods can include topical administration (such as a lotion, a cream, an ointment) to an external surface of a surface, such as a skin. These methods can include parenteral administration (including intravenous, subcutaneous, intrathecal, intraperitoneal, intramuscular, intravascular or infusion), oral administration, inhalation administration, intraduodenal administration, and rectal administration. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition. In some cases, a cosmetic professional can administer the composition.

Administration or application of a composition disclosed herein can be performed for a treatment duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration or application of composition disclosed herein can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a composition can be administered or applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

An in vitro half-life of a circular RNA sequence may be at least about: 1×, 1.5×, 2×, 2.5×, 3×, 3.5×, 4×, 5×, 10×, 20× longer or more as compared to a substantially comparable linear RNA sequence. An in vivo half-life of a circular RNA sequence may be at least about: 1×, 1.5×, 2×, 2.5×, 3×, 3.5×, 4×, 5×, 10×, 20× longer or more as compared to a substantially comparable linear RNA sequence. A dosage of a composition comprising a circular RNA sequence administered to a subject in need thereof may be at least about: 1×, 1.5×, 2×, 2.5×, 3×, 3.5×, 4×, 5×, 10×, 20× less as compared to a composition comprising a substantially comparable linear RNA sequence administered to the subject in need thereof. A composition comprising a circular RNA sequence administered to a subject in need thereof may be given as a single time treatment as compared to a composition comprising a substantially comparable linear RNA sequence given as a two-time treatment or more.

A composition may comprise a combination of the active agent, e.g., an engineered guide RNA of this disclosure, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

In some embodiments, a kit can comprise a guide RNA. In some instances, a kit can comprise an engineered polynucleotide, a engineered guide RNA, a construct for forming a circular guide RNA sequence, a vector comprising an engineered polynucleotide, a nucleic acid of the engineered polynucleotide, a pharmaceutical composition and a container. In some cases, a container can be sterile. In some instances, a container can be plastic, glass, metal, or any combination thereof. In some cases, a kit can comprise instructions for use, such as instructions for administration to a subject in need thereof. In some embodiments, a method of making a kit can comprise adding a polynucleotide described herein into a container.

A pharmaceutical composition can comprise a first active ingredient. The first active ingredient can comprise a vector as described herein, or an engineered guide RNA. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient.

In some embodiments, a pharmaceutical composition can be formulated in milligrams (mg), milligram per kilogram (mg/kg), copy number, or number of molecules.

A composition described herein can compromise an excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some cases, an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some cases, an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some cases, a pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The binders that can be used in a pharmaceutical formulation can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some cases, a pharmaceutical formulation can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some cases, a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isomorphous silicate, and microcrystalline cellulose as high HLB emulsifier surfactants.

In some cases, a pharmaceutical formulation can comprise a disintegrant as an excipient. In some cases, a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some cases, a disintegrant can be an effervescent disintegrant. Non-limiting examples of suitable effervescent disintegrants can include sodium bicarbonate in combination with citric acid, and sodium bicarbonate in combination with tartaric acid.

In some cases, an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some cases, a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some cases, an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

A subject, host, individual, and patient may be used interchangeably herein to refer to any organism eukaryotic or prokaryotic. In some cases, subject may refer to an animal, such as a mammal. A mammal can be administered a vector, engineered guide RNA, cell or composition as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder. In other embodiments, a subject has or is suspected of having a disease or disorder associated with aberrant protein expression. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

A disease or condition can comprise a neurodegenerative disease, a muscular disorder, a metabolic disorder, an ocular disorder, or any combination thereof. The disease or condition can comprise cystic fibrosis, albinism, alpha-1-antitrypsin deficiency, Alzheimer disease, Amyotrophic lateral sclerosis, Asthma, β-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Parkinson's disease, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, various forms of cancer (e.g. BRCA1 and 2 linked breast cancer and ovarian cancer). The disease or condition can comprise a muscular dystrophy, an ornithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, or any combination thereof. Administration of a composition can be sufficient to: (a) decrease expression of a gene relative to an expression of the gene prior to administration; (b) edit at least one point mutation in a subject, such as a subject in need thereof; (c) edit at least one stop codon in the subject to produce a readthrough of a stop codon; (d) produce an exon skip in the subject, or (e) any combination thereof. A disease or condition may comprise a muscular dystrophy. A muscular dystrophy may include myotonic, Duchenne, Becker, Limb-girdle, facioscapulohumeral, congenital, oculopharyngeal, distal, Emery-Dreifuss, or any combination thereof. A disease or condition may comprise pain, such as a chronic pain. Pain may include neuropathic pain, nociceptive pain, or a combination thereof. Nociceptive pain may include visceral pain, somatic pain, or a combination thereof.

### EXAMPLES

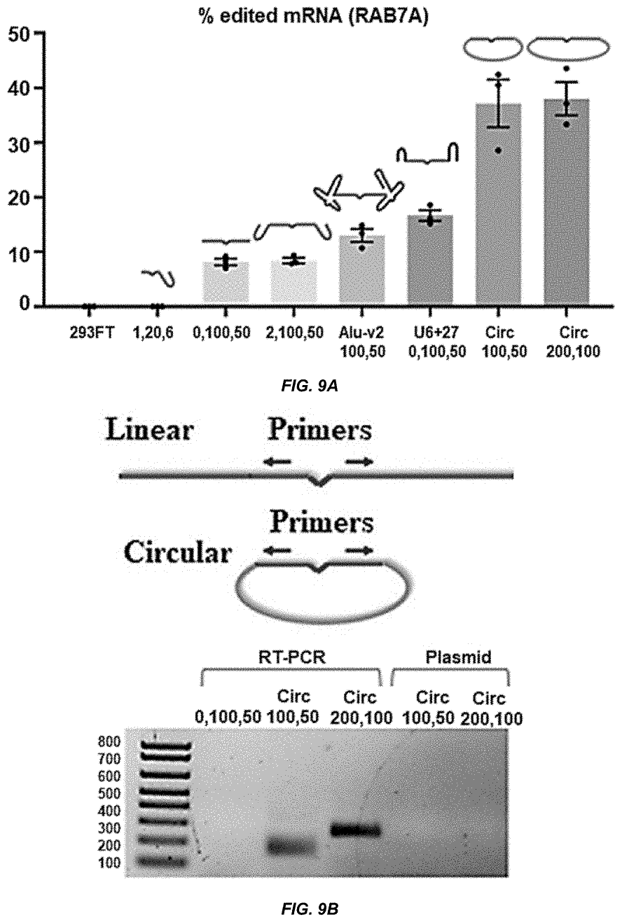

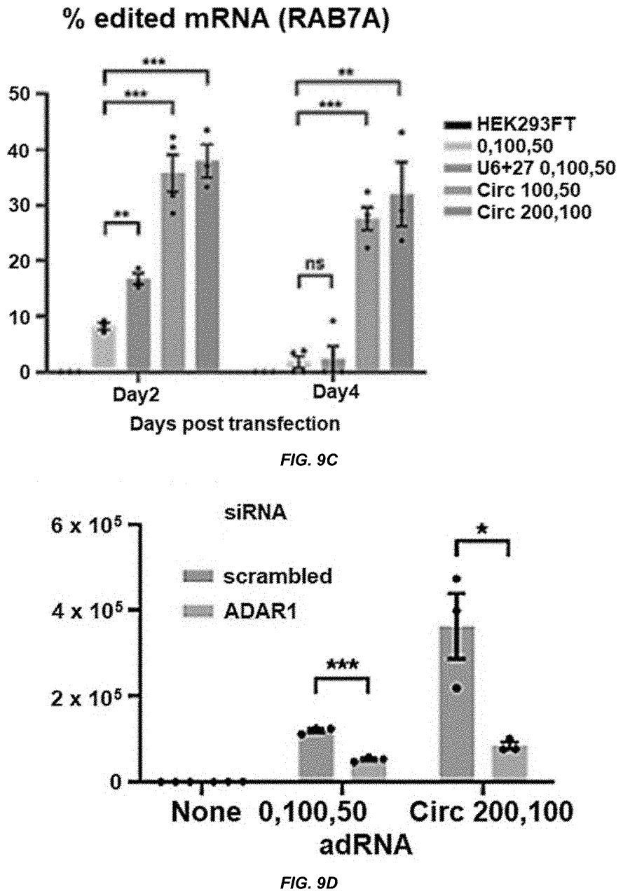

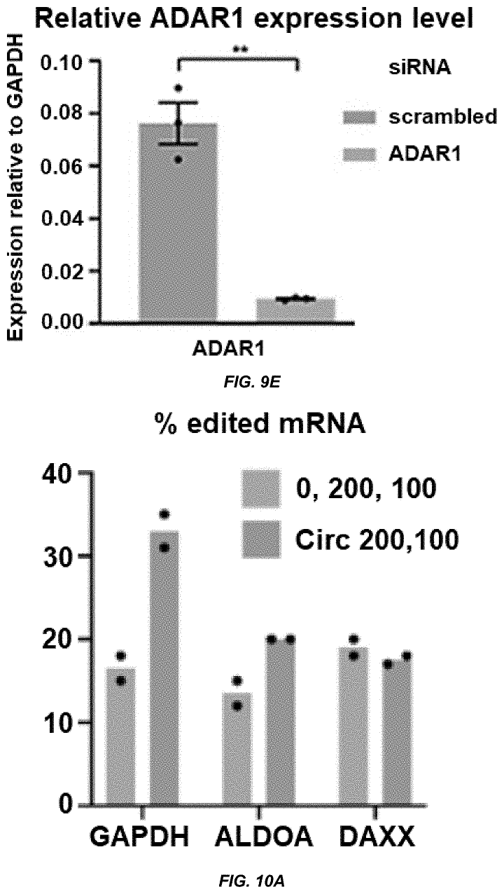

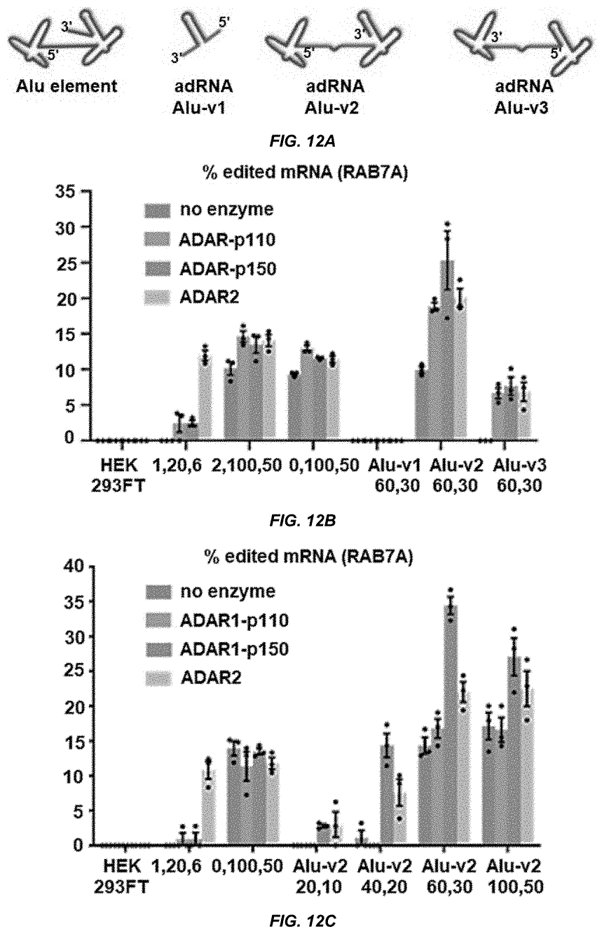

Example 1—The process of RNA editing via endogenous ADARs can be made more efficient either by improving the recruitment of ADAR1 to the target or by enabling longer persistence of the adRNA or using a combination of the two. Since Alu elements are known substrates for ADAR1, it was hypothesized that engineering adRNA bearing Alu domains might improve ADAR1 recruitment to the target. Thus, three versions of Alu adRNAs were created by carefully positioning the antisense domain at various locations in the Alu consensus sequence and assaying for editing at an adenosine in the 3′UTR of the RAB7A transcript in HE293FT cells (FIG. 9A, 12A-B). Additionally, different lengths of antisense domains were tested (FIG. 12C). The addition of Alu domains on the long antisense adRNA did not significantly improve the efficiency of RNA editing via endogenous ADARs (FIG. 9A); however, the Alu-v2 adRNAs were highly efficient at recruiting overexpressed ADAR1-150, the IFN inducible isoform of ADAR1 (FIGS. 12B-C).

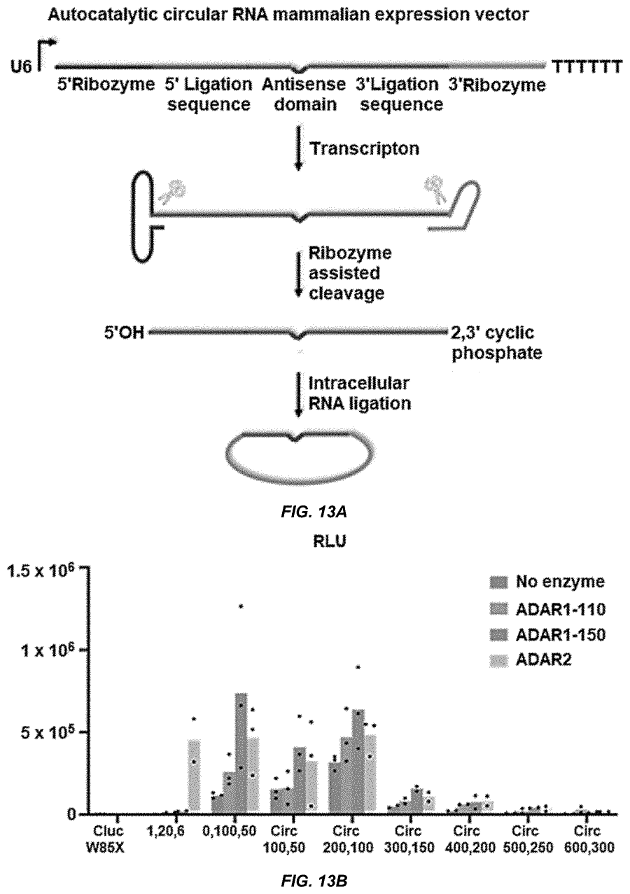

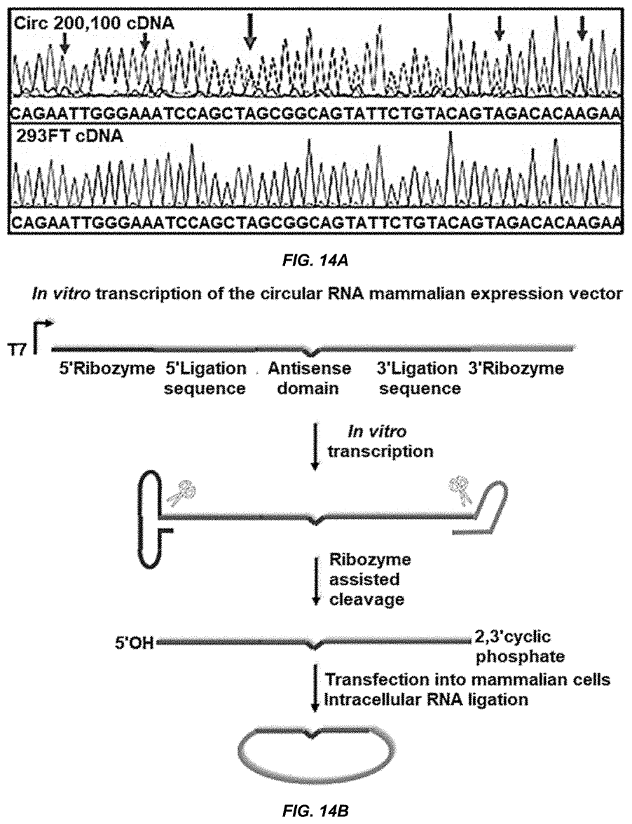

Unmodified RNAs typically have a short half-life. The disclosure provides methods and compositions towards enabling longer persistence of the adRNA, U6+27 cassette which has been shown to improve stability of siRNA were tested. The addition of U6+27 to the long antisense adRNA led to slight improvement in editing of the RAB7A transcript (FIG. 9A). Further, circular RNAs are known to persist in cells over a duration of several days thus it was questioned if it was possible to create a circular adRNA for recruiting endogenous ADARs. Studies looked at U6 transcribed circular RNAs in mammalian cells (FIG. 13A). A circular adRNA was created with varying antisense lengths and assayed for their ability to repair a premature stop codon in the cypridina luciferase (cluc) transcript (FIG. 13B). Circularization of the adRNA in HEK293FT cells was confirmed via RT-PCR by designing primers that selectively amplified only circularized RNA (FIG. 9B). Using circular adRNA with antisense lengths 100 bp and 200 bp, 35-38% editing of the RAB7A transcript was observed, a 3.5-fold improvement in efficiency over the long antisense adRNA as well as a 2-fold improvement over Alu adRNA and U6+27 adRNA, 2 days post transfections (FIG. 9A). Additionally, using circular adRNA persistence of significant levels RNA editing 4 days post transfections was also observed, at a time when editing via linear forms was almost non-existent (FIG. 9C). A luciferase assay was performed in the presence of scrambled and ADAR1 siRNA. A significant drop in luciferase activity was demonstrated in the presence of siRNA targeting ADAR1, confirming that RNA editing via delivery of long antisense adRNA and their circular variants occurred due to recruitment of endogenous ADAR1 (FIGS. 9D, 9E). One issue of using circular adRNA with long antisense domains, however, is the hyperediting seen in adenosines flanking the target (FIG. 14A). This was addressed using mismatches at hyperedited adenosines flacking the target.

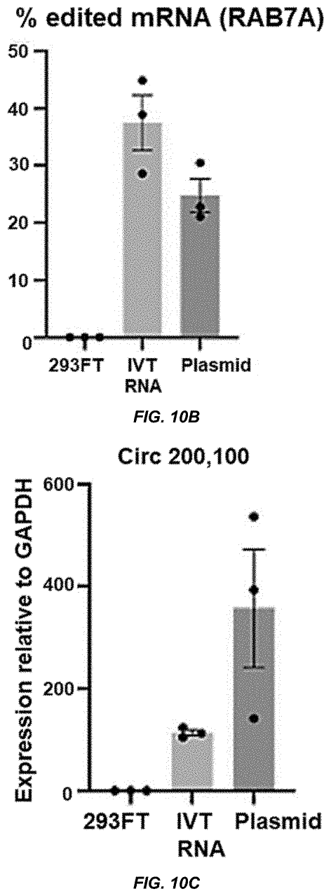

To confirm generalizability of the results, a circular and linear adRNA of with antisense lengths of 200 bp to edit adenosines in the coding sequence (CDS) of three transcripts—GAPDH, ALDOA and DAXX in HEK 293FT cells was performed (FIG. 10A). The circular adRNA were better at editing GAPDH and ALDOA transcripts while for DAXX a difference between the linear and circular constructs 2 days post transfections was not observed. Further, the ability of in vitro transcribed (IVT) circular adRNA, which are delivered to cells in a linear form but undergo circularization in the cell, to enable RNA editing was tested (FIG. 14B). Within a day post transfections, nearly 40% editing of the RAB7A transcript using IVT adRNA as compared to the 25% seen via plasmid delivery was observed (FIG. 10B). Circularization of the IVT adRNA and plasmid delivered adRNA in cells was also confirmed via qPCR (FIG. 10C).

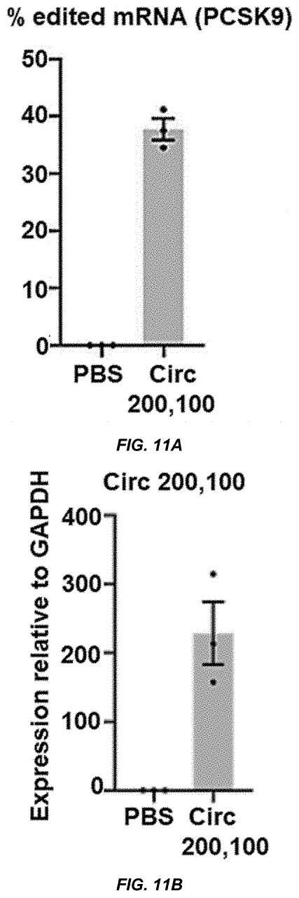

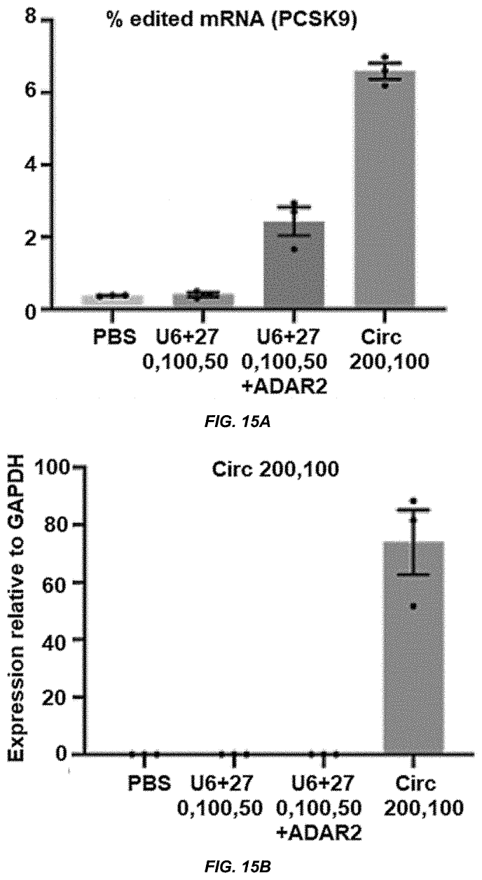

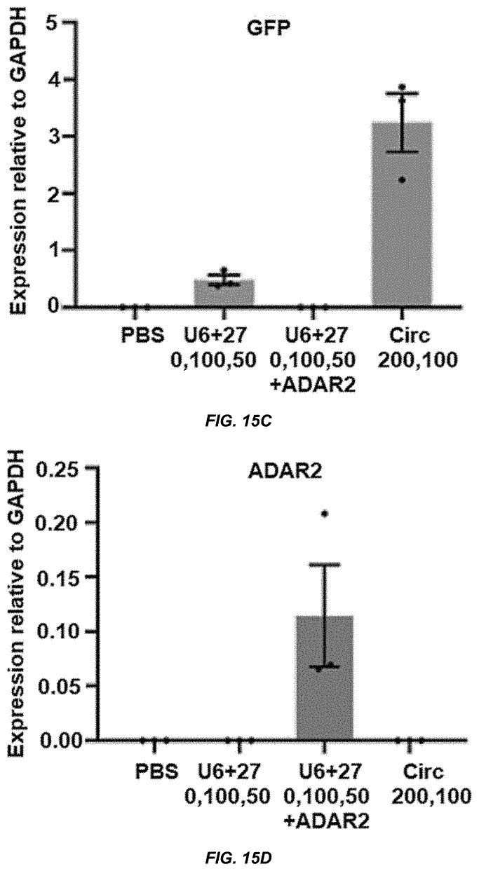

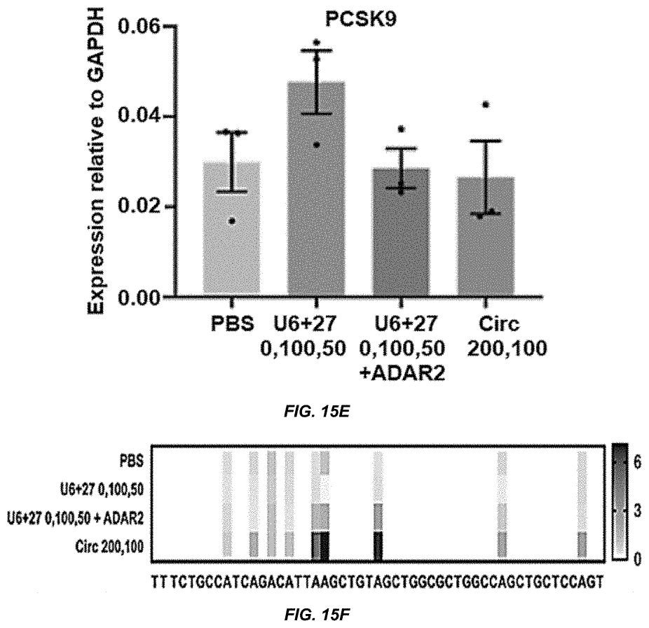

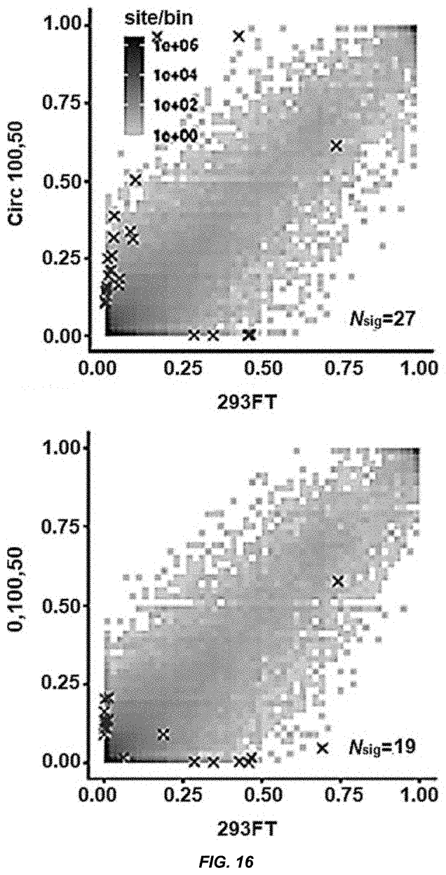

The efficiencies of linear and circular adRNA at editing an endogenous transcript in mice was compared. The U6+27 linear and circular adRNA, targeting an adenosine in the 3′ UTR of the PCSK9 transcript, were packaged into AAV8 which is known to have high liver tropism. As a positive control a third AAV was used having packaged the U6+27 adRNA as well as the ADAR2 enzyme. 2 weeks post injections, mice livers were harvested and showed 6-7% editing of the target site in the PCSK9 transcript via recruitment of endogenous ADARs as compared to the <1% seen with the U6+27 adRNA or 2-3% seen via the U6+27 adRNA in presence of overexpressed ADAR2 (FIG. 10A). qPCR was performed to confirm expression of the circular adRNA and the other AAV delivered constructs (FIGS. 15B-D). Additionally, AAV delivered linear and circular long antisense adRNA did not significantly alter the expression levels of PCSK9 in mice livers (FIG. 15E). However, using circular adRNA, equally high levels of editing at a non-target adenosine in the PCSK9 transcript located 6 bp upstream of the target was observed (FIG. 15F). It was hypothesized that it might be possible to improve the efficiency of RNA editing by increasing the expression of the circular adRNA. Thus, an AAV vector bearing two copies of the circular adRNA expressed via hU6 and mU6 promoters both of which were cloned downstream of a CMV promoter driven mCherry were tested. Additionally, it is believed that the long antisense RNA remained bound to the transcript of interest and thereby prevented efficient reverse transcription of the edited mRNA. To overcome this, prior to cDNA synthesis, a sense RNA was used to capture the long antisense RNA thereby freeing up the target mRNA for reverse transcription. Using this vector with a slight alteration to the cDNA synthesis protocol, within two weeks post injections, 35-40% editing of the PCSK9 locus was observed (FIG. 11A). Additionally, a robust expression of the circular adRNA which was about 3-fold higher than the original vector used was observed. (FIG. 11B and 15B).

Because the composition and methods of the disclosure achieved 35-40% of an endogenous target in vivo this approach opens the door to a potential gene therapy which could be used to efficiently repair G-to-A point mutations or premature stop codons. Although the innate immune response against delivery of circular adRNAs needs further assessment, AAV-based gene therapies are already in clinic and since this approach does not require the need for delivery of foreign effector proteins, this approach could have immense therapeutic potential.

Example 2—A subject will be diagnosed with a disease. The subject will be prescribed a dosing regimen of a pharmaceutical composition. The pharmaceutical composition will comprise a circular engineered guide RNA or a recombinant polynucleotide that encodes a circular engineered guide RNA. The circular engineered guide RNA will comprise a targeting domain and an RNA editing entity recruiting domain capable of recruiting an RNA editing entity that performs an editing of a nucleotide. The pharmaceutical composition will be administered to the subject by injection. A dosing regimen of the circular engineered guide RNA will be 2× less frequent than a subject receiving by injection a comparable engineered guide RNA that is not circular. The dosing regimen will comprise an effective amount to treat the disease.

Example 3—A circular engineered guide RNA will be formed by obtaining an engineered guide RNA. The engineered guide RNA will comprise a targeting domain and an RNA editing entity recruiting domain that is capable of recruiting an RNA editing entity that performs an editing of a nucleotide, while another construct will comprise a targeting domain but will not comprise a seperate RNA editing entity recruiting domain. An aptamer will be added to each end of the engineered guide RNA. A ligase will be contacted with the aptamers at each end of the engineered guide RNA to form a covalent linkage between the aptamers thereby forming the circular engineered guide RNA. The circular engineered guide RNA will substantially retain a secondary structure as compared to a secondary structure of a comparable engineered guide RNA that is not circular.

Example 4—A subject will be diagnosed with a disease. The subject will be prescribed a dosing regimen of a pharmaceutical composition. A pharmaceutical composition will comprise a circular engineered guide RNA or a recombinant polynucleotide that encodes a circular engineered guide RNA. The circular engineered guide RNA will comprise a targeting domain. The pharmaceutical composition will be co-administered to the subject with a recombinant RNA editing enzyme by injection. The dosing regimen will comprise an effective amount to treat the disease.

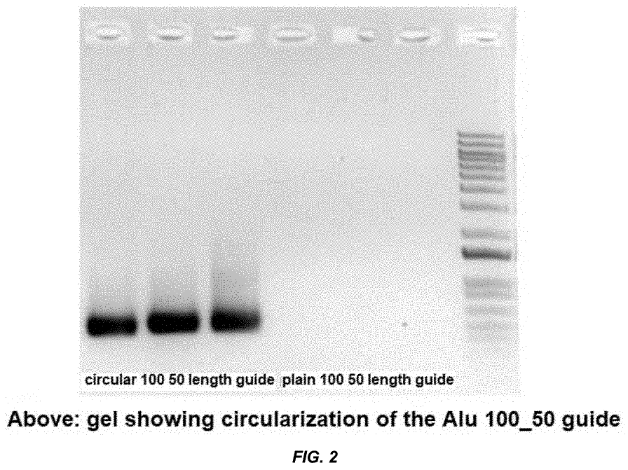

Example 5—In this example, one or more ribozyme domains were added onto a guide design to determine if circularization of the guide occurs. Guides were tested in vitro by transfecting 293FTs with lipofectamine, and either overexpressing an ADAR enzyme or using a balancing plasmid to determine the ability of the adRNA to recruit endogenous ADARs. Primers were designed to amplify only when circularization occurred. When primers were used, amplification occurs if circularization occurred. Referring to FIG. 2, a gel shows circularization of an Alu-v2-100-50 guides.

Example 6—Since circularization may require a longer guide, various longer guides will be tested against both the RAB7A and CLuc loci. These guides will be 100, 200, 300, 400, 500, and 600 bp in length, with a C mismatch designed to position across the target A. These guides will be tested against CLuc reporter plasmid, containing the luciferase gene with a premature TAG stop codon, under a pCAG promoter. When the ADAR converts the target A in TAG to an I (read as a G) the ribosome fully translated the protein as diagrammed in FIG. 4. Cells will be transfected and after 48 hours will be visualized with a luciferase reporter assay. The light readout from the cells will indicate restoration of luciferase activity. Referring to FIG. 4, a first scenario demonstrates an example in which a ribosome will reach a premature TAG stop codon in a luciferase gene, and will stop translation resulting in a truncated non-functional luciferase enzyme. In a second scenario, an ADAR will be recruited to a site by an adRNA where it will edit a TAG stop codon to a TGG codon for tryptophan that will allow for ribosomal read-through that will result in normal luciferase expression. Such a system will permit evaluation of appropriate ADAR recruitment by an adRNA. When luciferase expression will be detected, ADAR recruitment by an adRNA will have occurred. When luciferase expression will not be detected, ADAR recruitment will not have occurred.

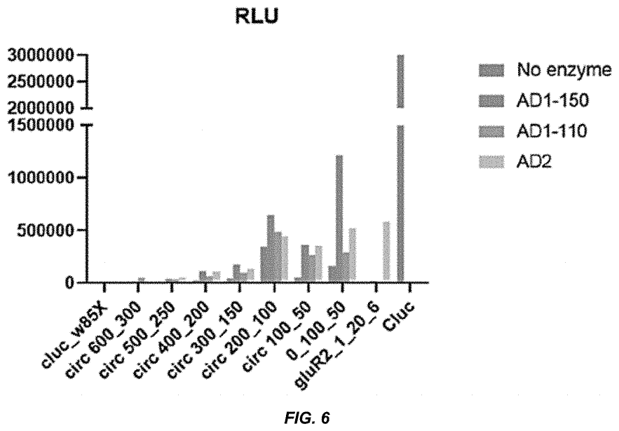

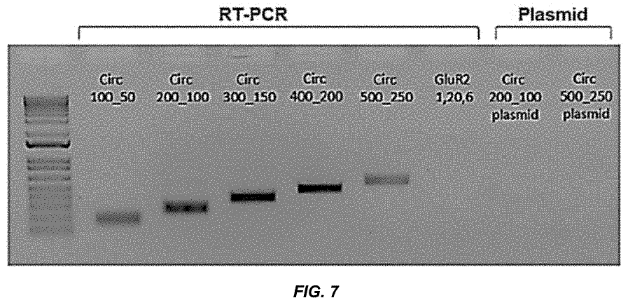

Example 7—Advantages to employing circular adRNAs for therapeutic administration: Half-life of current polIII transcripts may be a few minutes or less, but a circular adRNA may be exceptionally long-lived—may be deliverable as a chemically synthesized oligo and may not require repeat administration to a subject. A challenge to employing circular adRNAs for therapeutic administration may include: circular RNAs may be able to hybridize to a target RNA, and nucleic acids may coil around each other, so due to topological constraints a circular adRNAs may need to be pre-strained. As shown in FIG. 5, approach (1) shows a pre-strained circ adRNA. As shown in FIG. 5, approach (2) shows a large circular adRNA, which topologically may be more flexible. Data in FIG. 6 and FIG. 7 correspond to the construct of approach (2) in FIG. 5. May employ use of U6+27 promoter v. U6 alone. May employ use of Alu-adRNAs and long-adRNAs as the scaffold to engineered circular-adRNAs. For circular construct design, a U6+27 promoter may be employed. For circular construct design, an Alu-adRNA or long-adRNA may be employed as a scaffold upon which to engineer a circular-adRNA.

Example 8—Circular adRNA may affect RNA editing: circular adRNA of various lengths ranging from 100-600 (see x-axis of FIG. 6 for length of construct, i.e. 600_300 or 500_250, first number indicates length of circular adRNA, the number following the underscore indicates the position of the mismatched target nucleotide) were tested for their ability to restore luciferase activity in cells transfected with a cypridina luciferase (cluc W85X) reporter. Relative light units are indicated on the y-axis and higher values indicate restoration of luciferase activity. This experiment was carried out in the presence of the adRNA or with the addition of exogenous enzymes ADAR1-p110, ADAR1-p150 and ADAR2. Results are shown in FIG. 6.

Example 9—Confirmation that adRNA are circularized in cells is shown in FIG. 7. Briefly, RNA was extracted from cells transfected with the plasmids bearing linear antisense domains of varying lengths (i.e. 100_50, 200_100, 300_150 as shown at the top of each lane in FIG. 7) which were flanked by circularization domains. The extracted RNA was converted to cDNA followed by PCR using primers that were designed to confirm circularization; the primers would not yield an amplicon if linear RNA was the template. Cells transfected with the GluR2 adRNA or plasmids encoding the circularized variants served as negative controls. Results are shown in FIG. 7.

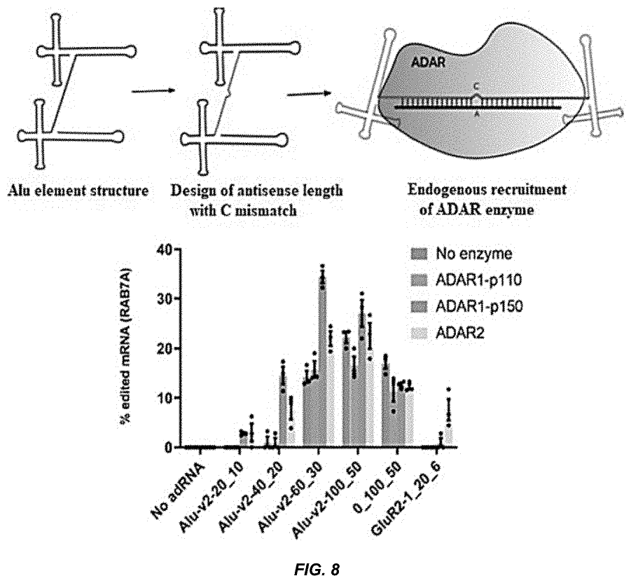

Example 10—In addition to the long adRNAs (e.g., 0-100-50, 0-200-100 etc.), Alu-adRNAs may be used as scaffolds for circular adRNA construction. Cells were transfected with plasmids encoding for various circular adRNAs. The percent RNA editing of the target RNA (RAB7A) was determined by Sanger sequencing after the target RNA was converted to cDNA by reverse transcriptase. Sanger traces were analyzed to assess the editing efficiency. In the data shown in FIG. 8, the long Alu-v2-100-50 guide showed improved editing in cells where no ADAR enzyme was overexpressed. The overexpression of ADAR1p150 resulted in significantly higher editing rates for the Alu constructs while editing rates were similar for the linear 100-50 guide. ADAR1p150 may preferentially bind Z-RNA due to its extra ds-RNA binding domain that the shorter isoform, ADAR1p110, lacks. The Alu elements with their high GC content may form Z-DNA and Z-RNA, aiding in the recruitment of ADAR1p150.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An engineered guide RNA for editing a nucleotide in an RNA sequence, the engineered guide RNA comprising:
an RNA editing entity recruiting domain,
wherein the RNA editing entity recruiting domain recruits an RNA editing entity that, when associated with the engineered guide RNA, performs a chemical transformation on a base of a nucleotide in the RNA sequence, thereby generating an edited RNA sequence, wherein the engineered guide RNA is circular.

2. The engineered guide RNA of claim 1, wherein the engineered guide RNA further comprises a targeting domain.

3. The engineered guide RNA of claim 2, wherein the targeting domain comprises a sequence length from about 20 nucleotides to about 1,000 nucleotides in length.

4. The engineered guide RNA of claim 2, wherein the targeting domain comprises a sequence length of at least about 100 nucleotides in length.

5. The engineered guide RNA of any one of claims 1-4, wherein the chemical transformation on the base results in at least a partial knockdown of the edited RNA sequence.

6. The engineered guide RNA of claim 5, wherein the partial knockdown comprises a reduced level of a protein or fragment thereof expressed from the edited RNA sequence.

7. The engineered guide RNA of claim 6, wherein the reduced level is from about 5% to 100%.

8. The engineered guide RNA of claim 7, wherein the reduced level is from about 60% to 100%.

9. The engineered guide RNA of any one of claims 5-8, wherein the partial knockdown or reduced level is determined compared to an otherwise identical unedited RNA sequence as determined in an vitro assay.

10. The engineered guide RNA of any one of claims 1-8, wherein the chemical transformation results in a sense codon read as a stop codon.

11. The engineered guide RNA of any one of claims 1-8, wherein the chemical transformation results in a stop codon read as a sense codon.

12. The engineered guide RNA of any one of claims 1-8, wherein the chemical transformation results in a first sense codon read as a second sense codon.

13. The engineered guide RNA of any one of claims 1-8, wherein the chemical transformation results in a first stop codon read as a second stop codon.

14. The engineered guide RNA of any one of claims 1-13, wherein the engineered guide RNA is configured to form a secondary structure comprising: a stem-loop, a cruciform, a toe hold, a mismatch bulge, or any combination thereof.

15. The engineered guide RNA of any one of claims 1-14, wherein the RNA editing entity recruiting domain comprises at least about 80% sequence homology to at least about 20 nucleic acids of: an Alu domain, an APOBEC recruiting domain, a GluR2 domain, or a Cas13 recruiting domain.

16. The engineered guide RNA of claim 15, wherein the RNA editing entity recruiting domain comprises at least about 80% sequence homology to at least about 20 nucleic acids of the Alu domain.

17. The engineered guide RNA of claim 16, wherein the RNA editing entity recruiting domain comprises at least about 80% sequence homology to the Alu domain.

18. The engineered guide RNA of claim 15, wherein the RNA editing entity recruiting domain comprises at least about 80% sequence homology to at least about 20 nucleic acids of the APOBEC recruiting domain.

19. The engineered guide RNA of claim 18, wherein the RNA editing entity recruiting domain comprises at least about 80% sequence homology to the APOBEC recruiting domain.

20. The engineered guide RNA of claim 15, wherein the RNA editing entity recruiting domain comprises the Cas13 recruiting domain that is a Cas13a recruiting domain, a Cas13b recruiting domain, a Cas13c recruiting domain, or a Cas13d recruiting domain.

21. The engineered guide RNA of claim 20, wherein the RNA editing entity recruiting domain comprises at least about 80% sequence homology to at least about 20 nucleic acids of the Cas13b recruiting domain.

22. The engineered guide RNA of claim 21, wherein the sequence comprises at least about 80% sequence homology to the Cas13b recruiting domain.

23. The engineered guide RNA of any one of claims 1-22, wherein the RNA editing entity is an endogenous enzyme.

24. The engineered guide RNA of any one of claims 1-22, wherein the RNA editing entity is a recombinant enzyme.

25. The engineered guide RNA of any one of claims 1-24, wherein the engineered guide RNA comprises a modification.

26. The engineered guide RNA of claim 25, wherein the modification comprises a sugar modification.

27. The engineered guide RNA of claim 25, wherein a nucleotide of the engineered guide RNA comprises a methyl group, a fluoro group, a methoxyethyl group, an ethyl group, a phosphate group, an amide group, an ester group, or any combination thereof.

28. The engineered guide RNA of any one of claims 1-27, wherein the engineered guide RNA comprises a protein coating.

29. The engineered guide RNA of any one of claims 1-28, wherein the engineered guide RNA is genetically encodable.

30. The engineered guide RNA of any one of claims 1-29, wherein the RNA editing entity is linked to the engineered guide RNA.

31. The engineered guide RNA of claim 30, wherein a linkage between the engineered guide RNA and the RNA editing entity is a direct or an indirect covalent linkage.

32. The engineered guide RNA of any one of claims 1-31, wherein the engineered guide RNA retains a half-life, in an aqueous solution at a physiological pH, that is at least about 4 times longer than a comparable guide RNA that is not circular.

33. The engineered guide RNA of any one of claims 1-32, wherein a therapeutically effective amount of the engineered guide RNA dosed to a subject in need thereof is at least about 4 times less than a comparable guide RNA that is not circular on a weight-to-weight basis.

34. An engineered guide RNA for editing a nucleotide in an RNA sequence, the engineered guide RNA comprising:
a RNA editing entity recruiting domain,
wherein the RNA editing entity recruiting domain recruits an RNA editing entity that, when associated with the engineered guide RNA, performs a chemical transformation on a base of a nucleotide in the RNA sequence, and
wherein the engineered guide RNA does not comprise a 5′ reducing hydroxyl capable of being exposed to a solvent, a 3′ reducing hydroxyl capable of being exposed to a solvent, or both.

35. An engineered guide RNA for editing a nucleotide in an RNA sequence, the engineered guide RNA comprising:
a RNA editing entity recruiting domain,
wherein the RNA editing entity recruiting domain recruits an RNA editing entity that, when associated with the engineered guide RNA, performs a chemical transformation on a base of a nucleotide in the RNA sequence, thereby generating an edited RNA sequence,
wherein the engineered guide RNA comprises a secondary structure that is less susceptible to hydrolytic degradation than an mRNA naturally present in a human cell.

36. The engineered guide RNA of claim 34 or claim 35, wherein the engineered guide RNA further comprises a targeting domain.

37. The engineered guide RNA of claim 36, wherein the targeting domain comprises a sequence length from about 20 nucleotides to about 1,000 nucleotides in length.

38. The engineered guide RNA of claim 36, wherein the targeting domain comprises a sequence length of at least about 100 nucleotides in length.

39. The engineered guide RNA of any one of claims 34-38, wherein the chemical transformation on the base results in at least a partial knockdown of the edited RNA sequence.

40. The engineered guide RNA of claim 39, wherein the at least partial knockdown comprises a reduced level of a protein or fragment thereof expressed from the edited RNA sequence.

41. The engineered guide RNA of claim 40, wherein the reduced level is from about 5% to 100%.

42. The engineered guide RNA of claim 41, wherein the reduced level is from about 60% to 100%.

43. The engineered guide RNA of any one of claims 34-42, wherein the chemical transformation results in a sense codon read as a stop codon.

44. The engineered guide RNA of any one of claims 34-42, wherein the chemical transformation results in a stop codon read as a sense codon

45. The engineered guide RNA of any one of claims 34-42, wherein the chemical transformation results in a first sense codon read as a second sense codon.

46. The engineered guide RNA of any one of claims 1-45, wherein the engineered guide RNA is a pre-strained circular RNA sequence.

47. The engineered guide RNA of any one of claims 46, wherein the engineered guide RNA comprises a reduced entropy as compared to a non-strained circular RNA sequence.

48. A vector comprising the engineered guide RNA of any one of claims 1-47.

49. The vector of claim 48, wherein the vector comprises a liposome, a viral vector, a nanoparticle, or any combination thereof.

50. The vector of claim 49, wherein the vector is the viral vector, and wherein the viral vector is an adeno-associated virus (AAV) vector.

51. The vector of any one of claims 48-50, wherein the vector comprises DNA.

52. The vector of claim 51, wherein the DNA is double stranded.

53. A nucleic acid encoding for the engineered guide RNA of any one of claims 1-47.

54. The nucleic acid of claim 53, wherein the nucleic acid is double stranded.

55. An isolated cell that comprises the engineered guide RNA of any one of claims 1-47 the vector of any one of claims 48-52, or the nucleic acid of claim 53 or claim 54.

56. A method of forming a circular RNA, the method comprising:
directly or indirectly forming a covalent linkage between more than one end of a sequence comprising an engineered guide RNA to form the circular RNA,
wherein the engineered guide RNA comprises:
a RNA editing entity recruiting domain, wherein the RNA editing entity recruiting domain recruits an RNA editing entity that, when associated with the engineered guide RNA, performs a chemical transformation on a base of a nucleotide in an RNA sequence thereby generating an edited RNA sequence.

57. The method of claim 56, wherein the method employs an enzyme to form the covalent linkage.

58. The method of claim 56 or claim 57, wherein the enzyme is a ligase.

59. The method of any one of claims 56-58, wherein the engineered guide RNA further comprises a targeting domain.

60. The method of claim 59, wherein the targeting domain comprises a sequence length from about 20 nucleotides to about 1,000 nucleotides in length.

61. The method of claim 59, wherein the targeting domain comprises a sequence length of at least about 100 nucleotides in length.

62. The method of any one of claims 56-61, wherein the chemical transformation on the base results in at least a partial knockdown of the edited RNA sequence.

63. The method of claim 62, wherein the at least partial knockdown comprises a reduced level of a protein or fragment thereof expressed from the edited RNA sequence.

64. The method of claim 63, wherein the reduced level is from about 5% to 100%.

65. The method of claim 64, wherein the reduced level is from about 60% to 100%.

66. The method of any one of claim 63-65, wherein the partial knockdown or reduced level is determined compared to an otherwise identical unedited RNA sequence as determined in an vitro assay.

67. The method of any one of claims 56-65, wherein the chemical transformation results in a sense codon read as a stop codon.

68. The method of any one of claims 56-65, wherein the chemical transformation results in a stop codon read as a sense codon.

69. The method of any one of claims 56-65, wherein the chemical transformation results in a first sense codon read as a second sense codon.

70. A pharmaceutical composition comprising the engineered guide RNA of any one of claims 1-47, the vector of any one of claims 48-52, or the nucleic acid of claim 53 or claim 54 and a pharmaceutically acceptable: excipient, diluent, or carrier.

71. The pharmaceutical composition of claim 69, in unit dose form.

72. The pharmaceutical composition of claim 70, further comprising an RNA editing entity.

73. The pharmaceutical composition of claim 71, wherein the RNA editing entity is a recombinant RNA editing entity.

74. The pharmaceutical composition of any one of claims 70-73, wherein the RNA editing entity is directly or indirectly linked to the engineered guide RNA.

75. The pharmaceutical composition of claim 74, wherein a linkage between the RNA editing entity and the engineered guide RNA is a covalent linkage.

76. A kit comprising the engineered guide RNA of any one of claims 1-47, the vector of any one of claims 48-52, or the pharmaceutical composition of any one of claims 70-75 and a container.

77. A method of making a kit, comprising at least partially packaging the engineered guide RNA of any one of claims 1-47, the vector of any one of claims 48-52, or the pharmaceutical composition of any one of claims 70-75 into a packaging.

78. A method of treating a subject in need thereof comprising: administering to the subject the engineered guide RNA of any one of claims 1-47, the vector of any one of claims 48-52, or the pharmaceutical composition of any one of claims 70-75.

79. The method of claim 78, further comprising administering a modified transfer RNA, an RNA editing entity, or a combination thereof to the subject in need thereof.

80. The method of claim 79, wherein the modified transfer RNA, the RNA editing entity, or the combination thereof is co-administered with the engineered guide RNA, the vector, or the pharmaceutical composition.

81. The method of claim 79 or claim 80, wherein the modified transfer RNA, the RNA editing entity, or the combination thereof are directly or indirectly linked to the engineered guide RNA, the vector, or the pharmaceutical composition.

82. The method of claim 81, wherein a linkage to the engineered guide RNA, the vector, or the pharmaceutical composition is a covalent linkage.

83. The method of any one of claims 78-82, wherein the administering is by intravenous injection, intramuscular injection, an intrathecal injection, an intraorbital injection, a subcutaneous injection, or any combination thereof.

84. The method of any one of claims 78-83, further comprising administering a second therapy to the subject.

85. The method of any one of claims 78-83, wherein the subject has or is suspected of having a disease or condition selected from the group consisting of: a neurodegenerative disorder, a muscular disorder, a metabolic disorder, an ocular disorder, and any combination thereof.

86. The method of claim 85, wherein the disease or condition is Alzheimer's disease, muscular dystrophy, retinitis pigmentosa, Parkinson's disease, pain, Stargardt macular dystrophy, Charcot-Marie-Tooth disease, or Rett syndrome.

87. The method of claim 86, wherein the disease or condition is the muscular dystrophy that is Duchenne muscular dystrophy (DMD).

88. The method of any one of claims 75-87, wherein the subject is a mammal.

89. The method of claim 88, wherein the mammal is a human.

90. The method of any one of claims 75-89, wherein the subject has been diagnosed with a disease or condition by a diagnostic.

91. A method of making the pharmaceutical composition of any one of claims 70-75, the method comprising formulating the pharmaceutical composition in unit dose form.

92. A method of making the engineered guide RNA of any one of claims 1-47, the method comprising genetically encoding the engineered guide RNA or chemically synthesizing the engineered guide RNA.

93. A method of making the engineered guide RNA of any one of claims 1-47, the method comprising directly or indirectly forming a covalent linkage between more than one end of the engineered guide RNA to form a circular RNA, wherein the engineered guide RNA is processed using a self-cleaving entity.

94. The method of claim 93, wherein the self-cleaving entity is a ribozyme.

95. The method of claim 94, wherein said ribozyme is a RNase P.

96. The method of claim 93, wherein the self-cleaving entity is a tRNA.

97. The method of claim 93, wherein the self-cleaving entity is an aptamer or catalytically active fragment thereof.

98. The method of claim 93, further comprising recruiting an enzyme to form the covalent bond between the more than one end of the engineered guide RNA.

99. A method of making the engineered guide RNA of any one of claims 1-47, the method comprising ligating more than one end of the engineered guide RNA using a linkage element.

100. The method of claim 99, wherein the linkage element employs click chemistry to form a circular sequence.

101. The method of claim 99, wherein the linkage element is an azide-based linkage.

102. A construct for forming a circular guide RNA sequence, the construct comprising: a nucleotide sequence encoding for:
(a) a guide RNA sequence for circularization comprising an RNA editing entity recruiting domain;
(b) a ligation sequence; and
(c) a ribozyme.

103. The construct of claim 102, wherein the engineered guide RNA further comprises a targeting domain.

104. The construct of claim 103, wherein the targeting domain comprises a sequence length from about 20 nucleotides to about 1,000 nucleotides in length.

105. The construct of claim 103, wherein the targeting domain comprises a sequence length of at least about 100 nucleotides in length.

106. The construct of claim 102, wherein the RNA editing entity recruiting domain comprises an Alu domain, an APOBEC recruiting domain, a GluR2 domain, a Cas13 recruiting domain, or any combination thereof.

107. The construct of claim 102, wherein the RNA editing entity recruiting domain comprises at least about 80% sequence homology to at least about 400 nucleotides of SEQ ID NO 1418 or SEQ ID NO 1419.

108. The construct of claim 107, wherein the RNA editing entity recruiting domain comprises at least about 80% sequence homology to SEQ ID NO 1418 or SEQ ID NO 1419.

109. The construct of any one of claims 102-108, wherein a 5′ end or a 3′ end of the guide RNA sequence is flanked by the ligation sequence.

110. The construct of claim 109, wherein the 5′ end or the 3′ end of the ligation sequence is flanked by the ribozyme.

111. The construct of any one of claims 102-110, wherein the nucleotide sequence encodes for at least 2 ribozymes, at least 2 ligation sequences, or a combination thereof.

112. The construct of any one of claims 102-111, wherein the nucleotide sequence comprises a sequence with at least about 80%, at least 85%, at least 90%, at least 92%, at least 95%, at least 97%, at least 99%, or 100% sequence homology to any one of the polynucleotides in Tables 1-12.

113. A construct for forming a circular RNA sequence, the construct comprising: a nucleotide sequence encoding for:
(a) an RNA sequence for circularization;
(b) a ligation sequence; and
(c) a tRNA.

114. The construct of claim 113, wherein a 5′ end or a 3′ end of the guide RNA sequence is flanked by the ligation sequence, and wherein the 5′ end or the 3′ end of the ligation sequence is flanked by the tRNA.

115. The construct of claim 114, wherein the nucleotide sequence encodes for at least 2 ribozymes, at least 2 ligation sequences, or a combination thereof.

116. A construct for forming a circular RNA sequence, the construct comprising: a nucleotide sequence encoding for:
(a) an RNA sequence for circularization;
(b) a ligation sequence; and
(c) an aptamer or catalytically active fragment thereof.

117. The construct of claim 116, wherein a 5′ end or a 3′ end of the guide RNA sequence is flanked by the ligation sequence, and wherein the 5′ end or the 3′ end of the ligation sequence is flanked by the aptamer or the catalytically active fragment thereof.

118. The construct of claim 117, wherein the nucleotide sequence encodes for at least 2 ribozymes, at least 2 ligation sequences, or a combination thereof.

119. An engineered polynucleotide comprising: a targeting domain that is at least partially complementary to a target RNA, wherein the engineered polynucleotide comprises a structure of Formula (I): 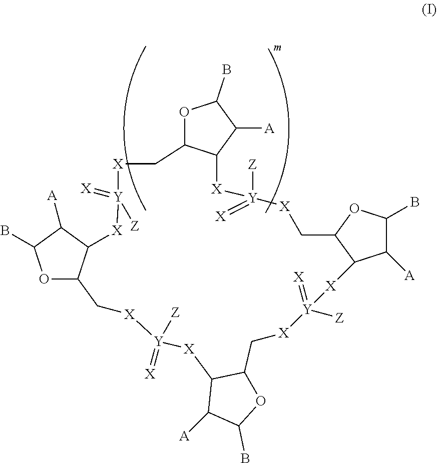
wherein:
each X is O;
each Y is P;
each Z is O, or S;
each A is independently H, D, halogen, OM, SM, NRM, or NRR′;
each B is independently uracil, thymine, adenine, cytosine, guanine, a salt of any of these, or a derivative of any of these;
each M is independently an inorganic or organic cation, H, or D; and
each R and R′ is independently H, D, halogen, or C₁-C₆ alkyl; and
m is independently an integer from 0-1,000;
wherein the targeting domain is configured to at least partially associate with a coding region of the target RNA, and wherein the association of the targeting domain with the coding region of the target RNA facilitates an edit of a base of a nucleotide of the target RNA by an RNA editing entity.

120. The engineered polynucleotide of claim 119, wherein the edit of the base of the nucleotide of the target RNA by the RNA editing entity is determined in an in vitro assay comprising:
(i) directly or indirectly introducing the target RNA into a primary cell line,
(ii) directly or indirectly introducing the engineered polynucleotide into the primary cell line, and
(iii) sequencing the target RNA

121. The engineered polynucleotide of any one of claims 119-120, wherein each unit m is independently in the (D)- or (L)- configuration.

122. The engineered polynucleotide of any one of claims 119-121, wherein Formula (I) is according to Formula (II).

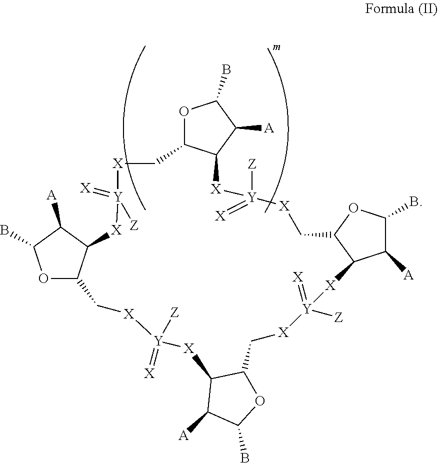

123. The engineered polynucleotide any one of claims 119-122, wherein each Z is O and each R is H.

124. The engineered polynucleotide of any one of claims 119-123, wherein m is an independent integer from about 30-600.

125. The engineered polynucleotide of any one of claims 119-124, wherein at least partially complementary comprises the targeting domain that comprises a polynucleotide sequence with at least about 80% sequence homology to a reverse complement to the target RNA.

126. The engineered polynucleotide of any one of claims 119-125, wherein the RNA editing entity comprises an ADAR protein, an APOBEC protein, or both.

127. The engineered polynucleotide of any one of claims 119-126, wherein the RNA editing entity comprises ADAR and wherein ADAR comprises ADAR1 or ADAR2.

128. The engineered polynucleotide of any one of claims 119-127, wherein the edit of a base converts a sense codon into a stop codon.

129. The engineered polynucleotide of any one of claims 119-128, wherein the edit of a base converts a stop codon into a sense codon.

130. The engineered polynucleotide of any one of claims 119-129, wherein the edit of a base converts a first sense codon into a second sense codon.

131. The engineered polynucleotide of any one of claims 119-130, wherein the edit of a base, coverts a sense codon specifying a first amino acid into a second sense codon specifying a second amino acid.

132. The engineered polynucleotide of claim 131, wherein the first amino acid is a protease cleavage site.

133. An engineered polynucleotide comprising a targeting domain that is at least partially complementary to a target RNA; wherein the engineered polynucleotide comprises a structure of Formula (III): 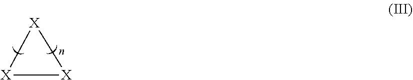
wherein in the engineered polynucleotide, each X is a nucleotide comprising a base that is independently uracil, thymine, adenine, cytosine, guanine, a salt of any of these, or a derivative of any of these;
n is independently an integer from 0-1,000; and
wherein each nucleotide is connected to two adjacent nucleotides by, independently for each connection, a phosphoester, phosphothioester, phosphothioate, or phosphoramidite linkage; and
wherein the targeting domain is configured to at least partially associate with a coding region of the target RNA, wherein the association of the targeting domain with the coding region of the target RNA facilitates an edit of a base of a nucleotide of the target RNA by an RNA editing entity.

134. The engineered polynucleotide of claim 133, wherein the edit of the base of the nucleotide of the target RNA by the RNA editing entity is determined in an in vitro assay comprising:
(i) directly or indirectly introducing the target RNA into a primary cell line,
(ii) directly or indirectly introducing the engineered polynucleotide into the primary cell line, and
(iii) sequencing the target RNA.

135. The engineered polynucleotide of claim 133 or 134, wherein the RNA editing entity comprises an ADAR protein, an APOBEC protein, or both.

136. The engineered polynucleotide any one of claims 134-135, wherein the RNA editing entity comprises ADAR and wherein ADAR comprises ADAR1 or ADAR2.

137. The engineered polynucleotide of any one of claims 119-136, wherein the primary cell line comprises a neuron cell, a photoreceptor cell, a retinal pigment epithelium cell, a glia cell, a myoblast cell, a myotube cell, a hepatocyte, a lung epithelial cell, or a fibroblast cell.

138. The engineered polynucleotide of any one of claims 119-137, wherein the engineered polynucleotide does not comprise a 5′ reducing hydroxyl, a 3′ reducing hydroxyl, or both, capable of being exposed to a solvent.

139. The engineered polynucleotide of claim 138, wherein each 5′ hydroxyl, and each 3′ hydroxyl is independently bonded to a phosphorous by a covalent oxygen phosphorous bond.

140. The engineered polynucleotide of claim 139, wherein the phosphorous is contained in a phosphodiester group.

141. The engineered polynucleotide of any one of claims 119-140, wherein the engineered polynucleotide further comprises an RNA editing entity recruiting domain.

142. The engineered polynucleotide of any one of claims 119-141, wherein the targeting domain is about 20 nucleotides to about 150 nucleotides.

143. The engineered polynucleotide of any one of claims 119-142, wherein the target RNA comprises a nonsense mutation.

144. The engineered polynucleotide of any one of claims 119-143, wherein the targeting domain comprises at least a single nucleotide that is mismatched to the target RNA.

145. The engineered polynucleotide of claim 144, wherein the mismatched nucleotide is adjacent to two nucleotides, one on each side of the mismatched nucleotide, that are complementary to the target RNA.

146. The engineered polynucleotide of any one of claims 119-145, wherein the targeting domain at least partially binds to a target RNA that is implemented in a disease or condition.

147. The engineered polynucleotide of claim 146, comprising the disease or condition which comprises Rett syndrome, Huntington's disease, Parkinson's Disease, Alzheimer's disease, a muscular dystrophy, or Tay-Sachs Disease.

148. The engineered polynucleotide of any one of claims 119-147, wherein the edit of a base results in an increased level of a protein or fragment thereof, an increased length of a protein or fragment thereof, an increased functionality of a protein or fragment thereof, increased stability of a protein or fragment thereof, or any combination thereof after translation of the target RNA with the edit of the base, relative to a translated protein of an otherwise comparable target RNA lacking the edit.

149. The engineered polynucleotide of claim 148, wherein the increased level is from about 5% to about 100%.

150. The engineered polynucleotide of claim 148, wherein the increased length is from about 5% to about 100% of the protein or fragment thereof.

151. The engineered polynucleotide of claim 148, wherein the increased stability is an increased half-life of the protein or fragment thereof.

152. The engineered polynucleotide of any one of claims 119-151, wherein the engineered polynucleotide comprises a polynucleotide sequence with at least about 80%, at least 85%, at least 90%, at least 92%, at least 95%, at least 97%, at least 99%, or 100% sequence homology to any one of the polynucleotides in Tables 1-12.

153. The engineered polynucleotide of any one of claims 119-152, wherein the engineered polynucleotide comprises a polynucleotide sequence having a length that is at least about 80%, at least 85%, at least 90%, at least 92%, at least 95%, at least 97%, at least 99%, or 100% of the sequence length of any one of the polynucleotides in Tables 1-12.

154. The engineered polynucleotide of any one of claims 119-153, wherein the engineered polynucleotide comprises a polynucleotide sequence with at least about 80%, at least 85%, at least 90%, at least 92%, at least 95%, at least 97%, at least 99%, or 100% sequence homology and at least about 80%, at least 85%, at least 90%, at least 92%, at least 95%, at least 97%, at least 99%, or 100% sequence length to any one of the polynucleotides in Tables 1-12.

155. An engineered guide RNA comprising a targeting domain that is at least partially complementary to a target RNA, wherein the engineered guide RNA comprises a backbone comprising a plurality of sugar and phosphate moieties covalently linked together, and wherein the backbone does not comprise a 5′ reducing hydroxyl, a 3′ reducing hydroxyl, or both, capable of being exposed to a solvent, wherein the targeting domain is configured to at least partially associate with a coding region of the target RNA, wherein the association of the targeting domain with the coding region of the target RNA facilitates an edit of a base of a nucleotide of the target RNA by an RNA editing entity.

156. An engineered guide RNA comprising: a targeting domain that is at least partially complementary to a target RNA; and an RNA editing entity recruiting domain, wherein the RNA editing entity recruiting domain is configured to at least transiently associate with an RNA editing entity; wherein the engineered guide RNA comprises a backbone comprising a plurality of sugar and phosphate moieties covalently linked together, and wherein the backbone does not comprise a 5′ reducing hydroxyl, a 3′ reducing hydroxyl, or both, capable of being exposed to a solvent, wherein the targeting domain is configured to at least partially associate with a coding region of the target RNA, wherein the association of the targeting domain with the coding region of the target RNA facilitates an edit of a base of a nucleotide of the target RNA by an RNA editing entity.

157. The engineered polynucleotide of claim 155 or 156, wherein at least partially complementary comprises the targeting domain that comprises a polynucleotide sequence with at least about 80%, at least about 85%, at least about 90%, at least about 92%, or at least about 95% sequence homology to a reverse complement to the target RNA.

158. The engineered polynucleotide of any one of claims 155-157, wherein the edit of the base of a nucleotide of the target RNA by an RNA editing entity is determined in an in vitro assay comprising:
(i) transfecting the target RNA into a primary cell line,
(ii) transfecting the engineered polynucleotide into a primary cell line, and
(iii) sequencing the target RNA.


SEQUENCE LISTING (1419 sequences)

SEQ ID NO: 1 - DNA, 20 bp, Artificial Sequence
  Description of Artificial Sequence Synthetic oligonucleotide
gggcacaagggcacagactt

SEQ ID NO: 2 - DNA, 20 bp, Artificial Sequence
  Description of Artificial Sequence Synthetic oligonucleotide
ggcacaagggcacagacttc

SEQ ID NO: 3 - DNA, 20 bp, Artificial Sequence
  Description of Artificial Sequence Synthetic oligonucleotide
gcacaagggcacagacttcc

SEQ ID NO: 4 - DNA, 20 bp, Artificial Sequence
  Description of Artificial Sequence Synthetic oligonucleotide
cacaagggcacagacttcca

SEQ ID NO: 5 - DNA, 20 bp, Artificial Sequence
  Description of Artificial Sequence Synthetic oligonucleotide
acaagggcacagacttccaa

SEQ ID NO: 6 - DNA, 20 bp, Artificial Sequence
  Description of Artificial Sequence Synthetic oligonucleotide
caagggcacagacttccaaa

SEQ ID NO: 7 - DNA, 20 bp, Artificial Sequence
  Description of Artificial Sequence Synthetic oligonucleotide
gggcacaagggcacagactt

SEQ ID NO: 8 - DNA, 20 bp, Artificial Sequence
  Description of Artificial Sequence Synthetic oligonucleotide
ggcacaagggcacagacttc

SEQ ID NO: 9 - DNA, 20 bp, Artificial Sequence
  Description of Artificial Sequence Synthetic oligonucleotide
  Description of Combined DNA/RNA Molecule Synthetic oligonucleotide
gcauaagggcacagacttcc

SEQ ID NO: 10 - DNA, 20 bp, Artificial Sequence
  Description of Artificial Sequence Synthetic oligonucleotide
cacaagggcacagacttcca

SEQ ID NO: 11 - DNA, 20 bp, Artificial Sequence
  Description of Artificial Sequence Synthetic oligonucleotide
acaagggcacagacttccaa

SEQ ID NO: 12 - DNA, 20 bp, Artificial Sequence
  Description of Artificial Sequence Synthetic oligonucleotide
caagggcacagacttccaaa

SEQ ID NO: 13 - RNA, 20 nt, Artificial Sequence
  Description of Artificial Sequence Synthetic oligonucleotide
gggcacaagggcacagacuu

SEQ ID NO: 14 - RNA, 20 nt, Artificial Sequence
  Description of Artificial Sequence Synthetic oligonucleotide
ggcacaagggcacagacuuc

SEQ ID NO: 15 - RNA, 20 nt, Artificial Sequence
  Description of Artificial Sequence Synthetic oligonucleotide
gcacaagggcacagacuucc

SEQ ID NO: 16 - RNA, 20 nt, Artificial Sequence
  Description of Artificial Sequence Synthetic oligonucleotide
cauaagggcacagacuucca

SEQ ID NO: 17 - DNA, 20 bp, Artificial Sequence
  Description of Artificial Sequence Synthetic oligonucleotide
  Description of Combined DNA/RNA Molecule Synthetic oligonucleotide
acaagggcacagactuccaa

SEQ ID NO: 18 - DNA, 20 bp, Artificial Sequence
  Description of Artificial Sequence Synthetic oligonucleotide
caagggcacagacttccaaa

SEQ ID NO: 19 - RNA, 20 nt, Artificial Sequence
  Description of Artificial Sequence Synthetic oligonucleotide
gcacaagggcacagacuucc

SEQ ID NO: 20 - RNA, 20 nt, Artificial Sequence
  Description of Artificial Sequence Synthetic oligonucleotide
cacaagggcacagacuucca

SEQ ID NO: 21 - RNA, 20 nt, Artificial Sequence
  Description of Artificial Sequence Synthetic oligonucleotide
acaagggcacagacuuccaa

SEQ ID NO: 22 - RNA, 20 nt, Artificial Sequence
  Description of Artificial Sequence Synthetic oligonucleotide
caagggcacagacuuccaaa

SEQ ID NO: 23 - RNA, 20 nt, Artificial Sequence
  Description of Artificial Sequence Synthetic oligonucleotide
gcacaagggcacagacuucc

SEQ ID NO: 24 - RNA, 20 nt, Artificial Sequence
  Description of Artificial Sequence Synthetic oligonucleotide
cacaagggcacagacuucca

SEQ ID NO: 25 - RNA, 20 nt, Artificial Sequence
  Description of Artificial Sequence Synthetic oligonucleotide
acaagggcacagacuuccaa

SEQ ID NO: 26 - RNA, 20 nt, Artificial Sequence
  Description of Artificial Sequence Synthetic oligonucleotide
caagggcacagacuuccaaa

SEQ ID NO: 27 - RNA, 20 nt, Artificial Sequence
  Description of Artificial Sequence Synthetic oligonucleotide
gggcacaagggcacagacuu

SEQ ID NO: 28 - RNA, 20 nt, Artificial Sequence
  Description of Artificial Sequence Synthetic oligonucleotide
ggcacaagggcacagacuuc

SEQ ID NO: 29 - RNA, 20 nt, Artificial Sequence
  Description of Artificial Sequence Synthetic oligonucleotide
gcacaagggcacagacuucc

SEQ ID NO: 30 - RNA, 20 nt, Artificial Sequence
  Description of Artificial Sequence Synthetic oligonucleotide
cacaagggcacagacuucca

SEQ ID NO: 31 - DNA, 20 bp, Artificial Sequence
  Description of Artificial Sequence Synthetic oligonucleotide
  Description of Combined DNA/RNA Molecule Synthetic oligonucleotide
acaagggcacagactuccaa

SEQ ID NO: 32 - DNA, 20 bp, Artificial Sequence
  Description of Artificial Sequence Synthetic oligonucleotide
caagggcacagacttccaaa

SEQ ID NO: 33 - DNA, 20 bp, Artificial Sequence
  Description of Artificial Sequence Synthetic oligonucleotide
gggcacaagggcacagactt

SEQ ID NO: 34 - DNA, 20 bp, Artificial Sequence
  Description of Artificial Sequence Synthetic oligonucleotide
ggcacaagggcacagacttc

SEQ ID NO: 35 - DNA, 20 bp, Artificial Sequence
  Description of Artificial Sequence Synthetic oligonucleotide
gcacaagggcacagacttcc

SEQ ID NO: 36 - DNA, 20 bp, Artificial Sequence
  Description of Artificial Sequence Synthetic oligonucleotide
cacaagggcacagacttcca

SEQ ID NO: 37 - DNA, 20 bp, Artificial Sequence
  Description of Artificial Sequence Synthetic oligonucleotide
acaagggcacagacttccaa

SEQ ID NO: 38 - DNA, 20 bp, Artificial Sequence
  Description of Artificial Sequence Synthetic oligonucleotide
caagggcacagacttccaaa

SEQ ID NO: 39 - RNA, 20 nt, Artificial Sequence
  Description of Artificial Sequence Synthetic oligonucleotide
ggcacaagggcacagacuuc

SEQ ID NO: 40 - RNA, 20 nt, Artificial Sequence
  Description of Artificial Sequence Synthetic oligonucleotide
ggcacaagggcacagacuuc

SEQ ID NO: 41 - RNA, 20 nt, Artificial Sequence
  Description of Artificial Sequence Synthetic oligonucleotide
ggcacaagggcacagacuuc

SEQ ID NO: 42 - DNA, 20 bp, Artificial Sequence
  Description of Artificial Sequence Synthetic oligonucleotide
ggcacaagggcacagacttc

SEQ ID NO: 43 - DNA, 20 bp, Artificial Sequence
  Description of Artificial Sequence Synthetic oligonucleotide
  Description of Combined DNA/RNA Molecule Synthetic oligonucleotide
ggcauaagggcacagacttc

SEQ ID NO: 44 - DNA, 20 bp, Artificial Sequence
  Description of Artificial Sequence Synthetic oligonucleotide
ggcacaagggcacagacttc

SEQ ID NO: 45 - RNA, 20 nt, Artificial Sequence
  Description of Artificial Sequence Synthetic oligonucleotide
ggcacaagggcacagacuuc

SEQ ID NO: 46 - RNA, 20 nt, Artificial Sequence
  Description of Artificial Sequence Synthetic oligonucleotide
ggcacaagggcacagacuuc

SEQ ID NO: 47 - RNA, 20 nt, Artificial Sequence
  Description of Artificial Sequence Synthetic oligonucleotide
gcagggcacaagggcacaga

SEQ ID NO: 48 - RNA, 20 nt, Artificial Sequence
  Description of Artificial Sequence Synthetic oligonucleotide
cagggcacaagggcacagac

SEQ ID NO: 49 - DNA, 20 bp, Artificial Sequence
  Description of Artificial Sequence Synthetic oligonucleotide
agggcacaagggcacagact

SEQ ID NO: 50 - DNA, 20 bp, Artificial Sequence
  Description of Artificial Sequence Synthetic oligonucleotide
aagggcacagacttccaaag

SEQ ID NO: 51 - DNA, 20 bp, Artificial Sequence
  Description of Artificial Sequence Synthetic oligonucleotide
agggcacagacttccaaagg

SEQ ID NO: 52 - DNA, 20 bp, Artificial Sequence
  Description of Artificial Sequence Synthetic oligonucleotide
gggcacagacttccaaaggc

SEQ ID NO: 53 - RNA, 20 nt, Artificial Sequence
  Description of Artificial Sequence Synthetic oligonucleotide
ggcacaagggcacagacuuc

SEQ ID NO: 54 - RNA, 20 nt, Artificial Sequence
  Description of Artificial Sequence Synthetic oligonucleotide
ggcacaagggcacagacuuc

SEQ ID NO: 55 - DNA, 20 bp, Artificial Sequence
  Description of Artificial Sequence Synthetic oligonucleotide
  modified_base (13)..(13): 2-amino purine
gggcacaagggcncagactt

SEQ ID NO: 56 - DNA, 20 bp, Artificial Sequence
  Description of Artificial Sequence Synthetic oligonucleotide
  modified_base (12)..(12): 2-amino purine
ggcacaagggcncagacttc

SEQ ID NO: 57 - DNA, 20 bp, Artificial Sequence
  Description of Artificial Sequence Synthetic oligonucleotide
  modified_base (11)..(11): 2-amino purine
gcacaagggcncagacttcc

SEQ ID NO: 58 - DNA, 20 bp, Artificial Sequence
  Description of Artificial Sequence Synthetic oligonucleotide
  modified_base (13)..(13): 2,6-diamino purine
gggcacaagggcncagactt

SEQ ID NO: 59 - DNA, 20 bp, Artificial Sequence
  Description of Artificial Sequence Synthetic oligonucleotide
  modified_base (12)..(12): 2,6-diamino purine
ggcacaagggcncagacttc

SEQ ID NO: 60 - DNA, 20 bp, Artificial Sequence
  Description of Artificial Sequence Synthetic oligonucleotide
  modified_base (11)..(11): 2,6-diamino purine
gcacaagggcncagacttcc

SEQ ID NO: 61 - RNA, 20 nt, Artificial Sequence
  Description of Artificial Sequence Synthetic oligonucleotide
ggcacaagggcacagacuuc

SEQ ID NO: 62 - RNA, 20 nt, Artificial Sequence
  Description of Artificial Sequence Synthetic oligonucleotide
ggcacaagggcacagacuuc

SEQ ID NO: 63 - RNA, 20 nt, Artificial Sequence
  Description of Artificial Sequence Synthetic oligonucleotide
ggcacaagggcacagacuuc

SEQ ID NO: 64 - DNA, 20 bp, Artificial Sequence
  Description of Artificial Sequence Synthetic oligonucleotide
ggcacaagggcacagacttc

SEQ ID NO: 65 - DNA, 20 bp, Artificial Sequence
  Description of Artificial Sequence Synthetic oligonucleotide
gagcagctgcaacctggcaa

SEQ ID NO: 66 - DNA, 20 bp, Artificial Sequence
  Description of Artificial Sequence Synthetic oligonucleotide
agcagctgcaacctggcaac

SEQ ID NO: 67 - DNA, 20 bp, Artificial Sequence
  Description of Artificial Sequence Synthetic oligonucleotide
gcagctgcaacctggcaaca

SEQ ID NO: 68 - DNA, 20 bp, Artificial Sequence
  Description of Artificial Sequence Synthetic oligonucleotide
cagctgcaacctggcaacaa

SEQ ID NO: 69 - DNA, 20 bp, Artificial Sequence
  Description of Artificial Sequence Synthetic oligonucleotide
agctgcaacctggcaacaac

SEQ ID NO: 70 - DNA, 20 bp, Artificial Sequence
  Description of Artificial Sequence Synthetic oligonucleotide
gctgcaacctggcaacaacc

SEQ ID NO: 71 - DNA, 20 bp, Artificial Sequence
  Description of Artificial Sequence Synthetic oligonucleotide
gagcagctgcaacctggcaa

SEQ ID NO: 72 - DNA, 20 bp, Artificial Sequence
  Description of Artificial Sequence Synthetic oligonucleotide
agcagctgcaacctggcaac

SEQ ID NO: 73 - DNA, 20 bp, Artificial Sequence
  Description of Artificial Sequence Synthetic oligonucleotide
gcagctgcaacctggcaaca

SEQ ID NO: 74 - DNA, 20 bp, Artificial Sequence
  Description of Artificial Sequence Synthetic oligonucleotide
  Description of Combined DNA/RNA Molecule Synthetic oligonucleotide
cagcugcaacctggcaacaa

SEQ ID NO: 75 - DNA, 20 bp, Artificial Sequence
  Description of Artificial Sequence Synthetic oligonucleotide
  Description of Combined DNA/RNA Molecule Synthetic oligonucleotide
agcugcaacctggcaacaac

SEQ ID NO: 76 - DNA, 20 bp, Artificial Sequence
  Description of Artificial Sequence Synthetic oligonucleotide
  Description of Combined DNA/RNA Molecule Synthetic oligonucleotide
gcugcaacctggcaacaacc

SEQ ID NO: 77 - DNA, 20 bp, Artificial Sequence
  Description of Artificial Sequence Synthetic oligonucleotide
gagcagctgcaacctggcaa

SEQ ID NO: 78 - DNA, 20 bp, Artificial Sequence
  Description of Artificial Sequence Synthetic oligonucleotide
agcagctgcaacctggcaac

SEQ ID NO: 79 - DNA, 20 bp, Artificial Sequence
  Description of Artificial Sequence Synthetic oligonucleotide
gcagctgcaacctggcaaca

SEQ ID NO: 80 - DNA, 20 bp, Artificial Sequence
  Description of Artificial Sequence Synthetic oligonucleotide
  Description of Combined DNA/RNA Molecule Synthetic oligonucleotide
cagcugcaacctggcaacaa

SEQ ID NO: 81 - DNA, 20 bp, Artificial Sequence
  Description of Artificial Sequence Synthetic oligonucleotide
  Description of Combined DNA/RNA Molecule Synthetic oligonucleotide
agcugcaacctggcaacaac

SEQ ID NO: 82 - DNA, 20 bp, Artificial Sequence
  Description of Artificial Sequence Synthetic oligonucleotide
  Description of Combined DNA/RNA Molecule Synthetic oligonucleotide
gcuggaacctggcaacaacc

SEQ ID NO: 83 - DNA, 20 bp, Artificial Sequence
  Description of Artificial Sequence Synthetic oligonucleotide
  Description of Combined DNA/RNA Molecule Synthetic oligonucleotide
gagcagctgcaaccuggcaa

SEQ ID NO: 84 - DNA, 20 bp, Artificial Sequence
  Description of Artificial Sequence Synthetic oligonucleotide
  Description of Combined DNA/RNA Molecule Synthetic oligonucleotide
gagcagctgcaaccuggcaa

SEQ ID NO: 85 - DNA, 20 bp, Artificial Sequence
  Description of Artificial Sequence Synthetic oligonucleotide
agcagctgcaacctggcaac

SEQ ID NO: 86 - DNA, 20 bp, Artificial Sequence
  Description of Artificial Sequence Synthetic oligonucleotide
agcagctgcaacctggcaac

SEQ ID NO: 87 - DNA, 20 bp, Artificial Sequence
  Description of Artificial Sequence Synthetic oligonucleotide
ggagctgcaaccgggcaaca

SEQ ID NO: 88 - DNA, 20 bp, Artificial Sequence
  Description of Artificial Sequence Synthetic oligonucleotide
  Description of Combined DNA/RNA Molecule Synthetic oligonucleotide
gcagctgcaaccuggcaaca

SEQ ID NO: 89 - DNA, 20 bp, Artificial Sequence
  Description of Artificial Sequence Synthetic oligonucleotide
gagcagctgcaacctggcaa

SEQ ID NO: 90 - DNA, 20 bp, Artificial Sequence
  Description of Artificial Sequence Synthetic oligonucleotide
agcagctgcaacctggcaac

SEQ ID NO: 91 - DNA, 20 bp, Artificial Sequence
  Description of Artificial Sequence Synthetic oligonucleotide
gcagctgcaacctggcaaca

SEQ ID NO: 92 - DNA, 20 bp, Artificial Sequence
  Description of Artificial Sequence Synthetic oligonucleotide
  Description of Combined DNA/RNA Molecule Synthetic oligonucleotide
cagcugcaacctggcaacaa

SEQ ID NO: 93 - DNA, 20 bp, Artificial Sequence
  Description of Artificial Sequence Synthetic oligonucleotide
  Description of Combined DNA/RNA Molecule Synthetic oligonucleotide
agcugcaacctggcaacaac

SEQ ID NO: 94 - DNA, 20 bp, Artificial Sequence
  Description of Artificial Sequence Synthetic oligonucleotide
  Description of Combined DNA/RNA Molecule Synthetic oligonucleotide
gcugcaacctggcaacaacc

SEQ ID NO: 95 - DNA, 20 bp, Artificial Sequence
  Description of Artificial Sequence Synthetic oligonucleotide
gagcagctgcaacctggcaa

SEQ ID NO: 96 - DNA, 19 bp, Artificial Sequence
  Description of Artificial Sequence Synthetic oligonucleotide
agcagcgcaacctggcaac

SEQ ID NO: 97 - DNA, 20 bp, Artificial Sequence
  Description of Artificial Sequence Synthetic oligonucleotide
gcagctgcaacctggcaaca

SEQ ID NO: 98 - DNA, 20 bp, Artificial Sequence
  Description of Artificial Sequence Synthetic oligonucleotide
cagctgcaacctggcaacaa

SEQ ID NO: 99 - DNA, 20 bp, Artificial Sequence
  Description of Artificial Sequence Synthetic oligonucleotide
agctgcaacctggcaacaac

SEQ ID NO: 100 - DNA, 20 bp, Artificial Sequence
  Description of Artificial Sequence Synthetic oligonucleotide
gctgcaacctggcaacaacc

SEQ ID NO: 101 - DNA, 20 bp, Artificial Sequence
  Description of Artificial Sequence Synthetic oligonucleotide
gggccaacagccagcctgca

SEQ ID NO: 102 - DNA, 20 bp, Artificial Sequence
  Description of Artificial Sequence Synthetic oligonucleotide
ggccaacagccagcctgcag

SEQ ID NO: 103 - DNA, 20 bp, Artificial Sequence
  Description of Artificial Sequence Synthetic oligonucleotide
gccaacagccagcctgtagg

SEQ ID NO: 104 - DNA, 20 bp, Artificial Sequence
  Description of Artificial Sequence Synthetic oligonucleotide
ccaacagccagcctgcagga

SEQ ID NO: 105 - DNA, 20 bp, Artificial Sequence
  Description of Artificial Sequence Synthetic oligonucleotide
caacagccagcctgcaggag

SEQ ID NO: 106 - DNA, 20 bp, Artificial Sequence
  Description of Artificial Sequence Synthetic oligonucleotide
aacagctagcctgcaggagg

SEQ ID NO: 107 - DNA, 20 bp, Artificial Sequence
  Description of Artificial Sequence Synthetic oligonucleotide
gggccaacagccagcctgca

SEQ ID NO: 108 - DNA, 20 bp, Artificial Sequence
  Description of Artificial Sequence Synthetic oligonucleotide
ggccaacagccagcctgcag

SEQ ID NO: 109 - DNA, 20 bp, Artificial Sequence
  Description of Artificial Sequence Synthetic oligonucleotide
gccaacagccagcctgcagg

SEQ ID NO: 110 - DNA, 20 bp, Artificial Sequence
  Description of Artificial Sequence Synthetic oligonucleotide
ccaacagccagcctgcagga

SEQ ID NO: 111 - DNA, 20 bp, Artificial Sequence
  Description of Artificial Sequence Synthetic oligonucleotide
caacagccagcctgcaggag

SEQ ID NO: 112 - DNA, 20 bp, Artificial Sequence
  Description of Artificial Sequence Synthetic oligonucleotide
aacagtcagcctgcaggagg

SEQ ID NO: 113 - RNA, 20 nt, Artificial Sequence
  Description of Artificial Sequence Synthetic oligonucleotide
gggccaacagccagccugca

SEQ ID NO: 114 - RNA, 20 nt, Artificial Sequence
  Description of Artificial Sequence Synthetic oligonucleotide
ggccaacagccagccugcag

SEQ ID NO: 115 - DNA, 20 bp, Artificial Sequence
  Description of Artificial Sequence Synthetic oligonucleotide
  Description of Combined DNA/RNA Molecule Synthetic oligonucleotide
gccaacagccagcctguagg

SEQ ID NO: 116 - DNA, 20 bp, Artificial Sequence
  Description of Artificial Sequence Synthetic oligonucleotide
ccaacagccagcctgcagga

SEQ ID NO: 117 - DNA, 20 bp, Artificial Sequence
  Description of Artificial Sequence Synthetic oligonucleotide
  Description of Combined DNA/RNA Molecule Synthetic oligonucleotide
caauagccagcctgcaggag

SEQ ID NO: 118 - DNA, 20 bp, Artificial Sequence
  Description of Artificial Sequence Synthetic oligonucleotide
aacagccagcctgcaggagg

SEQ ID NO: 119 - RNA, 20 nt, Artificial Sequence
  Description of Artificial Sequence Synthetic oligonucleotide
gggccaacagccagccugca

SEQ ID NO: 120 - RNA, 20 nt, Artificial Sequence
  Description of Artificial Sequence Synthetic oligonucleotide
gggccaacagccagccugca

SEQ ID NO: 121 - RNA, 20 nt, Artificial Sequence
  Description of Artificial Sequence Synthetic oligonucleotide
ggccaacagccagccugcag

SEQ ID NO: 122 - RNA, 20 nt, Artificial Sequence
  Description of Artificial Sequence Synthetic oligonucleotide
ggccaacagccagccugcag

SEQ ID NO: 123 - RNA, 20 nt, Artificial Sequence
  Description of Artificial Sequence Synthetic oligonucleotide
gccaacagccagccugcagg

SEQ ID NO: 124 - RNA, 20 nt, Artificial Sequence
  Description of Artificial Sequence Synthetic oligonucleotide
gccaacagccagccugcagg

SEQ ID NO: 125 - RNA, 20 nt, Artificial Sequence
  Description of Artificial Sequence Synthetic oligonucleotide
gggccaacagccagccugca

SEQ ID NO: 126 - RNA, 20 nt, Artificial Sequence
  Description of Artificial Sequence Synthetic oligonucleotide
ggccaacagccagccugcag

SEQ ID NO: 127 - DNA, 20 bp, Artificial Sequence
  Description of Artificial Sequence Synthetic oligonucleotide
gccaacagccagcctgcagg

SEQ ID NO: 128 - DNA, 20 bp, Artificial Sequence
  Description of Artificial Sequence Synthetic oligonucleotide
ccaacagccagcctgcagga

SEQ ID NO: 129 - DNA, 20 bp, Artificial Sequence
  Description of Artificial Sequence Synthetic oligonucleotide
caacagccagcctgcaggag

SEQ ID NO: 130 - DNA, 20 bp, Artificial Sequence
  Description of Artificial Sequence Synthetic oligonucleotide
aacagccagcctgcaggagg

SEQ ID NO: 131 - DNA, 20 bp, Artificial Sequence
  Description of Artificial Sequence Synthetic oligonucleotide
gggccaacagccagcctgca

SEQ ID NO: 132 - DNA, 20 bp, Artificial Sequence
  Description of Artificial Sequence Synthetic oligonucleotide
ggccaacagccagcctgcag

SEQ ID NO: 133 - DNA, 20 bp, Artificial Sequence
  Description of Artificial Sequence Synthetic oligonucleotide
gccaacagccagcctgcagg

SEQ ID NO: 134 - DNA, 20 bp, Artificial Sequence
  Description of Artificial Sequence Synthetic oligonucleotide
ccaacagccagcctgcagga

SEQ ID NO: 135 - DNA, 20 bp, Artificial Sequence
  Description of Artificial Sequence Synthetic oligonucleotide
caacagccagcctgcaggag

SEQ ID NO: 136 - DNA, 20 bp, Artificial Sequence
  Description of Artificial Sequence Synthetic oligonucleotide
aacagccagcctgcaggagg

SEQ ID NO: 137 - DNA, 20 bp, Artificial Sequence
  Description of Artificial Sequence Synthetic oligonucleotide
attaataaattgtcatcacc

SEQ ID NO: 138 - DNA, 20 bp, Artificial Sequence
  Description of Artificial Sequence Synthetic oligonucleotide
attaataaattgtcatcacc

SEQ ID NO: 139 - DNA, 20 bp, Artificial Sequence
  Description of Artificial Sequence Synthetic oligonucleotide
attaataaattgtcatcacc

SEQ ID NO: 140 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 141 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 142 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 143 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 144 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 145 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 146 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 147 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 148 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 149 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 150 - DNA, 20 bp, Artificial Sequence
  Description of Artificial Sequence Synthetic oligonucleotide
  Description of Combined DNA/RNA Molecule Synthetic oligonucleotide
tgtcatcaccagaaaaaguc

SEQ ID NO: 151 - DNA, 20 bp, Artificial Sequence
  Description of Artificial Sequence Synthetic oligonucleotide
  Description of Combined DNA/RNA Molecule Synthetic oligonucleotide
utgtcatcaccagaaaaagu

SEQ ID NO: 152 - DNA, 20 bp, Artificial Sequence
  Description of Artificial Sequence Synthetic oligonucleotide
  Description of Combined DNA/RNA Molecule Synthetic oligonucleotide
ttgtcatcaccagaaaaagu

SEQ ID NO: 153 - DNA, 20 bp, Artificial Sequence
  Description of Artificial Sequence Synthetic oligonucleotide
  Description of Combined DNA/RNA Molecule Synthetic oligonucleotide
autgtcatcaccagaaaaag

SEQ ID NO: 154 - DNA, 20 bp, Artificial Sequence
  Description of Artificial Sequence Synthetic oligonucleotide
attgtcatcaccagaaaaag

SEQ ID NO: 155 - DNA, 20 bp, Artificial Sequence
  Description of Artificial Sequence Synthetic oligonucleotide
  Description of Combined DNA/RNA Molecule Synthetic oligonucleotide
aautgtcatcaccagaaaaa

SEQ ID NO: 156 - DNA, 20 bp, Artificial Sequence
  Description of Artificial Sequence Synthetic oligonucleotide
aattgtcatcaccagaaaaa

SEQ ID NO: 157 - DNA, 20 bp, Artificial Sequence
  Description of Artificial Sequence Synthetic oligonucleotide
aaattgtcatcaccagaaaa

SEQ ID NO: 158 - DNA, 20 bp, Artificial Sequence
  Description of Artificial Sequence Synthetic oligonucleotide
  Description of Combined DNA/RNA Molecule Synthetic oligonucleotide
aaautgtcatcaccagaaaa

SEQ ID NO: 159 - DNA, 19 bp, Artificial Sequence
  Description of Artificial Sequence Synthetic oligonucleotide
  Description of Combined DNA/RNA Molecule Synthetic oligonucleotide
aaautgtcatcaccagaaa

SEQ ID NO: 160 - DNA, 19 bp, Artificial Sequence
  Description of Artificial Sequence Synthetic oligonucleotide
  Description of Combined DNA/RNA Molecule Synthetic oligonucleotide
aaautgtcatcaccagaaa

SEQ ID NO: 161 - DNA, 20 bp, Artificial Sequence
  Description of Artificial Sequence Synthetic oligonucleotide
  Description of Combined DNA/RNA Molecule Synthetic oligonucleotide
auaaattgtcatcaccagaa

SEQ ID NO: 162 - DNA, 20 bp, Artificial Sequence
  Description of Artificial Sequence Synthetic oligonucleotide
  Description of Combined DNA/RNA Molecule Synthetic oligonucleotide
auaaattgtcatcaccagaa

SEQ ID NO: 163 - DNA, 20 bp, Artificial Sequence
  Description of Artificial Sequence Synthetic oligonucleotide
  Description of Combined DNA/RNA Molecule Synthetic oligonucleotide
aauaaattgtcatcaccaga

SEQ ID NO: 164 - DNA, 20 bp, Artificial Sequence
  Description of Artificial Sequence Synthetic oligonucleotide
  Description of Combined DNA/RNA Molecule Synthetic oligonucleotide
aauaaattgtcatcaccaga

SEQ ID NO: 165 - DNA, 20 bp, Artificial Sequence
  Description of Artificial Sequence Synthetic oligonucleotide
  Description of Combined DNA/RNA Molecule Synthetic oligonucleotide
aauaaattgtcatcaccaga

SEQ ID NO: 166 - DNA, 20 bp, Artificial Sequence
  Description of Artificial Sequence Synthetic oligonucleotide
  Description of Combined DNA/RNA Molecule Synthetic oligonucleotide
uaauaaattgtcatcaccag

SEQ ID NO: 167 - DNA, 20 bp, Artificial Sequence
  Description of Artificial Sequence Synthetic oligonucleotide
  Description of Combined DNA/RNA Molecule Synthetic oligonucleotide
uaauaaattgtcatcaccag

SEQ ID NO: 168 - DNA, 20 bp, Artificial Sequence
  Description of Artificial Sequence Synthetic oligonucleotide
  Description of Combined DNA/RNA Molecule Synthetic oligonucleotide
uaauaaattgtcatcaccag

SEQ ID NO: 169 - DNA, 20 bp, Artificial Sequence
  Description of Artificial Sequence Synthetic oligonucleotide
  Description of Combined DNA/RNA Molecule Synthetic oligonucleotide
uaauaaattgtcatcaccag

SEQ ID NO: 170 - DNA, 20 bp, Artificial Sequence
  Description of Artificial Sequence Synthetic oligonucleotide
  Description of Combined DNA/RNA Molecule Synthetic oligonucleotide
uuaauaaattgtcatcacca

SEQ ID NO: 171 - DNA, 20 bp, Artificial Sequence
  Description of Artificial Sequence Synthetic oligonucleotide
  Description of Combined DNA/RNA Molecule Synthetic oligonucleotide
uuaauaaattgtcatcacca

SEQ ID NO: 172 - DNA, 20 bp, Artificial Sequence
  Description of Artificial Sequence Synthetic oligonucleotide
  Description of Combined DNA/RNA Molecule Synthetic oligonucleotide
uuaauaaattgtcatcacca

SEQ ID NO: 173 - DNA, 20 bp, Artificial Sequence
  Description of Artificial Sequence Synthetic oligonucleotide
  Description of Combined DNA/RNA Molecule Synthetic oligonucleotide
uuaauaaattgtcatcacca

SEQ ID NO: 174 - DNA, 20 bp, Artificial Sequence
  Description of Artificial Sequence Synthetic oligonucleotide
  Description of Combined DNA/RNA Molecule Synthetic oligonucleotide
auuaataaattgtcatcacc

SEQ ID NO: 175 - DNA, 20 bp, Artificial Sequence
  Description of Artificial Sequence Synthetic oligonucleotide
  Description of Combined DNA/RNA Molecule Synthetic oligonucleotide
auuaataaattgtcatcacc

SEQ ID NO: 176 - DNA, 20 bp, Artificial Sequence
  Description of Artificial Sequence Synthetic oligonucleotide
  Description of Combined DNA/RNA Molecule Synthetic oligonucleotide
auuaataaattgtcatcacc

SEQ ID NO: 177 - DNA, 20 bp, Artificial Sequence
  Description of Artificial Sequence Synthetic oligonucleotide
  Description of Combined DNA/RNA Molecule Synthetic oligonucleotide
auuaataaattgtcatcacc

SEQ ID NO: 178 - DNA, 20 bp, Artificial Sequence
  Description of Artificial Sequence Synthetic oligonucleotide
  Description of Combined DNA/RNA Molecule Synthetic oligonucleotide
uauuaataaattgtcatcac

SEQ ID NO: 179 - DNA, 20 bp, Artificial Sequence
  Description of Artificial Sequence Synthetic oligonucleotide
  Description of Combined DNA/RNA Molecule Synthetic oligonucleotide
uauuaataaattgtcatcac

SEQ ID NO: 180 - DNA, 20 bp, Artificial Sequence
  Description of Artificial Sequence Synthetic oligonucleotide
  Description of Combined DNA/RNA Molecule Synthetic oligonucleotide
uauuaataaattgtcatcac

SEQ ID NO: 181 - DNA, 20 bp, Artificial Sequence
  Description of Artificial Sequence Synthetic oligonucleotide
  Description of Combined DNA/RNA Molecule Synthetic oligonucleotide
cuauuaataaattgtcatca

SEQ ID NO: 182 - DNA, 20 bp, Artificial Sequence
  Description of Artificial Sequence Synthetic oligonucleotide
  Description of Combined DNA/RNA Molecule Synthetic oligonucleotide
cuauuaataaattgtcatca

SEQ ID NO: 183 - DNA, 20 bp, Artificial Sequence
  Description of Artificial Sequence Synthetic oligonucleotide
  Description of Combined DNA/RNA Molecule Synthetic oligonucleotide
acuautaataaattgtcatc

SEQ ID NO: 184 - DNA, 20 bp, Artificial Sequence
  Description of Artificial Sequence Synthetic oligonucleotide
  Description of Combined DNA/RNA Molecule Synthetic oligonucleotide
tgtcatcaccagaaaaaguc

SEQ ID NO: 185 - DNA, 20 bp, Artificial Sequence
  Description of Artificial Sequence Synthetic oligonucleotide
  Description of Combined DNA/RNA Molecule Synthetic oligonucleotide
utgtcatcaccagaaaaagu

SEQ ID NO: 186 - DNA, 20 bp, Artificial Sequence
  Description of Artificial Sequence Synthetic oligonucleotide
  Description of Combined DNA/RNA Molecule Synthetic oligonucleotide
ttgtcatcaccagaaaaagu

SEQ ID NO: 187 - DNA, 20 bp, Artificial Sequence
  Description of Artificial Sequence Synthetic oligonucleotide
  Description of Combined DNA/RNA Molecule Synthetic oligonucleotide
autgtcatcaccagaaaaag

SEQ ID NO: 188 - DNA, 20 bp, Artificial Sequence
  Description of Artificial Sequence Synthetic oligonucleotide
attgtcatcaccagaaaaag

SEQ ID NO: 189 - DNA, 20 bp, Artificial Sequence
  Description of Artificial Sequence Synthetic oligonucleotide
  Description of Combined DNA/RNA Molecule Synthetic oligonucleotide
aautgtcatcaccagaaaaa

SEQ ID NO: 190 - DNA, 20 bp, Artificial Sequence
  Description of Artificial Sequence Synthetic oligonucleotide
aattgtcatcaccagaaaaa

SEQ ID NO: 191 - DNA, 20 bp, Artificial Sequence
  Description of Artificial Sequence Synthetic oligonucleotide
aaattgtcatcaccagaaaa

SEQ ID NO: 192 - DNA, 20 bp, Artificial Sequence
  Description of Artificial Sequence Synthetic oligonucleotide
  Description of Combined DNA/RNA Molecule Synthetic oligonucleotide
aaautgtcatcaccagaaaa

SEQ ID NO: 193 - DNA, 20 bp, Artificial Sequence
  Description of Artificial Sequence Synthetic oligonucleotide
  Description of Combined DNA/RNA Molecule Synthetic oligonucleotide
uaaautgtcatcaccagaaa

SEQ ID NO: 194 - DNA, 20 bp, Artificial Sequence
  Description of Artificial Sequence Synthetic oligonucleotide
  Description of Combined DNA/RNA Molecule Synthetic oligonucleotide
uaaautgtcatcaccagaaa

SEQ ID NO: 195 - DNA, 20 bp, Artificial Sequence
  Description of Artificial Sequence Synthetic oligonucleotide
  Description of Combined DNA/RNA Molecule Synthetic oligonucleotide
auaaattgtcatcaccagaa

SEQ ID NO: 196 - DNA, 20 bp, Artificial Sequence
  Description of Artificial Sequence Synthetic oligonucleotide
  Description of Combined DNA/RNA Molecule Synthetic oligonucleotide
auaaattgtcatcaccagaa

SEQ ID NO: 197 - DNA, 20 bp, Artificial Sequence
  Description of Artificial Sequence Synthetic oligonucleotide
  Description of Combined DNA/RNA Molecule Synthetic oligonucleotide
aauaaattgtcatcaccaga

SEQ ID NO: 198 - DNA, 20 bp, Artificial Sequence
  Description of Artificial Sequence Synthetic oligonucleotide
  Description of Combined DNA/RNA Molecule Synthetic oligonucleotide
aauaaattgtcatcaccaga

SEQ ID NO: 199 - DNA, 20 bp, Artificial Sequence
  Description of Artificial Sequence Synthetic oligonucleotide
  Description of Combined DNA/RNA Molecule Synthetic oligonucleotide
aauaaattgtcatcaccaga

SEQ ID NO: 200 - DNA, 20 bp, Artificial Sequence
  Description of Artificial Sequence Synthetic oligonucleotide
  Description of Combined DNA/RNA Molecule Synthetic oligonucleotide
uaauaaattgtcatcaccag

SEQ ID NO: 201 - DNA, 20 bp, Artificial Sequence
  Description of Artificial Sequence Synthetic oligonucleotide
  Description of Combined DNA/RNA Molecule Synthetic oligonucleotide
uaauaaattgtcatcaccag

SEQ ID NO: 202 - DNA, 20 bp, Artificial Sequence
  Description of Artificial Sequence Synthetic oligonucleotide
  Description of Combined DNA/RNA Molecule Synthetic oligonucleotide
uaauaaattgtcatcaccag

SEQ ID NO: 203 - DNA, 20 bp, Artificial Sequence
  Description of Artificial Sequence Synthetic oligonucleotide
  Description of Combined DNA/RNA Molecule Synthetic oligonucleotide
uaauaaattgtcatcaccag

SEQ ID NO: 204 - DNA, 20 bp, Artificial Sequence
  Description of Artificial Sequence Synthetic oligonucleotide
  Description of Combined DNA/RNA Molecule Synthetic oligonucleotide
uuaauaaattgtcatcacca

SEQ ID NO: 205 - DNA, 20 bp, Artificial Sequence
  Description of Artificial Sequence Synthetic oligonucleotide
  Description of Combined DNA/RNA Molecule Synthetic oligonucleotide
uuaauaaattgtcatcacca

SEQ ID NO: 206 - DNA, 20 bp, Artificial Sequence
  Description of Artificial Sequence Synthetic oligonucleotide
  Description of Combined DNA/RNA Molecule Synthetic oligonucleotide
uuaauaaattgtcatcacca

SEQ ID NO: 207 - DNA, 20 bp, Artificial Sequence
  Description of Artificial Sequence Synthetic oligonucleotide
  Description of Combined DNA/RNA Molecule Synthetic oligonucleotide
uuaauaaattgtcatcacca

SEQ ID NO: 208 - DNA, 20 bp, Artificial Sequence
  Description of Artificial Sequence Synthetic oligonucleotide
  Description of Combined DNA/RNA Molecule Synthetic oligonucleotide
auuaataaattgtcatcacc

SEQ ID NO: 209 - DNA, 20 bp, Artificial Sequence
  Description of Artificial Sequence Synthetic oligonucleotide
  Description of Combined DNA/RNA Molecule Synthetic oligonucleotide
auuaataaattgtcatcacc

SEQ ID NO: 210 - DNA, 20 bp, Artificial Sequence
  Description of Artificial Sequence Synthetic oligonucleotide
  Description of Combined DNA/RNA Molecule Synthetic oligonucleotide
auuaataaattgtcatcacc

SEQ ID NO: 211 - DNA, 20 bp, Artificial Sequence
  Description of Artificial Sequence Synthetic oligonucleotide
  Description of Combined DNA/RNA Molecule Synthetic oligonucleotide
auuaataaattgtcatcacc

SEQ ID NO: 212 - DNA, 20 bp, Artificial Sequence
  Description of Artificial Sequence Synthetic oligonucleotide
  Description of Combined DNA/RNA Molecule Synthetic oligonucleotide
uauuaataaattgtcatcac

SEQ ID NO: 213 - DNA, 20 bp, Artificial Sequence
  Description of Artificial Sequence Synthetic oligonucleotide
  Description of Combined DNA/RNA Molecule Synthetic oligonucleotide
uauuaataaattgtcatcac

SEQ ID NO: 214 - DNA, 20 bp, Artificial Sequence
  Description of Artificial Sequence Synthetic oligonucleotide
  Description of Combined DNA/RNA Molecule Synthetic oligonucleotide
uauuaataaattgtcatcac

SEQ ID NO: 215 - DNA, 20 bp, Artificial Sequence
  Description of Artificial Sequence Synthetic oligonucleotide
  Description of Combined DNA/RNA Molecule Synthetic oligonucleotide
cuauuaataaattgtcatca

SEQ ID NO: 216 - DNA, 20 bp, Artificial Sequence
  Description of Artificial Sequence Synthetic oligonucleotide
  Description of Combined DNA/RNA Molecule Synthetic oligonucleotide
cuauuaataaattgtcatca

SEQ ID NO: 217 - DNA, 20 bp, Artificial Sequence
  Description of Artificial Sequence Synthetic oligonucleotide
  Description of Combined DNA/RNA Molecule Synthetic oligonucleotide
acuautaataaattgtcatc

SEQ ID NO: 218 - DNA, 20 bp, Artificial Sequence
  Description of Artificial Sequence Synthetic oligonucleotide
gggcacaagggcacagactt

SEQ ID NO: 219 - DNA, 20 bp, Artificial Sequence
  Description of Artificial Sequence Synthetic oligonucleotide
ggcacaagggcacagacttc

SEQ ID NO: 220 - DNA, 20 bp, Artificial Sequence
  Description of Artificial Sequence Synthetic oligonucleotide
gcacaagggcacagacttcc

SEQ ID NO: 221 - DNA, 20 bp, Artificial Sequence
  Description of Artificial Sequence Synthetic oligonucleotide
cacaagggcacagacttcca

SEQ ID NO: 222 - DNA, 20 bp, Artificial Sequence
  Description of Artificial Sequence Synthetic oligonucleotide
acaagggcacagacttccaa

SEQ ID NO: 223 - DNA, 20 bp, Artificial Sequence
  Description of Artificial Sequence Synthetic oligonucleotide
caaggccacagacttccaaa

SEQ ID NO: 224 - RNA, 20 nt, Artificial Sequence
  Description of Artificial Sequence Synthetic oligonucleotide
ggcacaagggcacagacuuc

SEQ ID NO: 225 - RNA, 20 nt, Artificial Sequence
  Description of Artificial Sequence Synthetic oligonucleotide
ggcacaagggcacagacuuc

SEQ ID NO: 226 - RNA, 20 nt, Artificial Sequence
  Description of Artificial Sequence Synthetic oligonucleotide
ggcacaagggcacagacuuc

SEQ ID NO: 227 - DNA, 20 bp, Artificial Sequence
  Description of Artificial Sequence Synthetic oligonucleotide
ggcacaagggcacagacttc

SEQ ID NO: 228 - DNA, 20 bp, Artificial Sequence
  Description of Artificial Sequence Synthetic oligonucleotide
ggcacaagggcacagacttc

SEQ ID NO: 229 - DNA, 20 bp, Artificial Sequence
  Description of Artificial Sequence Synthetic oligonucleotide
ggcacaagggcacagacttc

SEQ ID NO: 230 - RNA, 20 nt, Artificial Sequence
  Description of Artificial Sequence Synthetic oligonucleotide
ggcacaagggcacagacuuc

SEQ ID NO: 231 - RNA, 20 nt, Artificial Sequence
  Description of Artificial Sequence Synthetic oligonucleotide
ggcacaagggcacagacuuc

SEQ ID NO: 232 - RNA, 20 nt, Artificial Sequence
  Description of Artificial Sequence Synthetic oligonucleotide
gcagggcacaagggcacaga

SEQ ID NO: 233 - RNA, 20 nt, Artificial Sequence
  Description of Artificial Sequence Synthetic oligonucleotide
cagggcacaagggcacagac

SEQ ID NO: 234 - DNA, 20 bp, Artificial Sequence
  Description of Artificial Sequence Synthetic oligonucleotide
agggcacaagggcacagact

SEQ ID NO: 235 - DNA, 20 bp, Artificial Sequence
  Description of Artificial Sequence Synthetic oligonucleotide
aagggcacagacttccaaag

SEQ ID NO: 236 - DNA, 20 bp, Artificial Sequence
  Description of Artificial Sequence Synthetic oligonucleotide
agggcacagacttccaaagg

SEQ ID NO: 237 - DNA, 20 bp, Artificial Sequence
  Description of Artificial Sequence Synthetic oligonucleotide
gggcacagacttccaaaggc

SEQ ID NO: 238 - RNA, 20 nt, Artificial Sequence
  Description of Artificial Sequence Synthetic oligonucleotide
ggcacaagggcacagacuuc

SEQ ID NO: 239 - DNA, 20 bp, Artificial Sequence
  Description of Artificial Sequence Synthetic oligonucleotide
gggccaacagccagcctgca

SEQ ID NO: 240 - DNA, 20 bp, Artificial Sequence
  Description of Artificial Sequence Synthetic oligonucleotide
ggccaacagccagcctgcag

SEQ ID NO: 241 - DNA, 20 bp, Artificial Sequence
  Description of Artificial Sequence Synthetic oligonucleotide
gccaacagccagcctgcagg

SEQ ID NO: 242 - DNA, 20 bp, Artificial Sequence
  Description of Artificial Sequence Synthetic oligonucleotide
ccaacagccagcctgcagga

SEQ ID NO: 243 - DNA, 20 bp, Artificial Sequence
  Description of Artificial Sequence Synthetic oligonucleotide
caacagccagcctgcaggag

SEQ ID NO: 244 - DNA, 20 bp, Artificial Sequence
  Description of Artificial Sequence Synthetic oligonucleotide
aacagccagcctgcaggagg

SEQ ID NO: 245 - DNA, 20 bp, Artificial Sequence
  Description of Artificial Sequence Synthetic oligonucleotide
gagcagctgcaacctggcaa

SEQ ID NO: 246 - DNA, 20 bp, Artificial Sequence
  Description of Artificial Sequence Synthetic oligonucleotide
agcagctgcaacctggcaac

SEQ ID NO: 247 - DNA, 19 bp, Artificial Sequence
  Description of Artificial Sequence Synthetic oligonucleotide
gcagctgcacctggcaaca

SEQ ID NO: 248 - DNA, 20 bp, Artificial Sequence
  Description of Artificial Sequence Synthetic oligonucleotide
cagctgcaacctggcaacaa

SEQ ID NO: 249 - DNA, 20 bp, Artificial Sequence
  Description of Artificial Sequence Synthetic oligonucleotide
agctgcaacctggcaacaac

SEQ ID NO: 250 - DNA, 20 bp, Artificial Sequence
  Description of Artificial Sequence Synthetic oligonucleotide
gctgcaacctggcaacaacc

SEQ ID NO: 251 - DNA, 20 bp, Artificial Sequence
  Description of Artificial Sequence Synthetic oligonucleotide
attaataaattgtcatcacc

SEQ ID NO: 252 - DNA, 20 bp, Artificial Sequence
  Description of Artificial Sequence Synthetic oligonucleotide
attaataaattgtcatcacc

SEQ ID NO: 253 - DNA, 20 bp, Artificial Sequence
  Description of Artificial Sequence Synthetic oligonucleotide
attaataaattgtcatcacc

SEQ ID NO: 254 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 255 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 256 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 257 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 258 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 259 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 260 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 261 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 262 - DNA, 20 bp, Artificial Sequence
  Description of Artificial Sequence Synthetic oligonucleotide
  Description of Combined DNA/RNA Molecule Synthetic oligonucleotide
auuaauaaattgtcatcacc

SEQ ID NO: 263 - DNA, 20 bp, Artificial Sequence
  Description of Artificial Sequence Synthetic oligonucleotide
ccgtcgcccttcagcacgca

SEQ ID NO: 264 - DNA, 20 bp, Artificial Sequence
  Description of Artificial Sequence Synthetic oligonucleotide
ccgtcgcccttcagcacgca

SEQ ID NO: 265 - DNA, 20 bp, Artificial Sequence
  Description of Artificial Sequence Synthetic oligonucleotide
ccgtcgcccttcagcacgca

SEQ ID NO: 266 - DNA, 20 bp, Artificial Sequence
  Description of Artificial Sequence Synthetic oligonucleotide
ccgtcgcccttcagcacgca

SEQ ID NO: 267 - DNA, 20 bp, Artificial Sequence
  Description of Artificial Sequence Synthetic oligonucleotide
ccgtcgcccttcagcacgca

SEQ ID NO: 268 - DNA, 20 bp, Artificial Sequence
  Description of Artificial Sequence Synthetic oligonucleotide
ccgtcgcccttcagcacgca

SEQ ID NO: 269 - DNA, 20 bp, Artificial Sequence
  Description of Artificial Sequence Synthetic oligonucleotide
gtccctgaagatgtcaatgc

SEQ ID NO: 270 - DNA, 20 bp, Artificial Sequence
  Description of Artificial Sequence Synthetic oligonucleotide
gtccctgaagatgtcaatgc

SEQ ID NO: 271 - DNA, 20 bp, Artificial Sequence
  Description of Artificial Sequence Synthetic oligonucleotide
gtccctgaagatgtcaatgc

SEQ ID NO: 272 - DNA, 20 bp, Artificial Sequence
  Description of Artificial Sequence Synthetic oligonucleotide
gtccctgaagatgtcaatgc

SEQ ID NO: 273 - DNA, 20 bp, Artificial Sequence
  Description of Artificial Sequence Synthetic oligonucleotide
gtccctgaagatgtcaatgc

SEQ ID NO: 274 - RNA, 20 nt, Artificial Sequence
  Description of Artificial Sequence Synthetic oligonucleotide
ggcacaagggcacagacuuc

SEQ ID NO: 275 - RNA, 20 nt, Artificial Sequence
  Description of Artificial Sequence Synthetic oligonucleotide
ggcacaagggcacagacuuc

SEQ ID NO: 276 - RNA, 20 nt, Artificial Sequence
  Description of Artificial Sequence Synthetic oligonucleotide
ggcacaagggcacagacuuc

SEQ ID NO: 277 - RNA, 20 nt, Artificial Sequence
  Description of Artificial Sequence Synthetic oligonucleotide
ggcacaagggcacagacuuc

SEQ ID NO: 278 - RNA, 20 nt, Artificial Sequence
  Description of Artificial Sequence Synthetic oligonucleotide
ggcacaagggcacagacuuc

SEQ ID NO: 279 - RNA, 20 nt, Artificial Sequence
  Description of Artificial Sequence Synthetic oligonucleotide
ggcacaagggcacagacuuc

SEQ ID NO: 280 - RNA, 20 nt, Artificial Sequence
  Description of Artificial Sequence Synthetic oligonucleotide
ggcacaagggcacagacuuc

SEQ ID NO: 281 - RNA, 20 nt, Artificial Sequence
  Description of Artificial Sequence Synthetic oligonucleotide
ggcacaagggcacagacuuc

SEQ ID NO: 282 - RNA, 20 nt, Artificial Sequence
  Description of Artificial Sequence Synthetic oligonucleotide
ggcacaagggcacagacuuc

SEQ ID NO: 283 - DNA, 20 bp, Artificial Sequence
  Description of Artificial Sequence Synthetic oligonucleotide
ggcacaagggcacagacttc

SEQ ID NO: 284 - RNA, 20 nt, Artificial Sequence
  Description of Artificial Sequence Synthetic oligonucleotide
ucaaggaagauggcauuucu

SEQ ID NO: 285 - RNA, 20 nt, Artificial Sequence
  Description of Artificial Sequence Synthetic oligonucleotide
ucaaggaagauggcauuucu

SEQ ID NO: 286 - RNA, 20 nt, Artificial Sequence
  Description of Artificial Sequence Synthetic oligonucleotide
ucaaggaagauggcauuucu

SEQ ID NO: 287 - RNA, 20 nt, Artificial Sequence
  Description of Artificial Sequence Synthetic oligonucleotide
ucaaggaagauggcauuucu

SEQ ID NO: 288 - RNA, 20 nt, Artificial Sequence
  Description of Artificial Sequence Synthetic oligonucleotide
ucaaggaagauggcauuucu

SEQ ID NO: 289 - RNA, 20 nt, Artificial Sequence
  Description of Artificial Sequence Synthetic oligonucleotide
ucaaggaagauggcauuucu

SEQ ID NO: 290 - RNA, 20 nt, Artificial Sequence
  Description of Artificial Sequence Synthetic oligonucleotide
ucaaggaagauggcauuucu

SEQ ID NO: 291 - RNA, 20 nt, Artificial Sequence
  Description of Artificial Sequence Synthetic oligonucleotide
ucaaggaagauggcauuucu

SEQ ID NO: 292 - RNA, 20 nt, Artificial Sequence
  Description of Artificial Sequence Synthetic oligonucleotide
ucaaggaagauggcauuucu

SEQ ID NO: 293 - RNA, 20 nt, Artificial Sequence
  Description of Artificial Sequence Synthetic oligonucleotide
ucaaggaagauggcauuucu

SEQ ID NO: 294 - RNA, 20 nt, Artificial Sequence
  Description of Artificial Sequence Synthetic oligonucleotide
ucaaggaagauggcauuucu

SEQ ID NO: 295 - RNA, 20 nt, Artificial Sequence
  Description of Artificial Sequence Synthetic oligonucleotide
ucaaggaagauggcauuucu

SEQ ID NO: 296 - RNA, 20 nt, Artificial Sequence
  Description of Artificial Sequence Synthetic oligonucleotide
ucaaggaagauggcauuucu

SEQ ID NO: 297 - RNA, 20 nt, Artificial Sequence
  Description of Artificial Sequence Synthetic oligonucleotide
ucaaggaagauggcauuucu

SEQ ID NO: 298 - RNA, 20 nt, Artificial Sequence
  Description of Artificial Sequence Synthetic oligonucleotide
ucaaggaagauggcauuucu

SEQ ID NO: 299 - RNA, 20 nt, Artificial Sequence
  Description of Artificial Sequence Synthetic oligonucleotide
ucaaggaagauggcauuucu

SEQ ID NO: 300 - DNA, 20 bp, Artificial Sequence
  Description of Artificial Sequence Synthetic oligonucleotide
tcaaggaagatggcatttct

SEQ ID NO: 301 - RNA, 20 nt, Artificial Sequence
  Description of Artificial Sequence Synthetic oligonucleotide
ucaaggaagauggcauuucu

SEQ ID NO: 302 - DNA, 20 bp, Artificial Sequence
  Description of Artificial Sequence Synthetic oligonucleotide
tcaaggaagatggcatttct

SEQ ID NO: 303 - DNA, 20 bp, Artificial Sequence
  Description of Artificial Sequence Synthetic oligonucleotide
tcaaggaagatggcatttct

SEQ ID NO: 304 - DNA, 20 bp, Artificial Sequence
  Description of Artificial Sequence Synthetic oligonucleotide
  Description of Combined DNA/RNA Molecule Synthetic oligonucleotide
tcaaggaagauggcatttct

SEQ ID NO: 305 - DNA, 20 bp, Artificial Sequence
  Description of Artificial Sequence Synthetic oligonucleotide
  Description of Combined DNA/RNA Molecule Synthetic oligonucleotide
ucaaggaagatggcatuucu

SEQ ID NO: 306 - DNA, 20 bp, Artificial Sequence
  Description of Artificial Sequence Synthetic oligonucleotide
  Description of Combined DNA/RNA Molecule Synthetic oligonucleotide
tcaaggaagatggcautucu

SEQ ID NO: 307 - DNA, 20 bp, Artificial Sequence
  Description of Artificial Sequence Synthetic oligonucleotide
  Description of Combined DNA/RNA Molecule Synthetic oligonucleotide
ucaaggaagauggcatutcu

SEQ ID NO: 308 - DNA, 20 bp, Artificial Sequence
  Description of Artificial Sequence Synthetic oligonucleotide
  Description of Combined DNA/RNA Molecule Synthetic oligonucleotide
tcaaggaagatggcauutcu

SEQ ID NO: 309 - DNA, 20 bp, Artificial Sequence
  Description of Artificial Sequence Synthetic oligonucleotide
  Description of Combined DNA/RNA Molecule Synthetic oligonucleotide
tcaaggaagauggcauttcu

SEQ ID NO: 310 - DNA, 20 bp, Artificial Sequence
  Description of Artificial Sequence Synthetic oligonucleotide
  Description of Combined DNA/RNA Molecule Synthetic oligonucleotide
tcaaggaagatggcatttcu

SEQ ID NO: 311 - DNA, 20 bp, Artificial Sequence
  Description of Artificial Sequence Synthetic oligonucleotide
  Description of Combined DNA/RNA Molecule Synthetic oligonucleotide
tcaaggaagatggcauttcu

SEQ ID NO: 312 - DNA, 20 bp, Artificial Sequence
  Description of Artificial Sequence Synthetic oligonucleotide
  Description of Combined DNA/RNA Molecule Synthetic oligonucleotide
ucaaggaagauggcatttcu

SEQ ID NO: 313 - DNA, 20 bp, Artificial Sequence
  Description of Artificial Sequence Synthetic oligonucleotide
  Description of Combined DNA/RNA Molecule Synthetic oligonucleotide
tcaaggaagatggcatttcu

SEQ ID NO: 314 - DNA, 20 bp, Artificial Sequence
  Description of Artificial Sequence Synthetic oligonucleotide
  Description of Combined DNA/RNA Molecule Synthetic oligonucleotide
tcaaggaagauggcatttct

SEQ ID NO: 315 - DNA, 20 bp, Artificial Sequence
  Description of Artificial Sequence Synthetic oligonucleotide
  Description of Combined DNA/RNA Molecule Synthetic oligonucleotide
ucaaggaagatggcatuucu

SEQ ID NO: 316 - DNA, 20 bp, Artificial Sequence
  Description of Artificial Sequence Synthetic oligonucleotide
  Description of Combined DNA/RNA Molecule Synthetic oligonucleotide
tcaaggaagatggcautucu

SEQ ID NO: 317 - DNA, 20 bp, Artificial Sequence
  Description of Artificial Sequence Synthetic oligonucleotide
  Description of Combined DNA/RNA Molecule Synthetic oligonucleotide
ucaaggaagauggcatutcu

SEQ ID NO: 318 - DNA, 20 bp, Artificial Sequence
  Description of Artificial Sequence Synthetic oligonucleotide
  Description of Combined DNA/RNA Molecule Synthetic oligonucleotide
tcaaggaagatggcauutcu

SEQ ID NO: 319 - DNA, 20 bp, Artificial Sequence
  Description of Artificial Sequence Synthetic oligonucleotide
  Description of Combined DNA/RNA Molecule Synthetic oligonucleotide
tcaaggaagauggcauttcu

SEQ ID NO: 320 - DNA, 20 bp, Artificial Sequence
  Description of Artificial Sequence Synthetic oligonucleotide
  Description of Combined DNA/RNA Molecule Synthetic oligonucleotide
tcaaggaagatggcatttcu

SEQ ID NO: 321 - DNA, 20 bp, Artificial Sequence
  Description of Artificial Sequence Synthetic oligonucleotide
  Description of Combined DNA/RNA Molecule Synthetic oligonucleotide
tcaaggaagatggcauttcu

SEQ ID NO: 322 - DNA, 20 bp, Artificial Sequence
  Description of Artificial Sequence Synthetic oligonucleotide
  Description of Combined DNA/RNA Molecule Synthetic oligonucleotide
ucaaggaagauggcatttcu

SEQ ID NO: 323 - DNA, 20 bp, Artificial Sequence
  Description of Artificial Sequence Synthetic oligonucleotide
  Description of Combined DNA/RNA Molecule Synthetic oligonucleotide
tcaaggaagatggcatttcu

SEQ ID NO: 324 - RNA, 20 nt, Artificial Sequence
  Description of Artificial Sequence Synthetic oligonucleotide
ucaaggaagauggcauuucu

SEQ ID NO: 325 - RNA, 20 nt, Artificial Sequence
  Description of Artificial Sequence Synthetic oligonucleotide
ucaaggaagauggcauuucu

SEQ ID NO: 326 - RNA, 20 nt, Artificial Sequence
  Description of Artificial Sequence Synthetic oligonucleotide
ucaaggaagauggcauuucu

SEQ ID NO: 327 - RNA, 20 nt, Artificial Sequence
  Description of Artificial Sequence Synthetic oligonucleotide
ucaaggaagauggcauuucu

SEQ ID NO: 328 - RNA, 20 nt, Artificial Sequence
  Description of Artificial Sequence Synthetic oligonucleotide
ucaaggaagauggcauuucu

SEQ ID NO: 329 - RNA, 20 nt, Artificial Sequence
  Description of Artificial Sequence Synthetic oligonucleotide
ucaaggaagauggcauuucu

SEQ ID NO: 330 - RNA, 20 nt, Artificial Sequence
  Description of Artificial Sequence Synthetic oligonucleotide
ucaaggaagauggcauuucu

SEQ ID NO: 331 - RNA, 20 nt, Artificial Sequence
  Description of Artificial Sequence Synthetic oligonucleotide
ucaaggaagauggcauuucu

SEQ ID NO: 332 - RNA, 20 nt, Artificial Sequence
  Description of Artificial Sequence Synthetic oligonucleotide
ucaaggaagauggcauuucu

SEQ ID NO: 333 - RNA, 20 nt, Artificial Sequence
  Description of Artificial Sequence Synthetic oligonucleotide
ucaaggaagauggcauuucu

SEQ ID NO: 334 - RNA, 20 nt, Artificial Sequence
  Description of Artificial Sequence Synthetic oligonucleotide
ucaaggaagauggcauuucu

SEQ ID NO: 335 - RNA, 20 nt, Artificial Sequence
  Description of Artificial Sequence Synthetic oligonucleotide
ucaaggaagauggcauuucu

SEQ ID NO: 336 - RNA, 20 nt, Artificial Sequence
  Description of Artificial Sequence Synthetic oligonucleotide
ucaaggaagauggcauuucu

SEQ ID NO: 337 - RNA, 20 nt, Artificial Sequence
  Description of Artificial Sequence Synthetic oligonucleotide
ucaaggaagauggcauuucu

SEQ ID NO: 338 - RNA, 20 nt, Artificial Sequence
  Description of Artificial Sequence Synthetic oligonucleotide
ucaaggaagauggcauuucu

SEQ ID NO: 339 - RNA, 20 nt, Artificial Sequence
  Description of Artificial Sequence Synthetic oligonucleotide
ucaaggaagauggcauuucu

SEQ ID NO: 340 - RNA, 20 nt, Artificial Sequence
  Description of Artificial Sequence Synthetic oligonucleotide
ucaaggaagauggcauuucu

SEQ ID NO: 341 - RNA, 20 nt, Artificial Sequence
  Description of Artificial Sequence Synthetic oligonucleotide
ucaaggaagauggcauuucu

SEQ ID NO: 342 - RNA, 20 nt, Artificial Sequence
  Description of Artificial Sequence Synthetic oligonucleotide
ucaaggaagauggcauuucu

SEQ ID NO: 343 - RNA, 20 nt, Artificial Sequence
  Description of Artificial Sequence Synthetic oligonucleotide
ucaaggaagauggcauuucu

SEQ ID NO: 344 - RNA, 20 nt, Artificial Sequence
  Description of Artificial Sequence Synthetic oligonucleotide
ucaaggaagauggcauuucu

SEQ ID NO: 345 - RNA, 20 nt, Artificial Sequence
  Description of Artificial Sequence Synthetic oligonucleotide
ucaaggaagauggcauuucu

SEQ ID NO: 346 - RNA, 20 nt, Artificial Sequence
  Description of Artificial Sequence Synthetic oligonucleotide
ucaaggaagauggcauuucu

SEQ ID NO: 347 - RNA, 20 nt, Artificial Sequence
  Description of Artificial Sequence Synthetic oligonucleotide
ucaaggaagauggcauuucu

SEQ ID NO: 348 - RNA, 20 nt, Artificial Sequence
  Description of Artificial Sequence Synthetic oligonucleotide
ucaaggaagauggcauuucu

SEQ ID NO: 349 - RNA, 20 nt, Artificial Sequence
  Description of Artificial Sequence Synthetic oligonucleotide
ucaaggaagauggcauuucu

SEQ ID NO: 350 - RNA, 20 nt, Artificial Sequence
  Description of Artificial Sequence Synthetic oligonucleotide
ucaaggaagauggcauuucu

SEQ ID NO: 351 - RNA, 20 nt, Artificial Sequence
  Description of Artificial Sequence Synthetic oligonucleotide
ucaaggaagauggcauuucu

SEQ ID NO: 352 - RNA, 20 nt, Artificial Sequence
  Description of Artificial Sequence Synthetic oligonucleotide
ucaaggaagauggcauuucu

SEQ ID NO: 353 - RNA, 20 nt, Artificial Sequence
  Description of Artificial Sequence Synthetic oligonucleotide
ucaaggaagauggcauuucu

SEQ ID NO: 354 - RNA, 20 nt, Artificial Sequence
  Description of Artificial Sequence Synthetic oligonucleotide
ucaaggaagauggcauuucu

SEQ ID NO: 355 - RNA, 20 nt, Artificial Sequence
  Description of Artificial Sequence Synthetic oligonucleotide
ucaaggaagauggcauuucu

SEQ ID NO: 356 - RNA, 20 nt, Artificial Sequence
  Description of Artificial Sequence Synthetic oligonucleotide
ucaaggaagauggcauuucu

SEQ ID NO: 357 - RNA, 20 nt, Artificial Sequence
  Description of Artificial Sequence Synthetic oligonucleotide
ucaaggaagauggcauuucu

SEQ ID NO: 358 - RNA, 20 nt, Artificial Sequence
  Description of Artificial Sequence Synthetic oligonucleotide
ucaaggaagauggcauuucu

SEQ ID NO: 359 - RNA, 20 nt, Artificial Sequence
  Description of Artificial Sequence Synthetic oligonucleotide
ucaaggaagauggcauuucu

SEQ ID NO: 360 - RNA, 20 nt, Artificial Sequence
  Description of Artificial Sequence Synthetic oligonucleotide
ucaaggaagauggcauuucu

SEQ ID NO: 361 - RNA, 20 nt, Artificial Sequence
  Description of Artificial Sequence Synthetic oligonucleotide
ucaaggaagauggcauuucu

SEQ ID NO: 362 - RNA, 20 nt, Artificial Sequence
  Description of Artificial Sequence Synthetic oligonucleotide
ucaaggaagauggcauuucu

SEQ ID NO: 363 - RNA, 20 nt, Artificial Sequence
  Description of Artificial Sequence Synthetic oligonucleotide
ucaaggaagauggcauuucu

SEQ ID NO: 364 - RNA, 20 nt, Artificial Sequence
  Description of Artificial Sequence Synthetic oligonucleotide
ucaaggaagauggcauuucu

SEQ ID NO: 365 - RNA, 20 nt, Artificial Sequence
  Description of Artificial Sequence Synthetic oligonucleotide
ucaaggaagauggcauuucu

SEQ ID NO: 366 - RNA, 20 nt, Artificial Sequence
  Description of Artificial Sequence Synthetic oligonucleotide
ucaaggaagauggcauuucu

SEQ ID NO: 367 - RNA, 20 nt, Artificial Sequence
  Description of Artificial Sequence Synthetic oligonucleotide
ucaaggaagauggcauuucu

SEQ ID NO: 368 - RNA, 20 nt, Artificial Sequence
  Description of Artificial Sequence Synthetic oligonucleotide
ucaaggaagauggcauuucu

SEQ ID NO: 369 - RNA, 20 nt, Artificial Sequence
  Description of Artificial Sequence Synthetic oligonucleotide
ucaaggaagauggcauuucu

SEQ ID NO: 370 - RNA, 20 nt, Artificial Sequence
  Description of Artificial Sequence Synthetic oligonucleotide
ucaaggaagauggcauuucu

SEQ ID NO: 371 - RNA, 20 nt, Artificial Sequence
  Description of Artificial Sequence Synthetic oligonucleotide
ucaaggaagauggcauuucu

SEQ ID NO: 372 - RNA, 20 nt, Artificial Sequence
  Description of Artificial Sequence Synthetic oligonucleotide
ucaaggaagauggcauuucu

SEQ ID NO: 373 - RNA, 20 nt, Artificial Sequence
  Description of Artificial Sequence Synthetic oligonucleotide
ucaaggaagauggcauuucu

SEQ ID NO: 374 - RNA, 20 nt, Artificial Sequence
  Description of Artificial Sequence Synthetic oligonucleotide
ucaaggaagauggcauuucu

SEQ ID NO: 375 - RNA, 20 nt, Artificial Sequence
  Description of Artificial Sequence Synthetic oligonucleotide
ucaaggaagauggcauuucu

SEQ ID NO: 376 - RNA, 20 nt, Artificial Sequence
  Description of Artificial Sequence Synthetic oligonucleotide
ucaaggaagauggcauuucu

SEQ ID NO: 377 - RNA, 20 nt, Artificial Sequence
  Description of Artificial Sequence Synthetic oligonucleotide
ucaaggaagauggcauuucu

SEQ ID NO: 378 - RNA, 20 nt, Artificial Sequence
  Description of Artificial Sequence Synthetic oligonucleotide
ucaaggaagauggcauuucu

SEQ ID NO: 379 - RNA, 20 nt, Artificial Sequence
  Description of Artificial Sequence Synthetic oligonucleotide
ucaaggaagauggcauuucu

SEQ ID NO: 380 - RNA, 20 nt, Artificial Sequence
  Description of Artificial Sequence Synthetic oligonucleotide
ucaaggaagauggcauuucu

SEQ ID NO: 381 - RNA, 20 nt, Artificial Sequence
  Description of Artificial Sequence Synthetic oligonucleotide
ucaaggaagauggcauuucu

SEQ ID NO: 382 - RNA, 20 nt, Artificial Sequence
  Description of Artificial Sequence Synthetic oligonucleotide
ucaaggaagauggcauuucu

SEQ ID NO: 383 - RNA, 20 nt, Artificial Sequence
  Description of Artificial Sequence Synthetic oligonucleotide
ucaaggaagauggcauuucu

SEQ ID NO: 384 - RNA, 20 nt, Artificial Sequence
  Description of Artificial Sequence Synthetic oligonucleotide
ucaaggaagauggcauuucu

SEQ ID NO: 385 - RNA, 20 nt, Artificial Sequence
  Description of Artificial Sequence Synthetic oligonucleotide
ucaaggaagauggcauuucu

SEQ ID NO: 386 - RNA, 20 nt, Artificial Sequence
  Description of Artificial Sequence Synthetic oligonucleotide
ucaaggaagauggcauuucu

SEQ ID NO: 387 - RNA, 20 nt, Artificial Sequence
  Description of Artificial Sequence Synthetic oligonucleotide
ucaaggaagauggcauuucu

SEQ ID NO: 388 - RNA, 20 nt, Artificial Sequence
  Description of Artificial Sequence Synthetic oligonucleotide
ucaaggaagauggcauuucu

SEQ ID NO: 389 - RNA, 20 nt, Artificial Sequence
  Description of Artificial Sequence Synthetic oligonucleotide
ucaaggaagauggcauuucu

SEQ ID NO: 390 - RNA, 20 nt, Artificial Sequence
  Description of Artificial Sequence Synthetic oligonucleotide
ucaaggaagauggcauuucu

SEQ ID NO: 391 - RNA, 20 nt, Artificial Sequence
  Description of Artificial Sequence Synthetic oligonucleotide
ucaaggaagauggcauuucu

SEQ ID NO: 392 - RNA, 20 nt, Artificial Sequence
  Description of Artificial Sequence Synthetic oligonucleotide
ucaaggaagauggcauuucu

SEQ ID NO: 393 - RNA, 20 nt, Artificial Sequence
  Description of Artificial Sequence Synthetic oligonucleotide
ucaaggaagauggcauuucu

SEQ ID NO: 394 - RNA, 20 nt, Artificial Sequence
  Description of Artificial Sequence Synthetic oligonucleotide
ucaaggaagauggcauuucu

SEQ ID NO: 395 - RNA, 20 nt, Artificial Sequence
  Description of Artificial Sequence Synthetic oligonucleotide
ggccaaaccucggcuuaccu

SEQ ID NO: 396 - RNA, 30 nt, Artificial Sequence
  Description of Artificial Sequence Synthetic oligonucleotide
cuccaacaucaaggaagauggcauuucuag

SEQ ID NO: 397 - RNA, 25 nt, Artificial Sequence
  Description of Artificial Sequence Synthetic oligonucleotide
accagaguaacagucugaguaggag

SEQ ID NO: 398 - RNA, 25 nt, Artificial Sequence
  Description of Artificial Sequence Synthetic oligonucleotide
caccagaguaacagucugaguagga

SEQ ID NO: 399 - RNA, 25 nt, Artificial Sequence
  Description of Artificial Sequence Synthetic oligonucleotide
ucaccagaguaacagucugaguagg

SEQ ID NO: 400 - RNA, 25 nt, Artificial Sequence
  Description of Artificial Sequence Synthetic oligonucleotide
gucaccagaguaacagucugaguag

SEQ ID NO: 401 - RNA, 25 nt, Artificial Sequence
  Description of Artificial Sequence Synthetic oligonucleotide
guugugucaccagaguaacagucug

SEQ ID NO: 402 - RNA, 25 nt, Artificial Sequence
  Description of Artificial Sequence Synthetic oligonucleotide
gguugugucaccagaguaacagucu

SEQ ID NO: 403 - RNA, 25 nt, Artificial Sequence
  Description of Artificial Sequence Synthetic oligonucleotide
agguugugucaccagaguaacaguc

SEQ ID NO: 404 - RNA, 25 nt, Artificial Sequence
  Description of Artificial Sequence Synthetic oligonucleotide
cagguugugucaccagaguaacagu

SEQ ID NO: 405 - RNA, 25 nt, Artificial Sequence
  Description of Artificial Sequence Synthetic oligonucleotide
acagguugugucaccagaguaacag

SEQ ID NO: 406 - RNA, 25 nt, Artificial Sequence
  Description of Artificial Sequence Synthetic oligonucleotide
ccacagguugugucaccagaguaac

SEQ ID NO: 407 - RNA, 25 nt, Artificial Sequence
  Description of Artificial Sequence Synthetic oligonucleotide
accacagguugugucaccagaguaa

SEQ ID NO: 408 - RNA, 25 nt, Artificial Sequence
  Description of Artificial Sequence Synthetic oligonucleotide
aaccacagguugugucaccagagua

SEQ ID NO: 409 - RNA, 25 nt, Artificial Sequence
  Description of Artificial Sequence Synthetic oligonucleotide
uaaccacagguugugucaccagagu

SEQ ID NO: 410 - RNA, 25 nt, Artificial Sequence
  Description of Artificial Sequence Synthetic oligonucleotide
guaaccacagguugugucaccagag

SEQ ID NO: 411 - RNA, 25 nt, Artificial Sequence
  Description of Artificial Sequence Synthetic oligonucleotide
aguaaccacagguugugucaccaga

SEQ ID NO: 412 - RNA, 25 nt, Artificial Sequence
  Description of Artificial Sequence Synthetic oligonucleotide
uaguaaccacagguugugucaccag

SEQ ID NO: 413 - RNA, 25 nt, Artificial Sequence
  Description of Artificial Sequence Synthetic oligonucleotide
uuaguaaccacagguugugucacca

SEQ ID NO: 414 - RNA, 25 nt, Artificial Sequence
  Description of Artificial Sequence Synthetic oligonucleotide
cuuaguaaccacagguugugucacc

SEQ ID NO: 415 - RNA, 25 nt, Artificial Sequence
  Description of Artificial Sequence Synthetic oligonucleotide
ccuuaguaaccacagguugugucac

SEQ ID NO: 416 - RNA, 25 nt, Artificial Sequence
  Description of Artificial Sequence Synthetic oligonucleotide
uccuuaguaaccacagguuguguca

SEQ ID NO: 417 - RNA, 25 nt, Artificial Sequence
  Description of Artificial Sequence Synthetic oligonucleotide
guuuccuuaguaaccacagguugug

SEQ ID NO: 418 - RNA, 25 nt, Artificial Sequence
  Description of Artificial Sequence Synthetic oligonucleotide
aguuuccuuaguaaccacagguugu

SEQ ID NO: 419 - RNA, 25 nt, Artificial Sequence
  Description of Artificial Sequence Synthetic oligonucleotide
caguuuccuuaguaaccacagguug

SEQ ID NO: 420 - RNA, 25 nt, Artificial Sequence
  Description of Artificial Sequence Synthetic oligonucleotide
gcaguuuccuuaguaaccacagguu

SEQ ID NO: 421 - RNA, 25 nt, Artificial Sequence
  Description of Artificial Sequence Synthetic oligonucleotide
ggcaguuuccuuaguaaccacaggu

SEQ ID NO: 422 - RNA, 25 nt, Artificial Sequence
  Description of Artificial Sequence Synthetic oligonucleotide
uggcaguuuccuuaguaaccacagg

SEQ ID NO: 423 - RNA, 25 nt, Artificial Sequence
  Description of Artificial Sequence Synthetic oligonucleotide
auggcaguuuccuuaguaaccacag

SEQ ID NO: 424 - RNA, 25 nt, Artificial Sequence
  Description of Artificial Sequence Synthetic oligonucleotide
agauggcaguuuccuuaguaaccac

SEQ ID NO: 425 - RNA, 25 nt, Artificial Sequence
  Description of Artificial Sequence Synthetic oligonucleotide
gagauggcaguuuccuuaguaacca

SEQ ID NO: 426 - RNA, 25 nt, Artificial Sequence
  Description of Artificial Sequence Synthetic oligonucleotide
ggagauggcaguuuccuuaguaacc

SEQ ID NO: 427 - RNA, 25 nt, Artificial Sequence
  Description of Artificial Sequence Synthetic oligonucleotide
uggagauggcaguuuccuuaguaac

SEQ ID NO: 428 - RNA, 25 nt, Artificial Sequence
  Description of Artificial Sequence Synthetic oligonucleotide
uuggagauggcaguuuccuuaguaa

SEQ ID NO: 429 - RNA, 25 nt, Artificial Sequence
  Description of Artificial Sequence Synthetic oligonucleotide
uuuggagauggcaguuuccuuagua

SEQ ID NO: 430 - RNA, 25 nt, Artificial Sequence
  Description of Artificial Sequence Synthetic oligonucleotide
aguuuggagauggcaguuuccuuag

SEQ ID NO: 431 - RNA, 25 nt, Artificial Sequence
  Description of Artificial Sequence Synthetic oligonucleotide
uaguuuggagauggcaguuuccuua

SEQ ID NO: 432 - RNA, 25 nt, Artificial Sequence
  Description of Artificial Sequence Synthetic oligonucleotide
cuaguuuggagauggcaguuuccuu

SEQ ID NO: 433 - RNA, 25 nt, Artificial Sequence
  Description of Artificial Sequence Synthetic oligonucleotide
ucuaguuuggagauggcaguuuccu

SEQ ID NO: 434 - RNA, 25 nt, Artificial Sequence
  Description of Artificial Sequence Synthetic oligonucleotide
uucuaguuuggagauggcaguuucc

SEQ ID NO: 435 - RNA, 25 nt, Artificial Sequence
  Description of Artificial Sequence Synthetic oligonucleotide
cauuucuaguuuggagauggcaguu

SEQ ID NO: 436 - RNA, 25 nt, Artificial Sequence
  Description of Artificial Sequence Synthetic oligonucleotide
gcauuucuaguuuggagauggcagu

SEQ ID NO: 437 - RNA, 25 nt, Artificial Sequence
  Description of Artificial Sequence Synthetic oligonucleotide
auggcauuucuaguuuggagauggc

SEQ ID NO: 438 - RNA, 25 nt, Artificial Sequence
  Description of Artificial Sequence Synthetic oligonucleotide
gaagauggcauuucuaguuuggaga

SEQ ID NO: 439 - RNA, 25 nt, Artificial Sequence
  Description of Artificial Sequence Synthetic oligonucleotide
aggaagauggcauuucuaguuugga

SEQ ID NO: 440 - RNA, 25 nt, Artificial Sequence
  Description of Artificial Sequence Synthetic oligonucleotide
aaggaagauggcauuucuaguuugg

SEQ ID NO: 441 - RNA, 25 nt, Artificial Sequence
  Description of Artificial Sequence Synthetic oligonucleotide
caaggaagauggcauuucuaguuug

SEQ ID NO: 442 - RNA, 25 nt, Artificial Sequence
  Description of Artificial Sequence Synthetic oligonucleotide
caucaaggaagauggcauuucuagu

SEQ ID NO: 443 - RNA, 25 nt, Artificial Sequence
  Description of Artificial Sequence Synthetic oligonucleotide
acaucaaggaagauggcauuucuag

SEQ ID NO: 444 - RNA, 25 nt, Artificial Sequence
  Description of Artificial Sequence Synthetic oligonucleotide
aacaucaaggaagauggcauuucua

SEQ ID NO: 445 - RNA, 25 nt, Artificial Sequence
  Description of Artificial Sequence Synthetic oligonucleotide
caacaucaaggaagauggcauuucu

SEQ ID NO: 446 - RNA, 25 nt, Artificial Sequence
  Description of Artificial Sequence Synthetic oligonucleotide
cuccaacaucaaggaagauggcauu

SEQ ID NO: 447 - RNA, 25 nt, Artificial Sequence
  Description of Artificial Sequence Synthetic oligonucleotide
accuccaacaucaaggaagauggca

SEQ ID NO: 448 - RNA, 25 nt, Artificial Sequence
  Description of Artificial Sequence Synthetic oligonucleotide
guaccuccaacaucaaggaagaugg

SEQ ID NO: 449 - RNA, 25 nt, Artificial Sequence
  Description of Artificial Sequence Synthetic oligonucleotide
agguaccuccaacaucaaggaagau

SEQ ID NO: 450 - RNA, 25 nt, Artificial Sequence
  Description of Artificial Sequence Synthetic oligonucleotide
agagcagguaccuccaacaucaagg

SEQ ID NO: 451 - RNA, 25 nt, Artificial Sequence
  Description of Artificial Sequence Synthetic oligonucleotide
cagagcagguaccuccaacaucaag

SEQ ID NO: 452 - RNA, 25 nt, Artificial Sequence
  Description of Artificial Sequence Synthetic oligonucleotide
cugccagagcagguaccuccaacau

SEQ ID NO: 453 - RNA, 25 nt, Artificial Sequence
  Description of Artificial Sequence Synthetic oligonucleotide
ucugccagagcagguaccuccaaca

SEQ ID NO: 454 - RNA, 25 nt, Artificial Sequence
  Description of Artificial Sequence Synthetic oligonucleotide
aucugccagagcagguaccuccaac

SEQ ID NO: 455 - RNA, 25 nt, Artificial Sequence
  Description of Artificial Sequence Synthetic oligonucleotide
aaucugccagagcagguaccuccaa

SEQ ID NO: 456 - RNA, 25 nt, Artificial Sequence
  Description of Artificial Sequence Synthetic oligonucleotide
aaaucugccagagcagguaccucca

SEQ ID NO: 457 - RNA, 25 nt, Artificial Sequence
  Description of Artificial Sequence Synthetic oligonucleotide
gaaaucugccagagcagguaccucc

SEQ ID NO: 458 - RNA, 25 nt, Artificial Sequence
  Description of Artificial Sequence Synthetic oligonucleotide
ugaaaucugccagagcagguaccuc

SEQ ID NO: 459 - RNA, 25 nt, Artificial Sequence
  Description of Artificial Sequence Synthetic oligonucleotide
uugaaaucugccagagcagguaccu

SEQ ID NO: 460 - RNA, 25 nt, Artificial Sequence
  Description of Artificial Sequence Synthetic oligonucleotide
cccgguugaaaucugccagagcagg

SEQ ID NO: 461 - RNA, 25 nt, Artificial Sequence
  Description of Artificial Sequence Synthetic oligonucleotide
ccaagcccgguugaaaucugccaga

SEQ ID NO: 462 - RNA, 25 nt, Artificial Sequence
  Description of Artificial Sequence Synthetic oligonucleotide
uccaagcccgguugaaaucugccag

SEQ ID NO: 463 - RNA, 25 nt, Artificial Sequence
  Description of Artificial Sequence Synthetic oligonucleotide
guccaagcccgguugaaaucugcca

SEQ ID NO: 464 - RNA, 25 nt, Artificial Sequence
  Description of Artificial Sequence Synthetic oligonucleotide
ucuguccaagcccgguugaaaucug

SEQ ID NO: 465 - RNA, 25 nt, Artificial Sequence
  Description of Artificial Sequence Synthetic oligonucleotide
uucuguccaagcccgguugaaaucu

SEQ ID NO: 466 - RNA, 25 nt, Artificial Sequence
  Description of Artificial Sequence Synthetic oligonucleotide
guucuguccaagcccgguugaaauc

SEQ ID NO: 467 - RNA, 25 nt, Artificial Sequence
  Description of Artificial Sequence Synthetic oligonucleotide
aguucuguccaagcccgguugaaau

SEQ ID NO: 468 - RNA, 25 nt, Artificial Sequence
  Description of Artificial Sequence Synthetic oligonucleotide
aaguucuguccaagcccgguugaaa

SEQ ID NO: 469 - RNA, 25 nt, Artificial Sequence
  Description of Artificial Sequence Synthetic oligonucleotide
uaaguucuguccaagcccgguugaa

SEQ ID NO: 470 - RNA, 25 nt, Artificial Sequence
  Description of Artificial Sequence Synthetic oligonucleotide
guaaguucuguccaagcccgguuga

SEQ ID NO: 471 - RNA, 25 nt, Artificial Sequence
  Description of Artificial Sequence Synthetic oligonucleotide
gguaaguucuguccaagcccgguug

SEQ ID NO: 472 - RNA, 25 nt, Artificial Sequence
  Description of Artificial Sequence Synthetic oligonucleotide
cgguaaguucuguccaagcccgguu

SEQ ID NO: 473 - RNA, 25 nt, Artificial Sequence
  Description of Artificial Sequence Synthetic oligonucleotide
ucgguaaguucuguccaagcccggu

SEQ ID NO: 474 - RNA, 25 nt, Artificial Sequence
  Description of Artificial Sequence Synthetic oligonucleotide
gucgguaaguucuguccaagcccgg

SEQ ID NO: 475 - RNA, 25 nt, Artificial Sequence
  Description of Artificial Sequence Synthetic oligonucleotide
agucgguaaguucuguccaagcccg

SEQ ID NO: 476 - RNA, 25 nt, Artificial Sequence
  Description of Artificial Sequence Synthetic oligonucleotide
cagucgguaaguucuguccaagccc

SEQ ID NO: 477 - RNA, 25 nt, Artificial Sequence
  Description of Artificial Sequence Synthetic oligonucleotide
aaagccagucgguaaguucugucca

SEQ ID NO: 478 - RNA, 25 nt, Artificial Sequence
  Description of Artificial Sequence Synthetic oligonucleotide
gaaagccagucgguaaguucugucc

SEQ ID NO: 479 - RNA, 25 nt, Artificial Sequence
  Description of Artificial Sequence Synthetic oligonucleotide
gucacccaccaucacccucugugau

SEQ ID NO: 480 - RNA, 25 nt, Artificial Sequence
  Description of Artificial Sequence Synthetic oligonucleotide
ggucacccaccaucacccucuguga

SEQ ID NO: 481 - RNA, 25 nt, Artificial Sequence
  Description of Artificial Sequence Synthetic oligonucleotide
aaggucacccaccaucacccucugu

SEQ ID NO: 482 - RNA, 25 nt, Artificial Sequence
  Description of Artificial Sequence Synthetic oligonucleotide
caaggucacccaccaucacccucug

SEQ ID NO: 483 - RNA, 25 nt, Artificial Sequence
  Description of Artificial Sequence Synthetic oligonucleotide
ucaaggucacccaccaucacccucu

SEQ ID NO: 484 - RNA, 25 nt, Artificial Sequence
  Description of Artificial Sequence Synthetic oligonucleotide
cucaaggucacccaccaucacccuc

SEQ ID NO: 485 - RNA, 25 nt, Artificial Sequence
  Description of Artificial Sequence Synthetic oligonucleotide
cuugaucaagcagagaaagccaguc

SEQ ID NO: 486 - RNA, 25 nt, Artificial Sequence
  Description of Artificial Sequence Synthetic oligonucleotide
auaacuugaucaagcagagaaagcc

SEQ ID NO: 487 - RNA, 20 nt, Artificial Sequence
  Description of Artificial Sequence Synthetic oligonucleotide
aguaacagucugaguaggag

SEQ ID NO: 488 - RNA, 20 nt, Artificial Sequence
  Description of Artificial Sequence Synthetic oligonucleotide
gaguaacagucugaguagga

SEQ ID NO: 489 - RNA, 20 nt, Artificial Sequence
  Description of Artificial Sequence Synthetic oligonucleotide
agaguaacagucugaguagg

SEQ ID NO: 490 - RNA, 20 nt, Artificial Sequence
  Description of Artificial Sequence Synthetic oligonucleotide
cagaguaacagucugaguag

SEQ ID NO: 491 - RNA, 20 nt, Artificial Sequence
  Description of Artificial Sequence Synthetic oligonucleotide
gucaccagaguaacagucug

SEQ ID NO: 492 - RNA, 20 nt, Artificial Sequence
  Description of Artificial Sequence Synthetic oligonucleotide
ugucaccagaguaacagucu

SEQ ID NO: 493 - RNA, 20 nt, Artificial Sequence
  Description of Artificial Sequence Synthetic oligonucleotide
gugucaccagaguaacaguc

SEQ ID NO: 494 - RNA, 20 nt, Artificial Sequence
  Description of Artificial Sequence Synthetic oligonucleotide
ugugucaccagaguaacagu

SEQ ID NO: 495 - RNA, 20 nt, Artificial Sequence
  Description of Artificial Sequence Synthetic oligonucleotide
uugugucaccagaguaacag

SEQ ID NO: 496 - RNA, 20 nt, Artificial Sequence
  Description of Artificial Sequence Synthetic oligonucleotide
gguugugucaccagaguaac

SEQ ID NO: 497 - RNA, 20 nt, Artificial Sequence
  Description of Artificial Sequence Synthetic oligonucleotide
agguugugucaccagaguaa

SEQ ID NO: 498 - RNA, 20 nt, Artificial Sequence
  Description of Artificial Sequence Synthetic oligonucleotide
cagguugugucaccagagua

SEQ ID NO: 499 - RNA, 20 nt, Artificial Sequence
  Description of Artificial Sequence Synthetic oligonucleotide
acagguugugucaccagagu

SEQ ID NO: 500 - RNA, 20 nt, Artificial Sequence
  Description of Artificial Sequence Synthetic oligonucleotide
cacagguugugucaccagag

SEQ ID NO: 501 - RNA, 20 nt, Artificial Sequence
  Description of Artificial Sequence Synthetic oligonucleotide
ccacagguugugucaccaga

SEQ ID NO: 502 - RNA, 20 nt, Artificial Sequence
  Description of Artificial Sequence Synthetic oligonucleotide
accacagguugugucaccag

SEQ ID NO: 503 - RNA, 20 nt, Artificial Sequence
  Description of Artificial Sequence Synthetic oligonucleotide
aaccacagguugugucacca

SEQ ID NO: 504 - RNA, 20 nt, Artificial Sequence
  Description of Artificial Sequence Synthetic oligonucleotide
uaaccacagguugugucacc

SEQ ID NO: 505 - RNA, 20 nt, Artificial Sequence
  Description of Artificial Sequence Synthetic oligonucleotide
guaaccacagguugugucac

SEQ ID NO: 506 - RNA, 20 nt, Artificial Sequence
  Description of Artificial Sequence Synthetic oligonucleotide
aguaaccacagguuguguca

SEQ ID NO: 507 - RNA, 20 nt, Artificial Sequence
  Description of Artificial Sequence Synthetic oligonucleotide
cuuaguaaccacagguugug

SEQ ID NO: 508 - RNA, 20 nt, Artificial Sequence
  Description of Artificial Sequence Synthetic oligonucleotide
ccuuaguaaccacagguugu

SEQ ID NO: 509 - RNA, 20 nt, Artificial Sequence
  Description of Artificial Sequence Synthetic oligonucleotide
uccuuaguaaccacagguug

SEQ ID NO: 510 - RNA, 20 nt, Artificial Sequence
  Description of Artificial Sequence Synthetic oligonucleotide
uuccuuaguaaccacagguu

SEQ ID NO: 511 - RNA, 20 nt, Artificial Sequence
  Description of Artificial Sequence Synthetic oligonucleotide
uuuccuuaguaaccacaggu

SEQ ID NO: 512 - RNA, 20 nt, Artificial Sequence
  Description of Artificial Sequence Synthetic oligonucleotide
guuuccuuaguaaccacagg

SEQ ID NO: 513 - RNA, 20 nt, Artificial Sequence
  Description of Artificial Sequence Synthetic oligonucleotide
aguuuccuuaguaaccacag

SEQ ID NO: 514 - RNA, 20 nt, Artificial Sequence
  Description of Artificial Sequence Synthetic oligonucleotide
gcaguuuccuuaguaaccac

SEQ ID NO: 515 - RNA, 20 nt, Artificial Sequence
  Description of Artificial Sequence Synthetic oligonucleotide
ggcaguuuccuuaguaacca

SEQ ID NO: 516 - RNA, 20 nt, Artificial Sequence
  Description of Artificial Sequence Synthetic oligonucleotide
uggcaguuuccuuaguaacc

SEQ ID NO: 517 - RNA, 20 nt, Artificial Sequence
  Description of Artificial Sequence Synthetic oligonucleotide
auggcaguuuccuuaguaac

SEQ ID NO: 518 - RNA, 20 nt, Artificial Sequence
  Description of Artificial Sequence Synthetic oligonucleotide
gauggcaguuuccuuaguaa

SEQ ID NO: 519 - RNA, 20 nt, Artificial Sequence
  Description of Artificial Sequence Synthetic oligonucleotide
agauggcaguuuccuuagua

SEQ ID NO: 520 - RNA, 20 nt, Artificial Sequence
  Description of Artificial Sequence Synthetic oligonucleotide
ggagauggcaguuuccuuag

SEQ ID NO: 521 - RNA, 20 nt, Artificial Sequence
  Description of Artificial Sequence Synthetic oligonucleotide
uggagauggcaguuuccuua

SEQ ID NO: 522 - RNA, 20 nt, Artificial Sequence
  Description of Artificial Sequence Synthetic oligonucleotide
uuggagauggcaguuuccuu

SEQ ID NO: 523 - RNA, 20 nt, Artificial Sequence
  Description of Artificial Sequence Synthetic oligonucleotide
uuuggagauggcaguuuccu

SEQ ID NO: 524 - RNA, 20 nt, Artificial Sequence
  Description of Artificial Sequence Synthetic oligonucleotide
guuuggagauggcaguuucc

SEQ ID NO: 525 - RNA, 20 nt, Artificial Sequence
  Description of Artificial Sequence Synthetic oligonucleotide
cuaguuuggagauggcaguu

SEQ ID NO: 526 - RNA, 20 nt, Artificial Sequence
  Description of Artificial Sequence Synthetic oligonucleotide
ucuaguuuggagauggcagu

SEQ ID NO: 527 - RNA, 20 nt, Artificial Sequence
  Description of Artificial Sequence Synthetic oligonucleotide
auuucuaguuuggagauggc

SEQ ID NO: 528 - RNA, 20 nt, Artificial Sequence
  Description of Artificial Sequence Synthetic oligonucleotide
uggcauuucuaguuuggaga

SEQ ID NO: 529 - RNA, 20 nt, Artificial Sequence
  Description of Artificial Sequence Synthetic oligonucleotide
gauggcauuucuaguuugga

SEQ ID NO: 530 - RNA, 20 nt, Artificial Sequence
  Description of Artificial Sequence Synthetic oligonucleotide
agauggcauuucuaguuugg

SEQ ID NO: 531 - RNA, 20 nt, Artificial Sequence
  Description of Artificial Sequence Synthetic oligonucleotide
aagauggcauuucuaguuug

SEQ ID NO: 532 - RNA, 20 nt, Artificial Sequence
  Description of Artificial Sequence Synthetic oligonucleotide
aggaagauggcauuucuagu

SEQ ID NO: 533 - RNA, 20 nt, Artificial Sequence
  Description of Artificial Sequence Synthetic oligonucleotide
aaggaagauggcauuucuag

SEQ ID NO: 534 - RNA, 20 nt, Artificial Sequence
  Description of Artificial Sequence Synthetic oligonucleotide
caaggaagauggcauuucua

SEQ ID NO: 535 - RNA, 20 nt, Artificial Sequence
  Description of Artificial Sequence Synthetic oligonucleotide
ucaaggaagauggcauuucu

SEQ ID NO: 536 - RNA, 20 nt, Artificial Sequence
  Description of Artificial Sequence Synthetic oligonucleotide
acaucaaggaagauggcauu

SEQ ID NO: 537 - RNA, 20 nt, Artificial Sequence
  Description of Artificial Sequence Synthetic oligonucleotide
caacaucaaggaagauggca

SEQ ID NO: 538 - RNA, 20 nt, Artificial Sequence
  Description of Artificial Sequence Synthetic oligonucleotide
uccaacaucaaggaagaugg

SEQ ID NO: 539 - RNA, 20 nt, Artificial Sequence
  Description of Artificial Sequence Synthetic oligonucleotide
ccuccaacaucaaggaagau

SEQ ID NO: 540 - RNA, 20 nt, Artificial Sequence
  Description of Artificial Sequence Synthetic oligonucleotide
agguaccuccaacaucaagg

SEQ ID NO: 541 - RNA, 20 nt, Artificial Sequence
  Description of Artificial Sequence Synthetic oligonucleotide
cagguaccuccaacaucaag

SEQ ID NO: 542 - RNA, 20 nt, Artificial Sequence
  Description of Artificial Sequence Synthetic oligonucleotide
agagcagguaccuccaacau

SEQ ID NO: 543 - RNA, 20 nt, Artificial Sequence
  Description of Artificial Sequence Synthetic oligonucleotide
cagagcagguaccuccaaca

SEQ ID NO: 544 - RNA, 20 nt, Artificial Sequence
  Description of Artificial Sequence Synthetic oligonucleotide
ccagagcagguaccuccaac

SEQ ID NO: 545 - RNA, 20 nt, Artificial Sequence
  Description of Artificial Sequence Synthetic oligonucleotide
gccagagcagguaccuccaa

SEQ ID NO: 546 - RNA, 20 nt, Artificial Sequence
  Description of Artificial Sequence Synthetic oligonucleotide
ugccagagcagguaccucca

SEQ ID NO: 547 - RNA, 20 nt, Artificial Sequence
  Description of Artificial Sequence Synthetic oligonucleotide
cugccagagcagguaccucc

SEQ ID NO: 548 - RNA, 20 nt, Artificial Sequence
  Description of Artificial Sequence Synthetic oligonucleotide
ucugccagagcagguaccuc

SEQ ID NO: 549 - RNA, 20 nt, Artificial Sequence
  Description of Artificial Sequence Synthetic oligonucleotide
aucugccagagcagguaccu

SEQ ID NO: 550 - RNA, 20 nt, Artificial Sequence
  Description of Artificial Sequence Synthetic oligonucleotide
uugaaaucugccagagcagg

SEQ ID NO: 551 - RNA, 20 nt, Artificial Sequence
  Description of Artificial Sequence Synthetic oligonucleotide
cccgguugaaaucugccaga

SEQ ID NO: 552 - RNA, 20 nt, Artificial Sequence
  Description of Artificial Sequence Synthetic oligonucleotide
gcccgguugaaaucugccag

SEQ ID NO: 553 - RNA, 20 nt, Artificial Sequence
  Description of Artificial Sequence Synthetic oligonucleotide
agcccgguugaaaucugcca

SEQ ID NO: 554 - RNA, 20 nt, Artificial Sequence
  Description of Artificial Sequence Synthetic oligonucleotide
ccaagcccgguugaaaucug

SEQ ID NO: 555 - RNA, 20 nt, Artificial Sequence
  Description of Artificial Sequence Synthetic oligonucleotide
uccaagcccgguugaaaucu

SEQ ID NO: 556 - RNA, 20 nt, Artificial Sequence
  Description of Artificial Sequence Synthetic oligonucleotide
guccaagcccgguugaaauc

SEQ ID NO: 557 - RNA, 20 nt, Artificial Sequence
  Description of Artificial Sequence Synthetic oligonucleotide
uguccaagcccgguugaaau

SEQ ID NO: 558 - RNA, 20 nt, Artificial Sequence
  Description of Artificial Sequence Synthetic oligonucleotide
cuguccaagcccgguugaaa

SEQ ID NO: 559 - RNA, 20 nt, Artificial Sequence
  Description of Artificial Sequence Synthetic oligonucleotide
ucuguccaagcccgguugaa

SEQ ID NO: 560 - RNA, 20 nt, Artificial Sequence
  Description of Artificial Sequence Synthetic oligonucleotide
uucuguccaagcccgguuga

SEQ ID NO: 561 - RNA, 20 nt, Artificial Sequence
  Description of Artificial Sequence Synthetic oligonucleotide
guucuguccaagcccgguug

SEQ ID NO: 562 - RNA, 20 nt, Artificial Sequence
  Description of Artificial Sequence Synthetic oligonucleotide
aguucuguccaagcccgguu

SEQ ID NO: 563 - RNA, 20 nt, Artificial Sequence
  Description of Artificial Sequence Synthetic oligonucleotide
aaguucuguccaagcccggu

SEQ ID NO: 564 - RNA, 20 nt, Artificial Sequence
  Description of Artificial Sequence Synthetic oligonucleotide
uaaguucuguccaagcccgg

SEQ ID NO: 565 - RNA, 20 nt, Artificial Sequence
  Description of Artificial Sequence Synthetic oligonucleotide
guaaguucuguccaagcccg

SEQ ID NO: 566 - RNA, 20 nt, Artificial Sequence
  Description of Artificial Sequence Synthetic oligonucleotide
gguaaguucuguccaagccc

SEQ ID NO: 567 - RNA, 20 nt, Artificial Sequence
  Description of Artificial Sequence Synthetic oligonucleotide
cagucgguaaguucugucca

SEQ ID NO: 568 - RNA, 20 nt, Artificial Sequence
  Description of Artificial Sequence Synthetic oligonucleotide
ccagucgguaaguucugucc

SEQ ID NO: 569 - RNA, 20 nt, Artificial Sequence
  Description of Artificial Sequence Synthetic oligonucleotide
ccaccaucacccucugugau

SEQ ID NO: 570 - RNA, 20 nt, Artificial Sequence
  Description of Artificial Sequence Synthetic oligonucleotide
cccaccaucacccucuguga

SEQ ID NO: 571 - RNA, 20 nt, Artificial Sequence
  Description of Artificial Sequence Synthetic oligonucleotide
cacccaccaucacccucugu

SEQ ID NO: 572 - RNA, 20 nt, Artificial Sequence
  Description of Artificial Sequence Synthetic oligonucleotide
ucacccaccaucacccucug

SEQ ID NO: 573 - RNA, 20 nt, Artificial Sequence
  Description of Artificial Sequence Synthetic oligonucleotide
gucacccaccaucacccucu

SEQ ID NO: 574 - RNA, 20 nt, Artificial Sequence
  Description of Artificial Sequence Synthetic oligonucleotide
ggucacccaccaucacccuc

SEQ ID NO: 575 - RNA, 20 nt, Artificial Sequence
  Description of Artificial Sequence Synthetic oligonucleotide
ucaagcagagaaagccaguc

SEQ ID NO: 576 - RNA, 20 nt, Artificial Sequence
  Description of Artificial Sequence Synthetic oligonucleotide
uugaucaagcagagaaagcc

SEQ ID NO: 577 - RNA, 25 nt, Artificial Sequence
  Description of Artificial Sequence Synthetic oligonucleotide
caaagaagauggcauuucuaguuug

SEQ ID NO: 578 - RNA, 20 nt, Artificial Sequence
  Description of Artificial Sequence Synthetic oligonucleotide
gcaaagaagauggcauuucu

SEQ ID NO: 579 - RNA, 20 nt, Artificial Sequence
  Description of Artificial Sequence Synthetic oligonucleotide
gcaaagaagauggcauuucu

SEQ ID NO: 580 - RNA, 20 nt, Artificial Sequence
  Description of Artificial Sequence Synthetic oligonucleotide
ucaaggaagauggcauuucu

SEQ ID NO: 581 - RNA, 20 nt, Artificial Sequence
  Description of Artificial Sequence Synthetic oligonucleotide
ucaaggaagauggcauuucu

SEQ ID NO: 582 - RNA, 20 nt, Artificial Sequence
  Description of Artificial Sequence Synthetic oligonucleotide
ucaaggaagauggcauuucu

SEQ ID NO: 583 - RNA, 20 nt, Artificial Sequence
  Description of Artificial Sequence Synthetic oligonucleotide
ucaaggaagauggcauuucu

SEQ ID NO: 584 - RNA, 20 nt, Artificial Sequence
  Description of Artificial Sequence Synthetic oligonucleotide
ucaaggaagauggcauuucu

SEQ ID NO: 585 - RNA, 20 nt, Artificial Sequence
  Description of Artificial Sequence Synthetic oligonucleotide
ucaaggaagauggcauuucu

SEQ ID NO: 586 - RNA, 20 nt, Artificial Sequence
  Description of Artificial Sequence Synthetic oligonucleotide
ucaaggaagauggcauuucu

SEQ ID NO: 587 - RNA, 20 nt, Artificial Sequence
  Description of Artificial Sequence Synthetic oligonucleotide
ucaaggaagauggcauuucu

SEQ ID NO: 588 - RNA, 20 nt, Artificial Sequence
  Description of Artificial Sequence Synthetic oligonucleotide
ucaaggaagauggcauuucu

SEQ ID NO: 589 - RNA, 20 nt, Artificial Sequence
  Description of Artificial Sequence Synthetic oligonucleotide
ucaaggaagauggcauuucu

SEQ ID NO: 590 - RNA, 20 nt, Artificial Sequence
  Description of Artificial Sequence Synthetic oligonucleotide
ucaaggaagauggcauuucu

SEQ ID NO: 591 - RNA, 20 nt, Artificial Sequence
  Description of Artificial Sequence Synthetic oligonucleotide
ucaaggaagauggcauuucu

SEQ ID NO: 592 - RNA, 20 nt, Artificial Sequence
  Description of Artificial Sequence Synthetic oligonucleotide
ucaaggaagauggcauuucu

SEQ ID NO: 593 - RNA, 20 nt, Artificial Sequence
  Description of Artificial Sequence Synthetic oligonucleotide
ucaaggaagauggcauuucu

SEQ ID NO: 594 - RNA, 20 nt, Artificial Sequence
  Description of Artificial Sequence Synthetic oligonucleotide
ucaaggaagauggcauuucu

SEQ ID NO: 595 - RNA, 20 nt, Artificial Sequence
  Description of Artificial Sequence Synthetic oligonucleotide
ucaaggaagauggcauuucu

SEQ ID NO: 596 - RNA, 20 nt, Artificial Sequence
  Description of Artificial Sequence Synthetic oligonucleotide
ucaaggaagauggcauuucu

SEQ ID NO: 597 - RNA, 20 nt, Artificial Sequence
  Description of Artificial Sequence Synthetic oligonucleotide
ucaaggaagauggcauuucu

SEQ ID NO: 598 - RNA, 20 nt, Artificial Sequence
  Description of Artificial Sequence Synthetic oligonucleotide
ucaaggaagauggcauuucu

SEQ ID NO: 599 - RNA, 20 nt, Artificial Sequence
  Description of Artificial Sequence Synthetic oligonucleotide
ucaaggaagauggcauuucu

SEQ ID NO: 600 - RNA, 20 nt, Artificial Sequence
  Description of Artificial Sequence Synthetic oligonucleotide
ucaaggaagauggcauuucu

SEQ ID NO: 601 - RNA, 20 nt, Artificial Sequence
  Description of Artificial Sequence Synthetic oligonucleotide
ucaaggaagauggcauuucu

SEQ ID NO: 602 - RNA, 20 nt, Artificial Sequence
  Description of Artificial Sequence Synthetic oligonucleotide
ucaaggaagauggcauuucu

SEQ ID NO: 603 - RNA, 20 nt, Artificial Sequence
  Description of Artificial Sequence Synthetic oligonucleotide
ucaaggaagauggcauuucu

SEQ ID NO: 604 - RNA, 20 nt, Artificial Sequence
  Description of Artificial Sequence Synthetic oligonucleotide
ucaaggaagauggcauuucu

SEQ ID NO: 605 - RNA, 20 nt, Artificial Sequence
  Description of Artificial Sequence Synthetic oligonucleotide
ucaaggaagauggcauuucu

SEQ ID NO: 606 - RNA, 20 nt, Artificial Sequence
  Description of Artificial Sequence Synthetic oligonucleotide
ucaaggaagauggcauuucu

SEQ ID NO: 607 - RNA, 20 nt, Artificial Sequence
  Description of Artificial Sequence Synthetic oligonucleotide
ucaaggaagauggcauuucu

SEQ ID NO: 608 - RNA, 20 nt, Artificial Sequence
  Description of Artificial Sequence Synthetic oligonucleotide
ucaaggaagauggcauuucu

SEQ ID NO: 609 - RNA, 20 nt, Artificial Sequence
  Description of Artificial Sequence Synthetic oligonucleotide
ggccaaaccucggcuuaccu

SEQ ID NO: 610 - RNA, 20 nt, Artificial Sequence
  Description of Artificial Sequence Synthetic oligonucleotide
ggccaaaccucggcuuaccu

SEQ ID NO: 611 - RNA, 20 nt, Artificial Sequence
  Description of Artificial Sequence Synthetic oligonucleotide
ggccaaaccucggcuuaccu

SEQ ID NO: 612 - RNA, 20 nt, Artificial Sequence
  Description of Artificial Sequence Synthetic oligonucleotide
ggccaaaccucggcuuaccu

SEQ ID NO: 613 - RNA, 20 nt, Artificial Sequence
  Description of Artificial Sequence Synthetic oligonucleotide
ggccaaaccucggcuuaccu

SEQ ID NO: 614 - RNA, 20 nt, Artificial Sequence
  Description of Artificial Sequence Synthetic oligonucleotide
ggccaaaccucggcuuaccu

SEQ ID NO: 615 - RNA, 20 nt, Artificial Sequence
  Description of Artificial Sequence Synthetic oligonucleotide
ggccaaaccucggcuuaccu

SEQ ID NO: 616 - RNA, 20 nt, Artificial Sequence
  Description of Artificial Sequence Synthetic oligonucleotide
ggccaaaccucggcuuaccu

SEQ ID NO: 617 - RNA, 20 nt, Artificial Sequence
  Description of Artificial Sequence Synthetic oligonucleotide
ucaaggaagauggcauuucu

SEQ ID NO: 618 - RNA, 20 nt, Artificial Sequence
  Description of Artificial Sequence Synthetic oligonucleotide
ucaaggaagauggcauuucu

SEQ ID NO: 619 - RNA, 20 nt, Artificial Sequence
  Description of Artificial Sequence Synthetic oligonucleotide
ucaaggaagauggcauuucu

SEQ ID NO: 620 - RNA, 20 nt, Artificial Sequence
  Description of Artificial Sequence Synthetic oligonucleotide
ucaaggaagauggcauuucu

SEQ ID NO: 621 - RNA, 20 nt, Artificial Sequence
  Description of Artificial Sequence Synthetic oligonucleotide
ucaaggaagauggcauuucu

SEQ ID NO: 622 - RNA, 20 nt, Artificial Sequence
  Description of Artificial Sequence Synthetic oligonucleotide
ucaaggaagauggcauuucu

SEQ ID NO: 623 - RNA, 20 nt, Artificial Sequence
  Description of Artificial Sequence Synthetic oligonucleotide
ucaaggaagauggcauuucu

SEQ ID NO: 624 - RNA, 20 nt, Artificial Sequence
  Description of Artificial Sequence Synthetic oligonucleotide
ucaaggaagauggcauuucu

SEQ ID NO: 625 - DNA, 20 bp, Artificial Sequence
  Description of Artificial Sequence Synthetic oligonucleotide
ggccaaacctcggcttacct

SEQ ID NO: 626 - RNA, 20 nt, Artificial Sequence
  Description of Artificial Sequence Synthetic oligonucleotide
ggccaaaccucggcuuaccu

SEQ ID NO: 627 - DNA, 20 bp, Artificial Sequence
  Description of Artificial Sequence Synthetic oligonucleotide
ggccaaacctcggcttacct

SEQ ID NO: 628 - DNA, 20 bp, Artificial Sequence
  Description of Artificial Sequence Synthetic oligonucleotide
ggccaaacctcggcttacct

SEQ ID NO: 629 - DNA, 20 bp, Artificial Sequence
  Description of Artificial Sequence Synthetic oligonucleotide
  Description of Combined DNA/RNA Molecule Synthetic oligonucleotide
ggccaaaccucggcttacct

SEQ ID NO: 630 - DNA, 20 bp, Artificial Sequence
  Description of Artificial Sequence Synthetic oligonucleotide
  Description of Combined DNA/RNA Molecule Synthetic oligonucleotide
ggccaaacctcggcttaccu

SEQ ID NO: 631 - DNA, 20 bp, Artificial Sequence
  Description of Artificial Sequence Synthetic oligonucleotide
  Description of Combined DNA/RNA Molecule Synthetic oligonucleotide
ggccaaaccucggctuaccu

SEQ ID NO: 632 - DNA, 20 bp, Artificial Sequence
  Description of Artificial Sequence Synthetic oligonucleotide
  Description of Combined DNA/RNA Molecule Synthetic oligonucleotide
ggccaaacctcggcutaccu

SEQ ID NO: 633 - DNA, 20 bp, Artificial Sequence
  Description of Artificial Sequence Synthetic oligonucleotide
  Description of Combined DNA/RNA Molecule Synthetic oligonucleotide
ggccaaacctcggctuaccu

SEQ ID NO: 634 - RNA, 20 nt, Artificial Sequence
  Description of Artificial Sequence Synthetic oligonucleotide
ggccaaaccucggcuuaccu

SEQ ID NO: 635 - DNA, 20 bp, Artificial Sequence
  Description of Artificial Sequence Synthetic oligonucleotide
  Description of Combined DNA/RNA Molecule Synthetic oligonucleotide
ggccaaacctcggcutaccu

SEQ ID NO: 636 - DNA, 20 bp, Artificial Sequence
  Description of Artificial Sequence Synthetic oligonucleotide
  Description of Combined DNA/RNA Molecule Synthetic oligonucleotide
ggccaaacctcggctuaccu

SEQ ID NO: 637 - DNA, 20 bp, Artificial Sequence
  Description of Artificial Sequence Synthetic oligonucleotide
  Description of Combined DNA/RNA Molecule Synthetic oligonucleotide
ggccaaaccucggcttaccu

SEQ ID NO: 638 - DNA, 20 bp, Artificial Sequence
  Description of Artificial Sequence Synthetic oligonucleotide
  Description of Combined DNA/RNA Molecule Synthetic oligonucleotide
ggccaaaccucggcttacct

SEQ ID NO: 639 - DNA, 20 bp, Artificial Sequence
  Description of Artificial Sequence Synthetic oligonucleotide
  Description of Combined DNA/RNA Molecule Synthetic oligonucleotide
ggccaaacctcggcttaccu

SEQ ID NO: 640 - DNA, 20 bp, Artificial Sequence
  Description of Artificial Sequence Synthetic oligonucleotide
  Description of Combined DNA/RNA Molecule Synthetic oligonucleotide
ggccaaaccucggctuaccu

SEQ ID NO: 641 - DNA, 20 bp, Artificial Sequence
  Description of Artificial Sequence Synthetic oligonucleotide
  Description of Combined DNA/RNA Molecule Synthetic oligonucleotide
ggccaaacctcggcutaccu

SEQ ID NO: 642 - DNA, 20 bp, Artificial Sequence
  Description of Artificial Sequence Synthetic oligonucleotide
  Description of Combined DNA/RNA Molecule Synthetic oligonucleotide
ggccaaacctcggctuaccu

SEQ ID NO: 643 - RNA, 20 nt, Artificial Sequence
  Description of Artificial Sequence Synthetic oligonucleotide
ggccaaaccucggcuuaccu

SEQ ID NO: 644 - DNA, 20 bp, Artificial Sequence
  Description of Artificial Sequence Synthetic oligonucleotide
  Description of Combined DNA/RNA Molecule Synthetic oligonucleotide
ggccaaacctcggcutaccu

SEQ ID NO: 645 - DNA, 20 bp, Artificial Sequence
  Description of Artificial Sequence Synthetic oligonucleotide
  Description of Combined DNA/RNA Molecule Synthetic oligonucleotide
ggccaaacctcggctuaccu

SEQ ID NO: 646 - DNA, 20 bp, Artificial Sequence
  Description of Artificial Sequence Synthetic oligonucleotide
  Description of Combined DNA/RNA Molecule Synthetic oligonucleotide
ggccaaaccucggcttaccu

SEQ ID NO: 647 - DNA, 20 bp, Artificial Sequence
  Description of Artificial Sequence Synthetic oligonucleotide
  Description of Combined DNA/RNA Molecule Synthetic oligonucleotide
ggccaaaccucggcutaccu

SEQ ID NO: 648 - RNA, 20 nt, Artificial Sequence
  Description of Artificial Sequence Synthetic oligonucleotide
ggccaaaccucggcuuaccu

SEQ ID NO: 649 - RNA, 20 nt, Artificial Sequence
  Description of Artificial Sequence Synthetic oligonucleotide
ggccaaaccucggcuuaccu

SEQ ID NO: 650 - RNA, 20 nt, Artificial Sequence
  Description of Artificial Sequence Synthetic oligonucleotide
ggccaaaccucggcuuaccu

SEQ ID NO: 651 - RNA, 20 nt, Artificial Sequence
  Description of Artificial Sequence Synthetic oligonucleotide
ggccaaaccucggcuuaccu

SEQ ID NO: 652 - RNA, 20 nt, Artificial Sequence
  Description of Artificial Sequence Synthetic oligonucleotide
ggccaaaccucggcuuaccu

SEQ ID NO: 653 - RNA, 20 nt, Artificial Sequence
  Description of Artificial Sequence Synthetic oligonucleotide
ggccaaaccucggcuuaccu

SEQ ID NO: 654 - RNA, 20 nt, Artificial Sequence
  Description of Artificial Sequence Synthetic oligonucleotide
ggccaaaccucggcuuaccu

SEQ ID NO: 655 - RNA, 20 nt, Artificial Sequence
  Description of Artificial Sequence Synthetic oligonucleotide
ggccaaaccucggcuuaccu

SEQ ID NO: 656 - RNA, 20 nt, Artificial Sequence
  Description of Artificial Sequence Synthetic oligonucleotide
ggccaaaccucggcuuaccu

SEQ ID NO: 657 - RNA, 20 nt, Artificial Sequence
  Description of Artificial Sequence Synthetic oligonucleotide
ggccaaaccucggcuuaccu

SEQ ID NO: 658 - RNA, 20 nt, Artificial Sequence
  Description of Artificial Sequence Synthetic oligonucleotide
ggccaaaccucggcuuaccu

SEQ ID NO: 659 - RNA, 35 nt, Artificial Sequence
  Description of Artificial Sequence Synthetic oligonucleotide
ggccaaaccucggcuuaccugaaaggccaaaccuc

SEQ ID NO: 660 - DNA, 20 bp, Artificial Sequence
  Description of Artificial Sequence Synthetic oligonucleotide
  Description of Combined DNA/RNA Molecule Synthetic oligonucleotide
ggccaaaccucggcutaccu

SEQ ID NO: 661 - DNA, 20 bp, Artificial Sequence
  Description of Artificial Sequence Synthetic oligonucleotide
tcaaggaagatggcatttct

SEQ ID NO: 662 - RNA, 20 nt, Artificial Sequence
  Description of Artificial Sequence Synthetic oligonucleotide
ucaaggaagauggcauuucu

SEQ ID NO: 663 - DNA, 20 bp, Artificial Sequence
  Description of Artificial Sequence Synthetic oligonucleotide
tcaaggaagatggcatttct

SEQ ID NO: 664 - DNA, 20 bp, Artificial Sequence
  Description of Artificial Sequence Synthetic oligonucleotide
tcaaggaagatggcatttct

SEQ ID NO: 665 - DNA, 20 bp, Artificial Sequence
  Description of Artificial Sequence Synthetic oligonucleotide
  Description of Combined DNA/RNA Molecule Synthetic oligonucleotide
tcaaggaagauggcatttct

SEQ ID NO: 666 - DNA, 20 bp, Artificial Sequence
  Description of Artificial Sequence Synthetic oligonucleotide
  Description of Combined DNA/RNA Molecule Synthetic oligonucleotide
ucaaggaagatggcatuucu

SEQ ID NO: 667 - DNA, 20 bp, Artificial Sequence
  Description of Artificial Sequence Synthetic oligonucleotide
  Description of Combined DNA/RNA Molecule Synthetic oligonucleotide
tcaaggaagatggcautucu

SEQ ID NO: 668 - DNA, 20 bp, Artificial Sequence
  Description of Artificial Sequence Synthetic oligonucleotide
  Description of Combined DNA/RNA Molecule Synthetic oligonucleotide
ucaaggaagauggcatutcu

SEQ ID NO: 669 - DNA, 20 bp, Artificial Sequence
  Description of Artificial Sequence Synthetic oligonucleotide
  Description of Combined DNA/RNA Molecule Synthetic oligonucleotide
tcaaggaagatggcauutcu

SEQ ID NO: 670 - DNA, 20 bp, Artificial Sequence
  Description of Artificial Sequence Synthetic oligonucleotide
  Description of Combined DNA/RNA Molecule Synthetic oligonucleotide
tcaaggaagauggcauttcu

SEQ ID NO: 671 - DNA, 20 bp, Artificial Sequence
  Description of Artificial Sequence Synthetic oligonucleotide
  Description of Combined DNA/RNA Molecule Synthetic oligonucleotide
tcaaggaagatggcatttcu

SEQ ID NO: 672 - DNA, 20 bp, Artificial Sequence
  Description of Artificial Sequence Synthetic oligonucleotide
  Description of Combined DNA/RNA Molecule Synthetic oligonucleotide
tcaaggaagatggcauttcu

SEQ ID NO: 673 - DNA, 20 bp, Artificial Sequence
  Description of Artificial Sequence Synthetic oligonucleotide
  Description of Combined DNA/RNA Molecule Synthetic oligonucleotide
ucaaggaagauggcatttcu

SEQ ID NO: 674 - DNA, 20 bp, Artificial Sequence
  Description of Artificial Sequence Synthetic oligonucleotide
  Description of Combined DNA/RNA Molecule Synthetic oligonucleotide
tcaaggaagatggcatttcu

SEQ ID NO: 675 - DNA, 20 bp, Artificial Sequence
  Description of Artificial Sequence Synthetic oligonucleotide
  Description of Combined DNA/RNA Molecule Synthetic oligonucleotide
tcaaggaagauggcatttct

SEQ ID NO: 676 - DNA, 20 bp, Artificial Sequence
  Description of Artificial Sequence Synthetic oligonucleotide
  Description of Combined DNA/RNA Molecule Synthetic oligonucleotide
ucaaggaagatggcatuucu

SEQ ID NO: 677 - DNA, 20 bp, Artificial Sequence
  Description of Artificial Sequence Synthetic oligonucleotide
  Description of Combined DNA/RNA Molecule Synthetic oligonucleotide
tcaaggaagatggcautucu

SEQ ID NO: 678 - DNA, 20 bp, Artificial Sequence
  Description of Artificial Sequence Synthetic oligonucleotide
  Description of Combined DNA/RNA Molecule Synthetic oligonucleotide
ucaaggaagauggcatutcu

SEQ ID NO: 679 - DNA, 20 bp, Artificial Sequence
  Description of Artificial Sequence Synthetic oligonucleotide
  Description of Combined DNA/RNA Molecule Synthetic oligonucleotide
tcaaggaagatggcauutcu

SEQ ID NO: 680 - DNA, 20 bp, Artificial Sequence
  Description of Artificial Sequence Synthetic oligonucleotide
  Description of Combined DNA/RNA Molecule Synthetic oligonucleotide
tcaaggaagauggcauttcu

SEQ ID NO: 681 - DNA, 20 bp, Artificial Sequence
  Description of Artificial Sequence Synthetic oligonucleotide
  Description of Combined DNA/RNA Molecule Synthetic oligonucleotide
tcaaggaagatggcatttcu

SEQ ID NO: 682 - DNA, 20 bp, Artificial Sequence
  Description of Artificial Sequence Synthetic oligonucleotide
  Description of Combined DNA/RNA Molecule Synthetic oligonucleotide
tcaaggaagatggcauttcu

SEQ ID NO: 683 - DNA, 20 bp, Artificial Sequence
  Description of Artificial Sequence Synthetic oligonucleotide
  Description of Combined DNA/RNA Molecule Synthetic oligonucleotide
ucaaggaagauggcatttcu

SEQ ID NO: 684 - DNA, 20 bp, Artificial Sequence
  Description of Artificial Sequence Synthetic oligonucleotide
  Description of Combined DNA/RNA Molecule Synthetic oligonucleotide
tcaaggaagatggcatttcu

SEQ ID NO: 685 - RNA, 20 nt, Artificial Sequence
  Description of Artificial Sequence Synthetic oligonucleotide
ucaaggaagauggcauuucu

SEQ ID NO: 686 - RNA, 20 nt, Artificial Sequence
  Description of Artificial Sequence Synthetic oligonucleotide
ucaaggaagauggcauuucu

SEQ ID NO: 687 - RNA, 20 nt, Artificial Sequence
  Description of Artificial Sequence Synthetic oligonucleotide
ucaaggaagauggcauuucu

SEQ ID NO: 688 - RNA, 20 nt, Artificial Sequence
  Description of Artificial Sequence Synthetic oligonucleotide
ucaaggaagauggcauuucu

SEQ ID NO: 689 - RNA, 20 nt, Artificial Sequence
  Description of Artificial Sequence Synthetic oligonucleotide
ucaaggaagauggcauuucu

SEQ ID NO: 690 - RNA, 20 nt, Artificial Sequence
  Description of Artificial Sequence Synthetic oligonucleotide
ucaaggaagauggcauuucu

SEQ ID NO: 691 - RNA, 20 nt, Artificial Sequence
  Description of Artificial Sequence Synthetic oligonucleotide
ucaaggaagauggcauuucu

SEQ ID NO: 692 - RNA, 20 nt, Artificial Sequence
  Description of Artificial Sequence Synthetic oligonucleotide
ucaaggaagauggcauuucu

SEQ ID NO: 693 - RNA, 20 nt, Artificial Sequence
  Description of Artificial Sequence Synthetic oligonucleotide
ucaaggaagauggcauuucu

SEQ ID NO: 694 - RNA, 20 nt, Artificial Sequence
  Description of Artificial Sequence Synthetic oligonucleotide
ucaaggaagauggcauuucu

SEQ ID NO: 695 - RNA, 20 nt, Artificial Sequence
  Description of Artificial Sequence Synthetic oligonucleotide
agaaaugccaucuuccuuga

SEQ ID NO: 696 - RNA, 20 nt, Artificial Sequence
  Description of Artificial Sequence Synthetic oligonucleotide
ucaaggaagauggcauuucu

SEQ ID NO: 697 - RNA, 20 nt, Artificial Sequence
  Description of Artificial Sequence Synthetic oligonucleotide
ucaaggaagauggcauuucu

SEQ ID NO: 698 - RNA, 20 nt, Artificial Sequence
  Description of Artificial Sequence Synthetic oligonucleotide
ucaaggaagauggcauuucu

SEQ ID NO: 699 - RNA, 20 nt, Artificial Sequence
  Description of Artificial Sequence Synthetic oligonucleotide
ucaaggaagauggcauuucu

SEQ ID NO: 700 - RNA, 20 nt, Artificial Sequence
  Description of Artificial Sequence Synthetic oligonucleotide
ucaaggaagauggcauuucu

SEQ ID NO: 701 - RNA, 20 nt, Artificial Sequence
  Description of Artificial Sequence Synthetic oligonucleotide
ucaaggaagauggcauuucu

SEQ ID NO: 702 - RNA, 20 nt, Artificial Sequence
  Description of Artificial Sequence Synthetic oligonucleotide
ucaaggaagauggcauuucu

SEQ ID NO: 703 - RNA, 20 nt, Artificial Sequence
  Description of Artificial Sequence Synthetic oligonucleotide
ucaaggaagauggcauuucu

SEQ ID NO: 704 - RNA, 20 nt, Artificial Sequence
  Description of Artificial Sequence Synthetic oligonucleotide
ucaaggaagauggcauuucu

SEQ ID NO: 705 - RNA, 20 nt, Artificial Sequence
  Description of Artificial Sequence Synthetic oligonucleotide
ucaaggaagauggcauuucu

SEQ ID NO: 706 - RNA, 20 nt, Artificial Sequence
  Description of Artificial Sequence Synthetic oligonucleotide
ucaaggaagauggcauuucu

SEQ ID NO: 707 - RNA, 20 nt, Artificial Sequence
  Description of Artificial Sequence Synthetic oligonucleotide
ucaaggaagauggcauuucu

SEQ ID NO: 708 - RNA, 20 nt, Artificial Sequence
  Description of Artificial Sequence Synthetic oligonucleotide
ucaaggaagauggcauuucu

SEQ ID NO: 709 - RNA, 20 nt, Artificial Sequence
  Description of Artificial Sequence Synthetic oligonucleotide
ucaaggaagauggcauuucu

SEQ ID NO: 710 - RNA, 20 nt, Artificial Sequence
  Description of Artificial Sequence Synthetic oligonucleotide
ucaaggaagauggcauuucu

SEQ ID NO: 711 - RNA, 20 nt, Artificial Sequence
  Description of Artificial Sequence Synthetic oligonucleotide
ucaaggaagauggcauuucu

SEQ ID NO: 712 - RNA, 20 nt, Artificial Sequence
  Description of Artificial Sequence Synthetic oligonucleotide
ucaaggaagauggcauuucu

SEQ ID NO: 713 - RNA, 20 nt, Artificial Sequence
  Description of Artificial Sequence Synthetic oligonucleotide
ucaaggaagauggcauuucu

SEQ ID NO: 714 - RNA, 20 nt, Artificial Sequence
  Description of Artificial Sequence Synthetic oligonucleotide
ucaaggaagauggcauuucu

SEQ ID NO: 715 - RNA, 20 nt, Artificial Sequence
  Description of Artificial Sequence Synthetic oligonucleotide
ucaaggaagauggcauuucu

SEQ ID NO: 716 - RNA, 20 nt, Artificial Sequence
  Description of Artificial Sequence Synthetic oligonucleotide
ucaaggaagauggcauuucu

SEQ ID NO: 717 - RNA, 20 nt, Artificial Sequence
  Description of Artificial Sequence Synthetic oligonucleotide
ucaaggaagauggcauuucu

SEQ ID NO: 718 - RNA, 20 nt, Artificial Sequence
  Description of Artificial Sequence Synthetic oligonucleotide
ucaaggaagauggcauuucu

SEQ ID NO: 719 - RNA, 20 nt, Artificial Sequence
  Description of Artificial Sequence Synthetic oligonucleotide
ucaaggaagauggcauuucu

SEQ ID NO: 720 - RNA, 20 nt, Artificial Sequence
  Description of Artificial Sequence Synthetic oligonucleotide
ucaaggaagauggcauuucu

SEQ ID NO: 721 - RNA, 20 nt, Artificial Sequence
  Description of Artificial Sequence Synthetic oligonucleotide
ucaaggaagauggcauuucu

SEQ ID NO: 722 - RNA, 20 nt, Artificial Sequence
  Description of Artificial Sequence Synthetic oligonucleotide
ucaaggaagauggcauuucu

SEQ ID NO: 723 - RNA, 20 nt, Artificial Sequence
  Description of Artificial Sequence Synthetic oligonucleotide
ucaaggaagauggcauuucu

SEQ ID NO: 724 - RNA, 20 nt, Artificial Sequence
  Description of Artificial Sequence Synthetic oligonucleotide
ucaaggaagauggcauuucu

SEQ ID NO: 725 - RNA, 20 nt, Artificial Sequence
  Description of Artificial Sequence Synthetic oligonucleotide
ucaaggaagauggcauuucu

SEQ ID NO: 726 - RNA, 20 nt, Artificial Sequence
  Description of Artificial Sequence Synthetic oligonucleotide
ucaaggaagauggcauuucu

SEQ ID NO: 727 - RNA, 20 nt, Artificial Sequence
  Description of Artificial Sequence Synthetic oligonucleotide
ucaaggaagauggcauuucu

SEQ ID NO: 728 - RNA, 20 nt, Artificial Sequence
  Description of Artificial Sequence Synthetic oligonucleotide
ucaaggaagauggcauuucu

SEQ ID NO: 729 - RNA, 20 nt, Artificial Sequence
  Description of Artificial Sequence Synthetic oligonucleotide
ucaaggaagauggcauuucu

SEQ ID NO: 730 - RNA, 20 nt, Artificial Sequence
  Description of Artificial Sequence Synthetic oligonucleotide
ucaaggaagauggcauuucu

SEQ ID NO: 731 - RNA, 20 nt, Artificial Sequence
  Description of Artificial Sequence Synthetic oligonucleotide
ucaaggaagauggcauuucu

SEQ ID NO: 732 - RNA, 20 nt, Artificial Sequence
  Description of Artificial Sequence Synthetic oligonucleotide
ucaaggaagauggcauuucu

SEQ ID NO: 733 - RNA, 20 nt, Artificial Sequence
  Description of Artificial Sequence Synthetic oligonucleotide
ucaaggaagauggcauuucu

SEQ ID NO: 734 - RNA, 20 nt, Artificial Sequence
  Description of Artificial Sequence Synthetic oligonucleotide
ucaaggaagauggcauuucu

SEQ ID NO: 735 - RNA, 20 nt, Artificial Sequence
  Description of Artificial Sequence Synthetic oligonucleotide
ucaaggaagauggcauuucu

SEQ ID NO: 736 - RNA, 20 nt, Artificial Sequence
  Description of Artificial Sequence Synthetic oligonucleotide
ucaaggaagauggcauuucu

SEQ ID NO: 737 - RNA, 20 nt, Artificial Sequence
  Description of Artificial Sequence Synthetic oligonucleotide
ucaaggaagauggcauuucu

SEQ ID NO: 738 - RNA, 20 nt, Artificial Sequence
  Description of Artificial Sequence Synthetic oligonucleotide
ucaaggaagauggcauuucu

SEQ ID NO: 739 - RNA, 20 nt, Artificial Sequence
  Description of Artificial Sequence Synthetic oligonucleotide
ucaaggaagauggcauuucu

SEQ ID NO: 740 - RNA, 20 nt, Artificial Sequence
  Description of Artificial Sequence Synthetic oligonucleotide
ucaaggaagauggcauuucu

SEQ ID NO: 741 - RNA, 20 nt, Artificial Sequence
  Description of Artificial Sequence Synthetic oligonucleotide
ucaaggaagauggcauuucu

SEQ ID NO: 742 - RNA, 20 nt, Artificial Sequence
  Description of Artificial Sequence Synthetic oligonucleotide
ucaaggaagauggcauuucu

SEQ ID NO: 743 - RNA, 20 nt, Artificial Sequence
  Description of Artificial Sequence Synthetic oligonucleotide
ucaaggaagauggcauuucu

SEQ ID NO: 744 - RNA, 20 nt, Artificial Sequence
  Description of Artificial Sequence Synthetic oligonucleotide
ucaaggaagauggcauuucu

SEQ ID NO: 745 - RNA, 20 nt, Artificial Sequence
  Description of Artificial Sequence Synthetic oligonucleotide
ucaaggaagauggcauuucu

SEQ ID NO: 746 - RNA, 20 nt, Artificial Sequence
  Description of Artificial Sequence Synthetic oligonucleotide
ucaaggaagauggcauuucu

SEQ ID NO: 747 - RNA, 20 nt, Artificial Sequence
  Description of Artificial Sequence Synthetic oligonucleotide
ucaaggaagauggcauuucu

SEQ ID NO: 748 - RNA, 20 nt, Artificial Sequence
  Description of Artificial Sequence Synthetic oligonucleotide
ucaaggaagauggcauuucu

SEQ ID NO: 749 - RNA, 20 nt, Artificial Sequence
  Description of Artificial Sequence Synthetic oligonucleotide
ucaaggaagauggcauuucu

SEQ ID NO: 750 - RNA, 20 nt, Artificial Sequence
  Description of Artificial Sequence Synthetic oligonucleotide
ucaaggaagauggcauuucu

SEQ ID NO: 751 - RNA, 20 nt, Artificial Sequence
  Description of Artificial Sequence Synthetic oligonucleotide
ucaaggaagauggcauuucu

SEQ ID NO: 752 - RNA, 20 nt, Artificial Sequence
  Description of Artificial Sequence Synthetic oligonucleotide
ucaaggaagauggcauuucu

SEQ ID NO: 753 - RNA, 20 nt, Artificial Sequence
  Description of Artificial Sequence Synthetic oligonucleotide
ucaaggaagauggcauuucu

SEQ ID NO: 754 - RNA, 20 nt, Artificial Sequence
  Description of Artificial Sequence Synthetic oligonucleotide
ucaaggaagauggcauuucu

SEQ ID NO: 755 - RNA, 20 nt, Artificial Sequence
  Description of Artificial Sequence Synthetic oligonucleotide
ucaaggaagauggcauuucu

SEQ ID NO: 756 - RNA, 20 nt, Artificial Sequence
  Description of Artificial Sequence Synthetic oligonucleotide
ucaaggaagauggcauuucu

SEQ ID NO: 757 - RNA, 20 nt, Artificial Sequence
  Description of Artificial Sequence Synthetic oligonucleotide
ucaaggaagauggcauuucu

SEQ ID NO: 758 - RNA, 20 nt, Artificial Sequence
  Description of Artificial Sequence Synthetic oligonucleotide
ucaaggaagauggcauuucu

SEQ ID NO: 759 - RNA, 20 nt, Artificial Sequence
  Description of Artificial Sequence Synthetic oligonucleotide
ucaaggaagauggcauuuuu

SEQ ID NO: 760 - RNA, 20 nt, Artificial Sequence
  Description of Artificial Sequence Synthetic oligonucleotide
ucaaggaagauggcauuucu

SEQ ID NO: 761 - RNA, 20 nt, Artificial Sequence
  Description of Artificial Sequence Synthetic oligonucleotide
ucaaggaagauggcauuucu

SEQ ID NO: 762 - RNA, 20 nt, Artificial Sequence
  Description of Artificial Sequence Synthetic oligonucleotide
ucaaggaagauggcauuucu

SEQ ID NO: 763 - RNA, 20 nt, Artificial Sequence
  Description of Artificial Sequence Synthetic oligonucleotide
ucaaggaagauggcauuucu

SEQ ID NO: 764 - RNA, 20 nt, Artificial Sequence
  Description of Artificial Sequence Synthetic oligonucleotide
ucaaggaagauggcauuucu

SEQ ID NO: 765 - RNA, 20 nt, Artificial Sequence
  Description of Artificial Sequence Synthetic oligonucleotide
ucaaggaagauggcauuucu

SEQ ID NO: 766 - RNA, 20 nt, Artificial Sequence
  Description of Artificial Sequence Synthetic oligonucleotide
ucaaggaagauggcauuucu

SEQ ID NO: 767 - RNA, 20 nt, Artificial Sequence
  Description of Artificial Sequence Synthetic oligonucleotide
ucaaggaagauggcauuucu

SEQ ID NO: 768 - RNA, 20 nt, Artificial Sequence
  Description of Artificial Sequence Synthetic oligonucleotide
ucaaggaagauggcauuucu

SEQ ID NO: 769 - RNA, 20 nt, Artificial Sequence
  Description of Artificial Sequence Synthetic oligonucleotide
ucaaggaagauggcauuucu

SEQ ID NO: 770 - RNA, 20 nt, Artificial Sequence
  Description of Artificial Sequence Synthetic oligonucleotide
ucaaggaagauggcauuucu

SEQ ID NO: 771 - RNA, 20 nt, Artificial Sequence
  Description of Artificial Sequence Synthetic oligonucleotide
ucaaggaagauggcauuucu

SEQ ID NO: 772 - RNA, 20 nt, Artificial Sequence
  Description of Artificial Sequence Synthetic oligonucleotide
ucaaggaagauggcauuucu

SEQ ID NO: 773 - RNA, 20 nt, Artificial Sequence
  Description of Artificial Sequence Synthetic oligonucleotide
ucaaggaagauggcauuucu

SEQ ID NO: 774 - RNA, 20 nt, Artificial Sequence
  Description of Artificial Sequence Synthetic oligonucleotide
ucaaggaagauggcauuucu

SEQ ID NO: 775 - RNA, 20 nt, Artificial Sequence
  Description of Artificial Sequence Synthetic oligonucleotide
ucaaggaagauggcauuucu

SEQ ID NO: 776 - RNA, 20 nt, Artificial Sequence
  Description of Artificial Sequence Synthetic oligonucleotide
ucaaggaagauggcauuucu

SEQ ID NO: 777 - RNA, 20 nt, Artificial Sequence
  Description of Artificial Sequence Synthetic oligonucleotide
ucaaggaagauggcauuucu

SEQ ID NO: 778 - RNA, 20 nt, Artificial Sequence
  Description of Artificial Sequence Synthetic oligonucleotide
ucaaggaagauggcauuucu

SEQ ID NO: 779 - RNA, 20 nt, Artificial Sequence
  Description of Artificial Sequence Synthetic oligonucleotide
ucaaggaagauggcauuucu

SEQ ID NO: 780 - RNA, 20 nt, Artificial Sequence
  Description of Artificial Sequence Synthetic oligonucleotide
ucaaggaagauggcauuucu

SEQ ID NO: 781 - RNA, 20 nt, Artificial Sequence
  Description of Artificial Sequence Synthetic oligonucleotide
ucaaggaagauggcauuucu

SEQ ID NO: 782 - RNA, 20 nt, Artificial Sequence
  Description of Artificial Sequence Synthetic oligonucleotide
ucaaggaagauggcauuucu

SEQ ID NO: 783 - RNA, 20 nt, Artificial Sequence
  Description of Artificial Sequence Synthetic oligonucleotide
ucaagcaagauggcauuucu

SEQ ID NO: 784 - RNA, 19 nt, Artificial Sequence
  Description of Artificial Sequence Synthetic oligonucleotide
ucaaggaagaugcauuucu

SEQ ID NO: 785 - RNA, 20 nt, Artificial Sequence
  Description of Artificial Sequence Synthetic oligonucleotide
ucaaggaagauggcauuucu

SEQ ID NO: 786 - RNA, 20 nt, Artificial Sequence
  Description of Artificial Sequence Synthetic oligonucleotide
ucaaggaagauggcauuucu

SEQ ID NO: 787 - RNA, 20 nt, Artificial Sequence
  Description of Artificial Sequence Synthetic oligonucleotide
ucaaggaagauggcauuucu

SEQ ID NO: 788 - RNA, 20 nt, Artificial Sequence
  Description of Artificial Sequence Synthetic oligonucleotide
ucaaggaagauggcauuucu

SEQ ID NO: 789 - RNA, 20 nt, Artificial Sequence
  Description of Artificial Sequence Synthetic oligonucleotide
ucaaggaacauggcauuucu

SEQ ID NO: 790 - RNA, 20 nt, Artificial Sequence
  Description of Artificial Sequence Synthetic oligonucleotide
ucaaggaagauggcauuucu

SEQ ID NO: 791 - RNA, 20 nt, Artificial Sequence
  Description of Artificial Sequence Synthetic oligonucleotide
ucaaggaagauggcauuucu

SEQ ID NO: 792 - RNA, 20 nt, Artificial Sequence
  Description of Artificial Sequence Synthetic oligonucleotide
ucaaggaagauggcauuucu

SEQ ID NO: 793 - RNA, 20 nt, Artificial Sequence
  Description of Artificial Sequence Synthetic oligonucleotide
ucaaggaagauggcauuucu

SEQ ID NO: 794 - RNA, 20 nt, Artificial Sequence
  Description of Artificial Sequence Synthetic oligonucleotide
ucaaggaagauggcauuucu

SEQ ID NO: 795 - RNA, 20 nt, Artificial Sequence
  Description of Artificial Sequence Synthetic oligonucleotide
ucaaggaagaugccauuucu

SEQ ID NO: 796 - RNA, 20 nt, Artificial Sequence
  Description of Artificial Sequence Synthetic oligonucleotide
ucaaggaagauggcauuucu

SEQ ID NO: 797 - RNA, 20 nt, Artificial Sequence
  Description of Artificial Sequence Synthetic oligonucleotide
ucaaggaagauggcauuucu

SEQ ID NO: 798 - RNA, 20 nt, Artificial Sequence
  Description of Artificial Sequence Synthetic oligonucleotide
ucaaggaagauggcacuucu

SEQ ID NO: 799 - RNA, 20 nt, Artificial Sequence
  Description of Artificial Sequence Synthetic oligonucleotide
ucaaggaagauggcauuucu

SEQ ID NO: 800 - RNA, 20 nt, Artificial Sequence
  Description of Artificial Sequence Synthetic oligonucleotide
ucaaggaagauggcauuucu

SEQ ID NO: 801 - RNA, 20 nt, Artificial Sequence
  Description of Artificial Sequence Synthetic oligonucleotide
ucaaggaagauggcauuucu

SEQ ID NO: 802 - RNA, 20 nt, Artificial Sequence
  Description of Artificial Sequence Synthetic oligonucleotide
ucaaggaagauggcauuucu

SEQ ID NO: 803 - RNA, 20 nt, Artificial Sequence
  Description of Artificial Sequence Synthetic oligonucleotide
ucaaggaagauggcauuucu

SEQ ID NO: 804 - RNA, 20 nt, Artificial Sequence
  Description of Artificial Sequence Synthetic oligonucleotide
ucaaggaagauggcauuucu

SEQ ID NO: 805 - RNA, 20 nt, Artificial Sequence
  Description of Artificial Sequence Synthetic oligonucleotide
ucaaggaagauggcauuucu

SEQ ID NO: 806 - RNA, 20 nt, Artificial Sequence
  Description of Artificial Sequence Synthetic oligonucleotide
ucaaggaagauggcauuucu

SEQ ID NO: 807 - RNA, 20 nt, Artificial Sequence
  Description of Artificial Sequence Synthetic oligonucleotide
ucaaggaagauggcauuucu

SEQ ID NO: 808 - RNA, 20 nt, Artificial Sequence
  Description of Artificial Sequence Synthetic oligonucleotide
ucaaggaagauggcauuucu

SEQ ID NO: 809 - RNA, 20 nt, Artificial Sequence
  Description of Artificial Sequence Synthetic oligonucleotide
ucaaggaagauggcauuucu

SEQ ID NO: 810 - RNA, 20 nt, Artificial Sequence
  Description of Artificial Sequence Synthetic oligonucleotide
ucaaggaagauggcauuucu

SEQ ID NO: 811 - RNA, 20 nt, Artificial Sequence
  Description of Artificial Sequence Synthetic oligonucleotide
ucaaggaagauggcauuucu

SEQ ID NO: 812 - RNA, 20 nt, Artificial Sequence
  Description of Artificial Sequence Synthetic oligonucleotide
ucaaggaagauggcauuucu

SEQ ID NO: 813 - RNA, 20 nt, Artificial Sequence
  Description of Artificial Sequence Synthetic oligonucleotide
ucaaggaagauggcauuucu

SEQ ID NO: 814 - RNA, 20 nt, Artificial Sequence
  Description of Artificial Sequence Synthetic oligonucleotide
ucaaggaagauggcauuucu

SEQ ID NO: 815 - RNA, 20 nt, Artificial Sequence
  Description of Artificial Sequence Synthetic oligonucleotide
ucaaggaagauggcauuucu

SEQ ID NO: 816 - RNA, 20 nt, Artificial Sequence
  Description of Artificial Sequence Synthetic oligonucleotide
ucaaggaagauggcauuucu

SEQ ID NO: 817 - RNA, 20 nt, Artificial Sequence
  Description of Artificial Sequence Synthetic oligonucleotide
ucaaggaagauggcauuucu

SEQ ID NO: 818 - RNA, 20 nt, Artificial Sequence
  Description of Artificial Sequence Synthetic oligonucleotide
ucaaggaagauggcauuucu

SEQ ID NO: 819 - RNA, 20 nt, Artificial Sequence
  Description of Artificial Sequence Synthetic oligonucleotide
ucaaggaagauggcauuucu

SEQ ID NO: 820 - RNA, 20 nt, Artificial Sequence
  Description of Artificial Sequence Synthetic oligonucleotide
ucaaggaagauggcauuucu

SEQ ID NO: 821 - RNA, 20 nt, Artificial Sequence
  Description of Artificial Sequence Synthetic oligonucleotide
ucaaggaagauggcauuucu

SEQ ID NO: 822 - RNA, 20 nt, Artificial Sequence
  Description of Artificial Sequence Synthetic oligonucleotide
ucaaggaagauggcauuucu

SEQ ID NO: 823 - RNA, 20 nt, Artificial Sequence
  Description of Artificial Sequence Synthetic oligonucleotide
ucaaggaagauggcauuucu

SEQ ID NO: 824 - RNA, 20 nt, Artificial Sequence
  Description of Artificial Sequence Synthetic oligonucleotide
ucaaggaagauggcauuucu

SEQ ID NO: 825 - RNA, 20 nt, Artificial Sequence
  Description of Artificial Sequence Synthetic oligonucleotide
ucaaggaagauggcauuucu

SEQ ID NO: 826 - RNA, 20 nt, Artificial Sequence
  Description of Artificial Sequence Synthetic oligonucleotide
ucaaggaagauggcauuucu

SEQ ID NO: 827 - RNA, 20 nt, Artificial Sequence
  Description of Artificial Sequence Synthetic oligonucleotide
ucaaggaagauggcauuucu

SEQ ID NO: 828 - RNA, 20 nt, Artificial Sequence
  Description of Artificial Sequence Synthetic oligonucleotide
ucaaggaagauggcauuucu

SEQ ID NO: 829 - RNA, 20 nt, Artificial Sequence
  Description of Artificial Sequence Synthetic oligonucleotide
ucaaggaagauggcauuucu

SEQ ID NO: 830 - RNA, 20 nt, Artificial Sequence
  Description of Artificial Sequence Synthetic oligonucleotide
ucaaggaagauggcauuucu

SEQ ID NO: 831 - RNA, 20 nt, Artificial Sequence
  Description of Artificial Sequence Synthetic oligonucleotide
ucaaggaagauggcauuucu

SEQ ID NO: 832 - RNA, 20 nt, Artificial Sequence
  Description of Artificial Sequence Synthetic oligonucleotide
ucaaggaagauggcauuucu

SEQ ID NO: 833 - RNA, 20 nt, Artificial Sequence
  Description of Artificial Sequence Synthetic oligonucleotide
ucaaggaagauggcauuucu

SEQ ID NO: 834 - RNA, 20 nt, Artificial Sequence
  Description of Artificial Sequence Synthetic oligonucleotide
ucaaggaagauggcauuucu

SEQ ID NO: 835 - RNA, 20 nt, Artificial Sequence
  Description of Artificial Sequence Synthetic oligonucleotide
ucaaggaagauggcauuucu

SEQ ID NO: 836 - RNA, 20 nt, Artificial Sequence
  Description of Artificial Sequence Synthetic oligonucleotide
ucaaggaagauggcauuucu

SEQ ID NO: 837 - RNA, 20 nt, Artificial Sequence
  Description of Artificial Sequence Synthetic oligonucleotide
ucaaggaagauggcauuucu

SEQ ID NO: 838 - RNA, 20 nt, Artificial Sequence
  Description of Artificial Sequence Synthetic oligonucleotide
ucaaggaagauggcauuucu

SEQ ID NO: 839 - RNA, 20 nt, Artificial Sequence
  Description of Artificial Sequence Synthetic oligonucleotide
ucaaggaagauggcauuucu

SEQ ID NO: 840 - RNA, 20 nt, Artificial Sequence
  Description of Artificial Sequence Synthetic oligonucleotide
ucaaggaagauggcauuucu

SEQ ID NO: 841 - RNA, 20 nt, Artificial Sequence
  Description of Artificial Sequence Synthetic oligonucleotide
ucaaggaagauggcauuucu

SEQ ID NO: 842 - RNA, 20 nt, Artificial Sequence
  Description of Artificial Sequence Synthetic oligonucleotide
ucaaggaagauggcauuucu

SEQ ID NO: 843 - RNA, 20 nt, Artificial Sequence
  Description of Artificial Sequence Synthetic oligonucleotide
ucaaggaagauggcauuucu

SEQ ID NO: 844 - RNA, 20 nt, Artificial Sequence
  Description of Artificial Sequence Synthetic oligonucleotide
ucaaggaagauggcauuucu

SEQ ID NO: 845 - RNA, 20 nt, Artificial Sequence
  Description of Artificial Sequence Synthetic oligonucleotide
ucaaggaagauggcauuucu

SEQ ID NO: 846 - RNA, 20 nt, Artificial Sequence
  Description of Artificial Sequence Synthetic oligonucleotide
ucaaggaagauggcauuucu

SEQ ID NO: 847 - RNA, 20 nt, Artificial Sequence
  Description of Artificial Sequence Synthetic oligonucleotide
ucaaggaagauggcauuucu

SEQ ID NO: 848 - RNA, 20 nt, Artificial Sequence
  Description of Artificial Sequence Synthetic oligonucleotide
ucaaggaagauggcauuucu

SEQ ID NO: 849 - RNA, 20 nt, Artificial Sequence
  Description of Artificial Sequence Synthetic oligonucleotide
ucaaggaagauggcauuucu

SEQ ID NO: 850 - RNA, 20 nt, Artificial Sequence
  Description of Artificial Sequence Synthetic oligonucleotide
ucaaggaagauggcauuucu

SEQ ID NO: 851 - RNA, 20 nt, Artificial Sequence
  Description of Artificial Sequence Synthetic oligonucleotide
ucaaggaagauggcauuucu

SEQ ID NO: 852 - RNA, 20 nt, Artificial Sequence
  Description of Artificial Sequence Synthetic oligonucleotide
ucaaggaagauggcauuucu

SEQ ID NO: 853 - RNA, 20 nt, Artificial Sequence
  Description of Artificial Sequence Synthetic oligonucleotide
ucaaggaagauggcauuucu

SEQ ID NO: 854 - RNA, 20 nt, Artificial Sequence
  Description of Artificial Sequence Synthetic oligonucleotide
ucaaggaagauggcauuucu

SEQ ID NO: 855 - RNA, 20 nt, Artificial Sequence
  Description of Artificial Sequence Synthetic oligonucleotide
ucaaggaagauggcauuucu

SEQ ID NO: 856 - RNA, 20 nt, Artificial Sequence
  Description of Artificial Sequence Synthetic oligonucleotide
ucaaggaagauggcauuucu

SEQ ID NO: 857 - RNA, 20 nt, Artificial Sequence
  Description of Artificial Sequence Synthetic oligonucleotide
ucaaggaagauggcauuucu

SEQ ID NO: 858 - RNA, 20 nt, Artificial Sequence
  Description of Artificial Sequence Synthetic oligonucleotide
ucaaggaagauggcauuucu

SEQ ID NO: 859 - RNA, 20 nt, Artificial Sequence
  Description of Artificial Sequence Synthetic oligonucleotide
ucaaggaagauggcauuucu

SEQ ID NO: 860 - RNA, 20 nt, Artificial Sequence
  Description of Artificial Sequence Synthetic oligonucleotide
ucaaggaagauggcauuucu

SEQ ID NO: 861 - RNA, 20 nt, Artificial Sequence
  Description of Artificial Sequence Synthetic oligonucleotide
ucaaggaagauggcauuucu

SEQ ID NO: 862 - RNA, 20 nt, Artificial Sequence
  Description of Artificial Sequence Synthetic oligonucleotide
ucaaggaagauggcauuucu

SEQ ID NO: 863 - RNA, 20 nt, Artificial Sequence
  Description of Artificial Sequence Synthetic oligonucleotide
ucaaggaagauggcauuucu

SEQ ID NO: 864 - RNA, 20 nt, Artificial Sequence
  Description of Artificial Sequence Synthetic oligonucleotide
ucaaggaagauggcauuucu

SEQ ID NO: 865 - RNA, 20 nt, Artificial Sequence
  Description of Artificial Sequence Synthetic oligonucleotide
ucaaggaagauggcauuucu

SEQ ID NO: 866 - RNA, 20 nt, Artificial Sequence
  Description of Artificial Sequence Synthetic oligonucleotide
ucaaggaagauggcauuucu

SEQ ID NO: 867 - RNA, 20 nt, Artificial Sequence
  Description of Artificial Sequence Synthetic oligonucleotide
ucaaggaagauggcauuucu

SEQ ID NO: 868 - RNA, 20 nt, Artificial Sequence
  Description of Artificial Sequence Synthetic oligonucleotide
ucaaggaagauggcauuucu

SEQ ID NO: 869 - RNA, 20 nt, Artificial Sequence
  Description of Artificial Sequence Synthetic oligonucleotide
ucaaggaagauggcauuucu

SEQ ID NO: 870 - RNA, 20 nt, Artificial Sequence
  Description of Artificial Sequence Synthetic oligonucleotide
ucaaggaagauggcauuucu

SEQ ID NO: 871 - RNA, 20 nt, Artificial Sequence
  Description of Artificial Sequence Synthetic oligonucleotide
ucaaggaagauggcauuucu

SEQ ID NO: 872 - RNA, 20 nt, Artificial Sequence
  Description of Artificial Sequence Synthetic oligonucleotide
ucaaggaagauggcauuucu

SEQ ID NO: 873 - RNA, 20 nt, Artificial Sequence
  Description of Artificial Sequence Synthetic oligonucleotide
ucaaggaagauggcauuucu

SEQ ID NO: 874 - RNA, 20 nt, Artificial Sequence
  Description of Artificial Sequence Synthetic oligonucleotide
ucaaggaagauggcauuucu

SEQ ID NO: 875 - RNA, 20 nt, Artificial Sequence
  Description of Artificial Sequence Synthetic oligonucleotide
ucaaggaagauggcauuucu

SEQ ID NO: 876 - RNA, 20 nt, Artificial Sequence
  Description of Artificial Sequence Synthetic oligonucleotide
ucaaggaagauggcauuucu

SEQ ID NO: 877 - RNA, 20 nt, Artificial Sequence
  Description of Artificial Sequence Synthetic oligonucleotide
ucaaggaagauggcauuucu

SEQ ID NO: 878 - RNA, 20 nt, Artificial Sequence
  Description of Artificial Sequence Synthetic oligonucleotide
ucaaggaagauggcauuucu

SEQ ID NO: 879 - RNA, 20 nt, Artificial Sequence
  Description of Artificial Sequence Synthetic oligonucleotide
ucaaggaagauggcauuucu

SEQ ID NO: 880 - RNA, 20 nt, Artificial Sequence
  Description of Artificial Sequence Synthetic oligonucleotide
ucaaggaagauggcauuucu

SEQ ID NO: 881 - RNA, 20 nt, Artificial Sequence
  Description of Artificial Sequence Synthetic oligonucleotide
ucaaggaagauggcauuucu

SEQ ID NO: 882 - RNA, 20 nt, Artificial Sequence
  Description of Artificial Sequence Synthetic oligonucleotide
ucaaggaagauggcauuucu

SEQ ID NO: 883 - RNA, 20 nt, Artificial Sequence
  Description of Artificial Sequence Synthetic oligonucleotide
ucaaggaagauggcauuucu

SEQ ID NO: 884 - RNA, 20 nt, Artificial Sequence
  Description of Artificial Sequence Synthetic oligonucleotide
ucaaggaagauggcauuucu

SEQ ID NO: 885 - RNA, 17 nt, Artificial Sequence
  Description of Artificial Sequence Synthetic oligonucleotide
ucaaggaagauggcucu

SEQ ID NO: 886 - RNA, 19 nt, Artificial Sequence
  Description of Artificial Sequence Synthetic oligonucleotide
ucaagaagauggcauuucu

SEQ ID NO: 887 - RNA, 20 nt, Artificial Sequence
  Description of Artificial Sequence Synthetic oligonucleotide
ucaaggaagauggcauuucu

SEQ ID NO: 888 - RNA, 20 nt, Artificial Sequence
  Description of Artificial Sequence Synthetic oligonucleotide
ucaaggaagauggcauuucu

SEQ ID NO: 889 - RNA, 20 nt, Artificial Sequence
  Description of Artificial Sequence Synthetic oligonucleotide
ucaaggaagauggcauuucu

SEQ ID NO: 890 - RNA, 20 nt, Artificial Sequence
  Description of Artificial Sequence Synthetic oligonucleotide
ucaaggaagauggcauuucu

SEQ ID NO: 891 - RNA, 20 nt, Artificial Sequence
  Description of Artificial Sequence Synthetic oligonucleotide
ucaaggaagauggcauuucu

SEQ ID NO: 892 - RNA, 20 nt, Artificial Sequence
  Description of Artificial Sequence Synthetic oligonucleotide
ucaaggaagauggcauuucu

SEQ ID NO: 893 - RNA, 20 nt, Artificial Sequence
  Description of Artificial Sequence Synthetic oligonucleotide
ucaaggaagauggcauuucu

SEQ ID NO: 894 - RNA, 20 nt, Artificial Sequence
  Description of Artificial Sequence Synthetic oligonucleotide
ucaaggaagauggcauuucu

SEQ ID NO: 895 - RNA, 20 nt, Artificial Sequence
  Description of Artificial Sequence Synthetic oligonucleotide
ucaaggaagauggcauuucu

SEQ ID NO: 896 - RNA, 20 nt, Artificial Sequence
  Description of Artificial Sequence Synthetic oligonucleotide
ucaaggaagauggcauuucu

SEQ ID NO: 897 - RNA, 20 nt, Artificial Sequence
  Description of Artificial Sequence Synthetic oligonucleotide
ucaaggaagauggcauuucu

SEQ ID NO: 898 - RNA, 20 nt, Artificial Sequence
  Description of Artificial Sequence Synthetic oligonucleotide
ucaaggaagauggcauuucu

SEQ ID NO: 899 - RNA, 20 nt, Artificial Sequence
  Description of Artificial Sequence Synthetic oligonucleotide
ucaaggaagauggcauuucu

SEQ ID NO: 900 - RNA, 20 nt, Artificial Sequence
  Description of Artificial Sequence Synthetic oligonucleotide
ucaaggaagauggcauuucu

SEQ ID NO: 901 - RNA, 20 nt, Artificial Sequence
  Description of Artificial Sequence Synthetic oligonucleotide
ucaaggaagauggcauuucu

SEQ ID NO: 902 - RNA, 20 nt, Artificial Sequence
  Description of Artificial Sequence Synthetic oligonucleotide
ucaaggaagauggcauuucu

SEQ ID NO: 903 - RNA, 20 nt, Artificial Sequence
  Description of Artificial Sequence Synthetic oligonucleotide
ucaaggaagauggcauuucu

SEQ ID NO: 904 - RNA, 20 nt, Artificial Sequence
  Description of Artificial Sequence Synthetic oligonucleotide
ucaaggaagauggcauuucu

SEQ ID NO: 905 - RNA, 20 nt, Artificial Sequence
  Description of Artificial Sequence Synthetic oligonucleotide
ucaaggaagauggcauuucu

SEQ ID NO: 906 - RNA, 20 nt, Artificial Sequence
  Description of Artificial Sequence Synthetic oligonucleotide
ucaaggaagauggcauuucu

SEQ ID NO: 907 - RNA, 20 nt, Artificial Sequence
  Description of Artificial Sequence Synthetic oligonucleotide
ucaaggaagauggcauuucu

SEQ ID NO: 908 - RNA, 20 nt, Artificial Sequence
  Description of Artificial Sequence Synthetic oligonucleotide
ucaaggaagauggcauuucu

SEQ ID NO: 909 - RNA, 20 nt, Artificial Sequence
  Description of Artificial Sequence Synthetic oligonucleotide
ucaaggaagauggcauuucu

SEQ ID NO: 910 - RNA, 20 nt, Artificial Sequence
  Description of Artificial Sequence Synthetic oligonucleotide
ucaaggaagauggcauuucu

SEQ ID NO: 911 - RNA, 20 nt, Artificial Sequence
  Description of Artificial Sequence Synthetic oligonucleotide
ucaaggaagauggcauuucu

SEQ ID NO: 912 - RNA, 20 nt, Artificial Sequence
  Description of Artificial Sequence Synthetic oligonucleotide
ucaaggaagauggcauuucu

SEQ ID NO: 913 - RNA, 20 nt, Artificial Sequence
  Description of Artificial Sequence Synthetic oligonucleotide
ucaaggaagauggcauuucu

SEQ ID NO: 914 - RNA, 20 nt, Artificial Sequence
  Description of Artificial Sequence Synthetic oligonucleotide
ucaaggaagauggcauuucu

SEQ ID NO: 915 - RNA, 20 nt, Artificial Sequence
  Description of Artificial Sequence Synthetic oligonucleotide
ucaaggaagauggcauuucu

SEQ ID NO: 916 - RNA, 19 nt, Artificial Sequence
  Description of Artificial Sequence Synthetic oligonucleotide
ucaaggaagauggcauuuc

SEQ ID NO: 917 - RNA, 20 nt, Artificial Sequence
  Description of Artificial Sequence Synthetic oligonucleotide
ucaaggaagauggcauuucu

SEQ ID NO: 918 - RNA, 20 nt, Artificial Sequence
  Description of Artificial Sequence Synthetic oligonucleotide
ucaaggaagauggcauuucu

SEQ ID NO: 919 - RNA, 20 nt, Artificial Sequence
  Description of Artificial Sequence Synthetic oligonucleotide
ucaaggaagauggcauuucu

SEQ ID NO: 920 - RNA, 20 nt, Artificial Sequence
  Description of Artificial Sequence Synthetic oligonucleotide
ucaaggaagauggcauuucu

SEQ ID NO: 921 - RNA, 20 nt, Artificial Sequence
  Description of Artificial Sequence Synthetic oligonucleotide
ucaaggaagauggcauuucu

SEQ ID NO: 922 - RNA, 20 nt, Artificial Sequence
  Description of Artificial Sequence Synthetic oligonucleotide
ucaaggaagauggcauuucu

SEQ ID NO: 923 - RNA, 20 nt, Artificial Sequence
  Description of Artificial Sequence Synthetic oligonucleotide
ucaaggaagauggcauuucu

SEQ ID NO: 924 - RNA, 20 nt, Artificial Sequence
  Description of Artificial Sequence Synthetic oligonucleotide
ucaaggaagauggcauuucu

SEQ ID NO: 925 - RNA, 20 nt, Artificial Sequence
  Description of Artificial Sequence Synthetic oligonucleotide
ucaaggaagauggcauuucu

SEQ ID NO: 926 - RNA, 20 nt, Artificial Sequence
  Description of Artificial Sequence Synthetic oligonucleotide
ucaaggaagauggcauuucu

SEQ ID NO: 927 - RNA, 30 nt, Artificial Sequence
  Description of Artificial Sequence Synthetic oligonucleotide
gccaacugggagcuggagcgcaccaaccag

SEQ ID NO: 928 - RNA, 20 nt, Artificial Sequence
  Description of Artificial Sequence Synthetic oligonucleotide
ucaaggaagauggcauuucu

SEQ ID NO: 929 - RNA, 20 nt, Artificial Sequence
  Description of Artificial Sequence Synthetic oligonucleotide
ucaaggaagauggcauuucu

SEQ ID NO: 930 - RNA, 20 nt, Artificial Sequence
  Description of Artificial Sequence Synthetic oligonucleotide
ucaaggaagauggcauuucu

SEQ ID NO: 931 - RNA, 20 nt, Artificial Sequence
  Description of Artificial Sequence Synthetic oligonucleotide
ucaaggaagauggcauuucu

SEQ ID NO: 932 - RNA, 20 nt, Artificial Sequence
  Description of Artificial Sequence Synthetic oligonucleotide
ccuucccugaagguuccucc

SEQ ID NO: 933 - RNA, 20 nt, Artificial Sequence
  Description of Artificial Sequence Synthetic oligonucleotide
ccuucccugaagguuccucc

SEQ ID NO: 934 - RNA, 20 nt, Artificial Sequence
  Description of Artificial Sequence Synthetic oligonucleotide
ccuucccugaagguuccucc

SEQ ID NO: 935 - RNA, 20 nt, Artificial Sequence
  Description of Artificial Sequence Synthetic oligonucleotide
ccuucccugaagguuccucc

SEQ ID NO: 936 - RNA, 20 nt, Artificial Sequence
  Description of Artificial Sequence Synthetic oligonucleotide
ccuucccugaagguuccucc

SEQ ID NO: 937 - RNA, 20 nt, Artificial Sequence
  Description of Artificial Sequence Synthetic oligonucleotide
ccuucccugaagguuccucc

SEQ ID NO: 938 - RNA, 20 nt, Artificial Sequence
  Description of Artificial Sequence Synthetic oligonucleotide
ucaaggaagauggcauuucu

SEQ ID NO: 939 - RNA, 20 nt, Artificial Sequence
  Description of Artificial Sequence Synthetic oligonucleotide
ucaaggaagauggcauuucu

SEQ ID NO: 940 - RNA, 19 nt, Artificial Sequence
  Description of Artificial Sequence Synthetic oligonucleotide
ucaaggaagauggcauuuc

SEQ ID NO: 941 - RNA, 19 nt, Artificial Sequence
  Description of Artificial Sequence Synthetic oligonucleotide
ucaaggaagauggcauuuc

SEQ ID NO: 942 - RNA, 18 nt, Artificial Sequence
  Description of Artificial Sequence Synthetic oligonucleotide
ucaaggaagauggcauuu

SEQ ID NO: 943 - RNA, 19 nt, Artificial Sequence
  Description of Artificial Sequence Synthetic oligonucleotide
caaggaagauggcauuucu

SEQ ID NO: 944 - RNA, 20 nt, Artificial Sequence
  Description of Artificial Sequence Synthetic oligonucleotide
ggccaaaccucggcuuaccu

SEQ ID NO: 945 - RNA, 20 nt, Artificial Sequence
  Description of Artificial Sequence Synthetic oligonucleotide
ggccaaaccucggcuuaccu

SEQ ID NO: 946 - RNA, 20 nt, Artificial Sequence
  Description of Artificial Sequence Synthetic oligonucleotide
uucuguaagguuuuuaugug

SEQ ID NO: 947 - RNA, 20 nt, Artificial Sequence
  Description of Artificial Sequence Synthetic oligonucleotide
auuucuguaagguuuuuaug

SEQ ID NO: 948 - RNA, 20 nt, Artificial Sequence
  Description of Artificial Sequence Synthetic oligonucleotide
ccauuucuguaagguuuuua

SEQ ID NO: 949 - RNA, 20 nt, Artificial Sequence
  Description of Artificial Sequence Synthetic oligonucleotide
auccauuucuguaagguuuu

SEQ ID NO: 950 - RNA, 20 nt, Artificial Sequence
  Description of Artificial Sequence Synthetic oligonucleotide
cauccauuucuguaagguuu

SEQ ID NO: 951 - RNA, 20 nt, Artificial Sequence
  Description of Artificial Sequence Synthetic oligonucleotide
ccauccauuucuguaagguu

SEQ ID NO: 952 - RNA, 20 nt, Artificial Sequence
  Description of Artificial Sequence Synthetic oligonucleotide
gccauccauuucuguaaggu

SEQ ID NO: 953 - RNA, 20 nt, Artificial Sequence
  Description of Artificial Sequence Synthetic oligonucleotide
agccauccauuucuguaagg

SEQ ID NO: 954 - RNA, 20 nt, Artificial Sequence
  Description of Artificial Sequence Synthetic oligonucleotide
cagccauccauuucuguaag

SEQ ID NO: 955 - RNA, 20 nt, Artificial Sequence
  Description of Artificial Sequence Synthetic oligonucleotide
ucagccauccauuucuguaa

SEQ ID NO: 956 - RNA, 20 nt, Artificial Sequence
  Description of Artificial Sequence Synthetic oligonucleotide
uucagccauccauuucugua

SEQ ID NO: 957 - RNA, 20 nt, Artificial Sequence
  Description of Artificial Sequence Synthetic oligonucleotide
cuucacccauccauuucugu

SEQ ID NO: 958 - RNA, 20 nt, Artificial Sequence
  Description of Artificial Sequence Synthetic oligonucleotide
acuucagccauccauuucug

SEQ ID NO: 959 - RNA, 20 nt, Artificial Sequence
  Description of Artificial Sequence Synthetic oligonucleotide
aacuucagccauccauuucu

SEQ ID NO: 960 - RNA, 20 nt, Artificial Sequence
  Description of Artificial Sequence Synthetic oligonucleotide
caacuucagccauccauuuc

SEQ ID NO: 961 - RNA, 20 nt, Artificial Sequence
  Description of Artificial Sequence Synthetic oligonucleotide
ucaacuucagccauccauuu

SEQ ID NO: 962 - RNA, 20 nt, Artificial Sequence
  Description of Artificial Sequence Synthetic oligonucleotide
ccagggcaggccauuccucu

SEQ ID NO: 963 - RNA, 20 nt, Artificial Sequence
  Description of Artificial Sequence Synthetic oligonucleotide
cccagggcaggccauuccuc

SEQ ID NO: 964 - RNA, 20 nt, Artificial Sequence
  Description of Artificial Sequence Synthetic oligonucleotide
ccccagggcaggccauuccu

SEQ ID NO: 965 - RNA, 20 nt, Artificial Sequence
  Description of Artificial Sequence Synthetic oligonucleotide
cccccagggcaggccauucc

SEQ ID NO: 966 - RNA, 20 nt, Artificial Sequence
  Description of Artificial Sequence Synthetic oligonucleotide
ucccccagggcaggccauuc

SEQ ID NO: 967 - RNA, 20 nt, Artificial Sequence
  Description of Artificial Sequence Synthetic oligonucleotide
aucccccagggcaggccauu

SEQ ID NO: 968 - RNA, 20 nt, Artificial Sequence
  Description of Artificial Sequence Synthetic oligonucleotide
caucccccagggcaggccau

SEQ ID NO: 969 - RNA, 20 nt, Artificial Sequence
  Description of Artificial Sequence Synthetic oligonucleotide
gcaucccccagggcaggcca

SEQ ID NO: 970 - RNA, 20 nt, Artificial Sequence
  Description of Artificial Sequence Synthetic oligonucleotide
agcaucccccagggcaggcc

SEQ ID NO: 971 - RNA, 20 nt, Artificial Sequence
  Description of Artificial Sequence Synthetic oligonucleotide
cagcaucccccagggcaggc

SEQ ID NO: 972 - RNA, 20 nt, Artificial Sequence
  Description of Artificial Sequence Synthetic oligonucleotide
ucagcaucccccagggcagg

SEQ ID NO: 973 - RNA, 20 nt, Artificial Sequence
  Description of Artificial Sequence Synthetic oligonucleotide
uucagcaucccccagggcag

SEQ ID NO: 974 - RNA, 20 nt, Artificial Sequence
  Description of Artificial Sequence Synthetic oligonucleotide
uuucagcaucccccagggca

SEQ ID NO: 975 - RNA, 20 nt, Artificial Sequence
  Description of Artificial Sequence Synthetic oligonucleotide
auuucagcaucccccagggc

SEQ ID NO: 976 - RNA, 20 nt, Artificial Sequence
  Description of Artificial Sequence Synthetic oligonucleotide
gauuucagcaucccccaggg

SEQ ID NO: 977 - RNA, 20 nt, Artificial Sequence
  Description of Artificial Sequence Synthetic oligonucleotide
ggauuucagcaucccccagg

SEQ ID NO: 978 - RNA, 20 nt, Artificial Sequence
  Description of Artificial Sequence Synthetic oligonucleotide
aggauuucagcaucccccag

SEQ ID NO: 979 - RNA, 20 nt, Artificial Sequence
  Description of Artificial Sequence Synthetic oligonucleotide
caggauuucagcauccccca

SEQ ID NO: 980 - RNA, 20 nt, Artificial Sequence
  Description of Artificial Sequence Synthetic oligonucleotide
ucaggauuucagcauccccc

SEQ ID NO: 981 - RNA, 20 nt, Artificial Sequence
  Description of Artificial Sequence Synthetic oligonucleotide
uucaggauuucagcaucccc

SEQ ID NO: 982 - RNA, 20 nt, Artificial Sequence
  Description of Artificial Sequence Synthetic oligonucleotide
uuucaggauuucagcauccc

SEQ ID NO: 983 - RNA, 20 nt, Artificial Sequence
  Description of Artificial Sequence Synthetic oligonucleotide
uuuucaggauuucagcaucc

SEQ ID NO: 984 - RNA, 20 nt, Artificial Sequence
  Description of Artificial Sequence Synthetic oligonucleotide
uuuuucaggauuucagcauc

SEQ ID NO: 985 - RNA, 20 nt, Artificial Sequence
  Description of Artificial Sequence Synthetic oligonucleotide
uuuuuucaggauuucagcau

SEQ ID NO: 986 - RNA, 20 nt, Artificial Sequence
  Description of Artificial Sequence Synthetic oligonucleotide
guuuuuucaggauuucagca

SEQ ID NO: 987 - RNA, 20 nt, Artificial Sequence
  Description of Artificial Sequence Synthetic oligonucleotide
uguuuuuucaggauuucagc

SEQ ID NO: 988 - RNA, 20 nt, Artificial Sequence
  Description of Artificial Sequence Synthetic oligonucleotide
cuguuuuuucaggauuucag

SEQ ID NO: 989 - RNA, 20 nt, Artificial Sequence
  Description of Artificial Sequence Synthetic oligonucleotide
gcuguuuuuucaggauuuca

SEQ ID NO: 990 - RNA, 20 nt, Artificial Sequence
  Description of Artificial Sequence Synthetic oligonucleotide
agcuguuuuuucaggauuuc

SEQ ID NO: 991 - RNA, 20 nt, Artificial Sequence
  Description of Artificial Sequence Synthetic oligonucleotide
gagcuguuuuuucaggauuu

SEQ ID NO: 992 - RNA, 20 nt, Artificial Sequence
  Description of Artificial Sequence Synthetic oligonucleotide
ugagcuguuuuuucaggauu

SEQ ID NO: 993 - RNA, 20 nt, Artificial Sequence
  Description of Artificial Sequence Synthetic oligonucleotide
uugagcuguuuuuucaggau

SEQ ID NO: 994 - RNA, 20 nt, Artificial Sequence
  Description of Artificial Sequence Synthetic oligonucleotide
uuugagcuguuuuuucagga

SEQ ID NO: 995 - RNA, 20 nt, Artificial Sequence
  Description of Artificial Sequence Synthetic oligonucleotide
guuugagcuguuuuuucagg

SEQ ID NO: 996 - RNA, 20 nt, Artificial Sequence
  Description of Artificial Sequence Synthetic oligonucleotide
uuguuugagcuguuuuuuca

SEQ ID NO: 997 - RNA, 20 nt, Artificial Sequence
  Description of Artificial Sequence Synthetic oligonucleotide
cauuguuugagcuguuuuuu

SEQ ID NO: 998 - RNA, 20 nt, Artificial Sequence
  Description of Artificial Sequence Synthetic oligonucleotide
gcauuguuugagcuguuuuu

SEQ ID NO: 999 - RNA, 20 nt, Artificial Sequence
  Description of Artificial Sequence Synthetic oligonucleotide
ugcauuguuugagcuguuuu

SEQ ID NO: 1000 - RNA, 20 nt, Artificial Sequence
  Description of Artificial Sequence Synthetic oligonucleotide
cugcauuguuugagcuguuu

SEQ ID NO: 1001 - RNA, 20 nt, Artificial Sequence
  Description of Artificial Sequence Synthetic oligonucleotide
ucugcauuguuugagcuguu

SEQ ID NO: 1002 - RNA, 20 nt, Artificial Sequence
  Description of Artificial Sequence Synthetic oligonucleotide
cucugcauuguuugagcugu

SEQ ID NO: 1003 - RNA, 20 nt, Artificial Sequence
  Description of Artificial Sequence Synthetic oligonucleotide
acucugcauuguuugagcug

SEQ ID NO: 1004 - RNA, 20 nt, Artificial Sequence
  Description of Artificial Sequence Synthetic oligonucleotide
uacucugcauuguuugagcu

SEQ ID NO: 1005 - RNA, 20 nt, Artificial Sequence
  Description of Artificial Sequence Synthetic oligonucleotide
uuacucugcauuguuugagc

SEQ ID NO: 1006 - RNA, 20 nt, Artificial Sequence
  Description of Artificial Sequence Synthetic oligonucleotide
cuuacucugcauuguuugag

SEQ ID NO: 1007 - RNA, 20 nt, Artificial Sequence
  Description of Artificial Sequence Synthetic oligonucleotide
ucuuacucugcauuguuuga

SEQ ID NO: 1008 - RNA, 20 nt, Artificial Sequence
  Description of Artificial Sequence Synthetic oligonucleotide
aucuuacucugcauuguuug

SEQ ID NO: 1009 - RNA, 20 nt, Artificial Sequence
  Description of Artificial Sequence Synthetic oligonucleotide
aaucuuacucugcauuguuu

SEQ ID NO: 1010 - RNA, 20 nt, Artificial Sequence
  Description of Artificial Sequence Synthetic oligonucleotide
caaaucuuacucugcauugu

SEQ ID NO: 1011 - RNA, 20 nt, Artificial Sequence
  Description of Artificial Sequence Synthetic oligonucleotide
gauacaaaucuuacucugca

SEQ ID NO: 1012 - DNA, 20 bp, Artificial Sequence
  Description of Artificial Sequence Synthetic oligonucleotide
gggtcagctgccaatgctag

SEQ ID NO: 1013 - DNA, 20 bp, Artificial Sequence
  Description of Artificial Sequence Synthetic oligonucleotide
gggtcagctgccaatgctag

SEQ ID NO: 1014 - DNA, 20 bp, Artificial Sequence
  Description of Artificial Sequence Synthetic oligonucleotide
gggtcagctgccaatgctag

SEQ ID NO: 1015 - DNA, 20 bp, Artificial Sequence
  Description of Artificial Sequence Synthetic oligonucleotide
gggtcagctgccaatgctag

SEQ ID NO: 1016 - DNA, 20 bp, Artificial Sequence
  Description of Artificial Sequence Synthetic oligonucleotide
gggtcagctgccaatgctag

SEQ ID NO: 1017 - RNA, 70 nt, Artificial Sequence
  Description of Artificial Sequence Synthetic oligonucleotide
guggaauaguauaacaauaugcuaaauguuguuauaguaucccacacugagcaaugccguagucagcaau

SEQ ID NO: 1018 - RNA, 75 nt, Artificial Sequence
  Description of Artificial Sequence Synthetic oligonucleotide
guggaauaguauaacaauaugcuaaauguuguuauaguaucccacguacugagcaaugccguagucagcaaucuu

SEQ ID NO: 1019 - RNA, 66 nt, Artificial Sequence
  Description of Artificial Sequence Synthetic oligonucleotide
ggaauaguauaacaauaugcuaaauguuguuauaguaucccacugagcaaugccguagucagcaau

SEQ ID NO: 1020 - RNA, 71 nt, Artificial Sequence
  Description of Artificial Sequence Synthetic oligonucleotide
ggaauaguauaacaauaugcuaaauguuguuauaguaucccguacugagcaaugccguagucagcaaucuu

SEQ ID NO: 1021 - DNA, 31 bp, Artificial Sequence
  Description of Artificial Sequence Synthetic oligonucleotide
  Description of Combined DNA/RNA Molecule Synthetic oligonucleotide
cgcgcgttttcgcgcggcugaaccacugcac

SEQ ID NO: 1022 - DNA, 31 bp, Artificial Sequence
  Description of Artificial Sequence Synthetic oligonucleotide
  Description of Combined DNA/RNA Molecule Synthetic oligonucleotide
cgcgcgttttcgcgcggagauacucacaauu

SEQ ID NO: 1023 - DNA, 31 bp, Artificial Sequence
  Description of Artificial Sequence Synthetic oligonucleotide
  Description of Combined DNA/RNA Molecule Synthetic oligonucleotide
cgcgcgttttcgcgcgcguugaccuccacuc

SEQ ID NO: 1024 - RNA, 66 nt, Artificial Sequence
  Description of Artificial Sequence Synthetic oligonucleotide
ggaauaguauaacaauaugcuaaauguuguuauaguauccccagucccuuucucgucgauggucag

SEQ ID NO: 1025 - RNA, 66 nt, Artificial Sequence; identical to SEQ ID NO: 1024
  Description of Artificial Sequence Synthetic oligonucleotide

SEQ ID NO: 1026 - RNA, 44 nt, Artificial Sequence
  Description of Artificial Sequence Synthetic oligonucleotide
guguuggccauggaacauauaacaauaugcuaaauguuguuaua

SEQ ID NO: 1027 - RNA, 44 nt, Artificial Sequence
  Description of Artificial Sequence Synthetic oligonucleotide
uauaacaauaugcuaaauguuguuauaguguuggccauggaaca

SEQ ID NO: 1028 - RNA, 44 nt, Artificial Sequence; identical to SEQ ID NO: 1026
  Description of Artificial Sequence Synthetic oligonucleotide

SEQ ID NO: 1029 - RNA, 52 nt, Artificial Sequence
  Description of Artificial Sequence Synthetic oligonucleotide
guguuggccauggaacaauaguauaacaauaugcuaaauguuguuauaguau

SEQ ID NO: 1030 - RNA, 58 nt, Artificial Sequence
  Description of Artificial Sequence Synthetic oligonucleotide
ggaauaguauaacaauaugcuaaauguuguuauaguaucccguguuggccauggaaca

SEQ ID NO: 1031 - RNA, 58 nt, Artificial Sequence
  Description of Artificial Sequence Synthetic oligonucleotide
guguuggccauggaacaggaauaguauaacaauaugcuaaauguuguuauaguauccc

SEQ ID NO: 1032 - RNA, 58 nt, Artificial Sequence; identical to SEQ ID NO: 1030
  Description of Artificial Sequence Synthetic oligonucleotide

SEQ ID NO: 1033 - RNA, 31 nt, Artificial Sequence
  Description of Artificial Sequence Synthetic oligonucleotide
ucagucccuuucucgucgauggucagcacag

SEQ ID NO: 1034 - RNA, 31 nt, Artificial Sequence; identical to SEQ ID NO: 1033
  Description of Artificial Sequence Synthetic oligonucleotide

SEQ ID NO: 1035 - RNA, 31 nt, Artificial Sequence; identical to SEQ ID NO: 1033
  Description of Artificial Sequence Synthetic oligonucleotide

SEQ ID NO: 1036 - RNA, 31 nt, Artificial Sequence; identical to SEQ ID NO: 1033
  Description of Artificial Sequence Synthetic oligonucleotide

SEQ ID NO: 1037 - RNA, 31 nt, Artificial Sequence; identical to SEQ ID NO: 1033
  Description of Artificial Sequence Synthetic oligonucleotide

SEQ ID NO: 1038 - DNA, 31 bp, Artificial Sequence
  Description of Artificial Sequence Synthetic oligonucleotide
  Description of Combined DNA/RNA Molecule Synthetic oligonucleotide
ucagucccuuuctcgucgauggucagcacag

SEQ ID NO: 1039 - RNA, 31 nt, Artificial Sequence; identical to SEQ ID NO: 1033
  Description of Artificial Sequence Synthetic oligonucleotide

SEQ ID NO: 1040 - RNA, 31 nt, Artificial Sequence; identical to SEQ ID NO: 1033
  Description of Artificial Sequence Synthetic oligonucleotide

SEQ ID NO: 1041 - RNA, 31 nt, Artificial Sequence; identical to SEQ ID NO: 1033
  Description of Artificial Sequence Synthetic oligonucleotide

SEQ ID NO: 1042 - RNA, 31 nt, Artificial Sequence
  Description of Artificial Sequence Synthetic oligonucleotide
  modified_base (15)..(15): Inosine
ucagucccuuucucnucgauggucagcacag

SEQ ID NO: 1043 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1044 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1045 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1046 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1047 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1048 - RNA, 31 nt, Artificial Sequence
  Description of Artificial Sequence Synthetic oligonucleotide
  modified_base (13)..(13): 2,6-diamino purine
  modified_base (15)..(15): Inosine
ucagucccuuucncnucgauggucagcacag

SEQ ID NO: 1049 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1050 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1051 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1052 - RNA, 31 nt, Artificial Sequence; identical to SEQ ID NO: 1033
  Description of Artificial Sequence Synthetic oligonucleotide

SEQ ID NO: 1053 - RNA, 31 nt, Artificial Sequence; identical to SEQ ID NO: 1033
  Description of Artificial Sequence Synthetic oligonucleotide

SEQ ID NO: 1054 - RNA, 32 nt, Artificial Sequence
  Description of Artificial Sequence Synthetic oligonucleotide
ucagucccuuucucgvucgauggucagcacag

SEQ ID NO: 1055 - RNA, 31 nt, Artificial Sequence; identical to SEQ ID NO: 1033
  Description of Artificial Sequence Synthetic oligonucleotide

SEQ ID NO: 1056 - RNA, 34 nt, Artificial Sequence
  Description of Artificial Sequence Synthetic oligonucleotide
ucagucccuuucuacagaucgauggucagcacag

SEQ ID NO: 1057 - RNA, 31 nt, Artificial Sequence; identical to SEQ ID NO: 1033
  Description of Artificial Sequence Synthetic oligonucleotide

SEQ ID NO: 1058 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1059 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1060 - RNA, 31 nt, Artificial Sequence; identical to SEQ ID NO: 1042
  Description of Artificial Sequence Synthetic oligonucleotide
  modified_base (15)..(15): Inosine

SEQ ID NO: 1061 - RNA, 31 nt, Artificial Sequence; identical to SEQ ID NO: 1033
  Description of Artificial Sequence Synthetic oligonucleotide

SEQ ID NO: 1062 - RNA, 31 nt, Artificial Sequence; identical to SEQ ID NO: 1033
  Description of Artificial Sequence Synthetic oligonucleotide

SEQ ID NO: 1063 - RNA, 31 nt, Artificial Sequence; identical to SEQ ID NO: 1033
  Description of Artificial Sequence Synthetic oligonucleotide

SEQ ID NO: 1064 - RNA, 31 nt, Artificial Sequence; identical to SEQ ID NO: 1033
  Description of Artificial Sequence Synthetic oligonucleotide

SEQ ID NO: 1065 - RNA, 33 nt, Artificial Sequence
  Description of Artificial Sequence Synthetic oligonucleotide
ccugcgacacuucggcccagagcugcuccucau

SEQ ID NO: 1066 - RNA, 33 nt, Artificial Sequence; identical to SEQ ID NO: 1065
  Description of Artificial Sequence Synthetic oligonucleotide

SEQ ID NO: 1067 - RNA, 33 nt, Artificial Sequence; identical to SEQ ID NO: 1065
  Description of Artificial Sequence Synthetic oligonucleotide

SEQ ID NO: 1068 - RNA, 33 nt, Artificial Sequence; identical to SEQ ID NO: 1065
  Description of Artificial Sequence Synthetic oligonucleotide

SEQ ID NO: 1069 - RNA, 33 nt, Artificial Sequence; identical to SEQ ID NO: 1065
  Description of Artificial Sequence Synthetic oligonucleotide

SEQ ID NO: 1070 - RNA, 33 nt, Artificial Sequence; identical to SEQ ID NO: 1065
  Description of Artificial Sequence Synthetic oligonucleotide

SEQ ID NO: 1071 - RNA, 33 nt, Artificial Sequence
  Description of Artificial Sequence Synthetic oligonucleotide
  modified_base (19)..(19): Inosine
ccugcgacacuucggcccngagcugcuccucau

SEQ ID NO: 1072 - RNA, 53 nt, Artificial Sequence
  Description of Artificial Sequence Synthetic oligonucleotide
cauugaagaagauaagagaaaguacugagaaguguuggccauggaacagguag

SEQ ID NO: 1073 - RNA, 53 nt, Artificial Sequence
  Description of Artificial Sequence Synthetic oligonucleotide
  modified_base (41)..(41): 2-amino purine
cauugaagaagauaagagaaaguacugagaaguguuggccnuggaacagguag

SEQ ID NO: 1074 - RNA, 53 nt, Artificial Sequence; identical to SEQ ID NO: 1072
  Description of Artificial Sequence Synthetic oligonucleotide

SEQ ID NO: 1075 - RNA, 45 nt, Artificial Sequence
  Description of Artificial Sequence Synthetic oligonucleotide
gacugagguacuccuuagagaaaggugccacuucuuggcaaagga

SEQ ID NO: 1076 - RNA, 40 nt, Artificial Sequence
  Description of Artificial Sequence Synthetic oligonucleotide
guaggcaugggaggaaaaggugccacuucuuggcaaagga

SEQ ID NO: 1077 - RNA, 50 nt, Artificial Sequence
  Description of Artificial Sequence Synthetic oligonucleotide
cuguccaacacagccccagccuuugagaccucugcccagaguuguucucc

SEQ ID NO: 1078 - DNA, 51 bp, Artificial Sequence
  Description of Artificial Sequence Synthetic oligonucleotide
  Description of Combined DNA/RNA Molecule Synthetic oligonucleotide
cuguccaacacagccccagccuuugagaccucuguccagaguuguucucct

SEQ ID NO: 1079 - DNA, 51 bp, Artificial Sequence; identical to SEQ ID NO: 1078
  Description of Artificial Sequence Synthetic oligonucleotide
  Description of Combined DNA/RNA Molecule Synthetic oligonucleotide

SEQ ID NO: 1080 - DNA, 51 bp, Artificial Sequence; identical to SEQ ID NO: 1078
  Description of Artificial Sequence Synthetic oligonucleotide
  Description of Combined DNA/RNA Molecule Synthetic oligonucleotide

SEQ ID NO: 1081 - DNA, 51 bp, Artificial Sequence
  Description of Artificial Sequence Synthetic oligonucleotide
  Description of Combined DNA/RNA Molecule Synthetic oligonucleotide
  modified_base (38)..(38): deoxy 2-amino purine
cuguccaacacagccccagccuuugagaccucuguccngaguuguucucct

SEQ ID NO: 1082 - DNA, 51 bp, Artificial Sequence
  Description of Artificial Sequence Synthetic oligonucleotide
  Description of Combined DNA/RNA Molecule Synthetic oligonucleotide
cuguccaacacagccccagccuuugagaccucuguccagaauuguucucct

SEQ ID NO: 1083 - DNA, 51 bp, Artificial Sequence; identical to SEQ ID NO: 1078
  Description of Artificial Sequence Synthetic oligonucleotide
  Description of Combined DNA/RNA Molecule Synthetic oligonucleotide

SEQ ID NO: 1084 - DNA, 51 bp, Artificial Sequence; identical to SEQ ID NO: 1078
  Description of Artificial Sequence Synthetic oligonucleotide
  Description of Combined DNA/RNA Molecule Synthetic oligonucleotide

SEQ ID NO: 1085 - DNA, 51 bp, Artificial Sequence; identical to SEQ ID NO: 1078
  Description of Artificial Sequence Synthetic oligonucleotide
  Description of Combined DNA/RNA Molecule Synthetic oligonucleotide

SEQ ID NO: 1086 - DNA, 51 bp, Artificial Sequence; identical to SEQ ID NO: 1078
  Description of Artificial Sequence Synthetic oligonucleotide
  Description of Combined DNA/RNA Molecule Synthetic oligonucleotide

SEQ ID NO: 1087 - RNA, 50 nt, Artificial Sequence
  Description of Artificial Sequence Synthetic oligonucleotide
cuguccaacacagccccagccuuugagaccucuguccagaguuguucucc

SEQ ID NO: 1088 - RNA, 50 nt, Artificial Sequence; identical to SEQ ID NO: 1087
  Description of Artificial Sequence Synthetic oligonucleotide

SEQ ID NO: 1089 - RNA, 50 nt, Artificial Sequence; identical to SEQ ID NO: 1087
  Description of Artificial Sequence Synthetic oligonucleotide

SEQ ID NO: 1090 - RNA, 50 nt, Artificial Sequence; identical to SEQ ID NO: 1087
  Description of Artificial Sequence Synthetic oligonucleotide

SEQ ID NO: 1091 - RNA, 50 nt, Artificial Sequence; identical to SEQ ID NO: 1087
  Description of Artificial Sequence Synthetic oligonucleotide

SEQ ID NO: 1092 - RNA, 50 nt, Artificial Sequence; identical to SEQ ID NO: 1087
  Description of Artificial Sequence Synthetic oligonucleotide

SEQ ID NO: 1093 - RNA, 50 nt, Artificial Sequence; identical to SEQ ID NO: 1087
  Description of Artificial Sequence Synthetic oligonucleotide

SEQ ID NO: 1094 - RNA, 50 nt, Artificial Sequence; identical to SEQ ID NO: 1087
  Description of Artificial Sequence Synthetic oligonucleotide

SEQ ID NO: 1095 - DNA, 51 bp, Artificial Sequence; identical to SEQ ID NO: 1078
  Description of Artificial Sequence Synthetic oligonucleotide
  Description of Combined DNA/RNA Molecule Synthetic oligonucleotide

SEQ ID NO: 1096 - RNA, 43 nt, Artificial Sequence
  Description of Artificial Sequence Synthetic oligonucleotide
acacagcuccagccuuugagaccucugcccagaguuguucucc

SEQ ID NO: 1097 - RNA, 42 nt, Artificial Sequence
  Description of Artificial Sequence Synthetic oligonucleotide
cacagccccagccuuugagaccucuguccagaguuguucucc

SEQ ID NO: 1098 - RNA, 42 nt, Artificial Sequence; identical to SEQ ID NO: 1097
  Description of Artificial Sequence Synthetic oligonucleotide

SEQ ID NO: 1099 - RNA, 42 nt, Artificial Sequence
  Description of Artificial Sequence Synthetic oligonucleotide
cacagccccagccuuugagaccucugcccagaauuguucucc

SEQ ID NO: 1100 - RNA, 45 nt, Artificial Sequence
  Description of Artificial Sequence Synthetic oligonucleotide
gacugagguacuccauagggaaaggcaccacuucuuggcaaagga

SEQ ID NO: 1101 - RNA, 45 nt, Artificial Sequence; identical to SEQ ID NO: 1100
  Description of Artificial Sequence Synthetic oligonucleotide

SEQ ID NO: 1102 - RNA, 45 nt, Artificial Sequence; identical to SEQ ID NO: 1100
  Description of Artificial Sequence Synthetic oligonucleotide

SEQ ID NO: 1103 - RNA, 45 nt, Artificial Sequence; identical to SEQ ID NO: 1075
  Description of Artificial Sequence Synthetic oligonucleotide

SEQ ID NO: 1104 - RNA, 53 nt, Artificial Sequence; identical to SEQ ID NO: 1072
  Description of Artificial Sequence Synthetic oligonucleotide

SEQ ID NO: 1105 - RNA, 53 nt, Artificial Sequence; identical to SEQ ID NO: 1072
  Description of Artificial Sequence Synthetic oligonucleotide

SEQ ID NO: 1106 - RNA, 44 nt, Artificial Sequence
  Description of Artificial Sequence Synthetic oligonucleotide
guggauaguauaacaauaugcumaauguuguuauaguaucccac

SEQ ID NO: 1107 - RNA, 45 nt, Artificial Sequence
  Description of Artificial Sequence Synthetic oligonucleotide
guggasssgsasascaauaugcumaauguugsususgssscccac

SEQ ID NO: 1108 - RNA, 45 nt, Artificial Sequence
  Description of Artificial Sequence Synthetic oligonucleotide
guggaasagsasaacaauaugcumaauguuguususgusucccac

SEQ ID NO: 1109 - RNA, 44 nt, Artificial Sequence
  Description of Artificial Sequence Synthetic oligonucleotide
guggasasuauaacaauaugcumaauguuguuauagyaycccac

SEQ ID NO: 1110 - RNA, 45 nt, Artificial Sequence
  Description of Artificial Sequence Synthetic oligonucleotide
guggasssgssssssssuaugcumaaugssssssssgssscccac

SEQ ID NO: 1111 - RNA, 45 nt, Artificial Sequence
  Description of Artificial Sequence Synthetic oligonucleotide
guggaassgsasascaauaugcumaauguugsususgssucccac

SEQ ID NO: 1112 - RNA, 46 nt, Artificial Sequence
  Description of Artificial Sequence Synthetic oligonucleotide
guggsauaguauaacaauaugcumaauguuguuauaguauscccac

SEQ ID NO: 1113 - RNA, 16 nt, Artificial Sequence
  Description of Artificial Sequence Synthetic oligonucleotide
acgcaaccaagucaua

SEQ ID NO: 1114 - RNA, 16 nt, Artificial Sequence
  Description of Artificial Sequence Synthetic oligonucleotide
gcaaugccaucaccuc

SEQ ID NO: 1115 - RNA, 16 nt, Artificial Sequence
  Description of Artificial Sequence Synthetic oligonucleotide
agggguccacauggca

SEQ ID NO: 1116 - RNA, 16 nt, Artificial Sequence
  Description of Artificial Sequence Synthetic oligonucleotide
ggcuccccaggccccu

SEQ ID NO: 1117 - RNA, 16 nt, Artificial Sequence
  Description of Artificial Sequence Synthetic oligonucleotide
ugccguccaccaggau

SEQ ID NO: 1118 - RNA, 16 nt, Artificial Sequence
  Description of Artificial Sequence Synthetic oligonucleotide
cagauuccagguggga

SEQ ID NO: 1119 - RNA, 16 nt, Artificial Sequence
  Description of Artificial Sequence Synthetic oligonucleotide
ucccugccagaauaga

SEQ ID NO: 1120 - RNA, 16 nt, Artificial Sequence
  Description of Artificial Sequence Synthetic oligonucleotide
cuccgcccaccaaaug

SEQ ID NO: 1121 - RNA, 16 nt, Artificial Sequence
  Description of Artificial Sequence Synthetic oligonucleotide
cccaaaccacaacaga

SEQ ID NO: 1122 - RNA, 16 nt, Artificial Sequence
  Description of Artificial Sequence Synthetic oligonucleotide
acccacccacccaggu

SEQ ID NO: 1123 - RNA, 16 nt, Artificial Sequence
  Description of Artificial Sequence Synthetic oligonucleotide
cugccgccagcuggau

SEQ ID NO: 1124 - RNA, 16 nt, Artificial Sequence
  Description of Artificial Sequence Synthetic oligonucleotide
agggaaccagacaguu

SEQ ID NO: 1125 - RNA, 17 nt, Artificial Sequence
  Description of Artificial Sequence Synthetic oligonucleotide
uucacuuaguguaugcc

SEQ ID NO: 1126 - RNA, 17 nt, Artificial Sequence
  Description of Artificial Sequence Synthetic oligonucleotide
uucacuuaguguaugcc

SEQ ID NO: 1127 - RNA, 17 nt, Artificial Sequence
  Description of Artificial Sequence Synthetic oligonucleotide
accuccaucagugugau

SEQ ID NO: 1128 - RNA, 17 nt, Artificial Sequence
  Description of Artificial Sequence Synthetic oligonucleotide
uuuccucacuguugcaa

SEQ ID NO: 1129 - RNA, 17 nt, Artificial Sequence
  Description of Artificial Sequence Synthetic oligonucleotide
uguguauuugcugugag

SEQ ID NO: 1130 - RNA, 17 nt, Artificial Sequence
  Description of Artificial Sequence Synthetic oligonucleotide
gagguccugggggcgcu

SEQ ID NO: 1131 - RNA, 17 nt, Artificial Sequence
  Description of Artificial Sequence Synthetic oligonucleotide
gaucuucugauggccac

SEQ ID NO: 1132 - RNA, 17 nt, Artificial Sequence
  Description of Artificial Sequence Synthetic oligonucleotide
agccacaacuccgucag

SEQ ID NO: 1133 - RNA, 17 nt, Artificial Sequence
  Description of Artificial Sequence Synthetic oligonucleotide
gauuuucugauagcuac

SEQ ID NO: 1134 - RNA, 17 nt, Artificial Sequence
  Description of Artificial Sequence Synthetic oligonucleotide
ggccacaauucugucag

SEQ ID NO: 1135 - RNA, 17 nt, Artificial Sequence
  Description of Artificial Sequence Synthetic oligonucleotide
gaucuucugauggccac

SEQ ID NO: 1136 - RNA, 17 nt, Artificial Sequence
  Description of Artificial Sequence Synthetic oligonucleotide
ggccacaacuccgucag

SEQ ID NO: 1137 - RNA, 17 nt, Artificial Sequence
  Description of Artificial Sequence Synthetic oligonucleotide
gauuuucugauagcaac

SEQ ID NO: 1138 - RNA, 17 nt, Artificial Sequence
  Description of Artificial Sequence Synthetic oligonucleotide
ggcuacgacucugucaa

SEQ ID NO: 1139 - RNA, 17 nt, Artificial Sequence
  Description of Artificial Sequence Synthetic oligonucleotide
aggccgcgucguggcgg

SEQ ID NO: 1140 - RNA, 19 nt, Artificial Sequence
  Description of Artificial Sequence Synthetic oligonucleotide
ccgcuccucucagcccguc

SEQ ID NO: 1141 - RNA, 17 nt, Artificial Sequence
  Description of Artificial Sequence Synthetic oligonucleotide
acgccacagcuccaacu

SEQ ID NO: 1142 - RNA, 17 nt, Artificial Sequence
  Description of Artificial Sequence Synthetic oligonucleotide
gucucacaauugcucuc

SEQ ID NO: 1143 - RNA, 17 nt, Artificial Sequence
  Description of Artificial Sequence Synthetic oligonucleotide
gaaauacaucagauuug

SEQ ID NO: 1144 - RNA, 17 nt, Artificial Sequence
  Description of Artificial Sequence Synthetic oligonucleotide
aauuagcuucuggccau

SEQ ID NO: 1145 - RNA, 17 nt, Artificial Sequence
  Description of Artificial Sequence Synthetic oligonucleotide
gaucagcuccuggccau

SEQ ID NO: 1146 - RNA, 17 nt, Artificial Sequence
  Description of Artificial Sequence Synthetic oligonucleotide
gaucagcuucuggccau

SEQ ID NO: 1147 - RNA, 17 nt, Artificial Sequence
  Description of Artificial Sequence Synthetic oligonucleotide
gaucagcuucuggccau

SEQ ID NO: 1148 - RNA, 16 nt, Artificial Sequence
  Description of Artificial Sequence Synthetic oligonucleotide
acugccaggcaucagc

SEQ ID NO: 1149 - RNA, 17 nt, Artificial Sequence
  Description of Artificial Sequence Synthetic oligonucleotide
cacugccgggcaucagc

SEQ ID NO: 1150 - RNA, 17 nt, Artificial Sequence
  Description of Artificial Sequence Synthetic oligonucleotide
uccgcccgauccacgau

SEQ ID NO: 1151 - RNA, 17 nt, Artificial Sequence
  Description of Artificial Sequence Synthetic oligonucleotide
ccuuucugucgaugguc

SEQ ID NO: 1152 - RNA, 17 nt, Artificial Sequence
  Description of Artificial Sequence Synthetic oligonucleotide
ccuuucugucgaugguc

SEQ ID NO: 1153 - RNA, 17 nt, Artificial Sequence
  Description of Artificial Sequence Synthetic oligonucleotide
cuugauaauccaguucc

SEQ ID NO: 1154 - RNA, 17 nt, Artificial Sequence
  Description of Artificial Sequence Synthetic oligonucleotide
uuucaggauuuccuccg

SEQ ID NO: 1155 - RNA, 17 nt, Artificial Sequence
  Description of Artificial Sequence Synthetic oligonucleotide
cuucaggauggggcagc

SEQ ID NO: 1156 - RNA, 17 nt, Artificial Sequence
  Description of Artificial Sequence Synthetic oligonucleotide
aggaacaaaccuuuguc

SEQ ID NO: 1157 - RNA, 17 nt, Artificial Sequence
  Description of Artificial Sequence Synthetic oligonucleotide
uuucacaauccaucaac

SEQ ID NO: 1158 - RNA, 17 nt, Artificial Sequence
  Description of Artificial Sequence Synthetic oligonucleotide
cuucacgauccaucaac

SEQ ID NO: 1159 - RNA, 17 nt, Artificial Sequence
  Description of Artificial Sequence Synthetic oligonucleotide
ugggacaaaccccugcc

SEQ ID NO: 1160 - RNA, 17 nt, Artificial Sequence
  Description of Artificial Sequence Synthetic oligonucleotide
cgacuccucuggauguu

SEQ ID NO: 1161 - RNA, 17 nt, Artificial Sequence
  Description of Artificial Sequence Synthetic oligonucleotide
cgacucuucuggauguu

SEQ ID NO: 1162 - RNA, 85 nt, Artificial Sequence
  Description of Artificial Sequence Synthetic oligonucleotide
guggaauaguauaacaauaugcuaaauguuguuauaguaucccacgugcagccagccguccucuagagggcccugaagagggccc

SEQ ID NO: 1163 - RNA, 85 nt, Artificial Sequence
  Description of Artificial Sequence Synthetic oligonucleotide
guggaagaggagaacaauaugcuaaauguuguucucgucucccacgugcagccagccguccucuagagggcccugaagagggccc

SEQ ID NO: 1164 - RNA, 95 nt, Artificial Sequence
  Description of Artificial Sequence Synthetic oligonucleotide
guggucgagaagaggagaacaauaugcuaaauguuguucucgucuccucgaccacgugcagccagccguccucuagagggcccugaagagggccc

SEQ ID NO: 1165 - RNA, 18 nt, Artificial Sequence
  Description of Artificial Sequence Synthetic oligonucleotide
gcaaugccaucaccuccc

SEQ ID NO: 1166 - RNA, 63 nt, Artificial Sequence
  Description of Artificial Sequence Synthetic oligonucleotide
ggugaauaguauaacaauaugcuaaauguuguuauaguauccaccgcaaugccaucaccuccc

SEQ ID NO: 1167 - RNA, 63 nt, Artificial Sequence
  Description of Artificial Sequence Synthetic oligonucleotide
ggugaagaggagaacaauaugcuaaauguuguucucgucuccaccgcaaugccaucaccuccc

SEQ ID NO: 1168 - RNA, 73 nt, Artificial Sequence
  Description of Artificial Sequence Synthetic oligonucleotide
ggugucgagaagaggagaacaauaugcuaaauguuguucucgucuccucgacaccgcaaugccaucaccuccc

SEQ ID NO: 1169 - RNA, 18 nt, Artificial Sequence
  Description of Artificial Sequence Synthetic oligonucleotide
agggguccacauggcaac

SEQ ID NO: 1170 - RNA, 63 nt, Artificial Sequence
  Description of Artificial Sequence Synthetic oligonucleotide
ggugaauaguauaacaauaugcuaaauguuguuauaguauccaccagggguccacauggcaac

SEQ ID NO: 1171 - RNA, 63 nt, Artificial Sequence
  Description of Artificial Sequence Synthetic oligonucleotide
ggugaagaggagaacaauaugcuaaauguuguucucgucuccaccagggguccacauggcaac

SEQ ID NO: 1172 - RNA, 73 nt, Artificial Sequence
  Description of Artificial Sequence Synthetic oligonucleotide
ggugucgagaagaggagaacaauaugcuaaauguuguucucgucuccucgacaccagggguccacauggcaac

SEQ ID NO: 1173 - RNA, 23 nt, Artificial Sequence
  Description of Artificial Sequence Synthetic oligonucleotide
ggugucgagaagaggagaacaau

SEQ ID NO: 1174 - RNA, 18 nt, Artificial Sequence
  Description of Artificial Sequence Synthetic oligonucleotide
ggggugccaagcaguugg

SEQ ID NO: 1175 - RNA, 73 nt, Artificial Sequence
  Description of Artificial Sequence Synthetic oligonucleotide
ggugucgagaagaggagaacaauaugcuaaauguuguucucgucuccucgacaccggggugccaagcaguugg

SEQ ID NO: 1176 - RNA, 18 nt, Artificial Sequence
  Description of Artificial Sequence Synthetic oligonucleotide
ggggugccaagcaguugg

SEQ ID NO: 1177 - RNA, 73 nt, Artificial Sequence
  Description of Artificial Sequence Synthetic oligonucleotide
ggugucgagaagaggagaacaauaugcuaaauguuguucucgucuccucgacaccguuuuuccagacggcagg

SEQ ID NO: 1178 - RNA, 23 nt, Artificial Sequence
  Description of Artificial Sequence Synthetic oligonucleotide
ggugucgagaagaggagaacaau

SEQ ID NO: 1179 - RNA, 29 nt, Artificial Sequence
  Description of Artificial Sequence Synthetic oligonucleotide
ggugucgagaagaggagaacaauaugcua

SEQ ID NO: 1180 - RNA, 73 nt, Artificial Sequence
  Description of Artificial Sequence Synthetic oligonucleotide
ggugucgagaagaggagaacaauaugcuaaauguuguucucgucuccucgacaccccuuucucgucgaugguc

SEQ ID NO: 1181 - RNA, 31 nt, Artificial Sequence
  Description of Artificial Sequence Synthetic oligonucleotide
cauggccccagcagcuucagucccuuucucg

SEQ ID NO: 1182 - DNA, 91 bp, Artificial Sequence
  Description of Artificial Sequence Synthetic oligonucleotide
  Description of Combined DNA/RNA Molecule Synthetic oligonucleotide
ggugucgagaagaggagaacaauaugcuaaauguuguucuuccucgacacccauggccccagcagcuucagucccuuucucgucgatggtc

SEQ ID NO: 1183 - DNA, 95 bp, Artificial Sequence
  Description of Artificial Sequence Synthetic oligonucleotide
  Description of Combined DNA/RNA Molecule Synthetic oligonucleotide
ggugucgagaagaggagaacaauaugcuaaauguuguucucgucuccucgacacccauggccccagcagcuucagucccuuucucgucgatggtc

SEQ ID NO: 1184 - RNA, 23 nt, Artificial Sequence
  Description of Artificial Sequence Synthetic oligonucleotide
ggugucgagaagaggagaacaau

SEQ ID NO: 1185 - RNA, 63 nt, Artificial Sequence; identical to SEQ ID NO: 1170
  Description of Artificial Sequence Synthetic oligonucleotide

SEQ ID NO: 1186 - RNA, 63 nt, Artificial Sequence; identical to SEQ ID NO: 1171
  Description of Artificial Sequence Synthetic oligonucleotide

SEQ ID NO: 1187 - RNA, 73 nt, Artificial Sequence; identical to SEQ ID NO: 1172
  Description of Artificial Sequence Synthetic oligonucleotide

SEQ ID NO: 1188 - RNA, 64 nt, Artificial Sequence
  Description of Artificial Sequence Synthetic oligonucleotide
ggaccaacugcuuggcaccccuggccaaggucauccaugacaacuuugguaucguggaaggacc

SEQ ID NO: 1189 - RNA, 61 nt, Artificial Sequence
  Description of Artificial Sequence Synthetic oligonucleotide
gggaacuggaucuaucaagacugaguugauuucugugucugaaguguaagugaacacagaa

SEQ ID NO: 1190 - DNA, 61 bp, Artificial Sequence
  Description of Artificial Sequence Synthetic oligonucleotide
ggaccatcgacgagaaagggactgaagctgctggggccatgtttttagaggccatacccat

SEQ ID NO: 1191 - RNA, 22 nt, Artificial Sequence
  Description of Artificial Sequence Synthetic oligonucleotide
ucauuaaacgccagaguccgga

SEQ ID NO: 1192 - RNA, 22 nt, Artificial Sequence
  Description of Artificial Sequence Synthetic oligonucleotide
ucugaauaauccaggaaaagca

SEQ ID NO: 1193 - RNA, 22 nt, Artificial Sequence
  Description of Artificial Sequence Synthetic oligonucleotide
uauaggggugccaagcaguugg

SEQ ID NO: 1194 - RNA, 22 nt, Artificial Sequence
  Description of Artificial Sequence Synthetic oligonucleotide
uaugguuuuuccagacggcagg

SEQ ID NO: 1195 - RNA, 22 nt, Artificial Sequence
  Description of Artificial Sequence Synthetic oligonucleotide
ggugcagauuccaggugggacg

SEQ ID NO: 1196 - RNA, 22 nt, Artificial Sequence
  Description of Artificial Sequence Synthetic oligonucleotide
acagacuuggccacugaguggg

SEQ ID NO: 1197 - RNA, 22 nt, Artificial Sequence
  Description of Artificial Sequence Synthetic oligonucleotide
uaugugucggccacggaacagg

SEQ ID NO: 1198 - RNA, 22 nt, Artificial Sequence
  Description of Artificial Sequence Synthetic oligonucleotide
aauaagggguccacauggcaac

SEQ ID NO: 1199 - RNA, 22 nt, Artificial Sequence
  Description of Artificial Sequence Synthetic oligonucleotide
ucgagcaaugccaucaccuccc

SEQ ID NO: 1200 - RNA, 22 nt, Artificial Sequence
  Description of Artificial Sequence Synthetic oligonucleotide
uauuucccugccagaauagaug

SEQ ID NO: 1201 - RNA, 22 nt, Artificial Sequence
  Description of Artificial Sequence Synthetic oligonucleotide
gaugcuccaaccaccacaaguu

SEQ ID NO: 1202 - RNA, 22 nt, Artificial Sequence
  Description of Artificial Sequence Synthetic oligonucleotide
cgucucuugcccacgccaccag

SEQ ID NO: 1203 - RNA, 22 nt, Artificial Sequence
  Description of Artificial Sequence Synthetic oligonucleotide
gucucuugauacauccaguucc

SEQ ID NO: 1204 - RNA, 22 nt, Artificial Sequence
  Description of Artificial Sequence Synthetic oligonucleotide
cacaugggauucccauugauga

SEQ ID NO: 1205 - RNA, 22 nt, Artificial Sequence
  Description of Artificial Sequence Synthetic oligonucleotide
uaucgaccaaacccguugacuc

SEQ ID NO: 1206 - RNA, 22 nt, Artificial Sequence
  Description of Artificial Sequence Synthetic oligonucleotide
cacgucaugagcccuuccacga

SEQ ID NO: 1207 - RNA, 22 nt, Artificial Sequence
  Description of Artificial Sequence Synthetic oligonucleotide
aacgagggaucccgcuccugga

SEQ ID NO: 1208 - RNA, 22 nt, Artificial Sequence
  Description of Artificial Sequence Synthetic oligonucleotide
gaagaggcugucgucauacuuc

SEQ ID NO: 1209 - RNA, 22 nt, Artificial Sequence
  Description of Artificial Sequence Synthetic oligonucleotide
caagaggucaacgaagggguca

SEQ ID NO: 1210 - RNA, 22 nt, Artificial Sequence
  Description of Artificial Sequence Synthetic oligonucleotide
aacgccaggggcgcuaagcagu

SEQ ID NO: 1211 - RNA, 22 nt, Artificial Sequence
  Description of Artificial Sequence Synthetic oligonucleotide
uacgcauggaccguggucauga

SEQ ID NO: 1212 - RNA, 22 nt, Artificial Sequence
  Description of Artificial Sequence Synthetic oligonucleotide
uacaugacccucuuggcucccc

SEQ ID NO: 1213 - RNA, 22 nt, Artificial Sequence
  Description of Artificial Sequence Synthetic oligonucleotide
gacuagccaaacucguugucau

SEQ ID NO: 1214 - RNA, 22 nt, Artificial Sequence
  Description of Artificial Sequence Synthetic oligonucleotide
agucgccacagcuucccggagg

SEQ ID NO: 1215 - RNA, 22 nt, Artificial Sequence
  Description of Artificial Sequence Synthetic oligonucleotide
uguauauccaccuuaccagagu

SEQ ID NO: 1216 - RNA, 22 nt, Artificial Sequence
  Description of Artificial Sequence Synthetic oligonucleotide
aggaggggucucacuccuugga

SEQ ID NO: 1217 - RNA, 22 nt, Artificial Sequence
  Description of Artificial Sequence Synthetic oligonucleotide
cuaggcaacaacauccacuuua

SEQ ID NO: 1218 - RNA, 22 nt, Artificial Sequence
  Description of Artificial Sequence Synthetic oligonucleotide
ccgagcgccagcagaggcaggg

SEQ ID NO: 1219 - RNA, 22 nt, Artificial Sequence
  Description of Artificial Sequence Synthetic oligonucleotide
uaugguuuuuccagacggcagg

SEQ ID NO: 1220 - RNA, 22 nt, Artificial Sequence
  Description of Artificial Sequence Synthetic oligonucleotide
gaagaggcugucgucauacuuc

SEQ ID NO: 1221 - RNA, 22 nt, Artificial Sequence
  Description of Artificial Sequence Synthetic oligonucleotide
gaagaggcugucgucauacuuc

SEQ ID NO: 1222 - RNA, 22 nt, Artificial Sequence
  Description of Artificial Sequence Synthetic oligonucleotide
uacaugacccucuuggcucccc

SEQ ID NO: 1223 - RNA, 22 nt, Artificial Sequence
  Description of Artificial Sequence Synthetic oligonucleotide
uacaugacccucuuggcucccc

SEQ ID NO: 1224 - RNA, 22 nt, Artificial Sequence
  Description of Artificial Sequence Synthetic oligonucleotide
agucgccacagcuucccggagg

SEQ ID NO: 1225 - RNA, 22 nt, Artificial Sequence
  Description of Artificial Sequence Synthetic oligonucleotide
agucgccacagcuucccggagg

SEQ ID NO: 1226 - RNA, 22 nt, Artificial Sequence
  Description of Artificial Sequence Synthetic oligonucleotide
aggaggggucucacuccuugga

SEQ ID NO: 1227 - RNA, 22 nt, Artificial Sequence
  Description of Artificial Sequence Synthetic oligonucleotide
aggaggggucucacuccuugga

SEQ ID NO: 1228 - DNA, 55 bp, Artificial Sequence
  Description of Artificial Sequence Synthetic oligonucleotide
taatctaggaaaactgagaacagaggccctgaaaaagggccaaattcttccaccc

SEQ ID NO: 1229 - RNA, 70 nt, Artificial Sequence
  Description of Artificial Sequence Synthetic oligonucleotide
aaaccgagggaucauaggggacugaauccaccauucuucucccaaucccugcaacuccuucuuccccugc

SEQ ID NO: 1230 - RNA, 70 nt, Artificial Sequence
  Description of Artificial Sequence Synthetic oligonucleotide
ugaacagcuccucgcccuugcucacuggcagagcccuccagcaucgcgagcaggcgcugccuccuccgcc

SEQ ID NO: 1231 - RNA, 71 nt, Artificial Sequence
  Description of Artificial Sequence Synthetic oligonucleotide
ucucaguccaauguaugguccgagcacaagcucuaaucaaaguccgcggguguagaccgguugccauagga

SEQ ID NO: 1232 - RNA, 71 nt, Artificial Sequence
  Description of Artificial Sequence Synthetic oligonucleotide
acagcuccucgcccuugcucacuggcagagcccuccagcaucgcgagcaggcgcugccuccuccgccgcug

SEQ ID NO: 1233 - RNA, 71 nt, Artificial Sequence
  Description of Artificial Sequence Synthetic oligonucleotide
acagcuccucgcccuugcucacuggcagagcccucaagcaucgcgagcaggcgcugccuccuccgccgcug

SEQ ID NO: 1234 - RNA, 71 nt, Artificial Sequence
  Description of Artificial Sequence Synthetic oligonucleotide
acagcuccucgcccuugcucacuggcagagcccucuagcaucgcgagcaggcgcugccuccuccgccgcug

SEQ ID NO: 1235 - RNA, 71 nt, Artificial Sequence
  Description of Artificial Sequence Synthetic oligonucleotide
acagcuccucgcccuugcucacuggcagagcccucgagcaucgcgagcaggcgcugccuccuccgccgcug

SEQ ID NO: 1236 - RNA, 71 nt, Artificial Sequence
  Description of Artificial Sequence Synthetic oligonucleotide
acagcuccucgcccuugcucacuggcagagcccugcagcaucgcgagcaggcgcugccuccuccgccgcug

SEQ ID NO: 1237 - RNA, 71 nt, Artificial Sequence
  Description of Artificial Sequence Synthetic oligonucleotide
acagcuccucgcccuugcucacuggcagagcccuucagcaucgcgagcaggcgcugccuccuccgccgcug

SEQ ID NO: 1238 - RNA, 71 nt, Artificial Sequence
  Description of Artificial Sequence Synthetic oligonucleotide
acagcuccucgcccuugcucacuggcagagcccuacagcaucgcgagcaggcgcugccuccuccgccgcug

SEQ ID NO: 1239 - RNA, 71 nt, Artificial Sequence
  Description of Artificial Sequence Synthetic oligonucleotide
acagcuccucgcccuugcucacuggcagagcccuccugcaucgcgagcaggcgcugccuccuccgccgcug

SEQ ID NO: 1240 - RNA, 71 nt, Artificial Sequence
  Description of Artificial Sequence Synthetic oligonucleotide
acagcuccucgcccuugcucacuggcagagcccugcugcaucgcgagcaggcgcugccuccuccgccgcug

SEQ ID NO: 1241 - RNA, 71 nt, Artificial Sequence
  Description of Artificial Sequence Synthetic oligonucleotide
acagcuccucgcccuugcucacuggcagagcccuucugcaucgcgagcaggcgcugccuccuccgccgcug

SEQ ID NO: 1242 - RNA, 71 nt, Artificial Sequence
  Description of Artificial Sequence Synthetic oligonucleotide
acagcuccucgcccuugcucacuggcagagcccuacugcaucgcgagcaggcgcugccuccuccgccgcug

SEQ ID NO: 1243 - RNA, 71 nt, Artificial Sequence
  Description of Artificial Sequence Synthetic oligonucleotide
acagcuccucgcccuugcucacuggcagagcccucccgcaucgcgagcaggcgcugccuccuccgccgcug

SEQ ID NO: 1244 - RNA, 71 nt, Artificial Sequence
  Description of Artificial Sequence Synthetic oligonucleotide
acagcuccucgcccuugcucacuggcagagcccugccgcaucgcgagcaggcgcugccuccuccgccgcug

SEQ ID NO: 1245 - RNA, 71 nt, Artificial Sequence
  Description of Artificial Sequence Synthetic oligonucleotide
acagcuccucgcccuugcucacuggcagagcccuuccgcaucgcgagcaggcgcugccuccuccgccgcug

SEQ ID NO: 1246 - RNA, 71 nt, Artificial Sequence
  Description of Artificial Sequence Synthetic oligonucleotide
acagcuccucgcccuugcucacuggcagagcccuaccgcaucgcgagcaggcgcugccuccuccgccgcug

SEQ ID NO: 1247 - RNA, 71 nt, Artificial Sequence
  Description of Artificial Sequence Synthetic oligonucleotide
acagcuccucgcccuugcucacuggcagagcccuccggcaucgcgagcaggcgcugccuccuccgccgcug

SEQ ID NO: 1248 - RNA, 71 nt, Artificial Sequence; identical to SEQ ID NO: 1240
  Description of Artificial Sequence Synthetic oligonucleotide

SEQ ID NO: 1249 - RNA, 71 nt, Artificial Sequence
  Description of Artificial Sequence Synthetic oligonucleotide
acagcuccucgcccuugcucacuggcagagcccuucggcaucgcgagcaggcgcugccuccuccgccgcug

SEQ ID NO: 1250 - RNA, 71 nt, Artificial Sequence
  Description of Artificial Sequence Synthetic oligonucleotide
acagcuccucgcccuugcucacuggcagagcccuacggcaucgcgagcaggcgcugccuccuccgccgcug

SEQ ID NO: 1251 - RNA, 31 nt, Artificial Sequence
  Description of Artificial Sequence Synthetic oligonucleotide
acuggcagagcccuccagcaucgcgagcagg

SEQ ID NO: 1252 - RNA, 51 nt, Artificial Sequence
  Description of Artificial Sequence Synthetic oligonucleotide
gcccuugcucacuggcagagcccuccagcaucgcgagcaggcgcugccucc

SEQ ID NO: 1253 - RNA, 71 nt, Artificial Sequence; identical to SEQ ID NO: 1232
  Description of Artificial Sequence Synthetic oligonucleotide

SEQ ID NO: 1254 - RNA, 91 nt, Artificial Sequence
  Description of Artificial Sequence Synthetic oligonucleotide
accccggugaacagcuccucgcccuugcucacuggcagagcccuccagcaucgcgagcaggcgcugccuccuccgccgcugccuccuccgc

SEQ ID NO: 1255 - RNA, 282 nt, Artificial Sequence
  Description of Artificial Sequence Synthetic polynucleotide
gcucgaccaggaugggcaccaccccggugaacagcuccucgcccuugcucacuggcagagcccuccagcaucgcgagcaggcgcugccuccuccgccgcugccuccuccgccgcugccuccuccgcccugcucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguaca

SEQ ID NO: 1256 - RNA, 151 nt, Artificial Sequence
  Description of Artificial Sequence Synthetic polynucleotide
ucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguaca

SEQ ID NO: 1257 - RNA, 171 nt, Artificial Sequence
  Description of Artificial Sequence Synthetic polynucleotide
gccguuuacgucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguacagcucguccau

SEQ ID NO: 1258 - RNA, 191 nt, Artificial Sequence
  Description of Artificial Sequence Synthetic polynucleotide
ugaacuuguggccguuuacgucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguacagcucguccaugccgccggug

SEQ ID NO: 1259 - RNA, 211 nt, Artificial Sequence
  Description of Artificial Sequence Synthetic polynucleotide
ccggacacgcugaacuuguggccguuuacgucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguacagcucguccaugccgccgguggaguggcggc

SEQ ID NO: 1260 - RNA, 31 nt, Artificial Sequence
  Description of Artificial Sequence Synthetic oligonucleotide
gcgaccggggaucuccacagauucuuccggc

SEQ ID NO: 1261 - RNA, 51 nt, Artificial Sequence
  Description of Artificial Sequence Synthetic oligonucleotide
gcucacgguggcgaccggggaucuccacagauucuuccggcguguauaccu

SEQ ID NO: 1262 - RNA, 71 nt, Artificial Sequence
  Description of Artificial Sequence Synthetic oligonucleotide
ccucgcccuugcucacgguggcgaccggggaucuccacagauucuuccggcguguauaccuucugcugccu

SEQ ID NO: 1263 - RNA, 91 nt, Artificial Sequence
  Description of Artificial Sequence Synthetic oligonucleotide
gugaacagcuccucgcccuugcucacgguggcgaccggggaucuccacagauucuuccggcguguauaccuucugcugccuccuccgccgc

SEQ ID NO: 1264 - RNA, 111 nt, Artificial Sequence
  Description of Artificial Sequence Synthetic polynucleotide
caccaccccggugaacagcuccucgcccuugcucacgguggcgaccggggaucuccacagauucuuccggcguguauaccuucugcugccuccuccgccgcugccuccucc

SEQ ID NO: 1265 - RNA, 131 nt, Artificial Sequence
  Description of Artificial Sequence Synthetic polynucleotide
ccaggaugggcaccaccccggugaacagcuccucgcccuugcucacgguggcgaccggggaucuccacagauucuuccggcguguauaccuucugcugccuccuccgccgcugccuccuccgccgcugccu

SEQ ID NO: 1266 - RNA, 151 nt, Artificial Sequence
  Description of Artificial Sequence Synthetic polynucleotide
uccagcucgaccaggaugggcaccaccccggugaacagcuccucgcccuugcucacgguggcgaccggggaucuccacagauucuuccggcguguauaccuucugcugccuccuccgccgcugccuccuccgccgcugccuccuccgcccu

SEQ ID NO: 1267 - RNA, 171 nt, Artificial Sequence
  Description of Artificial Sequence Synthetic polynucleotide
cggcgacguauccagcucgaccaggaugggcaccaccccggugaacagcuccucgcccuugcucacgguggcgaccggggaucuccacagauucuuccggcguguauaccuucugcugccuccuccgccgcugccuccuccgccgcugccuccuccgcccugcagcuugua

SEQ ID NO: 1268 - RNA, 191 nt, Artificial Sequence
  Description of Artificial Sequence Synthetic polynucleotide
uguggccguuuacgucgccguccagcucgaccaggaugggcaccaccccggugaacagcuccucgcccuugcucacgguggcgaccggggaucuccacagauucuuccggcguguauaccuucugcugccuccuccgccgcugccuccuccgccgcugccuccuccgcccugcagcuuguacagcucgucc

SEQ ID NO: 1269 - RNA, 211 nt, Artificial Sequence
  Description of Artificial Sequence Synthetic polynucleotide
acgcugaacuuguggccguuuacgucgccguccagcucgaccaggaugggcaccaccccggugaacagcuccucgcccuugcucacgguggcgaccggggaucuccacagauucuuccggcguguauaccuucugcugccuccuccgccgcugccuccuccgccgcugccuccuccgcccugcagcuuguacagcucguccaugccgccgg

SEQ ID NO: 1270 - RNA, 71 nt, Artificial Sequence
  Description of Artificial Sequence Synthetic oligonucleotide
cagcaucgcgagcaggcgcugccuccuccgccgcugccuccuccgccgcugccuccuccgcccugcagcuu

SEQ ID NO: 1271 - RNA, 71 nt, Artificial Sequence
  Description of Artificial Sequence Synthetic oligonucleotide
cccuccagcaucgcgagcaggcgcugccuccuccgccgcugccuccuccgccgcugccuccuccgcccugc

SEQ ID NO: 1272 - RNA, 71 nt, Artificial Sequence
  Description of Artificial Sequence Synthetic oligonucleotide
cagagcccuccagcaucgcgagcaggcgcugccuccuccgccgcugccuccuccgccgcugccuccuccgc

SEQ ID NO: 1273 - RNA, 72 nt, Artificial Sequence
  Description of Artificial Sequence Synthetic oligonucleotide
acuggcagagcccucccagcaucgcgagcaggcgcugccuccuccgccgcugccuccuccgccgcugccucc

SEQ ID NO: 1274 - RNA, 71 nt, Artificial Sequence
  Description of Artificial Sequence Synthetic oligonucleotide
ugcucacuggcagagcccuccagcaucgcgagcaggcgcugccuccuccgccgcugccuccuccgccgcug

SEQ ID NO: 1275 - RNA, 71 nt, Artificial Sequence
  Description of Artificial Sequence Synthetic oligonucleotide
gcccuugcucacuggcagagcccuccagcaucgcgagcaggcgcugccuccuccgccgcugccuccuccgc

SEQ ID NO: 1276 - RNA, 71 nt, Artificial Sequence
  Description of Artificial Sequence Synthetic oligonucleotide
uccucgcccuugcucacuggcagagcccuccagcaucgcgagcaggcgcugccuccuccgccgcugccucc

SEQ ID NO: 1277 - RNA, 71 nt, Artificial Sequence
  Description of Artificial Sequence Synthetic oligonucleotide
ggugaacagcuccucgcccuugcucacuggcagagcccuccagcaucgcgagcaggcgcugccuccuccgc

SEQ ID NO: 1278 - RNA, 71 nt, Artificial Sequence
  Description of Artificial Sequence Synthetic oligonucleotide
accccggugaacagcuccucgcccuugcucacuggcagagcccuccagcaucgcgagcaggcgcugccucc

SEQ ID NO: 1279 - RNA, 71 nt, Artificial Sequence
  Description of Artificial Sequence Synthetic oligonucleotide
gcaccaccccggugaacagcuccucgcccuugcucacuggcagagcccuccagcaucgcgagcaggcgcug

SEQ ID NO: 1280 - RNA, 71 nt, Artificial Sequence
  Description of Artificial Sequence Synthetic oligonucleotide
gaugggcaccaccccggugaacagcuccucgcccuugcucacuggcagagcccuccagcaucgcgagcagg

SEQ ID NO: 1281 - RNA, 71 nt, Artificial Sequence
  Description of Artificial Sequence Synthetic oligonucleotide
accaggaugggcaccaccccggugaacagcuccucgcccuugcucacuggcagagcccuccagcaucgcga

SEQ ID NO: 1282 - RNA, 71 nt, Artificial Sequence
  Description of Artificial Sequence Synthetic oligonucleotide
gcucgaccaggaugggcaccaccccggugaacagcuccucgcccuugcucacuggcagagcccuccagcau

SEQ ID NO: 1283 - RNA, 71 nt, Artificial Sequence
  Description of Artificial Sequence Synthetic oligonucleotide
guccagcucgaccaggaugggcaccaccccggugaacagcuccucgcccuugcucacuggcagagcccucc

SEQ ID NO: 1284 - RNA, 71 nt, Artificial Sequence
  Description of Artificial Sequence Synthetic oligonucleotide
cacagauucuuccggcguguauaccuucugcugccuccuccgccgcugccuccuccgccgcugccuccucc

SEQ ID NO: 1285 - RNA, 71 nt, Artificial Sequence
  Description of Artificial Sequence Synthetic oligonucleotide
aucuccacagauucuuccggcguguauaccuucugcugccuccuccgccgcugccuccuccgccgcugccu

SEQ ID NO: 1286 - RNA, 71 nt, Artificial Sequence
  Description of Artificial Sequence Synthetic oligonucleotide
cggggaucuccacagauucuuccggcguguauaccuucugcugccuccuccgccgcugccuccuccgccgc

SEQ ID NO: 1287 - RNA, 71 nt, Artificial Sequence
  Description of Artificial Sequence Synthetic oligonucleotide
gcgaccggggaucuccacagauucuuccggcguguauaccuucugcugccuccuccgccgcugccuccucc

SEQ ID NO: 1288 - RNA, 71 nt, Artificial Sequence
  Description of Artificial Sequence Synthetic oligonucleotide
cgguggcgaccggggaucuccacagauucuuccggcguguauaccuucugcugccuccuccgccgcugccu

SEQ ID NO: 1289 - RNA, 71 nt, Artificial Sequence
  Description of Artificial Sequence Synthetic oligonucleotide
gcucacgguggcgaccggggaucuccacagauucuuccggcguguauaccuucugcugccuccuccgccgc

SEQ ID NO: 1290 - RNA, 71 nt, Artificial Sequence
  Description of Artificial Sequence Synthetic oligonucleotide
cccuugcucacgguggcgaccggggaucuccacagauucuuccggcguguauaccuucugcugccuccucc

SEQ ID NO: 1291 - RNA, 71 nt, Artificial Sequence
  Description of Artificial Sequence Synthetic oligonucleotide
cagcuccucgcccuugcucacgguggcgaccggggaucuccacagauucuuccggcguguauaccuucugc

SEQ ID NO: 1292 - RNA, 71 nt, Artificial Sequence
  Description of Artificial Sequence Synthetic oligonucleotide
gugaacagcuccucgcccuugcucacgguggcgaccggggaucuccacagauucuuccggcguguauaccu

SEQ ID NO: 1293 - RNA, 71 nt, Artificial Sequence
  Description of Artificial Sequence Synthetic oligonucleotide
ccccggugaacagcuccucgcccuugcucacgguggcgaccggggaucuccacagauucuuccggcgugua

SEQ ID NO: 1294 - RNA, 71 nt, Artificial Sequence
  Description of Artificial Sequence Synthetic oligonucleotide
caccaccccggugaacagcuccucgcccuugcucacgguggcgaccggggaucuccacagauucuuccggc

SEQ ID NO: 1295 - RNA, 71 nt, Artificial Sequence
  Description of Artificial Sequence Synthetic oligonucleotide
augggcaccaccccggugaacagcuccucgcccuugcucacgguggcgaccggggaucuccacagauucuu

SEQ ID NO: 1296 - RNA, 71 nt, Artificial Sequence
  Description of Artificial Sequence Synthetic oligonucleotide
ccaggaugggcaccaccccggugaacagcuccucgcccuugcucacgguggcgaccggggaucuccacaga

SEQ ID NO: 1297 - RNA, 71 nt, Artificial Sequence
  Description of Artificial Sequence Synthetic oligonucleotide
cucgaccaggaugggcaccaccccggugaacagcuccucgcccuugcucacgguggcgaccggggaucucc

SEQ ID NO: 1298 - RNA, 111 nt, Artificial Sequence
  Description of Artificial Sequence Synthetic polynucleotide
gaugggcaccaccccggugaacagcuccucgcccuugcucacuggcagagcccuccagcaucgcgagcaggcgcugccuccuccgcccugcagcuuguacagcucguccau

SEQ ID NO: 1299 - RNA, 111 nt, Artificial Sequence
  Description of Artificial Sequence Synthetic polynucleotide
gaugggcaccaccccggugaacagcuccucgcccuugcucacuggcagagcccugcagcaucgcgagcaggcgcugccuccuccgcccugcagcuuguacagcucguccau

SEQ ID NO: 1300 - RNA, 111 nt, Artificial Sequence
  Description of Artificial Sequence Synthetic polynucleotide
gaugggcaccaccccggugaacagcuccucgcccuugcucacuggcagagcccuucagcaucgcgagcaggcgcugccuccuccgcccugcagcuuguacagcucguccau

SEQ ID NO: 1301 - RNA, 111 nt, Artificial Sequence
  Description of Artificial Sequence Synthetic polynucleotide
gaugggcaccaccccggugaacagcuccucgcccuugcucacuggcagagcccuacagcaucgcgagcaggcgcugccuccuccgcccugcagcuuguacagcucguccau

SEQ ID NO: 1302 - RNA, 111 nt, Artificial Sequence
  Description of Artificial Sequence Synthetic polynucleotide
gaugggcaccaccccggugaacagcuccucgcccuugcucacuggcagagcccuccggcaucgcgagcaggcgcugccuccuccgcccugcagcuuguacagcucguccau

SEQ ID NO: 1303 - RNA, 111 nt, Artificial Sequence
  Description of Artificial Sequence Synthetic polynucleotide
gaugggcaccaccccggugaacagcuccucgcccuugcucacuggcagagcccugcggcaucgcgagcaggcgcugccuccuccgcccugcagcuuguacagcucguccau

SEQ ID NO: 1304 - RNA, 111 nt, Artificial Sequence
  Description of Artificial Sequence Synthetic polynucleotide
gaugggcaccaccccggugaacagcuccucgcccuugcucacuggcagagcccuucggcaucgcgagcaggcgcugccuccuccgcccugcagcuuguacagcucguccau

SEQ ID NO: 1305 - RNA, 111 nt, Artificial Sequence
  Description of Artificial Sequence Synthetic polynucleotide
gaugggcaccaccccggugaacagcuccucgcccuugcucacuggcagagcccuacggcaucgcgagcaggcgcugccuccuccgcccugcagcuuguacagcucguccau

SEQ ID NO: 1306 - RNA, 111 nt, Artificial Sequence
  Description of Artificial Sequence Synthetic polynucleotide
gaugggcaccaccccggugaacagcuccucgcccuugcucacuggcagagcccuccugcaucgcgagcaggcgcugccuccuccgcccugcagcuuguacagcucguccau

SEQ ID NO: 1307 - RNA, 111 nt, Artificial Sequence
  Description of Artificial Sequence Synthetic polynucleotide
gaugggcaccaccccggugaacagcuccucgcccuugcucacuggcagagcccugcugcaucgcgagcaggcgcugccuccuccgcccugcagcuuguacagcucguccau

SEQ ID NO: 1308 - RNA, 111 nt, Artificial Sequence
  Description of Artificial Sequence Synthetic polynucleotide
gaugggcaccaccccggugaacagcuccucgcccuugcucacuggcagagcccuacugcaucgcgagcaggcgcugccuccuccgcccugcagcuuguacagcucguccau

SEQ ID NO: 1309 - RNA, 111 nt, Artificial Sequence
  Description of Artificial Sequence Synthetic polynucleotide
gaugggcaccaccccggugaacagcuccucgcccuugcucacuggcagagcccuucugcaucgcgagcaggcgcugccuccuccgcccugcagcuuguacagcucguccau

SEQ ID NO: 1310 - RNA, 111 nt, Artificial Sequence
  Description of Artificial Sequence Synthetic polynucleotide
gaugggcaccaccccggugaacagcuccucgcccuugcucacuggcagagcccucccgcaucgcgagcaggcgcugccuccuccgcccugcagcuuguacagcucguccau

SEQ ID NO: 1311 - RNA, 111 nt, Artificial Sequence
  Description of Artificial Sequence Synthetic polynucleotide
gaugggcaccaccccggugaacagcuccucgcccuugcucacuggcagagcccugccgcaucgcgagcaggcgcugccuccuccgcccugcagcuuguacagcucguccau

SEQ ID NO: 1312 - RNA, 111 nt, Artificial Sequence
  Description of Artificial Sequence Synthetic polynucleotide
gaugggcaccaccccggugaacagcuccucgcccuugcucacuggcagagcccuuccgcaucgcgagcaggcgcugccuccuccgcccugcagcuuguacagcucguccau

SEQ ID NO: 1313 - RNA, 111 nt, Artificial Sequence
  Description of Artificial Sequence Synthetic polynucleotide
gaugggcaccaccccggugaacagcuccucgcccuugcucacuggcagagcccuaccgcaucgcgagcaggcgcugccuccuccgcccugcagcuuguacagcucguccau

SEQ ID NO: 1314 - RNA, 111 nt, Artificial Sequence
  Description of Artificial Sequence Synthetic polynucleotide
uaccgcuacagccacgcugauuucagcuauaccugcccgguauaaagggacguucacaccgcgauguucucugcuggggaauugcgcgauauucaggauuaaaagaagugc

SEQ ID NO: 1315 - RNA, 151 nt, Artificial Sequence
  Description of Artificial Sequence Synthetic polynucleotide
acuacaguugcuccgauauuuaggcuacgucaauaggcacuaacuuauuggcgcuggugaacggacuuccucucgaguaccagaagaugacuacaaaacuccuuuccauugcgaguaucggagucuggcucaguuuggccagggaggcacu

SEQ ID NO: 1316 - RNA, 51 nt, Artificial Sequence
  Description of Artificial Sequence Synthetic oligonucleotide
cggaagaggguggggccgcgguggccagggagccggcgccgccacgcgcgg

SEQ ID NO: 1317 - RNA, 71 nt, Artificial Sequence
  Description of Artificial Sequence Synthetic oligonucleotide
cagcugaggccggaagaggguggggccgcgguggccagggagccggcgccgccacgcgcggguggggggga

SEQ ID NO: 1318 - RNA, 111 nt, Artificial Sequence
  Description of Artificial Sequence Synthetic polynucleotide
ggaggcgaaagcagcccggacagcugaggccggaagaggguggggccgcgguggccagggagccggcgccgccacgcgcgggugggggggacugggguugcucgcgggcuc

SEQ ID NO: 1319 - RNA, 151 nt, Artificial Sequence
  Description of Artificial Sequence Synthetic polynucleotide
gaggcgcagcauccacaggcggaggcgaaagcagcccggacagcugaggccggaagaggguggggccgcgguggccagggagccggcgccgccacgcgcgggugggggggacugggguugcucgcgggcuccgggcgggcggcgggcgccg

SEQ ID NO: 1320 - RNA, 51 nt, Artificial Sequence
  Description of Artificial Sequence Synthetic oligonucleotide
ucuugccuacgccaccagcuccaaccaccacaaguuuauauucagucauuu

SEQ ID NO: 1321 - RNA, 111 nt, Artificial Sequence
  Description of Artificial Sequence Synthetic polynucleotide
gauucugaauuagcuguaucgucaaggcacucuugccuacgccaccagcuccaaccaccacaaguuuauauucagucauuuucagcaggccucucucccgcaccugggagc

SEQ ID NO: 1322 - RNA, 151 nt, Artificial Sequence
  Description of Artificial Sequence Synthetic polynucleotide
aucauauucguccacaaaaugauucugaauuagcuguaucgucaaggcacucuugccuacgccaccagcuccaaccaccacaaguuuauauucagucauuuucagcaggccucucucccgcaccugggagccgcugagccucuggccccgc

SEQ ID NO: 1323 - RNA, 51 nt, Artificial Sequence
  Description of Artificial Sequence Synthetic oligonucleotide
ucggcaugguaugaaguacuucguccaggagcuggagggcccgguguaagu

SEQ ID NO: 1324 - RNA, 71 nt, Artificial Sequence
  Description of Artificial Sequence Synthetic oligonucleotide
gggucugcaaucggcaugguaugaaguacuucguccaggagcuggagggcccgguguaagugaauuucaau

SEQ ID NO: 1325 - RNA, 111 nt, Artificial Sequence
  Description of Artificial Sequence Synthetic polynucleotide
gaccucagucuaaagguugugggucugcaaucggcaugguaugaaguacuucguccaggagcuggagggcccgguguaagugaauuucaauccagcaagguguuucuuuga

SEQ ID NO: 1326 - RNA, 151 nt, Artificial Sequence
  Description of Artificial Sequence Synthetic polynucleotide
uaagggccccaacgguaaaagaccucagucuaaagguugugggucugcaaucggcaugguaugaaguacuucguccaggagcuggagggcccgguguaagugaauuucaauccagcaagguguuucuuugaugcucugucuuggguaaucc

SEQ ID NO: 1327 - RNA, 51 nt, Artificial Sequence
  Description of Artificial Sequence Synthetic oligonucleotide
ugggggguucggcugccgacaucagcaauugcucugccaccaucucagccc

SEQ ID NO: 1328 - RNA, 71 nt, Artificial Sequence
  Description of Artificial Sequence Synthetic oligonucleotide
agcagggccgugggggguucggcugccgacaucagcaauugcucugccaccaucucagcccauccuccgaa

SEQ ID NO: 1329 - RNA, 111 nt, Artificial Sequence
  Description of Artificial Sequence Synthetic polynucleotide
aguagaaggccaagagccacagcagggccgugggggguucggcugccgacaucagcaauugcucugccaccaucucagcccauccuccgaagugaaugaacaggaaccagc

SEQ ID NO: 1330 - RNA, 151 nt, Artificial Sequence
  Description of Artificial Sequence Synthetic polynucleotide
ccucccaucacgggggccguaguagaaggccaagagccacagcagggccgugggggguucggcugccgacaucagcaauugcucugccaccaucucagcccauccuccgaagugaaugaacaggaaccagcucucaaagggaccuccgcag

SEQ ID NO: 1331 - DNA, 151 bp, Artificial Sequence
  Description of Artificial Sequence Synthetic polynucleotide
gccaaacaccacatgcttgccatctagccaggctgtcttgactgtcgtgatgaagaactgggagccgttggtgtccttgcctgcgttggccatgctcacccagccaggcccgtagtgcttcagtttgaagttctcatcggggaagcgctca

SEQ ID NO: 1332 - DNA, 151 bp, Artificial Sequence
  Description of Artificial Sequence Synthetic polynucleotide
gggagtgggtccgctccaccagatgccagcaccggggccagtgcagctcagagccctgtggcggactacagggcccgcacagacggtcactcaaagaaagatgtccctgtgccctactccttggcgatggcaaagggcttctccacctcga

SEQ ID NO: 1333 - DNA, 151 bp, Artificial Sequence
  Description of Artificial Sequence Synthetic polynucleotide
tgcattttgtaaaatagatactagcagattgtcccaagatgtgtacagctcattctcacagcccagcgagggcacctactccacaaatgcgtggccacaggtcatcacctgtcctgtggccctggcgagcctgatccctcacgccgggcac

SEQ ID NO: 1334 - DNA, 151 bp, Artificial Sequence
  Description of Artificial Sequence Synthetic polynucleotide
gctcattctcacagcccagcgagggcacttactccacaaatgcgtggccacaggtcatcacctgtcctgtggccccggcgagcctgatccctcacgccgggcacccacacggcctgcgtgccttctagacttgagttcgcagctctttaag

SEQ ID NO: 1335 - DNA, 151 bp, Artificial Sequence
  Description of Artificial Sequence Synthetic polynucleotide
tcggccgggccctgggggcggtgggcgctggccaggacgcccaccgtgtggttgctgtccaggacggtcccggcccgcgacacttcggcccagagctgctcctcatccagcagcgccagcagccccatggccgtgagcaccggcttgcgca

SEQ ID NO: 1336 - RNA, 111 nt, Artificial Sequence
  Description of Artificial Sequence Synthetic polynucleotide
ugaccagucuuaagaucuuucuugaccugcaccauaagaacuucuccaaagguaccaaaauacucuuucagguccuguucgguuguuuuccaugggagacccaacacuauu

SEQ ID NO: 1337 - RNA, 111 nt, Artificial Sequence
  Description of Artificial Sequence Synthetic polynucleotide
gaugggcaccaccccggugaacagcuccucgcccuugcucacuggcagagcccucgagcaucgcgagcaggcgcugccuccuccgcccugcagcuuguacagcucguccau

SEQ ID NO: 1338 - RNA, 111 nt, Artificial Sequence
  Description of Artificial Sequence Synthetic polynucleotide
gaugggcaccaccccggugaacagcuccucgcccuugcucacuggcagagcccuggagcaucgcgagcaggcgcugccuccuccgcccugcagcuuguacagcucguccau

SEQ ID NO: 1339 - RNA, 111 nt, Artificial Sequence
  Description of Artificial Sequence Synthetic polynucleotide
gaugggcaccaccccggugaacagcuccucgcccuugcucacuggcagagcccuugagcaucgcgagcaggcgcugccuccuccgcccugcagcuuguacagcucguccau

SEQ ID NO: 1340 - RNA, 111 nt, Artificial Sequence
  Description of Artificial Sequence Synthetic polynucleotide
gaugggcaccaccccggugaacagcuccucgcccuugcucacuggcagagcccuagagcaucgcgagcaggcgcugccuccuccgcccugcagcuuguacagcucguccau

SEQ ID NO: 1341 - RNA, 111 nt, Artificial Sequence
  Description of Artificial Sequence Synthetic polynucleotide
gaugggcaccaccccggugaacagcuccucgcccuugcucacuggcagagcccucgggcaucgcgagcaggcgcugccuccuccgcccugcagcuuguacagcucguccau

SEQ ID NO: 1342 - RNA, 111 nt, Artificial Sequence
  Description of Artificial Sequence Synthetic polynucleotide
gaugggcaccaccccggugaacagcuccucgcccuugcucacuggcagagcccuggggcaucgcgagcaggcgcugccuccuccgcccugcagcuuguacagcucguccau

SEQ ID NO: 1343 - RNA, 111 nt, Artificial Sequence
  Description of Artificial Sequence Synthetic polynucleotide
gaugggcaccaccccggugaacagcuccucgcccuugcucacuggcagagcccuugggcaucgcgagcaggcgcugccuccuccgcccugcagcuuguacagcucguccau

SEQ ID NO: 1344 - RNA, 111 nt, Artificial Sequence
  Description of Artificial Sequence Synthetic polynucleotide
gaugggcaccaccccggugaacagcuccucgcccuugcucacuggcagagcccuagggcaucgcgagcaggcgcugccuccuccgcccugcagcuuguacagcucguccau

SEQ ID NO: 1345 - RNA, 111 nt, Artificial Sequence
  Description of Artificial Sequence Synthetic polynucleotide
gaugggcaccaccccggugaacagcuccucgcccuugcucacuggcagagcccucgugcaucgcgagcaggcgcugccuccuccgcccugcagcuuguacagcucguccau

SEQ ID NO: 1346 - RNA, 111 nt, Artificial Sequence
  Description of Artificial Sequence Synthetic polynucleotide
gaugggcaccaccccggugaacagcuccucgcccuugcucacuggcagagcccuggugcaucgcgagcaggcgcugccuccuccgcccugcagcuuguacagcucguccau

SEQ ID NO: 1347 - RNA, 111 nt, Artificial Sequence
  Description of Artificial Sequence Synthetic polynucleotide
gaugggcaccaccccggugaacagcuccucgcccuugcucacuggcagagcccuagugcaucgcgagcaggcgcugccuccuccgcccugcagcuuguacagcucguccau

SEQ ID NO: 1348 - RNA, 111 nt, Artificial Sequence
  Description of Artificial Sequence Synthetic polynucleotide
gaugggcaccaccccggugaacagcuccucgcccuugcucacuggcagagcccuugugcaucgcgagcaggcgcugccuccuccgcccugcagcuuguacagcucguccau

SEQ ID NO: 1349 - RNA, 111 nt, Artificial Sequence
  Description of Artificial Sequence Synthetic polynucleotide
gaugggcaccaccccggugaacagcuccucgcccuugcucacuggcagagcccucgcgcaucgcgagcaggcgcugccuccuccgcccugcagcuuguacagcucguccau

SEQ ID NO: 1350 - RNA, 111 nt, Artificial Sequence
  Description of Artificial Sequence Synthetic polynucleotide
gaugggcaccaccccggugaacagcuccucgcccuugcucacuggcagagcccuggcgcaucgcgagcaggcgcugccuccuccgcccugcagcuuguacagcucguccau

SEQ ID NO: 1351 - RNA, 111 nt, Artificial Sequence
  Description of Artificial Sequence Synthetic polynucleotide
gaugggcaccaccccggugaacagcuccucgcccuugcucacuggcagagcccuugcgcaucgcgagcaggcgcugccuccuccgcccugcagcuuguacagcucguccau

SEQ ID NO: 1352 - RNA, 111 nt, Artificial Sequence
  Description of Artificial Sequence Synthetic polynucleotide
gaugggcaccaccccggugaacagcuccucgcccuugcucacuggcagagcccuagcgcaucgcgagcaggcgcugccuccuccgcccugcagcuuguacagcucguccau

SEQ ID NO: 1353 - RNA, 111 nt, Artificial Sequence; identical to SEQ ID NO: 1337
  Description of Artificial Sequence Synthetic polynucleotide

SEQ ID NO: 1354 - RNA, 111 nt, Artificial Sequence; identical to SEQ ID NO: 1338
  Description of Artificial Sequence Synthetic polynucleotide

SEQ ID NO: 1355 - RNA, 111 nt, Artificial Sequence; identical to SEQ ID NO: 1339
  Description of Artificial Sequence Synthetic polynucleotide

SEQ ID NO: 1356 - RNA, 111 nt, Artificial Sequence; identical to SEQ ID NO: 1340
  Description of Artificial Sequence Synthetic polynucleotide

SEQ ID NO: 1357 - RNA, 111 nt, Artificial Sequence; identical to SEQ ID NO: 1345
  Description of Artificial Sequence Synthetic polynucleotide

SEQ ID NO: 1358 - RNA, 111 nt, Artificial Sequence; identical to SEQ ID NO: 1341
  Description of Artificial Sequence Synthetic polynucleotide

SEQ ID NO: 1359 - RNA, 111 nt, Artificial Sequence; identical to SEQ ID NO: 1349
  Description of Artificial Sequence Synthetic polynucleotide

SEQ ID NO: 1360 - RNA, 111 nt, Artificial Sequence; identical to SEQ ID NO: 1345
  Description of Artificial Sequence Synthetic polynucleotide

SEQ ID NO: 1361 - RNA, 111 nt, Artificial Sequence; identical to SEQ ID NO: 1346
  Description of Artificial Sequence Synthetic polynucleotide

SEQ ID NO: 1362 - RNA, 111 nt, Artificial Sequence; identical to SEQ ID NO: 1348
  Description of Artificial Sequence Synthetic polynucleotide

SEQ ID NO: 1363 - RNA, 111 nt, Artificial Sequence; identical to SEQ ID NO: 1347
  Description of Artificial Sequence Synthetic polynucleotide

SEQ ID NO: 1364 - RNA, 111 nt, Artificial Sequence; identical to SEQ ID NO: 1337
  Description of Artificial Sequence Synthetic polynucleotide

SEQ ID NO: 1365 - RNA, 111 nt, Artificial Sequence; identical to SEQ ID NO: 1349
  Description of Artificial Sequence Synthetic polynucleotide

SEQ ID NO: 1366 - RNA, 111 nt, Artificial Sequence; identical to SEQ ID NO: 1341
  Description of Artificial Sequence Synthetic polynucleotide

SEQ ID NO: 1367 - RNA, 111 nt, Artificial Sequence
  Description of Artificial Sequence Synthetic polynucleotide
gauucugaauuagcuguaucgucaaggcacucgugccgacgccaccagcuccaaccaccacaaguggagagucagucauuuucagcaggccucucucccgcaccugggagc

SEQ ID NO: 1368 - RNA, 111 nt, Artificial Sequence
  Description of Artificial Sequence Synthetic polynucleotide
gauucugaauuagcuggaucgucaaggcacucgggccgacgccaccagcuccaaccaccacaaguggagagucagucauuuucagcaggccucucucccgcaccggggagc

SEQ ID NO: 1369 - RNA, 111 nt, Artificial Sequence
  Description of Artificial Sequence Synthetic polynucleotide
gggagcagccucuggcauucugggagcuucaucuggaccugggucuucagugaaccauuguucaauaucguccggggacagcaucaaaucauccauugcuugggacggcaa

SEQ ID NO: 1370 - RNA, 111 nt, Artificial Sequence
  Description of Artificial Sequence Synthetic polynucleotide
gggagcagccucuggcauucugggagcuucaucuggaccugggucuucagugaaccauuguucaagaucguccggggacagcaucaaaucauccauugcuugggacggcaa

SEQ ID NO: 1371 - RNA, 111 nt, Artificial Sequence
  Description of Artificial Sequence Synthetic polynucleotide
gggagcagccucuggcagucggggagcuucaucuggaccugggucuucagugaaccauuguucaagaucguccggggacagcaucaaaucauccagugcuugggacggcaa

SEQ ID NO: 1372 - RNA, 111 nt, Artificial Sequence
  Description of Artificial Sequence Synthetic polynucleotide
cauauuacagaauaccuugauagcauccaauuugcauccuugguuagggucaacccaguauucuccacucuugaguucaggauggcagaauuucaggucucugcaguuucu

SEQ ID NO: 1373 - RNA, 111 nt, Artificial Sequence
  Description of Artificial Sequence Synthetic polynucleotide
gugaagauaagccaguccucuaguaacagaaugagcaagacggcaagagcuuacccagucacuuguguggagacuuaaauacuugcauaaagauccauugggauaguacuc

SEQ ID NO: 1374 - RNA, 111 nt, Artificial Sequence
  Description of Artificial Sequence Synthetic polynucleotide
gugaacgucaaacugucggaccaauauggcagaaucuucucucaucucaacuuuccauauccguaucauggaaucauagcauccuguaacuacuagcucucuuacagcugg

SEQ ID NO: 1375 - RNA, 111 nt, Artificial Sequence
  Description of Artificial Sequence Synthetic polynucleotide
gccaaugaucucgugaguuaucucagcagugugagccaucagggugaugacaucccaggcgaucguguggccuccaggagcccagagcaggaaguugaggagaaggugccu

SEQ ID NO: 1376 - RNA, 111 nt, Artificial Sequence
  Description of Artificial Sequence Synthetic polynucleotide
caagacggugaaccacuccauggucuucuugucggcuuucugcacuguguacccccagagcuccguguugccgacauccugggguggcuuccacuccagagccacauuaag

SEQ ID NO: 1377 - RNA, 111 nt, Artificial Sequence
  Description of Artificial Sequence Synthetic polynucleotide
aggauucucuuuugaaguauugcucccccaguggauuggguggcuccauucacuccaaugcugagcacuuccacagaguggguuaaagcggcuccgaacacgaaacgugua

SEQ ID NO: 1378 - RNA, 111 nt, Artificial Sequence
  Description of Artificial Sequence Synthetic polynucleotide
gacgcccaccgugugguugcuguccaggacggucccggccugcgacacuucggcccagagcugcuccucauccagcagcgccagcagccccauggccgugagcaccggcuu

SEQ ID NO: 1379 - RNA, 111 nt, Artificial Sequence
  Description of Artificial Sequence Synthetic polynucleotide
gacgcccaccgugugguugcuguccaggacggucccggccugcgacacuucggcccagagcugcuccucaucugcggggcgggggggggccgucgccgcguggggucguug

SEQ ID NO: 1380 - DNA, 151 bp, Artificial Sequence
  Description of Artificial Sequence Synthetic polynucleotide
gggtgatgggtgctggccaggacacccactgtatgattgctgtccaacacagccccagcctttgagacctctgcccagagttgttctccatccaacagggccatgagccccatgactgtgagtactggctttcgcagcaactgcacatggg

SEQ ID NO: 1381 - DNA, 151 bp, Artificial Sequence
  Description of Artificial Sequence Synthetic polynucleotide
actacagttgctccgatatttaggctacgtcaataggcactaacttattggcgctggtgaacggacttcctctcgagtaccagaagatgactacaaaactcctttccattgcgagtatcggagtctggctcagtttggccagggaggcact

SEQ ID NO: 1382 - DNA, 99 bp, Artificial Sequence
  Description of Artificial Sequence Synthetic oligonucleotide
ctgcagggcggaggaggcagcggcggaggaggcagcggcggaggaggcagcagaaggtatacacgccggaagaatctgtagagatccccggtcgccacc

SEQ ID NO: 1383 - DNA, 81 bp, Artificial Sequence
  Description of Artificial Sequence Synthetic oligonucleotide
ctgcagggcggaggaggcagcggcggaggaggcagcggcggaggaggcagcgcctgctcgcgatgctagagggctctgcca

SEQ ID NO: 1384 - DNA, 51 bp, Artificial Sequence
  Description of Artificial Sequence Synthetic oligonucleotide
ctgcagggcggaggaggcagcgcctgctcgcgatgctagagggctctgcca

SEQ ID NO: 1385 - RNA, 70 nt, Artificial Sequence; identical to SEQ ID NO: 1229
  Description of Artificial Sequence Synthetic oligonucleotide

SEQ ID NO: 1386 - DNA, 111 bp, Artificial Sequence
  Description of Artificial Sequence Synthetic polynucleotide
atggacgagctgtacaagctgcagggcggaggaggcagcgcctgctcgcgatgctatagggctctgccagtgagcaagggcgaggagctgttcaccggggtggtgcccatc

SEQ ID NO: 1387 - RNA, 91 nt, Artificial Sequence
  Description of Artificial Sequence Synthetic oligonucleotide
uagcuguaucgucaaggcacucuugccuacgccaccagcuccaaccaccacaaguuuauauucagucauuuucagcaggccucucucccgc

SEQ ID NO: 1388 - RNA, 111 nt, Artificial Sequence
  Description of Artificial Sequence Synthetic polynucleotide
gauucugaauuagcuguaucgucaaggcacucuugccuacgccaccagcuccaacuaccacaaguuuauauucagucauuuucagcaggccucucucccgcaccugggagc

SEQ ID NO: 1389 - RNA, 131 nt, Artificial Sequence
  Description of Artificial Sequence Synthetic polynucleotide
uccacaaaaugauucugaauuagcuguaucgucaaggcacucuugccuacgccaccagcuccaacuaccacaaguuuauauucagucauuuucagcaggccucucucccgcaccugggagccgcugagccu

SEQ ID NO: 1390 - RNA, 151 nt, Artificial Sequence; identical to SEQ ID NO: 1322
  Description of Artificial Sequence Synthetic polynucleotide

SEQ ID NO: 1391 - RNA, 171 nt, Artificial Sequence
  Description of Artificial Sequence Synthetic polynucleotide
cuauuguuggaucauauucguccacaaaaugauucugaauuagcuguaucgucaaggcacucuugccuacgccaccagcuccaaccaccacaaguuuauauucagucauuuucagcaggccucucucccgcaccugggagccgcugagccucuggccccgccgccgccuuc

SEQ ID NO: 1392 - RNA, 191 nt, Artificial Sequence
  Description of Artificial Sequence Synthetic polynucleotide
uaggaauccucuauuguuggaucauauucguccacaaaaugauucugaauuagcuguaucgucaaggcacucuugccuacgccaccagcuccaaccaccacaaguuuauauucagucauuuucagcaggccucucucccgcaccugggagccgcugagccucuggccccgccgccgccuucagugccugcg

SEQ ID NO: 1393 - RNA, 151 nt, Artificial Sequence; identical to SEQ ID NO: 1319
  Description of Artificial Sequence Synthetic polynucleotide

SEQ ID NO: 1394 - RNA, 151 nt, Artificial Sequence
  Description of Artificial Sequence Synthetic polynucleotide
uccuguagcuaaggccacaaaauuauccacuguuuuuggaacagucuuuccgaagagaccaaagaucacccggcccacaucuucaucuccaauucguaggucaaaauacaccuugacggugacuuugggccccuucuucuucucaucggcc

SEQ ID NO: 1395 - RNA, 151 nt, Artificial Sequence
  Description of Artificial Sequence Synthetic polynucleotide
gcccuggaucaugaaguccuugauuacacgauggaauuugcuguuuuuguagccaaauccuuucucuccuguagccaaggccacaaaauuauccacuguuuuuggaacagucuuuccgaagagaccaaagaucacccggccuacaucuuca

SEQ ID NO: 1396 - RNA, 72 nt, Artificial Sequence
  Description of Artificial Sequence Synthetic oligonucleotide
gcgcaaguuagguuuugucaagaaaggguguaacgcaaccaagucauaguccgccuagaagcauuugcggug

SEQ ID NO: 1397 - RNA, 131 nt, Artificial Sequence
  Description of Artificial Sequence Synthetic polynucleotide
gccaugccaaucucaucuuguuuucugcgcaaguuagguuuugucaagaaaggguguaacgcaaccaagucauaguccgccuagaagcauuugcgguggacgauggaggggccggacucgucauacuccug

SEQ ID NO: 1398 - RNA, 70 nt, Artificial Sequence
  Description of Artificial Sequence Synthetic oligonucleotide
ggacuuccuguaacaacgcaucucauauuuggaaugaccauuaaaaaaacaacaaugugcaaucaaaguc

SEQ ID NO: 1399 - RNA, 71 nt, Artificial Sequence
  Description of Artificial Sequence Synthetic oligonucleotide
caaggugcggcuccggccccuccccucuucaagggguccacauggcaacugugaggaggggagauucagug

SEQ ID NO: 1400 - RNA, 91 nt, Artificial Sequence
  Description of Artificial Sequence Synthetic oligonucleotide
uagcuguaucgucaaggcacucgugccgacgccaccagcuccaaccaccacaaggggagagucagucagggucagcaggccucucucccgc

SEQ ID NO: 1401 - RNA, 91 nt, Artificial Sequence
  Description of Artificial Sequence Synthetic oligonucleotide
uagcuguaucgucaaggcacucuugccgacgccaccagcuccaaccaccacaaguguauagucagucauuuucagcaggccucucucccgc

SEQ ID NO: 1402 - RNA, 91 nt, Artificial Sequence
  Description of Artificial Sequence Synthetic oligonucleotide
uagcuggaucgucaaggcacucgugccgacgccaccagcuccaaccaccacaaggggagaggcagucagggucagcaggccucucucccgc

SEQ ID NO: 1403 - RNA, 111 nt, Artificial Sequence
  Description of Artificial Sequence Synthetic polynucleotide
gauucugaauuagcuguaucgucaaggcacucuugccgacgccaccagcuccaaccaccacaaguguauagucagucauuuucagcaggccucucucccgcaccugggagc

SEQ ID NO: 1404 - RNA, 111 nt, Artificial Sequence; identical to SEQ ID NO: 1367
  Description of Artificial Sequence Synthetic polynucleotide

SEQ ID NO: 1405 - RNA, 111 nt, Artificial Sequence
  Description of Artificial Sequence Synthetic polynucleotide
gauucugaauuagcuguaucgucaaggcacucgugccgacgccaccagcuccaaccaccacaaggggagagucagucagggucagcaggccucucucccgcaccugggagc

SEQ ID NO: 1406 - RNA, 111 nt, Artificial Sequence
  Description of Artificial Sequence Synthetic polynucleotide
gcuccccggugcgggagagaggccugcugacccugacugccucuccccuuguggugguuggagcugguggcgucggcacgagugccuugacgauccagcuaauucagaauc

SEQ ID NO: 1407 - RNA, 14 nt, Artificial Sequence
  Description of Artificial Sequence Synthetic oligonucleotide
gcagagccuccagc

SEQ ID NO: 1408 - RNA, 21 nt, Artificial Sequence
  Description of Artificial Sequence Synthetic oligonucleotide
cucacuggcagagccuccagc

SEQ ID NO: 1409 - RNA, 27 nt, Artificial Sequence
  Description of Artificial Sequence Synthetic oligonucleotide
cccuugcucacuggcagagccuccagc

SEQ ID NO: 1410 - RNA, 33 nt, Artificial Sequence
  Description of Artificial Sequence Synthetic oligonucleotide
cucucgcccuugcucacuggcagagccuccagc

SEQ ID NO: 1411 - RNA, 38 nt, Artificial Sequence
  Description of Artificial Sequence Synthetic oligonucleotide
cucucgcccuugcucacuggcagagccuccagcaucgc

SEQ ID NO: 1412 - RNA, 45 nt, Artificial Sequence
  Description of Artificial Sequence Synthetic oligonucleotide
ugaacagcucucgcccuugcucacuggcagagccuccagcaucgc

SEQ ID NO: 1413 - RNA, 70 nt, Artificial Sequence; identical to SEQ ID NO: 1230
  Description of Artificial Sequence Synthetic oligonucleotide

SEQ ID NO: 1414 - RNA, 30 nt, Artificial Sequence
  Description of Artificial Sequence Synthetic oligonucleotide
acaaauggggacgaggggggcggggcggcc

SEQ ID NO: 1415 - RNA, 20 nt, Artificial Sequence
  Description of Artificial Sequence Synthetic oligonucleotide
cggagagcagagagggagcg

SEQ ID NO: 1416 - DNA, 49 bp, Artificial Sequence
  Description of Artificial Sequence Synthetic oligonucleotide
aaaaaaaaaagatcttgaaactgttttaaggttggccgatcttaaaaaa

SEQ ID NO: 1417 - RNA, 51 nt, Artificial Sequence
  Description of Artificial Sequence Synthetic oligonucleotide
ucauaaucaauuuauuauuuucuuuuauuuuauucacauaauuuuguuuuu

SEQ ID NO: 1418 - DNA, 522 bp, Artificial Sequence
  Description of Artificial Sequence Synthetic polynucleotide
cttgtggaaaggacgaaacaccgccatcagtcgccggtcccaagcccggataaaatgggagggggcgggaaaccgcctaaccatgccgactgatggcagaaaaaaaaaaggccgggcgcggtggctcacgcctgtaatcccagcactttgggaggccgaggcggggagattgcttgagcccaggagttcgagaccagcctgggcaacatagcgagaccccgtctctcttgtgtctactgtacagaatactgccgccagctggatttcccaattctgagtaacacagccgggcgtggtggcgcgcgcctgtagtcccagctactcgggaggctgaggcaggaggatcgcttgagcccaggagttcgaggctgcagtgagctatgatcgcgccactgcactccagcctgggcgacagagcgagaccctgtctcaaaaaaaaaactgccatcagtcggcgtggactgtagaacactgccaatgccggtcccaagcccggataaaagtggagggtacagtccacgc

SEQ ID NO: 1419 - DNA, 563 bp, Artificial Sequence
  Description of Artificial Sequence Synthetic polynucleotide
cttgtggaaaggacgaaacaccgccatcagtcgccggtcccaagcccggataaaatgggagggggcgggaaaccgcctaaccatgccgactgatggcagaaaaaaaaaaggccgggcgcggtggctcacgcctgtaatcccagcactttgggaggccgaggcggggagattgcttgagcccaggagttcgagaccagcctgggcaacatagcgagaccccgtctctgataaaaggcgtacataattcttgtgtctactgtacagaatactgccgccagctggatttcccaattctgagtaacactctgcaatccaaacagggttcagccgggcgtggtggcgcgcgcctgtagtcccagctactcgggaggctgaggcaggaggatcgcttgagcccaggagttcgaggctgcagtgagctatgatcgcgccactgcactccagcctgggcgacagagcgagaccctgtctcaaaaaaaaaactgccatcagtcggcgtggactgtagaacactgccaatgccggtcccaagcccggataaaagtggagggtacagtccacgc